(19) United States
(12) Patent Application Publication
(10) Pub. No.: US 2013/0189679 A1
(43) Pub. Date: Jul. 25, 2013

(54) PSEUDOGENES AND USES THEREOF

(71) Applicants: Arul M. Chinnaiyan, Plymouth, MI (US); Chandan Kumar-Sinha, Ann Arbor, MI (US); Shanker Kalyana-Sundaram, Ann Arbor, MI (US)

(72) Inventors: Arul M. Chinnaiyan, Plymouth, MI (US); Chandan Kumar-Sinha, Ann Arbor, MI (US); Shanker Kalyana-Sundaram, Ann Arbor, MI (US)

(73) Assignee: THE REGENTS OF THE UNIVERSITY OF MICHIGAN, Ann Arbor, MI (US)

(21) Appl. No.: 13/720,411

(22) Filed: Dec. 19, 2012

Related U.S. Application Data
(60) Provisional application No. 61/577,767, filed on Dec. 20, 2011.

(51) Int. Cl. C12Q 1/68

(52) U.S. Cl. CPC C12Q 1/6886
USPC 435/6.11; 435/6.12; 436/501; 435/7.1

(57) ABSTRACT

The present invention relates to compositions and methods for cancer diagnosis, research and therapy, including but not limited to, cancer markers. In particular, the present invention relates to pseudogenes as diagnostic markers and clinical targets for prostate cancer.

This application claims priority U.S. Provisional Application No. 61/577,767, filed on Dec. 20, 2011, which is herein incorporated by reference in its entirety.

### STATEMENT REGARDING FEDERALLY SPONSORED RESEARCH OR DEVELOPMENT

This invention was made with government support under CA132874 and CA111275 awarded by the National Institutes of Health and W81XWH-08-1-0110 awarded by the Army Medical Research and Materiel Command. The government has certain rights in the invention.

### FIELD OF THE INVENTION

The present invention relates to compositions and methods for cancer diagnosis, research and therapy, including but not limited to, cancer markers. In particular, the present invention relates to pseudogenes as diagnostic markers and clinical targets for cancer.

### BACKGROUND OF THE INVENTION

Afflicting one out of nine men over age 65, prostate cancer (PCA) is a leading cause of male cancer-related death, second only to lung cancer (Abate-Shen and Shen, Genes Dev 14:2410 [2000]; Ruijter et al., Endocr Rev, 20:22 [1999]). The American Cancer Society estimates that about 184,500 American men will be diagnosed with prostate cancer and 39,200 will die in 2001.

Prostate cancer is typically diagnosed with a digital rectal exam and/or prostate specific antigen (PSA) screening. An elevated serum PSA level can indicate the presence of PCA. PSA is used as a marker for prostate cancer because it is secreted only by prostate cells. A healthy prostate will produce a stable amount—typically below 4 nanograms per milliliter, or a PSA reading of “4” or less—whereas cancer cells produce escalating amounts that correspond with the severity of the cancer. A level between 4 and 10 may raise a doctor's suspicion that a patient has prostate cancer, while amounts above 50 may show that the tumor has spread elsewhere in the body.

When PSA or digital tests indicate a strong likelihood that cancer is present, a transrectal ultrasound (TRUS) is used to map the prostate and show any suspicious areas. Biopsies of various sectors of the prostate are used to determine if prostate cancer is present. Treatment options depend on the stage of the cancer. Men with a 10-year life expectancy or less who have a low Gleason number and whose tumor has not spread beyond the prostate are often treated with watchful waiting (no treatment). Treatment options for more aggressive cancers include surgical treatments such as radical prostatectomy (RP), in which the prostate is completely removed (with or without nerve sparing techniques) and radiation, applied through an external beam that directs the dose to the prostate from outside the body or via low-dose radioactive seeds that are implanted within the prostate to kill cancer cells locally. Anti-androgen hormone therapy is also used, alone or in conjunction with surgery or radiation. Hormone therapy uses luteinizing hormone-releasing hormones (LH-RH) analogs, which block the pituitary from producing hormones that stimulate testosterone production. Patients must have injections of LH-RH analogs for the rest of their lives.

While surgical and hormonal treatments are often effective for localized PCA, advanced disease remains essentially incurable. Androgen ablation is the most common therapy for advanced PCA, leading to massive apoptosis of androgen-dependent malignant cells and temporary tumor regression. In most cases, however, the tumor reemerges with a vengeance and can proliferate independent of androgen signals.

The advent of prostate specific antigen (PSA) screening has led to earlier detection of PCA and significantly reduced PCA-associated fatalities. However, the impact of PSA screening on cancer-specific mortality is still unknown pending the results of prospective randomized screening studies (Etzioni et al., J. Natl. Cancer Inst., 91:1033 [1999]; Maattanen et al., Br. J. Cancer 79:1210 [1999]; Schroder et al., J. Natl. Cancer Inst., 90:1817 [1998]). A major limitation of the serum PSA test is a lack of prostate cancer sensitivity and specificity especially in the intermediate range of PSA detection (4-10 ng/ml). Elevated serum PSA levels are often detected in patients with non-malignant conditions such as benign prostatic hyperplasia (BPH) and prostatitis, and provide little information about the aggressiveness of the cancer detected. Coincident with increased serum PSA testing, there has been a dramatic increase in the number of prostate needle biopsies performed (Jacobsen et al., JAMA 274:1445 [1995]). This has resulted in a surge of equivocal prostate needle biopsies (Epstein and Potter J. Urol., 166:402 [2001]). Thus, development of additional serum and tissue biomarkers to supplement PSA screening is needed.

### SUMMARY OF THE INVENTION

The present invention relates to compositions and methods for cancer diagnosis, research and therapy, including but not limited to, cancer markers. In particular, the present invention relates to pseudogenes as diagnostic markers and clinical targets for cancer.

Embodiments of the present invention provide compositions, kits, and methods useful in the detection and screening of prostate and breast cancer.

For example, embodiments of the present invention provide a method of screening for the presence of breast cancer in a subject, comprising contacting a biological sample from a subject with a reagent for detecting the level of expression of a pseudogene (e.g., ATPase, aminophospholipid transporter, class I, type 8A, member 2 pseudogene (ATP8A2-Ψ) or dipeptidyl-peptidase 3 (DPP3)); and detecting the level of expression of the pseudogene in the sample using an in vitro assay, wherein an increased level of expression of the pseudogene in the sample relative to the level in normal breast cells is indicative of breast cancer in the subject. In some embodiments, the sample is, for example, blood, plasma, serum or breast cells. In some embodiments, detection is carried out utilizing a method selected from, for example, a sequencing technique, a nucleic acid hybridization technique, a nucleic acid amplification technique (e.g., polymerase chain reaction, reverse transcription polymerase chain reaction, transcription-mediated amplification, ligase chain reaction, strand displacement amplification or nucleic acid sequence based amplification) or an immunoassay. In some embodiments, the reagent is, for example, a pair of amplification oligonucleotides or an oligonucleotide probe. In some embodiments, the breast cancer is luminal breast cancer.

In some embodiments, the present invention provides a method of screening for the presence of prostate cancer in a subject, comprising contacting a biological sample from a subject with a reagent for detecting the level of expression of a pseudogene (e.g., coxsackie virus and adenovirus receptor pseudogene (CXADR-Ψ), NADH dehydrogenase (ubiquinone) 1 alpha subcomplex, 9 (NDUFA9), epithelial cell adhesion molecule (EPCAM), PDGFA associated protein 1 (PDAP1), RNA binding motif protein 17 (RBM17), carboxylesterase 5A (CES7) or kallikrein-related peptidase 4-kallikrein pseudogene 1 (KLK4-KLKP1)); and detecting the level or presence of expression of the pseudogene in the sample using an in vitro assay, wherein the presence or an increased level of expression of the pseudogene in the sample relative to the level in normal prostate cells is indicative of prostate cancer in the subject. In some embodiments, the sample is, for example, tissue, blood, plasma, serum, urine, urine supernatant, urine cell pellet, semen, prostatic secretions or prostate cells. In some embodiments, detection is carried out utilizing a method selected from, for example, a sequencing technique, a nucleic acid hybridization technique, a nucleic acid amplification technique (e.g., polymerase chain reaction, reverse transcription polymerase chain reaction, transcription-mediated amplification, ligase chain reaction, strand displacement amplification or nucleic acid sequence based amplification) or an immunoassay. In some embodiments, the reagent is, for example, a pair of amplification oligonucleotides or an oligonucleotide probe. In some embodiments, the cancer is localized prostate cancer or metastatic prostate cancer.

Additional embodiments are described herein.

### DESCRIPTION OF THE FIGURES

FIG. 1 shows the pseudogene expression analysis pipeline.

FIG. 2 shows a schematic representation of cluster alignments with pseudogene transcripts shown for ATP8A2-Ψ and CXADR-Ψ.

FIG. 3 shows tissue-specific pseudogene expression profiles. A heatmap of pseudogene expression sorted on the basis of tissue specific expression displays tissue specific (top), tissue-enriched/non-specific (middle) and ubiquitously expressed pseudogenes (bottom).

FIG. 4 shows cancer-specific pseudogene expression profiles. A heatmap of pseudogene expression sorted according to cancer-specific expression patterns displays pseudogene transcripts specific to individual cancers (top), common across multiple cancers (tissueenriched; middle) and non-specific (bottom).

FIG. 5 shows expression of CXADR-Ψ in prostate cancer. (A) A histogram of expression values (y-axis) of CXADR-Ψ (top) and CXADR-parental (bottom) across a panel of prostate and other tissue samples (x-axis). (B) A summary histogram of the expression values of CXADR-Ψ and CXADR-parental in prostate cancers either harboring or lacking an ETS transcription factor gene fusion, or in nonprostate samples (left). On the right, expression of CXADR-Ψ and CXADR-parental in matched pairs of tumor and benign samples from prostate cancer patients (right).

FIG. 6 shows expression of ATP8A2-Ψ in breast cancer. Histograms of Taqman assay based qRT-PCR expression profiles of (A) ATP8A2-Ψ and (B) ATP8A2-wild type, across a panel of samples from breast and other tissue types as indicated. (Inset) A summary histogram of the expression values of ATP8A2-Ψ and ATP8A2-parental in breast cancer samples relative to benign breast and other tissues (left) and luminal vs. basal breast cancer subtypes (right). (C) Cell proliferation assays following siRNA based knockdowns of ATP8A2-wild type and pseudogene as indicated. (D) Histogram of Boyden chamber assay showing cell migration (left) and invasion through matrigel (right). (E) The effect of ATP8A2 pseudogene overexpression in TERT-HMEC cells on cell proliferation (left) and cell migration based on Incucyte wound confluency assay (right) and (F) Chicken chorioallantoic membrane assay of HCC-1806 cells treated with either non-targeting control siRNA, ATP8A2-WT siRNA or ATP8A2-Ψ siRNA showing relative number of cells intravaseted in the lower CAM (left) and metastatic cells in chicken lung (right).

FIG. 7 shows samples comprising the transcriptome sequencing data compendium. (A) Pie chart showing relative distribution of samples from each tissue type (Right); Bar chart representing the numbers of samples in the indicated categories, in each tissue type. (B) Table indicating exact number of samples in each sub-category with a summary of passed purity filter (PF) read count. MPN: Multiple primary neoplasia.

FIG. 8 shows a comparision of pseudogene annotations with reference databases (Yale and ENCODE). A. Venn diagrams showing overlaps between clusters/probes (left), pseudogenes (middle) and genes (right) based on Yale, ENCODE, and BLAT based custom analysis. B. Clusters not overlapped with reference databases were assessed for proximity to existing pseudogenes. (Left) Graphical representation of number of pseudogenes (y-axis) corresponding to unmapped clusters that are in defined proximity (x-axis) to existing pseudogenes. (Right) UCSC tracks display CENTG2 and HNRNPA1 pseudogenes with chromosomal coordinates based on ENCODE Gencode Manual Gene Annotations, immediately flanked by pseudogene clusters identified by Custom BLAT analysis.

FIG. 9 shows a scatter plot reprsentation of the correlation between the total number of pseudogenes observed in a sequencing run (x-axis) with the sequence yield (Passed Filter reads) (y-axis).

FIG. 10 shows an intersection plot displaying the fraction of pseudogene transcripts with corresponding chromosomal loci enriched for H3K4me3 in breast cancer cell line MCF7.

FIG. 11 shows a correlation between gene and pseudogene pairs.

FIG. 12 shows a scatter plot representation of correlation between expression levels of (A) ATP8A2 and (B) CXADR pseudogenes as assessed by RNA-seq (y-axis) and qPCR (x-axis).

FIG. 13 shows a sequence alignment of ATP8A2-WT and -Ψ (top) and CXADR-WT and -Ψ (bottom) using Mulialign interface.

FIG. 14 shows cloning and sequencing of CXADR-Ψ from prostate cancer samples. A. Two prostate cancer tissues used to amplify ˜300 bp bands corresponding to CXADR-Ψ cDNA. B. The sequence of prostate cancer tissue derived CXADR-Ψ cDNA aligned to reference CXADR-Ψ sequence. C. The sequence of prostate cancer tissue derived CXADR-Ψ cDNA aligned to wild type CXADR-Ψ sequence.

FIG. 15 shows (A) Estimation of ATP8A2-WT and -pseudogene transcripts by Taqman qPCR assay. (B) Western blot analysis of ATP8A2 in the three breast cell lines. (C) qPCR analysis of levels of ATP8A2-wt and pseudogene transcripts after siRNA knockdown in Cama-1 and HCC1806 cells. (D) Cell Proliferation Assays following siRNA based knockdowns of ATP8A2-wild type and pseudogene in the pancreatic cancer cell line BXPC3. (E) Western blot analysis of breast cancer cell line HCC1806 knocked down for ATP8A2-wild type or pseudogene siRNA probed with ATP8A2 antibody.

FIG. 16 shows a prostate cancer specific chimeric transcript involving the pseudogene KLKP1. (a) Histogram of expression values of KLK4-KLKP1 chimera across a panel of prostate and other tissue samples as indicated. The inset shows KLK4-KLKP1 chimeric transcript architecture based on transcriptome sequencing. (b) Predicted amino acid sequence of the KLK4-KLKP1 open reading frame indicating the stretch of novel peptide encoded by KLKP1_.

FIG. 17 shows the nucleic acid sequences of CXADR_Ψ (SEQ ID NO:12), ATP8A2_Ψ_ENST00000420453 (SEQ ID NO:13), NDUFA9_Ψ_ENST00000436210 (SEQ ID NO:14), DPP3_Ψ_ENST00000416030 (SEQ ID NO:15), CES7_Ψ (SEQ ID NO:16), PDAP1_Ψ_ENST00000392530 (SEQ ID NO:17), CENTG2_Ψ_ENST00000427215 (SEQ ID NO:18), NF1_Ψ (SEQ ID NO:19), PGK1_Ψ_ENST00000431702 (SEQ ID NO:20), CRIP1_Ψ_ENST00000442565 (SEQ ID NO:21), MTCH1_Ψ (SEQ ID NO:22), PPP4R2_Ψ_ENST00000429866 (SEQ ID NO:23), TMEM161B_Ψ_ENST00000457388 (SEQ ID NO:24), PTPN2_Ψ_ENST00000433704 (SEQ ID NO:25), SCYL2_Ψ_ENST00000392253 (SEQ ID NO:26), KLK4-KLKP1 (Pseudogene Readthrough) (SEQ ID NO:27).

### DEFINITIONS

To facilitate an understanding of the present invention, a number of terms and phrases are defined below:

As used herein, the terms “detect”, “detecting” or “detection” may describe either the general act of discovering or discerning or the specific observation of a detectably labeled composition.

As used herein, the term “subject” refers to any organisms that are screened using the diagnostic methods described herein. Such organisms preferably include, but are not limited to, mammals (e.g., murines, simians, equines, bovines, porcines, canines, felines, and the like), and most preferably includes humans.

The term “diagnosed,” as used herein, refers to the recognition of a disease by its signs and symptoms, or genetic analysis, pathological analysis, histological analysis, and the like.

A “subject suspected of having cancer” encompasses an individual who has received an initial diagnosis (e.g., a CT scan showing a mass or increased PSA level) but for whom the stage of cancer or presence or absence of pseudogenes indicative of cancer is not known. The term further includes people who once had cancer (e.g., an individual in remission). In some embodiments, “subjects” are control subjects that are suspected of having cancer or diagnosed with cancer.

As used herein, the term “characterizing cancer in a subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the pseudogenes disclosed herein.

As used herein, the term “characterizing prostate tissue in a subject” refers to the identification of one or more properties of a prostate tissue sample (e.g., including but not limited to, the presence of cancerous tissue, the presence or absence of pseudogenes, the presence of pre-cancerous tissue that is likely to become cancerous, and the presence of cancerous tissue that is likely to metastasize). In some embodiments, tissues are characterized by the identification of the expression of one or more cancer marker genes, including but not limited to, the cancer markers disclosed herein.

As used herein, the term “stage of cancer” refers to a qualitative or quantitative assessment of the level of advancement of a cancer. Criteria used to determine the stage of a cancer include, but are not limited to, the size of the tumor and the extent of metastases (e.g., localized or distant).

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “oligonucleotide,” refers to a short length of single-stranded polynucleotide chain. Oligonucleotides are typically less than 200 residues long (e.g., between 15 and 100), however, as used herein, the term is also intended to encompass longer polynucleotide chains. Oligonucleotides are often referred to by their length. For example a 24 residue oligonucleotide is referred to as a “24-mer”. Oligonucleotides can form secondary and tertiary structures by self-hybridizing or by hybridizing to other polynucleotides. Such structures can include, but are not limited to, duplexes, hairpins, cruciforms, bends, and triplexes.

As used herein, the terms “complementary” or “complementarity” are used in reference to polynucleotides (i.e., a sequence of nucleotides) related by the base-pairing rules. For example, the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

The term “homology” refers to a degree of complementarity. There may be partial homology or complete homology (i.e., identity). A partially complementary sequence is a nucleic acid molecule that at least partially inhibits a completely complementary nucleic acid molecule from hybridizing to a target nucleic acid is “substantially homologous.” The inhibition of hybridization of the completely complementary sequence to the target sequence may be examined using a hybridization assay (Southern or Northern blot, solution hybridization and the like) under conditions of low stringency. A substantially homologous sequence or probe will compete for and inhibit the binding (i.e., the hybridization) of a completely homologous nucleic acid molecule to a target under conditions of low stringency. This is not to say that conditions of low stringency are such that non-specific binding is permitted; low stringency conditions require that the binding of two sequences to one another be a specific (i.e., selective) interaction. The absence of non-specific binding may be tested by the use of a second target that is substantially non-complementary (e.g., less than about 30% identity); in the absence of non-specific binding the probe will not hybridize to the second non-complementary target.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely relation sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

The term “isolated” when used in relation to a nucleic acid, as in “an isolated oligonucleotide” or “isolated polynucleotide” refers to a nucleic acid sequence that is identified and separated from at least one component or contaminant with which it is ordinarily associated in its natural source. Isolated nucleic acid is such present in a form or setting that is different from that in which it is found in nature. In contrast, non-isolated nucleic acids as nucleic acids such as DNA and RNA found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs that encode a multitude of proteins. However, isolated nucleic acid encoding a given protein includes, by way of example, such nucleic acid in cells ordinarily expressing the given protein where the nucleic acid is in a chromosomal location different from that of natural cells, or is otherwise flanked by a different nucleic acid sequence than that found in nature. The isolated nucleic acid, oligonucleotide, or polynucleotide may be present in single-stranded or double-stranded form. When an isolated nucleic acid, oligonucleotide or polynucleotide is to be utilized to express a protein, the oligonucleotide or polynucleotide will contain at a minimum the sense or coding strand (i.e., the oligonucleotide or polynucleotide may be single-stranded), but may contain both the sense and anti-sense strands (i.e., the oligonucleotide or polynucleotide may be double-stranded).

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Such examples are not however to be construed as limiting the sample types applicable to the present invention.

### DETAILED DESCRIPTION OF THE INVENTION

The present invention relates to compositions and methods for cancer diagnosis, research and therapy, including but not limited to, cancer markers. In particular, the present invention relates to pseudogenes as diagnostic markers and clinical targets for prostate cancer.

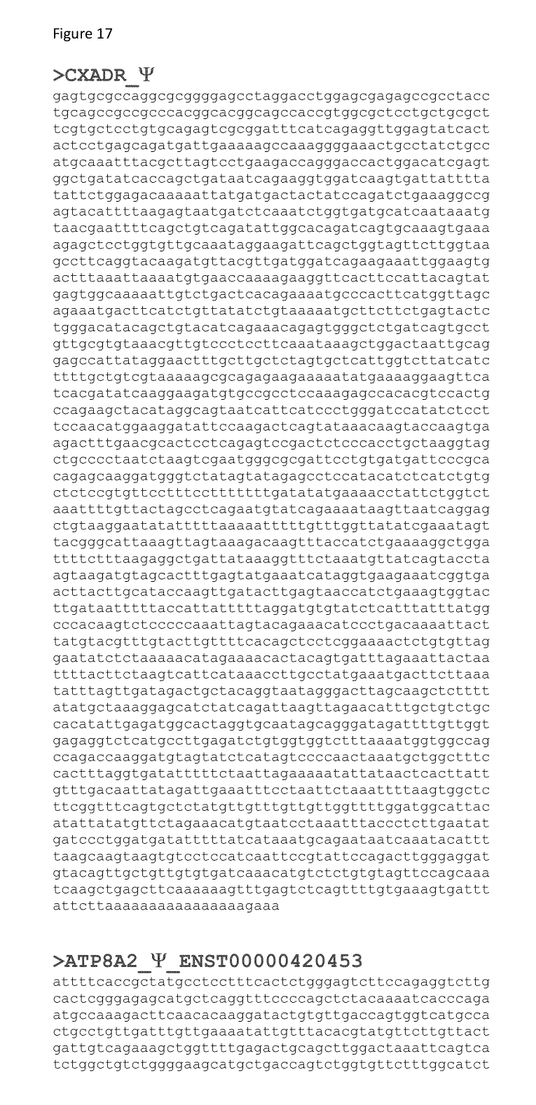

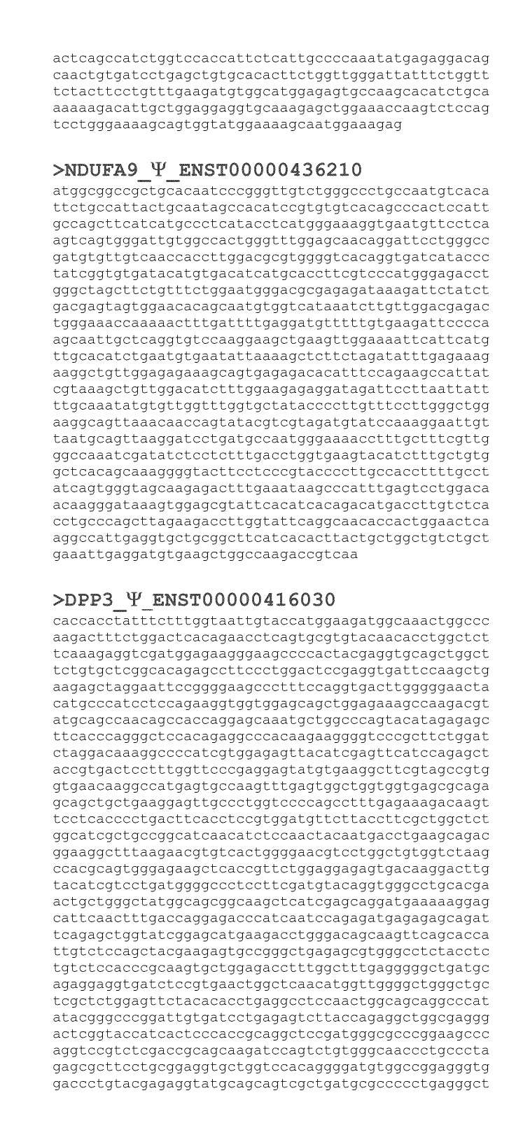

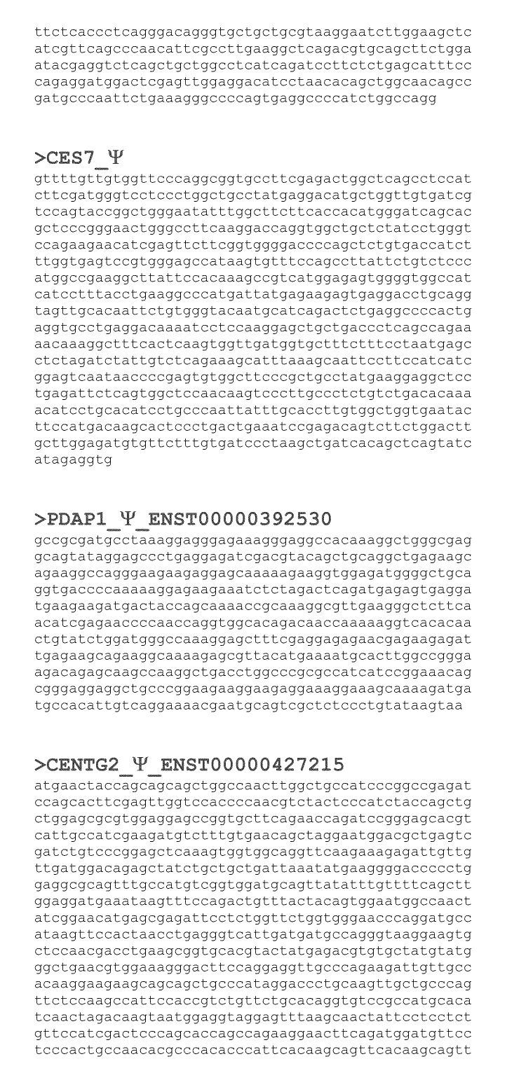

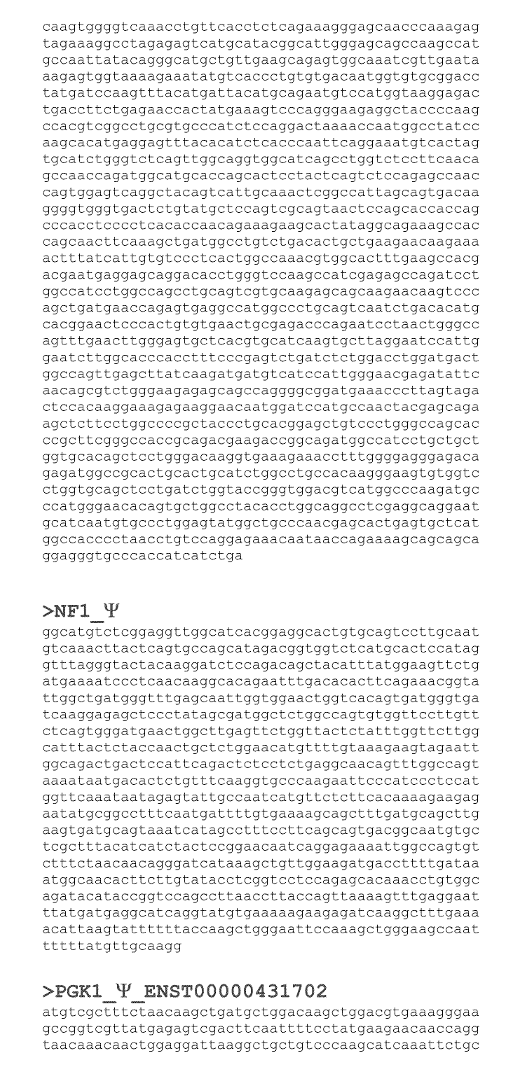

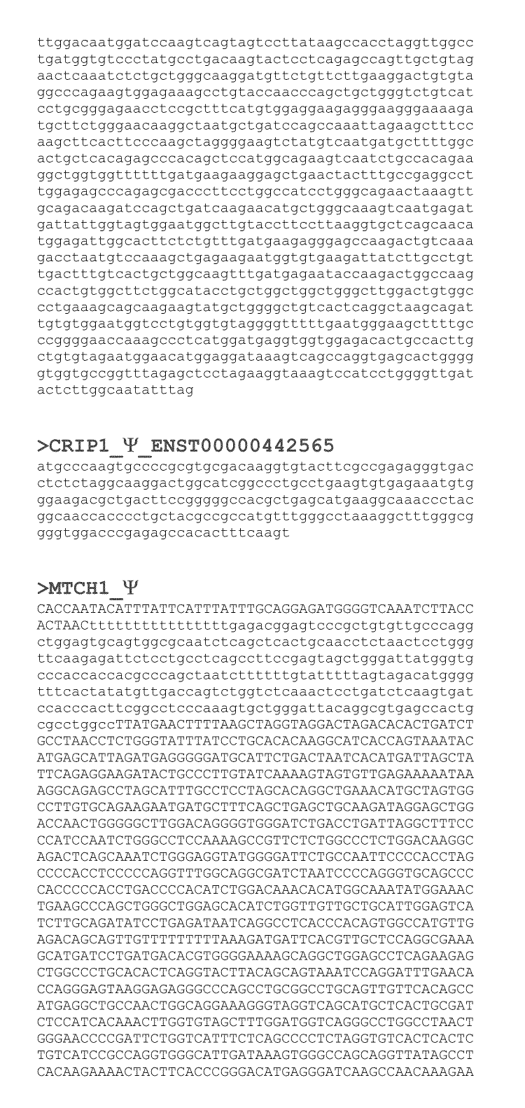

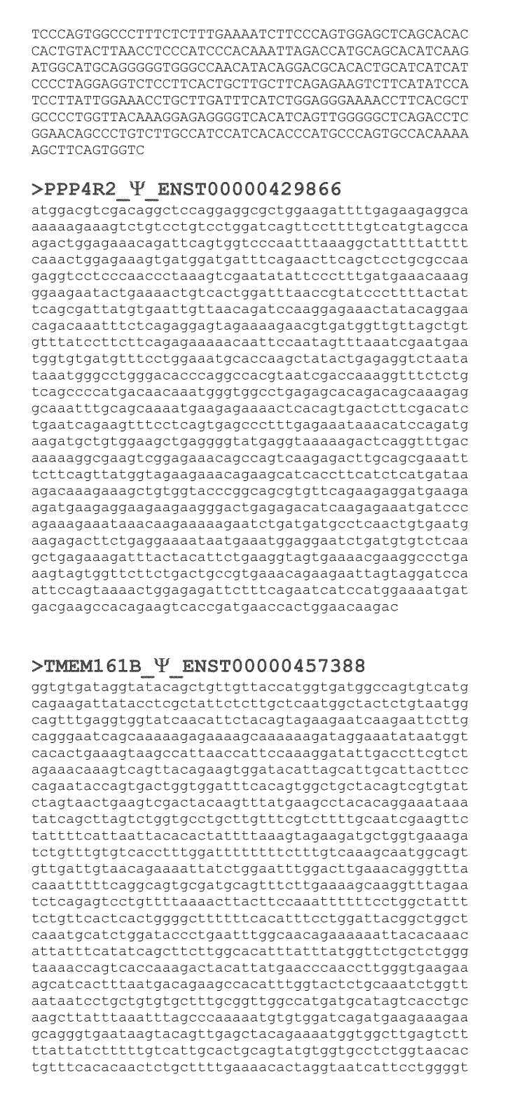

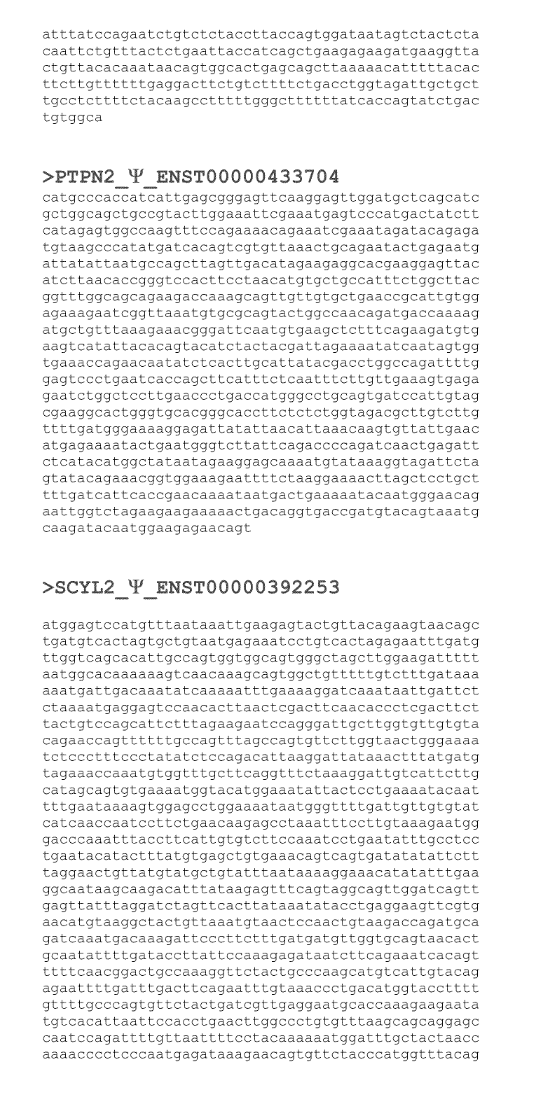

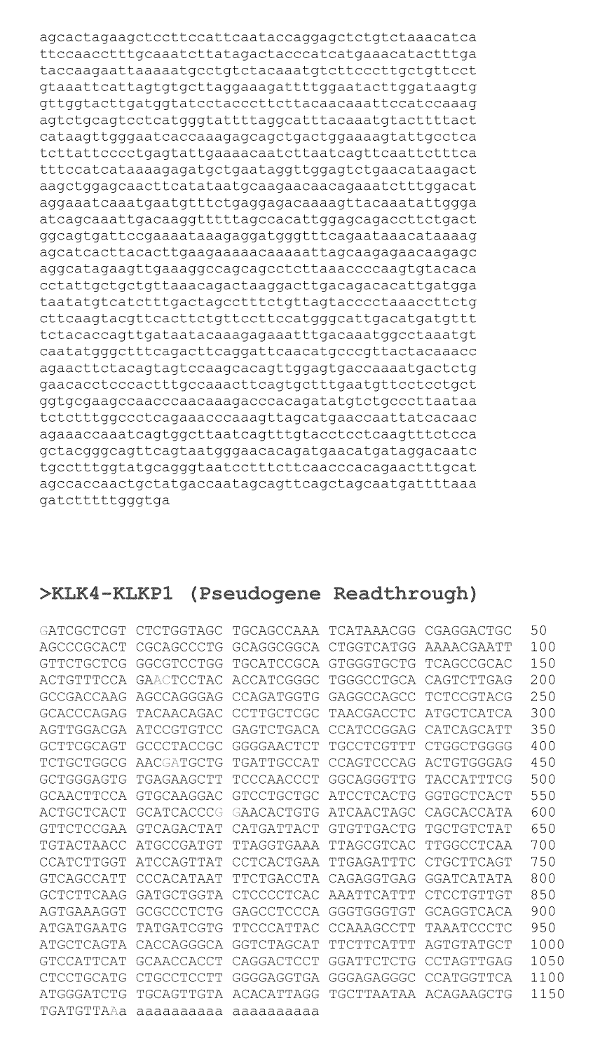

For example, in some embodiments, ATP8A2-Ψ and DPP3 pseudogenes were identified as being specific to breast cancer and CXADR-Ψ, NDUFA9, EPCAM, PDAP1, RBM17 and CES7 pseudogenes and the KLK4-KLKP1 fusion were identified as being specific to prostate cancer. Sequences of exemplary pseudogenes are shown in FIG. 17.

### I. Diagnostic and Screening Methods

As described above, embodiments of the present invention provide diagnostic and screening methods that utilize the detection of pseudogenes (e.g., ATP8A2-Ψ, DPP3, CXADR-Ψ, NDUFA9, EPCAM, PDAP1, RBM17, CES7 and KLK4-KLKP1). Exemplary, non-limiting methods are described below.

Any patient sample suspected of containing the pseudogenes may be tested according to methods of embodiments of the present invention. By way of non-limiting examples, the sample may be tissue (e.g., a prostate biopsy sample or a tissue sample obtained by prostatectomy), blood, urine, semen, prostatic secretions or a fraction thereof (e.g., plasma, serum, urine supernatant, urine cell pellet or prostate cells). A urine sample is preferably collected immediately following an attentive digital rectal examination (DRE), which causes prostate cells from the prostate gland to shed into the urinary tract.

In some embodiments, the patient sample is subjected to preliminary processing designed to isolate or enrich the sample for the pseudogenes or cells that contain the pseudogenes. A variety of techniques known to those of ordinary skill in the art may be used for this purpose, including but not limited to: centrifugation; immunocapture; cell lysis; and, nucleic acid target capture (See, e.g., EP Pat. No. 1 409 727, herein incorporated by reference in its entirety).

The pseudogenes may be detected along with other markers in a multiplex or panel format. Markers are selected for their predictive value alone or in combination with the pseudogenes. Exemplary prostate cancer markers include, but are not limited to: AMACR/P504S (U.S. Pat. No. 6,262,245); PCA3 (U.S. Pat. No. 7,008,765); PCGEM1 (U.S. Pat. No. 6,828,429); prostein/P501S, P503S, P504S, P509S, P510S, prostase/P703P, P710P (U.S. Publication No. 20030185830); RAS/KRAS (Bos, Cancer Res. 49:4682-89 (1989); Kranenburg, Biochimica et Biophysica Acta 1756:81-82 (2005)); and, those disclosed in U.S. Pat. Nos. 5,854,206 and 6,034,218, 7,229,774, each of which is herein incorporated by reference in its entirety. Markers for other cancers, diseases, infections, and metabolic conditions are also contemplated for inclusion in a multiplex or panel format.

i. DNA and RNA Detection

The pseudogenes of the present invention are detected using a variety of nucleic acid techniques known to those of ordinary skill in the art, including but not limited to: nucleic acid sequencing; nucleic acid hybridization; and, nucleic acid amplification.

1. Sequencing

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

A number of DNA sequencing techniques are known in the art, including fluorescence-based sequencing methodologies (See, e.g., Birren et al., Genome Analysis: Analyzing DNA, 1, Cold Spring Harbor, N.Y.; herein incorporated by reference in its entirety). In some embodiments, automated sequencing techniques understood in that art are utilized. In some embodiments, parallel sequencing of partitioned amplicons (PCT Publication No: WO2006084132 to Kevin McKernan et al., herein incorporated by reference in its entirety) is utilized. In some embodiments, bridge amplification (see, e.g., WO 2000/018957, U.S. Pat. Nos. 7,972,820; 7,790,418 and Adessi et al., Nucleic Acids Research (2000): 28(20): E87; each of which are herein incorporated by reference) is utilized. In some embodiments, DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties) is utilized. Additional examples of sequencing techniques include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

2. Hybridization

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts (e.g., pseudogenes) within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

In some embodiments, pseudogenes are detected using fluorescence in situ hybridization (FISH). In some embodiments, FISH assays utilize bacterial artificial chromosomes (BACs). These have been used extensively in the human genome sequencing project (see Nature 409: 953-958 (2001)) and clones containing specific BACs are available through distributors that can be located through many sources, e.g., NCBI. Each BAC clone from the human genome has been given a reference name that unambiguously identifies it. These names can be used to find a corresponding GenBank sequence and to order copies of the clone from a distributor.

The present invention further provides a method of performing a FISH assay on human prostate cells, human prostate tissue or on the fluid surrounding said human prostate cells or human prostate tissue. Specific protocols are well known in the art and can be readily adapted for the present invention. Guidance regarding methodology may be obtained from many references including: In situ Hybridization: Medical Applications (eds. G. R. Coulton and J. de Belleroche), Kluwer Academic Publishers, Boston (1992); In situ Hybridization: In Neurobiology; Advances in Methodology (eds. J. H. Eberwine, K. L. Valentino, and J. D. Barchas), Oxford University Press Inc., England (1994); In situ Hybridization: A Practical Approach (ed. D. G. Wilkinson), Oxford University Press Inc., England (1992)); Kuo, et al., Am. J. Hum. Genet. 49:112-119 (1991); Klinger, et al., Am. J. Hum. Genet. 51:55-65 (1992); and Ward, et al., Am. J. Hum. Genet. 52:854-865 (1993)). There are also kits that are commercially available and that provide protocols for performing FISH assays (available from e.g., Oncor, Inc., Gaithersburg, Md.). Patents providing guidance on methodology include U.S. Pat. Nos. 5,225,326; 5,545,524; 6,121,489 and 6,573,043. All of these references are hereby incorporated by reference in their entirety and may be used along with similar references in the art and with the information provided in the Examples section herein to establish procedural steps convenient for a particular laboratory.

3. Microarrays

Different kinds of biological assays are called microarrays including, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes or transcripts (e.g., pseudogenes) by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink jet printing; or, electrochemistry on microelectrode arrays.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

3. Amplification

Nucleic acids (e.g., pseudogenes) may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product. Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

4. Detection Methods

Non-amplified or amplified nucleic acids can be detected by any conventional means. For example, the pseudogenes can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

ii. Data Analysis

In some embodiments, a computer-based analysis program is used to translate the raw data generated by the detection assay (e.g., the presence, absence, or amount of a given marker or markers) into data of predictive value for a clinician. The clinician can access the predictive data using any suitable means. Thus, in some preferred embodiments, the present invention provides the further benefit that the clinician, who is not likely to be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information provides, medical personal, and subjects. For example, in some embodiments of the present invention, a sample (e.g., a biopsy or a serum or urine sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., expression data), specific for the diagnostic or prognostic information desired for the subject.

The profile data is then prepared in a format suitable for interpretation by a treating clinician. For example, rather than providing raw expression data, the prepared format may represent a diagnosis or risk assessment (e.g., presence or absence of a pseudogene) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease or as a companion diagnostic to determine a treatment course of action.

iii. In Vivo Imaging

Pseudogenes (e.g., ATP8A2-Ψ, DPP3, CXADR-Ψ, NDUFA9, EPCAM, PDAP1, RBM17, CES7 and KLK4-KLKP1) may also be detected using in vivo imaging techniques, including but not limited to: radionuclide imaging; positron emission tomography (PET); computerized axial tomography, X-ray or magnetic resonance imaging method, fluorescence detection, and chemiluminescent detection. In some embodiments, in vivo imaging techniques are used to visualize the presence of or expression of cancer markers in an animal (e.g., a human or non-human mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of embodiments of the present invention are useful in the identification of cancers that express pseudogenes (e.g., prostate cancer). In vivo imaging is used to visualize the presence or level of expression of a pseudogene. Such techniques allow for diagnosis without the use of an unpleasant biopsy. The in vivo imaging methods of embodiments of the present invention can further be used to detect metastatic cancers in other parts of the body.

In some embodiments, reagents (e.g., antibodies) specific for the cancer markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin Onc 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl) EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m which does not use chelation with DPTA is the pretinning method of Crockford et al., (U.S. Pat. No. 4,323,546, herein incorporated by reference).

A method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the pseudogene, to insure that the antigen binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

iv. Compositions & Kits

Compositions for use in the diagnostic methods described herein include, but are not limited to, probes, amplification oligonucleotides, and the like. In some embodiments, kits include all components necessary, sufficient or useful for detecting the markers described herein (e.g., reagents, controls, instructions, etc.). The kits described herein find use in research, therapeutic, screening, and clinical applications.

The probe and antibody compositions of the present invention may also be provided in the form of an array.

### II. Drug Screening Applications

In some embodiments, the present invention provides drug screening assays (e.g., to screen for anticancer drugs). The screening methods of the present invention utilize pseudogenes. For example, in some embodiments, the present invention provides methods of screening for compounds that alter (e.g., decrease) the expression or activity of pseudogenes. The compounds or agents may interfere with transcription, by interacting, for example, with the promoter region. The compounds or agents may interfere with mRNA (e.g., by RNA interference, antisense technologies, etc.). The compounds or agents may interfere with pathways that are upstream or downstream of the biological activity of pseudogenes. In some embodiments, candidate compounds are antisense or interfering RNA agents (e.g., oligonucleotides) directed against pseudogenes. In other embodiments, candidate compounds are antibodies or small molecules that specifically bind to a pseudogenes regulator or expression products inhibit its biological function.

In one screening method, candidate compounds are evaluated for their ability to alter pseudogenes expression by contacting a compound with a cell expressing a pseudogene and then assaying for the effect of the candidate compounds on expression. In some embodiments, the effect of candidate compounds on expression of pseudogenes is assayed for by detecting the level of pseudogene expressed by the cell. mRNA expression can be detected by any suitable method.

### EXPERIMENTAL

The following examples are provided in order to demonstrate and further illustrate certain preferred embodiments and aspects of the present invention and are not to be construed as limiting the scope thereof.

### Example 1

### A. Methods

### Dataset

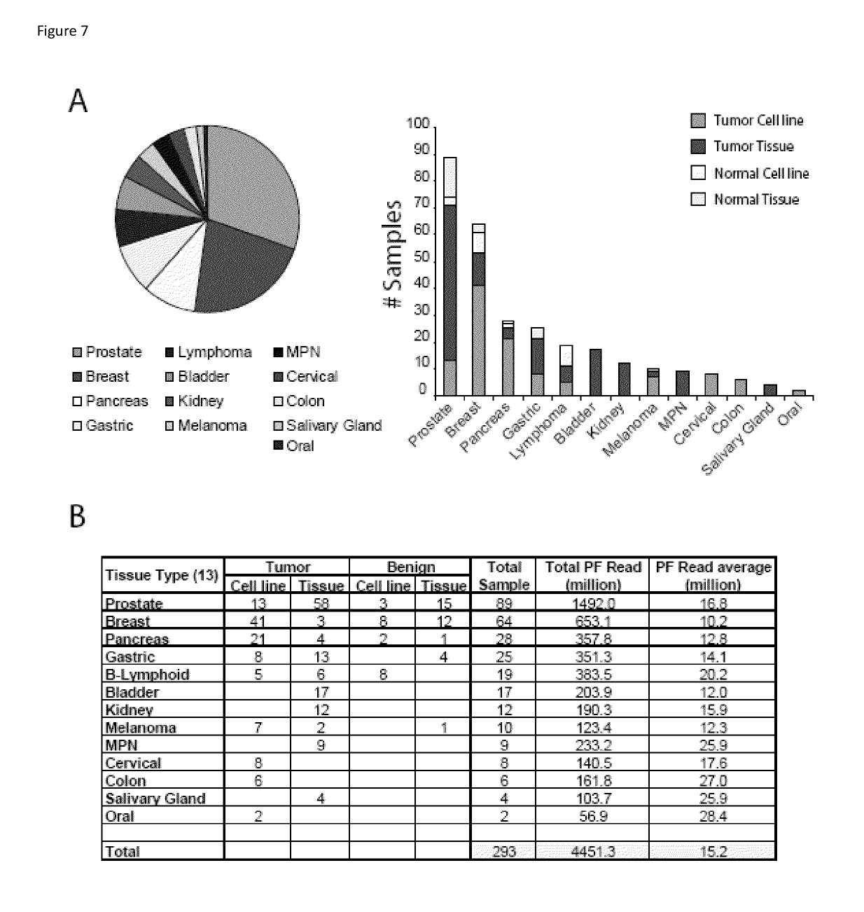

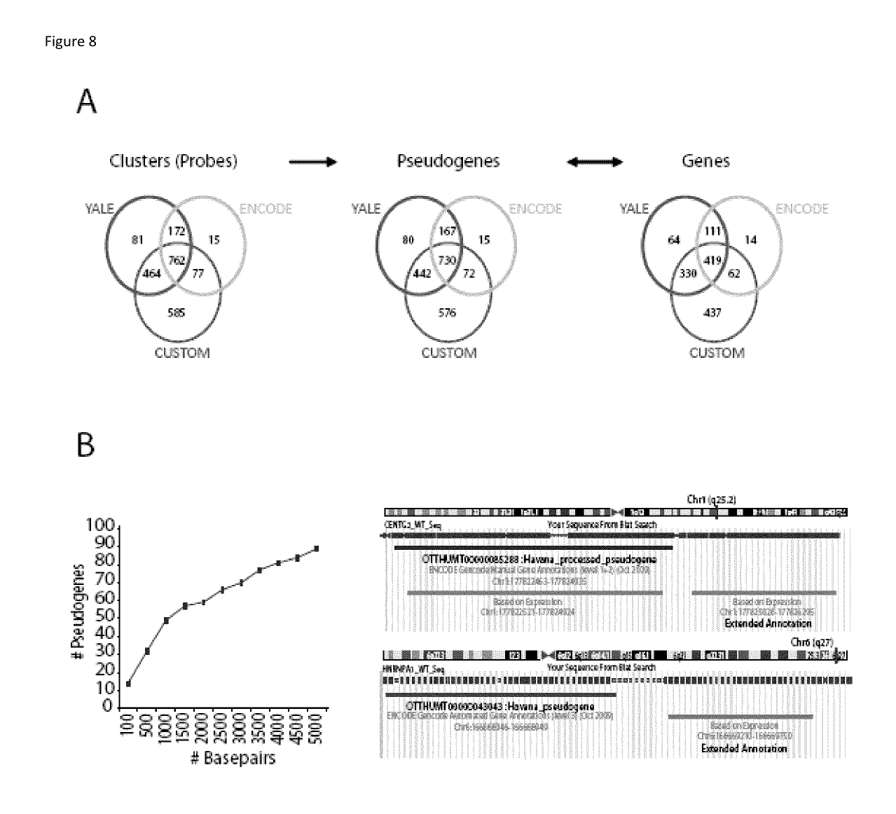

Paired end transcriptome sequence reads (2×40 and 2×80 base pairs) were obtained from 13 tissue types including breast, prostate, pancreas, gastric, melanoma, and other tissues comprising a total of over 293 individual samples (FIG. 7, Table 1). Each sample was sequenced on an Illumina Genome Analyzer I or II according to protocols provided by Illumina, described earlier (Maher et al., 2009b Proceedings of the National Academy of Sciences of the United States of America 106, 12353-12358).

### Pseudogene Analysis Pipeline

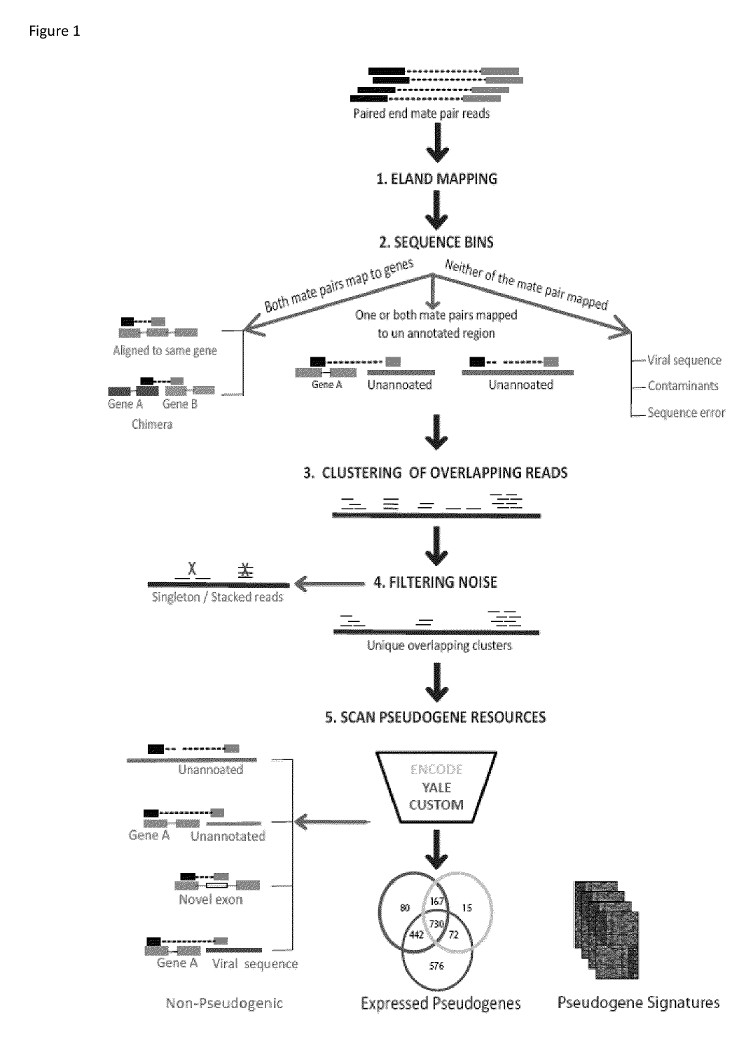

Paired end transcriptome reads were mapped to the human genome (NCBI36/hg18) and University of California Santa Cruz (UCSC) Genes using Efficient Alignment of Nucleotide Databases (ELAND) software of the Illumina Genome Analyzer Pipeline, using 32 bp seed length, allowing up to 2 mismatches; detailed mapping status are represented in Table 2. Table 2 shows primary mapping status of individual sequencing lanes. Flowcell and lane ID (Column A), total number of reads (Column B), purity filter reads (Column C), followed by mapping count for each chromosome (hs_ref_chr1-22, X, and Y) including mitochondrial (chrM) and ribosomal sequences (humRibosomal) (Column D-AC). Passed purity filter reads obtained from Illumina export and extended output files (as described before) were parsed and binned into three major categories: 1. Both of the paired reads map to annotated genes 2. One or both of the paired reads map to un-annotated regions in the genome, and 3. Neither of the reads map (these include viral, bacterial, and other contaminant reads, as well as sequencing errors). The paired reads with one or both partners mapping to an un-annotated region were clustered based on overlaps of aligned sequences using the chromosomal coordinates of the clusters. Singleton reads that did not cluster or stacked\duplicated reads with same start and stop genomic-coordinates (potential PCR artifacts) were filtered out. Passed filter ‘clusters’ were defined as units of transcript expression (analogous to a ‘probe’ on microarray platforms). These ‘clusters’ were screened against publicly available human pseudogene resources, Yale human pseudogene Build 53-processed, duplicate and fragment entries (Karro et al., 2007 Nucleic acids research 35, D55-60) and Gencode Manual Gene Annotations (level 1+2), Automated Gene Annotations (level 3) (October 2009) (Zheng et al., 2007 Genome research 17, 839-851) to identify and annotate pseudogene ‘clusters’. The clusters were also subjected to homology search using the alignment tool BLAT (Kent, 2002 Genome research 12, 656-664) for an independent annotation. Sequence reads from individual samples were queried against the resultant clusters defined by the union of Yale, ENCODE and BLAT output to assess the expression of pseudogenes (FIG. 1). A stringent cutoff value for pseudogene expression in a sample was set at five or more reads mapping to at least one cluster in a putative pseudogene transcript.

Data from individual samples was used to construct a matrix to carry out pseudogene expression profiling. Pseudogene transcripts (one or more probe(s) overlapping with either Yale or ENCODE) in 2 or more samples in a tissue type and its absence in all other tissue types was used to define it as tissue specific. If a pseudogene probe was detected in 10 out of 13 samples, it was designated as ubiquitous. All other cases were described as intermediate category. Pseudogene transcripts detected in 3 or more cancer samples and absent in all benign samples were assigned as cancer specific.

RNA Isolation and cDNA Synthesis

Total RNA was isolated using Trizol and an RNeasy Kit (Invitrogen) with DNase I digestion according to the manufacturer's instructions. RNA integrity was verified on an Agilent Bioanalyzer 2100 (Agilent Technologies, Palo Alto, Calif.). cDNA was synthesized from total RNA using Superscript III (Invitrogen) and random primers (Invitrogen).

### Quantitative Real-Time PCR

Quantitative Real-time PCR (qPCR) was performed using Taqman or SYBR green based assays (Applied Biosystems, Foster City, Calif.) on an Applied Biosystems 7900HT Real-Time PCR System, according to standard protocols. The Taqman assays for CXADR and ATP8A2 assays were custom designed based on regions of differences between the wild type and pseudogene sequences. Oligonucleotide primers for SYBR green assays were obtained from Integrated DNA Technologies (Coralville, Iowa). The housekeeping gene, GAPDH, was used as a loading control. Fold changes were calculated relative to GAPDH and normalized to the median value of the benign samples.

(SEQ ID NO: 1)
CXADR-Ψ_F CGGTTTCAGTGCTCTATGTTGTTTG
(SEQ ID NO: 2)
CXADR-Ψ_R TAAATTTAGGATTACATGTTTCTAGAACA
(SEQ ID NO: 3)
CXADR-Ψ_M 6FAM ATGCCATCCAAAACCA
(SEQ ID NO: 4)
ATP8A2-Ψ_F CTGGTGTTCTTTGGCATCTACTCA
(SEQ ID NO: 5)
ATP8A2-Ψ_R CAGCTCAGGATCACAGTTGCT
(SEQ ID NO: 6)
ATP8A2-Ψ_M 6FAM CTGGTCCACCATTCTC
(SEQ ID NO: 7)
ATP8A2-WT_F ATCCTATTGAAGGAGGACTCTTTGGA
(SEQ ID NO: 8)
ATP8A2-WT_R CCAGCAAATTCCCAAGGTCAGT
(SEQ ID NO: 9)
ATP8A2-WT_M 6FAM AAGGGCAGCCATTACT
(SEQ ID NO: 10)
KLK4-KLKP1_F ATGGAAAACGAATTGTTCTG
(SEQ ID NO: 11)
KLK4-KLKP1_R CAGTGTTCCGGGTGATGCAG

Additionally, inventoried Taqman assay for CXADR-WT (Hs00154661_m1) and ATP8A2-WT (assay ID hs00185259_m1) were used.

### B. Results

Development of a Bioinformatics Platform for the Analysis of Pseudogene Transcription

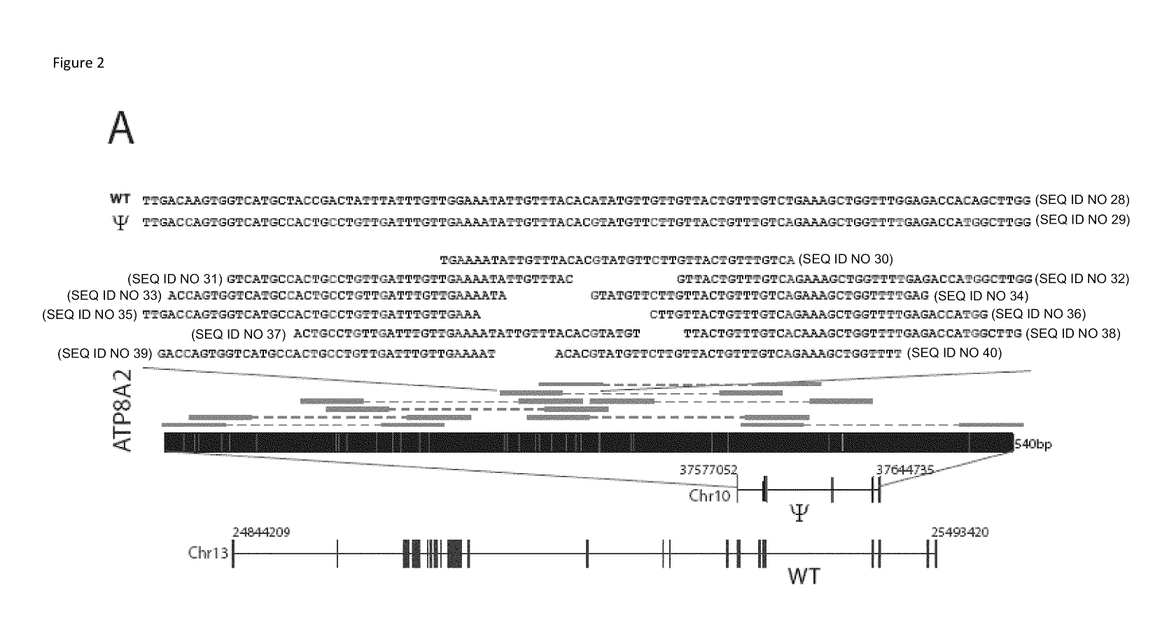

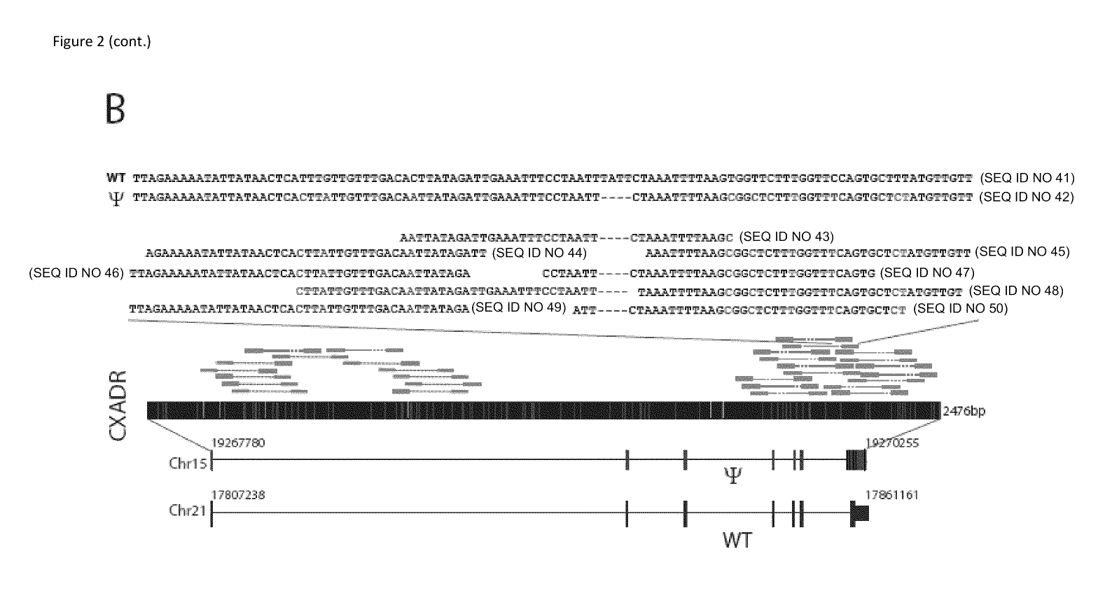

High throughput transcriptome sequencing always yields a fraction of reads that do not map perfectly to the reference genome/transcriptome. This sequence fraction has been actively mined for aberrant transcripts including mutations (Shah et al., 2009a N Engl J Med 360, 2719-2729; Shah et al., 2009b Nature 461, 809-813; Tuch et al. PloS one 5, e9317), chimeric RNAs (Maher et al., 2009a Nature 458, 97-101; Maher et al., 2009b Proceedings of the National Academy of Sciences of the United States of America 106, 12353-12358), xenobiotic sequences, and non-coding RNAs (Gupta et al. Nature 464, 1071-1076; Loewer et al. Nature genetics 42, 1113-1117; Wilhelm et al., 2008 Nature 453, 1239-1243). In this study the sequencing reads that showed a highdegree of homology but imperfect match to the reference genes, but mapped perfectly elsewhere in the genome, were used to serve as the primary data for pseudogene expression analysis (FIG. 1). Thus, paired-end RNA-Seq data from a compendium of 293 samples, representing both cancer and benign samples from 13 different tissue types was utilized to build the pseudogene analysis pipeline (FIG. 7, Table 1). Sequencing reads were mapped to the human genome (hg18) and University of California Santa Cruz (UCSC) Genes using Efficient Alignment of Nucleotide Databases (ELAND) software of the Illumina Genome Analyzer Pipeline. Reads that contained three or more mismatches to the reference genes but mapped perfectly to un-annotated regions, elsewhere in the genome, were used to generate de novo ‘clusters’ (that ranged from 40 to 5000 bps). All the clusters that contained two or more unique, high quality overlapping reads—nucleating at the loci of differences between wild type genes and pseudogenes were retained for further analysis of pseudogene transcripts (FIG. 1). The clusters were employed for gene expression analyses in a way analogous to the ‘probes’ used in microarray gene expression studies; albeit, unlike predesigned and fixed probes used in microarrays, the sequence clusters used here were formed de novo, solely based on the presence (and levels) of transcripts. Thus one or more clusters (like one or more probes in microarrays) represented a transcript, while the number of reads mapping to a cluster (analogous to fluorescence intensity due to probe hybridization on microarrays) provided a measure of expression of the corresponding genes. For example, FIG. 2 shows a schematic representation of the cluster alignments for two representative pseudogenes, ATP8A2-Ψ (top) and CXADR-Ψ (bottom), and as seen in the figure, mutation-dense regions in the transcripts provide loci of cluster formation.

Overall, 2156 unique clusters were defined in terms of their genomic coordinates (start and end points) that were compared against annotated pseudogenes in the ENCODE and the Yale Gerstein Group (referred to as Yale) (Kano et al., 2007 Nucleic acids research 35, D55-60) databases, the two most comprehensive pseudogene annotation resources. Of the 2156 initial clusters, 934 overlapped with both the Yale and ENCODE databases, whereas 81 were found only in the Yale database and 15 only in the ENCODE database, overall accounting for 1506 distinct pseudogene transcripts corresponding to 1000 unique genes (FIG. 8). Further, because the RNASeq compendium is comprised of 35-45 mer short sequence reads that largely generated short sequence clusters not optimal for pseudogene analysis tools such as Pseudopipe (Zhang et al., 2006 Bioinformatics (Oxford, England) 22, 1437-1439) and Pseudofam (Lam et al., 2009 Nucleic acids research 37, D738-743) used in generating ENCODE and Yale databases, we also carried out a direct query of individual clusters against the human genome (hg18) using the BLAT tool from UCSC, that is ideally suited for short sequence alignment searches (Kent, 2002). Using this BLAT analysis, also referred as the “custom” analysis (FIG. 8A), it was possible to independently assign 1888 clusters representing 1820 unique pseudogenes to a unique genomic location. Comparing the genomic locations of the pseudogene clusters identified by BLAT analysis to those identified by Yale and ENCODE databases (FIG. 8A), 762 clusters were found to be common to all the three resources, but a large set of 585 clusters were uniquely identified by BLAT analysis.

Some clusters were seen to be localized in the vicinity of known pseudogenes. 92 clusters that resided adjacent (within 5 kb) to previously annotated pseudogenes (FIG. 8B, left) were identified. It was hypothesized that these represent pseudogenes with inaccurate annotations in the current databases. For example, the chromosomal coordinates of CENTG2-Ψ (OTTHUMT00000085288, Havana processed pseudogene) are defined in ENCODE as Chr1:177822463-177824935. A cluster mapping to this locus was observed; a distinct cluster (Chr1:177825028-177826295) less than 100 base pairs away was also observed. Although un-annotated in the current databases, the sequence of this adjacent locus shows a high degree of homology to the CENTG2 parental gene (FIG. 8B, right), indicating that the novel cluster represents an extension of the existing genomic coordinates of CENTG2-Ψ annotation. Similar observations were made with HNRNPA1 and the HNRNPA1-Ψ on Chr6q27 (FIG. 8B, right). The majority of clusters in close proximity to annotated pseudogenes however, did not follow this pattern, and these likely represent putative novel pseudogenes currently missing in the database annotations. Overall, the pseudogene clusters common to the Yale, ENCODE and ‘custom’ datasets, as well as those common to any two of these subsets, were identified as representing ‘expressed pseudogenes’, that were taken up for further analyses.

Further, taking into account the instances of multiple clusters representing the same pseudogene transcript, the 2156 transcript clusters overall amounted to transcriptional evidence of 2082 distinct pseudogenes, of which 1506 transcripts correspond to specific genomic coordinates in Yale and/or ENCODE pseudogenes, and as many as 576 potentially novel transcripts (described below) (FIG. 8A). The 2082 pseudogene transcripts in turn correspond to 1437 wild type genes, clearly indicating that transcription of multiple pseudogenes arisen from the same wild type genes are also detected in our compendium. Taken together, the study provides evidence of widespread transcription of pseudogenes unraveled by high throughput transcriptome sequencing (Table 6A).

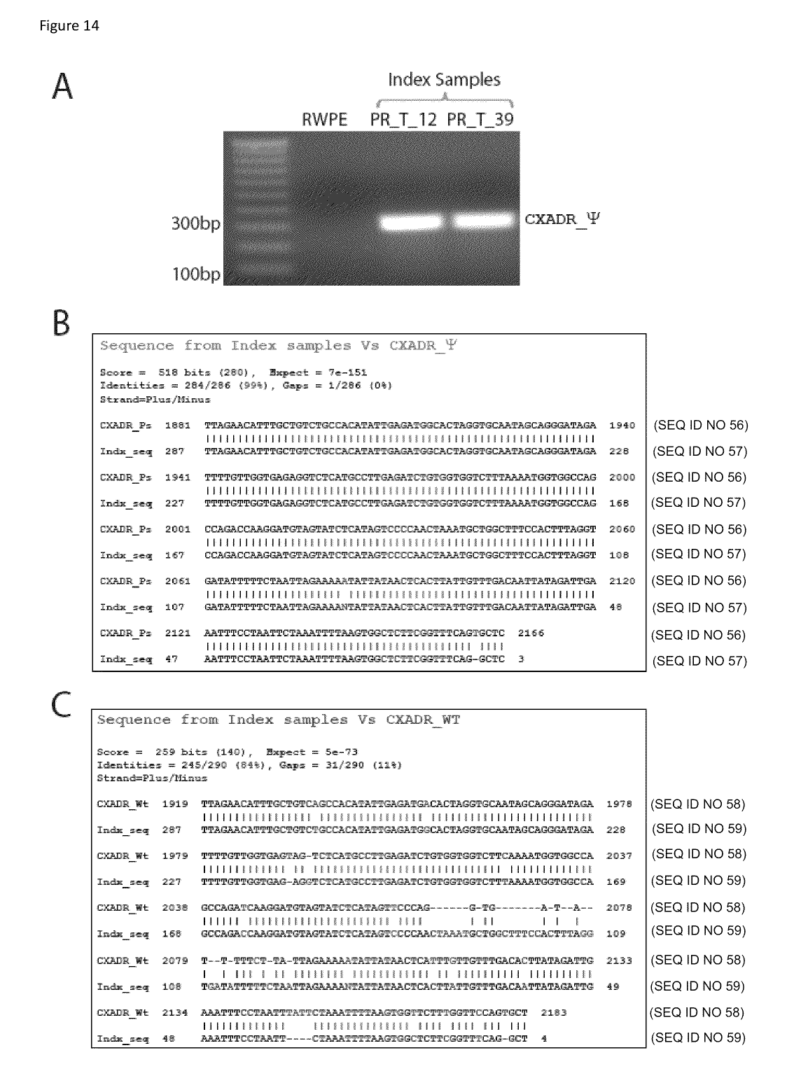

A closer look at the coverage of pseudogene clusters across the sample-wise compendium reveals that pseudogenes of housekeeping genes such as ribosomal and proteins are widely expressed across tissue types. Pseudogene transcripts corresponding to ribosomal proteins RPL-1, -3, -5, -6, -8, -9, -10, -11, -13, -18, -22, -23, -27, -28 and RPS-5, -6, -10, -11, -14, -16, -18, -20, -21 etc. were all observed in more than 50 samples each. Apart from housekeeping genes, several of the pseudogenes seen to be widely expressed included CALM2 (calmodulin 2 phosphorylase kinase, delta), TOMM40 (translocase of outer mitochondrial membrane 40), NONO (non-POU domain containing, octamer-binding), DUSP8 (dual specificity phosphatase 8), PERP (TP53 apoptosis effector), YES (v-yes-1 Yamaguchi sarcoma viral oncogene homolog 1) and others, were all found to be expressed in more than 50 of the samples examined by RNA-seq, as well as independently verified by pseudogene specific RT-PCR followed by validation through Sanger sequencing (Table 6A and 7, FIG. 14).

Further, because the RNA-Seq compendium is comprised of 35-45 mer short sequence reads that largely generated short sequence clusters not optimal for pseudogene analysis tools such as Pseudopipe (Zhang et al., 2006, supra) and Pseudofam (Lam et al., 2009, supra) used in generating ENCODE and Yale databases, we also carried out a direct query of individual clusters against the human genome (hg18) using the BLAT tool from UCSC, that is ideally suited for short sequence alignment searches (Kent, 2002 Genome research 12, 656-664). Using this BLAT analysis, also referred as the “custom” analysis or simply BLAT (FIG. 82A), it was possible to independently assign 1888 clusters representing 1820 unique pseudogenes to unique genomic locations. Comparing the genomic locations of the pseudogene clusters identified by BLAT analysis to those identified by Yale and ENCODE databases (FIG. 8A), 762 clusters were found to be common to all the three resources, but a remarkably large set of 585 clusters were uniquely defined by BLAT analysis alone. Some of the novel pseudogene transcripts thus identified including BAT1, BTBD1, COX7A2L, CTNND1, EIF5, PAPOLA, PARP11, SYT, and ZBTB12 and others (n=25) were validated by RT-PCR followed by Sanger sequencing (Table 7). Thus, analysis of RNA-seq data was producing reliable prediction of expressed pseudogenes.

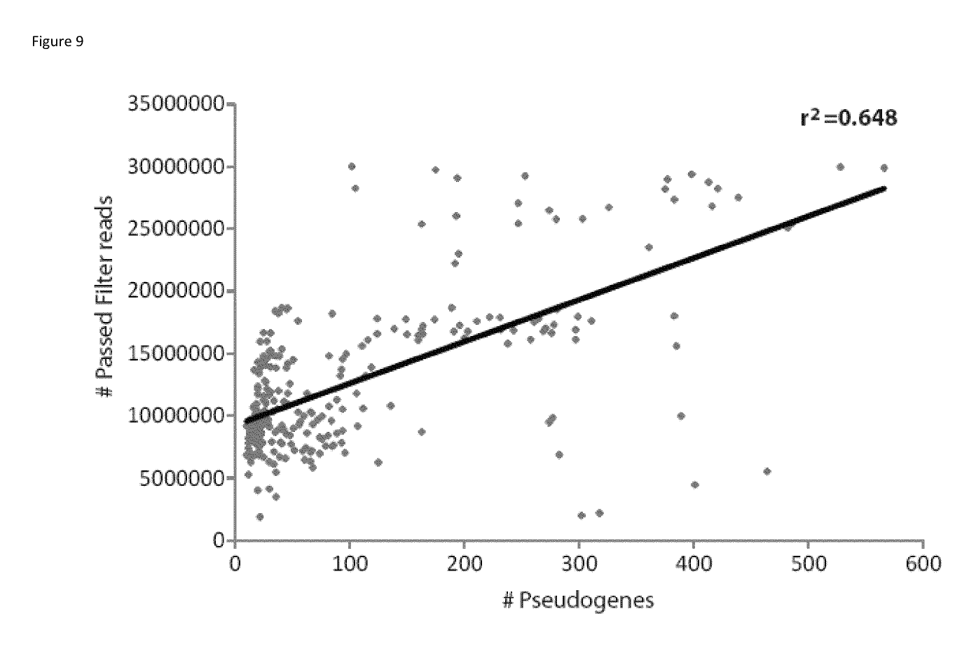

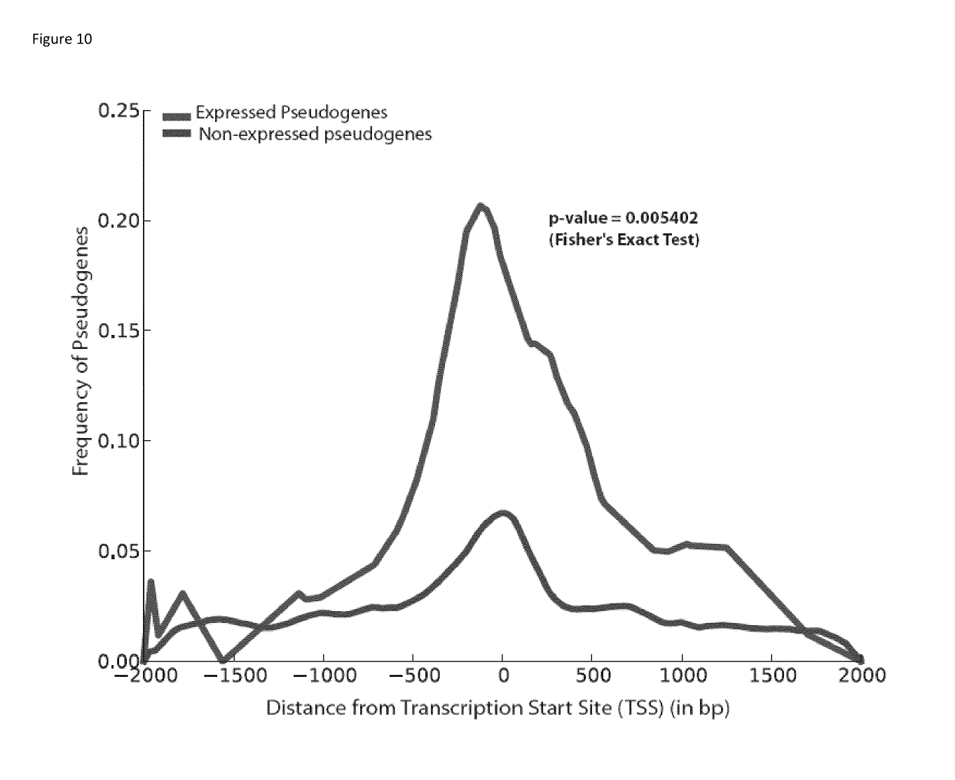

Given the utility of RNA-seq in unraveling pseudogene transcription, the technical and analytical factors influencing the yield of pseudogene transcripts were assesed through transcriptome sequencing. A positive correlation was observed between the sequencing depth and total number of pseudogene transcripts (correlation co-efficient, +0.65) (FIG. 9). On the other hand, no significant correlation was observed between the absolute measure of percent similarity between pseudogene 10 WT pairs and pseudogene yield (FIG. 10). The metric of overall percent similarity accounts for gap penalty and mismatches in BLAT search, but, it is the ‘distribution’ of the mismatches that is critical in resolving pseudogenes from nearly identical wild type sequences—for example, few mismatches, accumulated in a small stretch are more effective in confidently distinguishing pseudogene expression from wild types as compared to higher number of mismatches, but scattered over long stretches of sequence (FIG. 2). Thus, three primary factors determine the detection of pseudogene transcription by RNA-seq 1) the level of expression of the pseudogenes (e.g., the higher the level of expression, higher the likelihood of detection), 2) the depth of RNAsequencing and 3) overall distribution of mismatches with respect to the wild type.

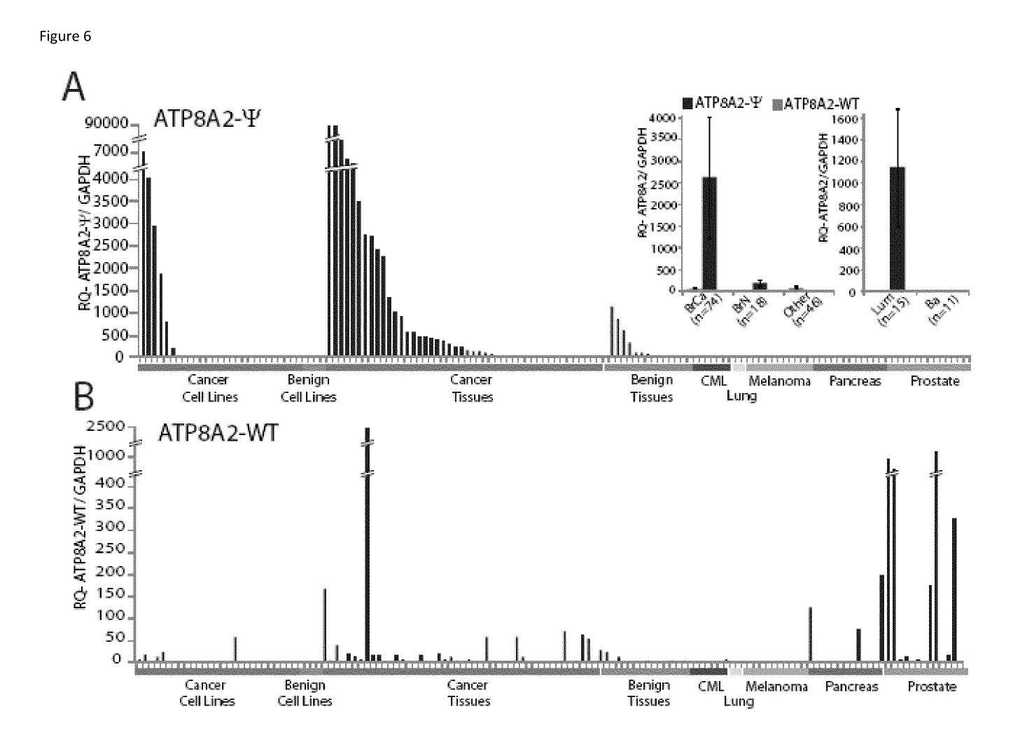

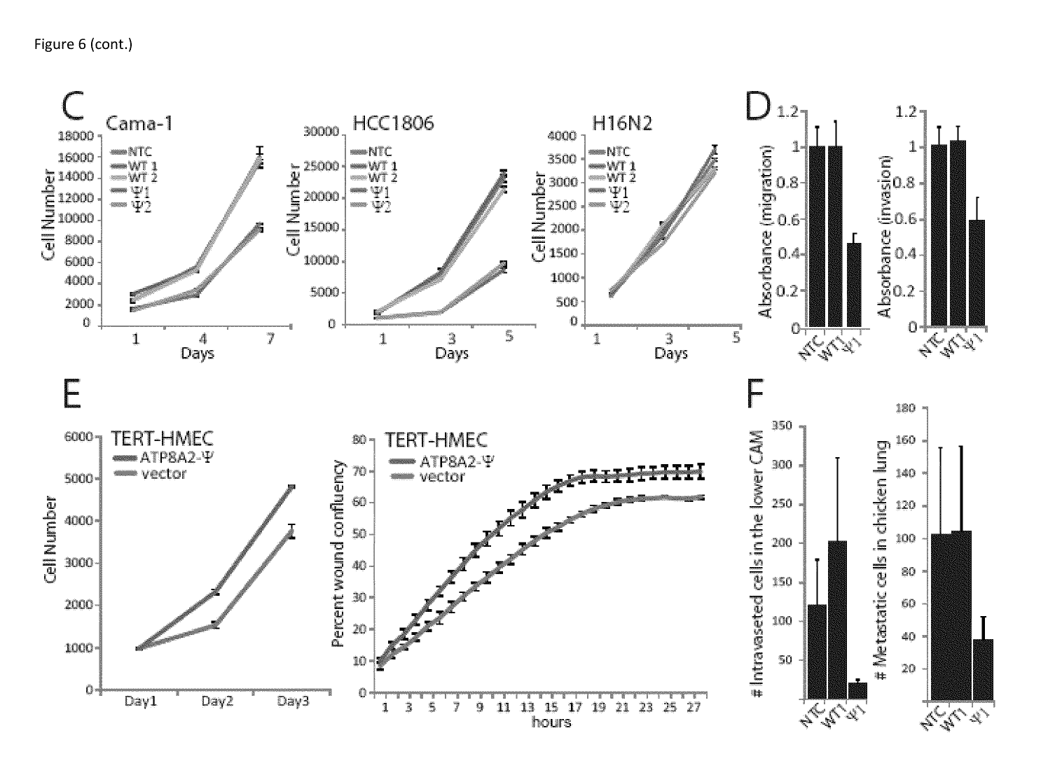

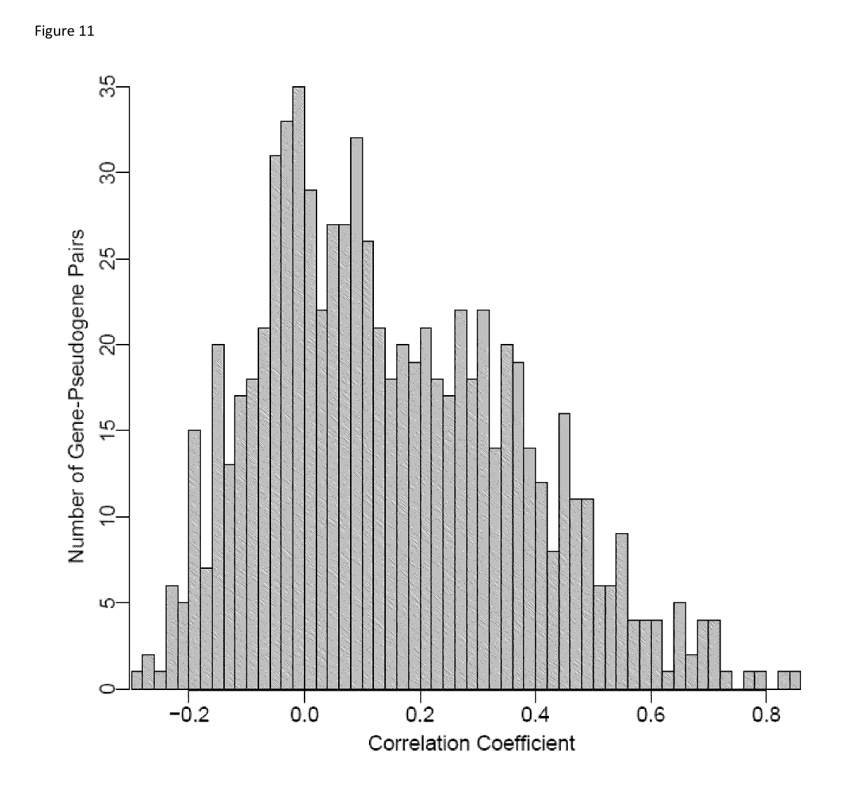

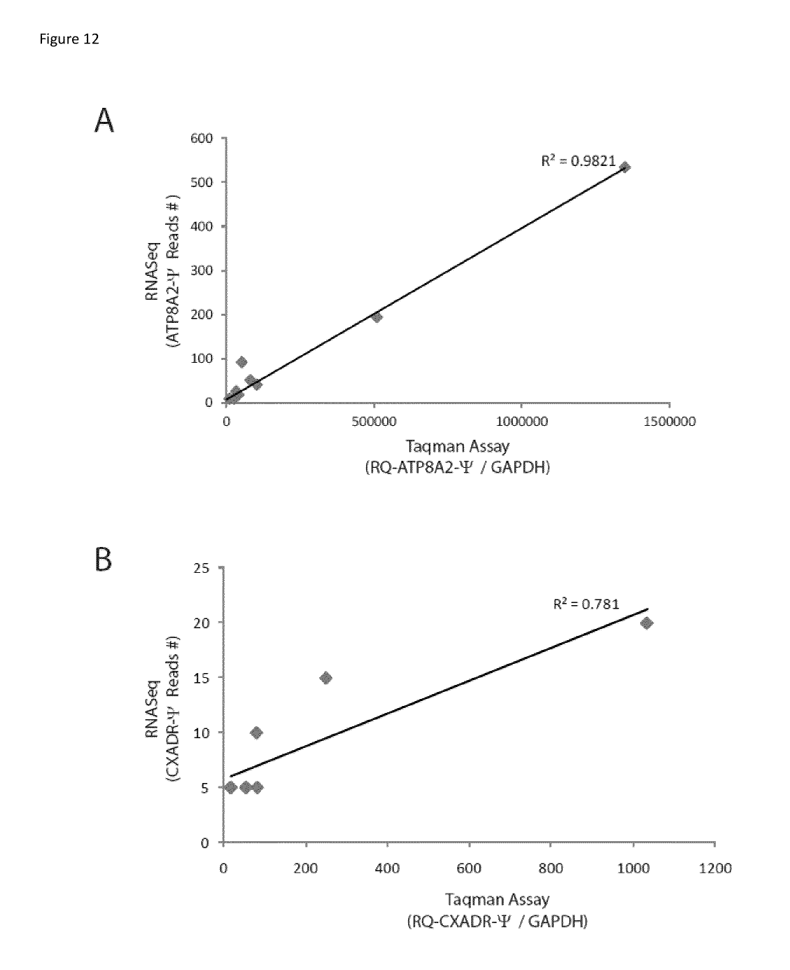

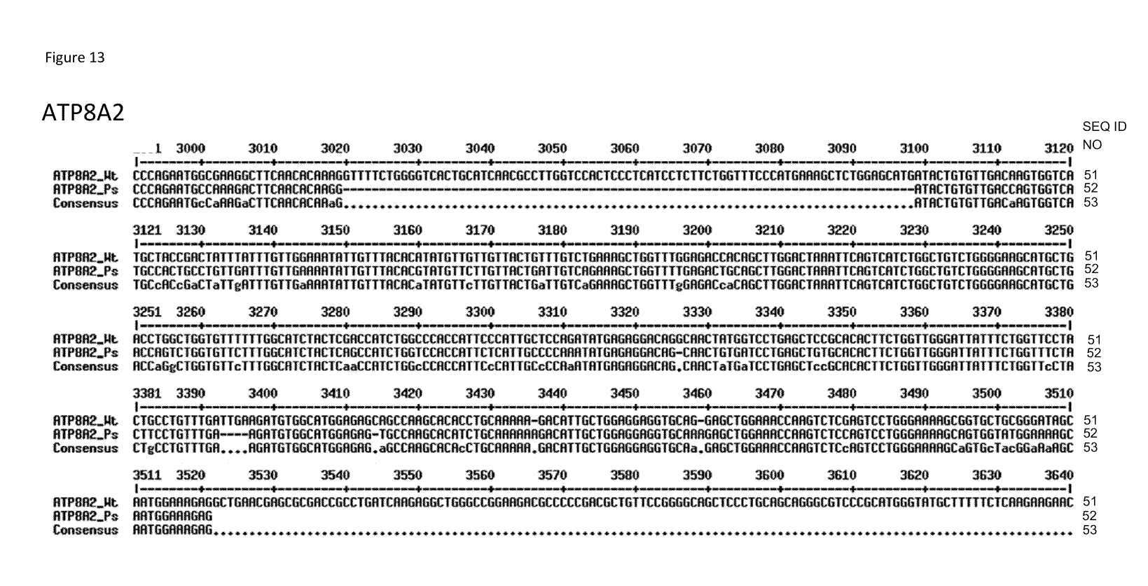

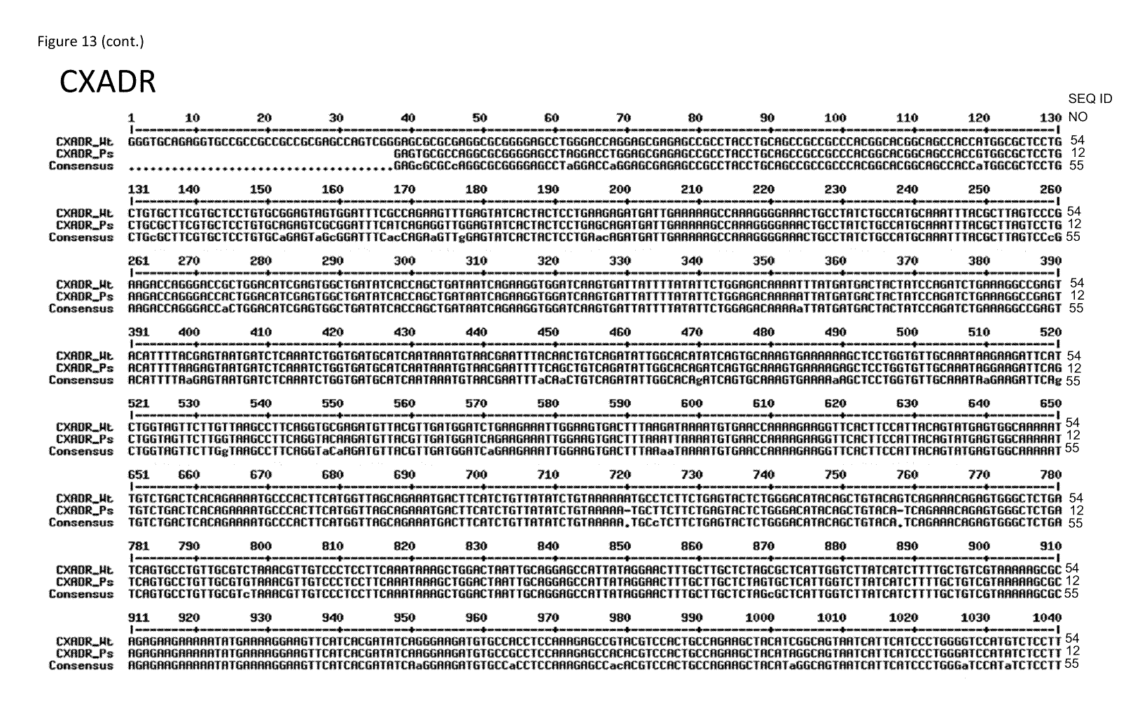

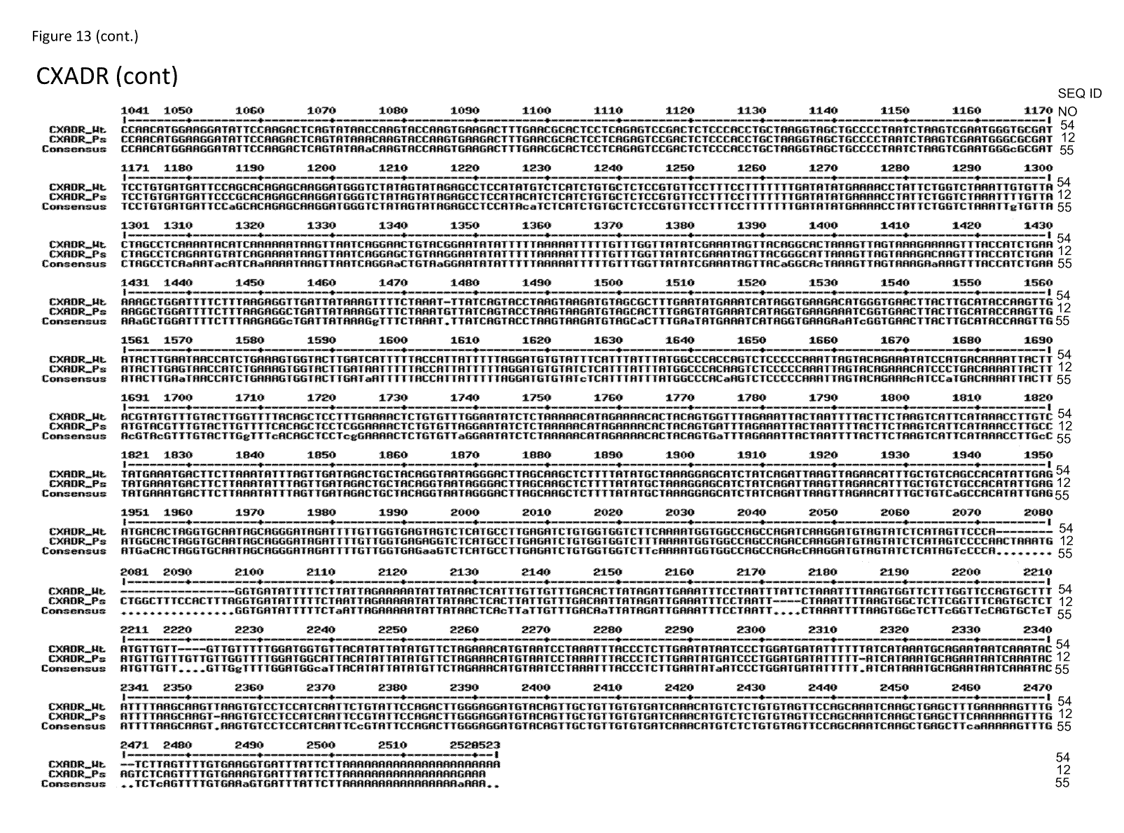

To explore regulatory elements associated with expressed pseudogenes, the pseudogene loci was subjected to two independent promoter analysis tools namely Transfac and Genomatix-Promoter Inspector. These tools provide information only on previously annotated promoter elements associated with known genes and were unable to identify potential promoter elements associated with the pseudogene loci solely based on query sequences. Therefore, ChIP-seq analysis of a breast cancer cell line MCF7 probed with H3K4me3, a histone mark associated with transcriptionally active chromosomal loci, was performed and the results were interogated with the MCF7 pseudogene transcript data. A statistically significant enrichment of H3K4me3 peaks at expressed pseudogene loci as compared to non-expressed pseudogenes (p-value=0.0145) was observed (FIG. S4), indicating that the pseudogene transcripts observed by RNA-seq are associated with transcriptionally active genomic loci. The pseudogene transcripts associated with H3K4Me3 peaks encompass both unprocessed and processed pseudogenes, with no discernible differences in the pattern of expression. Next, the correlation between the expression of pseudogenes present within the introns of unrelated, expressed genes, with their ‘host’ genes was assessed. No significant association was observed, indicating that pseudogenes are likely subject to independent regulatory mechanisms—even when residing within other transcriptionally active genes. Further, the observations with the breast specific unprocessed pseudogene ATP8A2 (likely arisen from duplication of wild type ATP8A2, thus likely harboring similar promoter elements) also indicate that there is no apparent correlation between the pseudogene expression with the wild type gene that is expressed ubiquitously (FIG. 6A, B). Next, the correlation between expression of pseudogenes with that of cognate wild type genes was assessed and no significant pattern of correlation was observed (FIG. 11).

### Patterns of Pseudogene Expression in Human Tissues

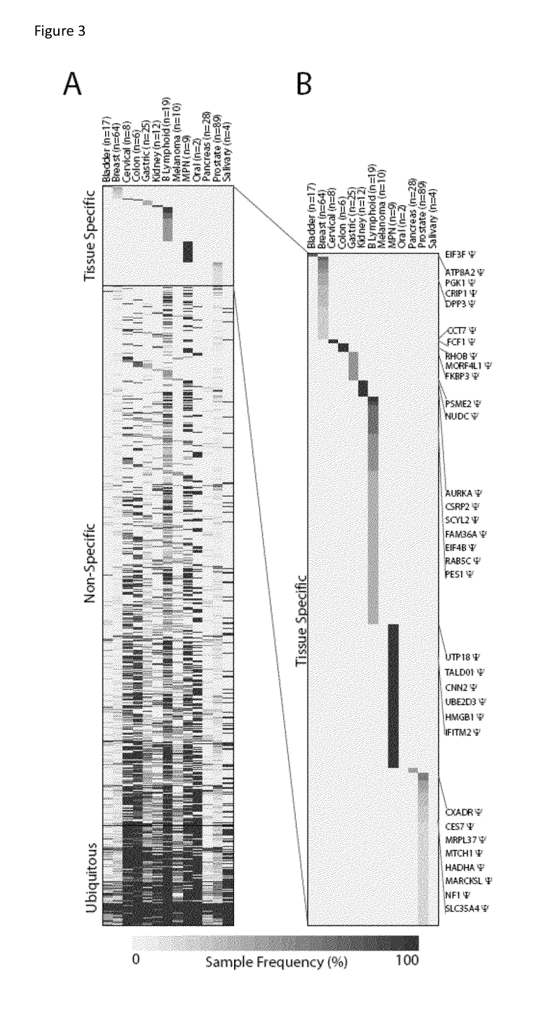

Next, the expression patterns of the pseudogene transcripts in the RNA-seq compendium comprising of data from 248 cancer and 45 benign samples from 13 different tissue types (total 293 samples) were analyzed. Broad patterns of pseudogene expression, including 1056 pseudogenes that were detected in multiple samples (Table 3) were observed, which supports the hypothesis that transcribed pseudogenes contribute to the typical transcriptional repertoire of cells. In addition, distinct patterns of pseudogene expression, akin to that of protein-coding genes, including 154 highly tissuespecific and 848 moderately tissue-specific (or tissue-enriched) pseudogenes (FIG. 3) were identified. Moreover, 165 pseudogenes exhibiting expression in more than 10 of the 13 tissue types examined were observed, and these were classified as ubiquitous pseudogenes whose transcription is characteristic of most cell types (FIG. 3, bottom).

Of the 165 ubiquitous pseudogenes, a majority belonged to housekeeping genes, such as GAPDH, ribosomal proteins, several cytokeratins, and other genes widely expressed in most cell types. These genes are known to have numerous pseudogenes, and it is likely that several of these pseudogenes retain the capacity for widespread transcription, mimicking their protein-coding counterparts.

A second set of pseudogenes exhibited near-ubiquitous expression, but were frequently transcribed at lower levels in most tissues and robustly transcribed in one or two tissues. These pseudogenes were termed “non-specific”, and this group harbors more than 870 pseudogenes, comprising a large portion of our dataset (FIG. 3, middle). Many of the pseudogenes previously shown to be expressed were found in this category, including some pseudogenes previously reported as tissue specific such as CYP4Z2P, a pseudogene previously reported to be expressed only in breast cancer tissues (Rieger et al., 2004 Cancer research 64, 2357-2364). Other candidates observed in this category include pseudogenes derived from Oct-4 (Kastler et al. The Prostate 70, 666-674), Connexin-43 (Bier et al., 2009 Molecular cancer therapeutics 8, 786-793; Kandouz et al., 2004 Oncogene 23, 4763-4770) and BRAF (Zou et al., 2009 Neoplasia New York, N.Y. 11, 57-65), among others (Table 3).

A notable pseudogene not observed is the recently described PTENP1, a pseudogene of PTEN recently implicated in the biology of the PI-3K signaling pathway (Poliseno et al. Nature 465, 1033-1038). No sequencing reads for PTENP1 were observed in the entire compendium—possibly due to the preponderance of cancer samples in the cohort, which tend to show low expression or deletion of this pseudogene (Poliseno et al. Nature 465, 1033-1038).

### Tissue- and Cancer-Specific Pseudogene Expression Signatures

154 pseudogenes with highly specific expression patterns were observed, including pseudogenes derived from AURKA (kidney samples), RHOB (colon samples), and HMGB1 (myeloproliferative neoplasms (MPNs)) (FIG. 3, right). Tissue-specific pseudogenes tended to represent a small fraction of all pseudogenes expressed in a given tissue type, and the total number of tissue-specific pseudogenes observed in a tissue type did not show a correlation with the total number of samples analyzed. For example, B-lymphocyte cells (n=19) and MPNs (n=9) showed more tissue-specific pseudogenes than breast (n=64) or prostate (n=89). More pseudogene transcripts were observed in samples with longer read lengths and deeper coverage. Together, these data both confirm and formalize previous anecdotal observations of tissue-specific pseudogene expression patterns by exploiting the power of RNA-Seq to resolve individual transcripts (FIG. 3, top) (Bier et al., 2009 Molecular cancer therapeutics 8, 786-793; Lu et al., 2006 The Prostate 66, 936-944; Rieger et al., 2004 Cancer research 64, 2357-2364).

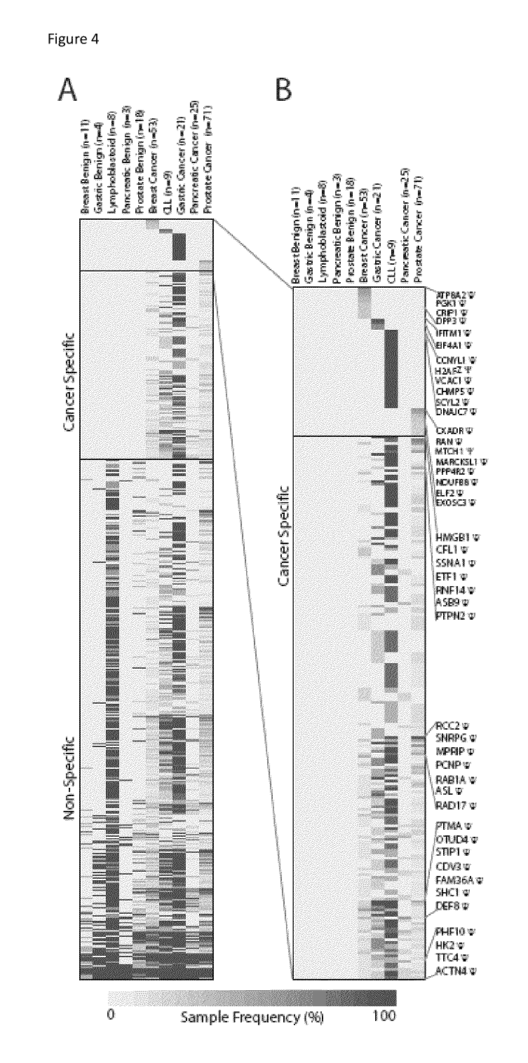

Because the sample compendium has a substantial number of cancer samples, pseudogenes with cancer-specific expression were next investigated. While a majority of the pseudogenes examined were found in both cancer and benign samples, 218 pseudogenes were expressed only in cancer samples, of which 178 were observed in multiple cancers and 40 were found to have highly-specific expression in a single cancer type only (FIG. 4; Table 4). The number of cancer-type-specific pseudogenes did not correlate with the number of samples sequenced in a given cancer type. These results indicate that cancer samples harbor transcriptional patterns of pseudogenes that are both lineage- and cancer-specific.

Among the cancer-specific pseudogenes, a few noteworthy examples included pseudogenes derived from the eukaryotic translation initiation factors EIF4A1 and EIF4H, the heterogeneous nuclear ribonucleoprotein HNRPH2, and the small nuclear ribonucleoprotein SNRPG (FIG. 4). Moreover, pseudogenes corresponding to known cancer-associated genes, including RAB-1, a Ras-related protein, VDAC1, a type-1 voltage dependent anion-selective channel/porin, RCC2, a regulator of chromosome condensation 2, and PTMA, prothymosin alpha were observed (FIG. 4). The parental protein-coding PTMA gene has given rise to five processed pseudogenes that retain consensus TATA elements, individual transcriptional start sites, and intact open reading frames that may potentially code for proteins closely related to the parental PTMA protein. Expression of PTMA-derived pseudogenes were found in more than 30 cancer samples, but not in any benign cells (FIG. 4), and these data indicate that PTMA-derived pseudogenes may not only contribute transcripts to cancer cell biology but potentially novel proteins as well.

### Breast Cancer Pseudogenes

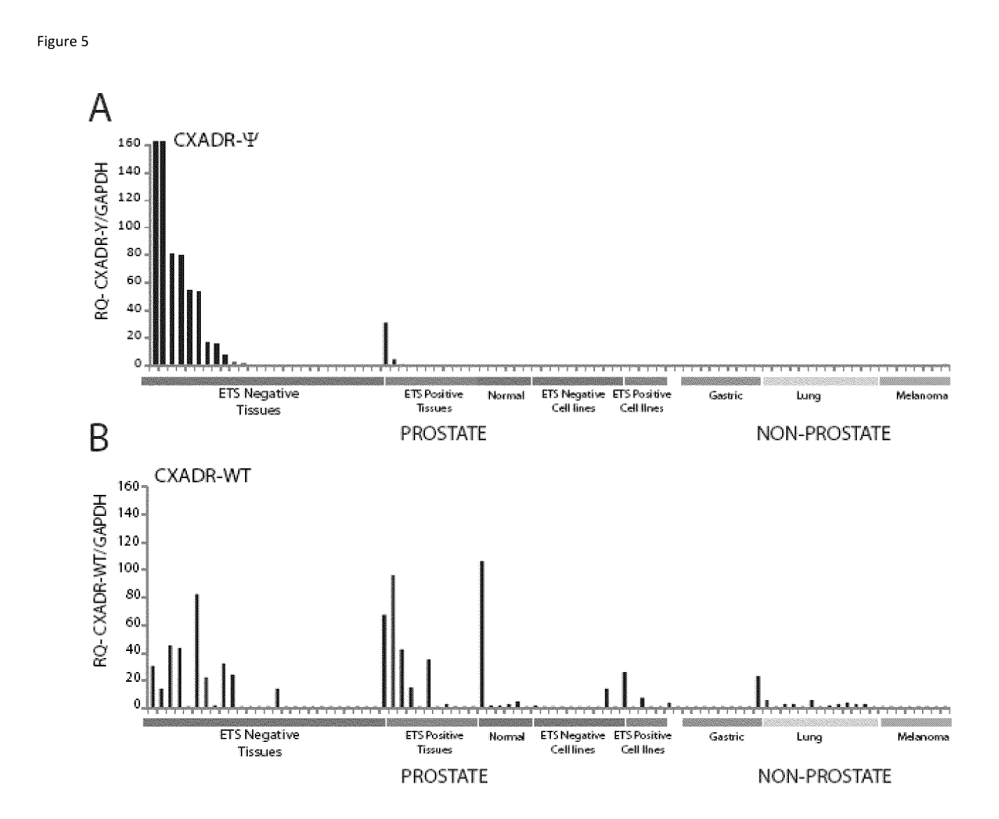

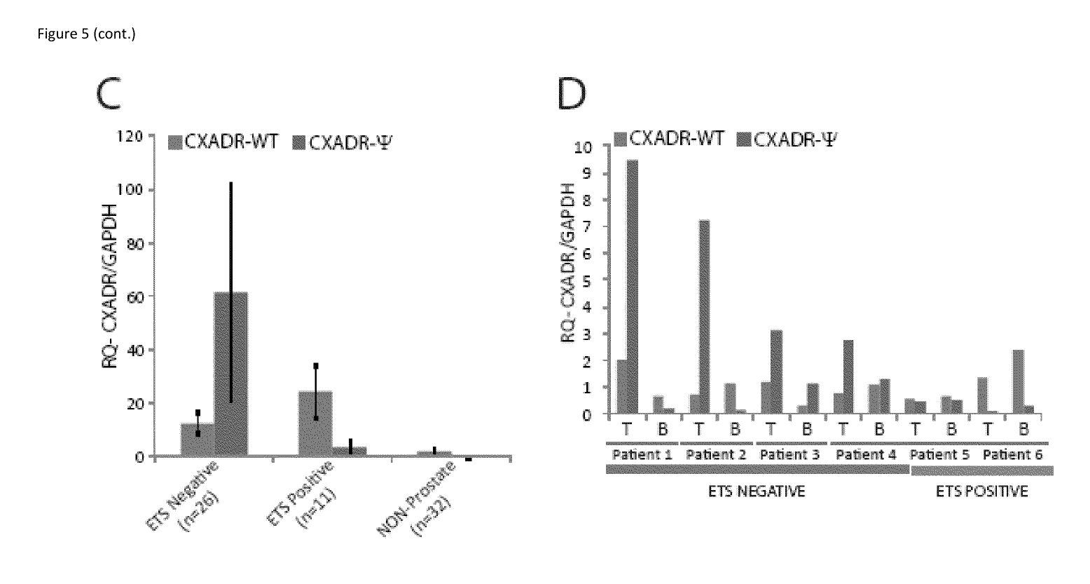

To investigate individual pseudogenes in greater detail, pseudogenes associated with breast and prostate cancer were investigated. Among the candidates in breast cancer, a novel unprocessed pseudogene cognate to ATP8A2, a LIM domain containing protein speculated to be associated with stress response and proliferative activity (Khoo et al., 1997 Protein expression and purification 9, 379-387), and DPP3, a metallopeptidase shown to have increased activity in endometrial and ovarian cancers (Simaga et al., 2003 Gynecologic oncology 91, 194-200) were investigated (FIG. 3, top; Table 3). Because ATP8A2-Ψ (on chromosome 10) displays substantial sequence divergence from the cognate ATP8A2 parental gene (on chromosome 13) thereby lending high confidence to the computational identification—this candidate was selected for further validation. Taqman assays were designed to distinguish wild type ATP8A2 transcripts from ATP8A2-Ψ and multiple tissue types (including breast, prostate, melanoma, lung, pancreatic cancer, and chronic myeloid leukemia (CML)) were assayed for expression. It was found that ATP8A2-Ψ expression was restricted to breast samples, and that this pseudogene displays profound upregulation in a subset of breast cancer tissues and cell lines (FIG. 5A,B). By contrast, expression of the parental ATP8A2 had no correlation with ATP8A2-Ψ expression and its expression was highly variable across different tissue types in the cohort, with the most pronounced expression intriguingly observed in prostate samples (FIG. 5A, lower panel). As ATP8A2-Ψ is an unprocessed pseudogene and has significant mappable difference with respect to its wild type, a significant correlation (R2=0.98) between the expression pattern obtained by RNA-seq and qPCR values was observed.

Approximately 25% of breast tumors demonstrate extremely high levels of this pseudogene, indicating that ATP8A2-Ψ may contribute to a particular subtype of breast cancer. ATP8A2-Ψexpression with respect to luminal and basal breast subtypes, two prominent categories of breast cancer with distinct molecular and clinical characteristics was analyzed. It was found that ATP8A2-Ψ expression was restricted to tumors with luminal histology, whereas basal tumors showed minimal expression of this pseudogene (FIG. 5B, right). The wild-type ATP8A2 transcript did not display this pattern of expression.

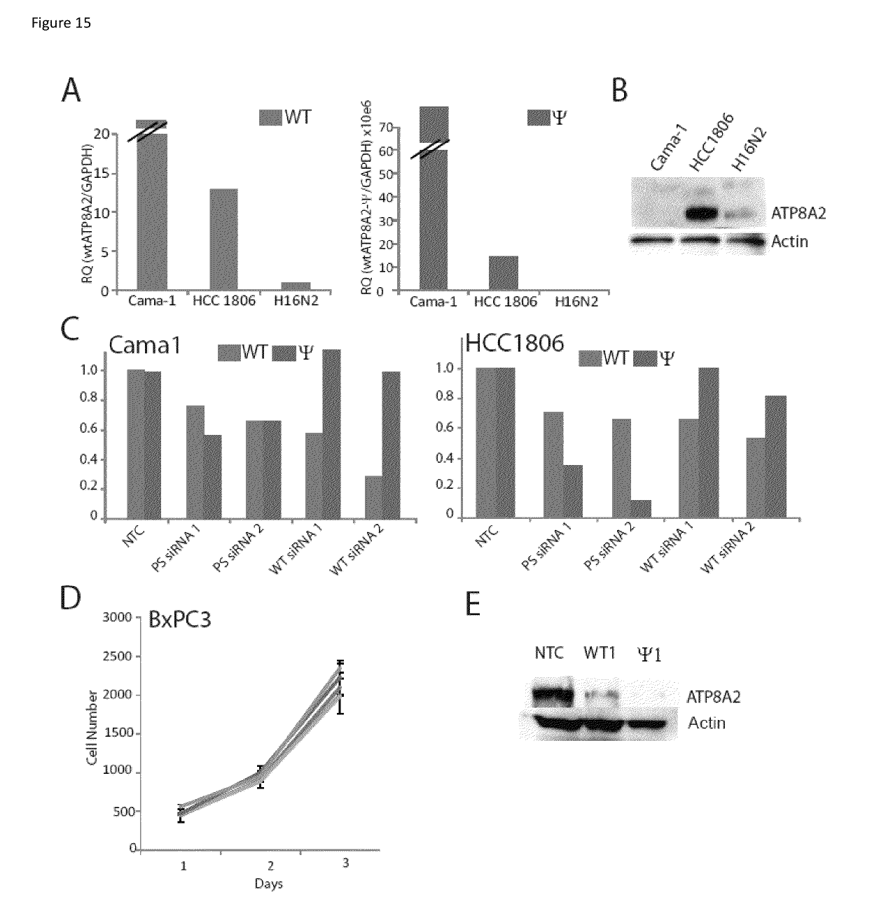

To investigate the role of ATP8A2-Ψ expression in breast cancer, siRNA based knockdown of both the wild type and pseudogene RNA was pereformed in two independent breast cancer cell lines that expressed both the transcripts (FIG. 15A). Knockdown of ATP8A2-Ψ, with two independent siRNAs was found to specifically inhibit the proliferation of overexpressing cell lines Cama1 and HCC1806 (FIG. 6C) but not the cell lines with no detectable levels of ATP8A2-Ψ for example, the benign breast epithelial cell line, H16N2 (FIG. 6C, right) and a pancreatic cancer cell line, BXPC3 (FIG. 15D). Knockdown of ATP8A2-Ψ but not ATP8A2-WT also resulted in reduced cell migration and invasion seen in in vitro Boyden Chamber assays (FIG. 6D) as well as in in vivo intravasation and metastasis in chicken chorioallantoic membrane xenograft assay (FIG. 6F). In contrast, knockdown of wild type ATP8A2 had no effect on the proliferation of any of the cell lines tested, indicating a novel growth regulatory role for ATP8A2-Ψ (FIG. 6C). While the knockdown of wild type ATP8A2 had minimal effect on the pseudogene transcript levels, ATP8A2-Ψ specific siRNAs apart from reducing the ATP8A2-Ψ transcript, also reduced the wild type protein levels (FIG. 15B, C). Thus clearly, unlike Oct4 and BRAF pseudogene transcripts having an inverse correlation with the wild type transcript levels, ATP8A2-Ψ and wild-type ATP8A2 transcripts (FIG. 6A,B) and protein (FIG. 15E) are not regulated in this manner. Subsequently, to assess the phenotypic effect of ATP8A2-Ψ overexpression in benign cells, it was cloned and overexpresssed in benign breast epithelial cell line TERT-HMEC. Two independent pooled populations of ATP8A2-Ψ overexpressing TERT-HMEC cells were found to undergo increased proliferation and migration (FIG. 6E) indicating an oncogenic nature of this novel breast specific pseudogene transcript.

### Prostate Cancer Pseudogenes

Analysis of tissue-specific pseudogenes restricted to prostate cancers identified numerous pseudogenes, including several derived from parental genes known to be altered or dysregulated in cancer (FIG. 3, right). For example, a prostate cancer pseudogene derived from NDUFA9, which encodes an NADH oxidoreductase component of mitochondrial complex I and is reported to be upregulated in testicular germ cell tumors (Dormeyer et al., 2008 Journal of proteome research 7, 2936-2951) was observed. Pseudogenes derived from EPCAM, whose parental protein-coding gene is an epithelial cell adhesion molecule involved in cancer and stem cells signaling (Munz et al., 2009 Cancer research 69, 5627-5629), PDAP1, that enhances the mitogenic effect of PDGF-A (Fischer and Schubert, 1996 Journal of neurochemistry 66, 2213-2216), RBM17, associated with drug resistance in numerous epithelial cancers (Perry et al., 2005 Cancer research 65, 6593-6600), (Sampath et al., 2003 The American journal of pathology 163, 1781-1790), and CES7, known to be expressed only in the male reproductive tract (Zhang et al., 2009 Acta biochimica et biophysica Sinica 41, 809-815) were also investigated (FIG. 3, right; Table 3). Among the prostate cancer specific pseudogenes, the processed pseudogene CXADR-Ψ, on chromosome 15, was of immediate interest, as the parental CXADR protein demonstrates putative tumor suppressor functions and its loss is implicated in α-catenin silencing (Pong et al., 2003 Cancer research 63, 8680-8686; Stecker et al., 2009 British journal of cancer 101, 1574-1579). This pseudogene was selected for further study in prostate cancer. Independent assays were used to evaluate CXADR-Ψ and parental CXADR gene expression levels using qPCR-based methods. As with ATP8A2-Ψ, upregulation of CXADR-Ψ in ˜25% of prostate cancer tissues, with minimal expression in benign prostate samples and non-prostate tissues was observed (FIG. 5A, top panel). There was no specific relationship between CXADR-Ψ and parental CXADR gene expression, although parental CXADR also had some proclivity for prostate cancer specific expression (FIG. 5A, lower panel). CXADR-Ψ expression was nearly restricted to prostate cancers lacking an ETS gene fusion (FIG. 5A, B), with few ETS-positive samples exhibiting expression of this pseudogene. By contrast, parental CXADR gene expression was found in both ETS-positive and ETSnegative samples (FIG. 5B). CXADR-Ψ and CXADR parental gene expression was assayed in a set of six prostate patients with matched cancer and benign tissues (including four ETS negative and two ETS positive pairs). Again, ETS-negative prostate cancer samples displayed marked upregulation of CXADR-Ψ compared to the ETS-positive patients, with CXADR parental gene expression fairly constant between this set of patients (FIG. 5B, right). As CXADR pseudogene is a processed pseudogene and also less significant mappable difference with respect to its wild type, a significant correlation (R2=0.78) was observed between the expression pattern of RNA-seq and qPCR values (FIG. 14B). As confirmation, the CXADR pseudogene cDNA was cloned and sequences from two index prostate cancer samples, where the resultant sequence mapped 99% mapped to CXADRP2 (pseudogene) and only 84% mapped to CXADR wild type gene.

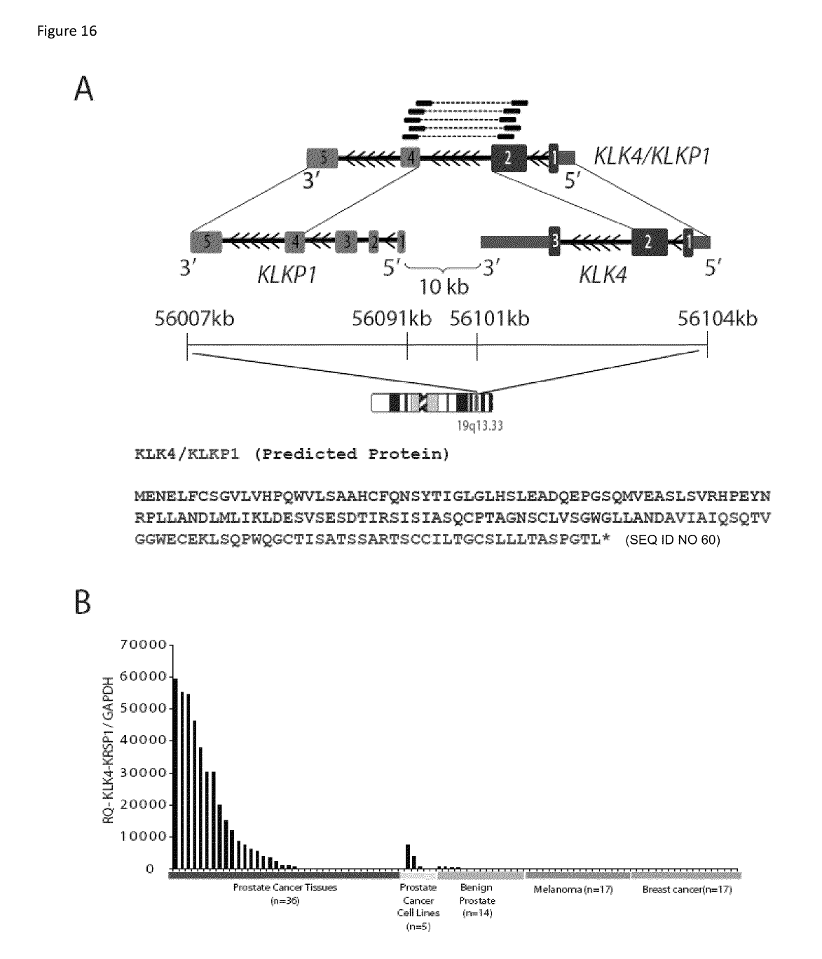

Lastly, in the course of these analyses, a prostate cancer specific read-through transcript between KLK4, an androgen-induced gene, and KLKP1, an adjacent pseudogene was identified. This read-through results in a chimeric RNA transcript combining the first two exons of KLK4 with the last two exons of KLKP1; and this KLK4-KLKP1 transcript is predicted to retain an open reading frame incorporating 54 amino acids encoded by the KLKP1 pseudogene, indicating that it may encode a chimeric protein (FIG. 16A). This read-through was also recently reported in the LNCaP prostate cancer cell line as a cis sense-antisense chimeric transcript. The KLK4-KLKP1 transcript was highly expressed in 30-50% of prostate cancer tissues, and again this expression was tissue- and cancer-specific, with minimal expression in benign prostate samples and other tissue types (FIG. 16B).

TABLE 1

| Sample_ID | Flowcell_ lane | Sample_Name | Tissue_Type | Status |
|---|---|---|---|---|
| BL_C_1 | 42Y2MAAXX.2 | BL_C_1 | Lymphoma | Tumor Tissue |
| BL_C_2 | 61VFCAAXX.1 | BC3 | Lymphoma | Tumor Cell Line |
| BL_C_3 | 61VFCAAXX.2 | BCP1 | Lymphoma | Tumor Cell Line |
| BL_C_4 | 61VFCAAXX.3 | BCBL1 | Lymphoma | Tumor Cell Line |
| BL_C_5 | 6286PAAXX.8 | RPMI_6666 | Lymphoma | Tumor Cell Line |
| BL_N_C_1 | 302LVAAXX.5 | HCC1428_BL | Lymphoblastoid | Benign Cell Line |
| BL_N_C_2 | 302LVAAXX.6 | HCC1937_BL | Lymphoblastoid | Benign Cell Line |
| BL_N_C_3 | 302LVAAXX.7 | HCC1187_BL | Lymphoblastoid | Benign Cell Line |
| BL_N_C_4 | 42TCLAAXX.1, | HCC38_BL | Lymphoblastoid | Benign Cell Line |
| | 42Y39AAXX.2 | | | |
| BL_N_C_5 | 42TCLAAXX.3, | HCC1599_BL | Lymphoblastoid | Benign Cell Line |
| | 42Y39AAXX.5 | | | |
| BL_N_C_6 | 42TCLAAXX.4, | HCC2157_BL | Lymphoblastoid | Benign Cell Line |
| | 42Y39AAXX.6 | | | |
| BL_N_C_7 | 42TCLAAXX.5, | HCC2218_BL | Lymphoblastoid | Benign Cell Line |
| | 42Y39AAXX.7 | | | |
| BL_N_C_8 | 42Y39AAXX.3, | HCC1143_BL | Lymphoblastoid | Benign Cell Line |
| | 42Y39AAXX.4 | | | |
| BL_T_1 | 42Y2MAAXX.1 | BL_T_1 | Chronic | Tumor Tissue |
| | | | Lymphocytic | |
| | | | Leukemia | |
| BL_T_2 | 42Y2MAAXX.3 | BL_T_2 | Chronic | Tumor Tissue |
| | | | Lymphocytic | |
| | | | Leukemia | |
| BL_T_3 | 42Y2MAAXX.4 | BL_T_3 | Chronic | Tumor Tissue |
| | | | Lymphocytic | |
| | | | Leukemia | |
| BL_T_4 | 42Y2MAAXX.5 | BL_T_4 | Chronic | Tumor Tissue |
| | | | Lymphocytic | |
| | | | Leukemia | |
| BL_T_5 | 42Y2MAAXX.6 | BL_T_5 | Chronic | Tumor Tissue |
| | | | Lymphocytic | |
| | | | Leukemia | |
| BL_T_6 | 42Y2MAAXX.7 | BL_T_6 | Chronic | Tumor Tissue |
| | | | Lymphocytic | |
| | | | Leukemia | |
| Bld_T_1 | 42RMPAAXX.5 | Bld_T_1 | Bladder | Tumor Tissue |
| Bld_T_2 | 42RMPAAXX.6 | Bld_T_2 | Bladder | Tumor Tissue |
| Bld_T_3 | 42TCBAAXX.1 | Bld_T_3 | Bladder | Tumor Tissue |
| Bld_T_4 | 42TCBAAXX.2 | Bld_T_4 | Bladder | Tumor Tissue |
| Bld_T_5 | 42TCBAAXX.3 | Bld_T_5 | Bladder | Tumor Tissue |
| Bld_T_6 | 42TCBAAXX.4 | Bld_T_6 | Bladder | Tumor Tissue |
| Bld_T_7 | 42TCBAAXX.5 | Bld_T_7 | Bladder | Tumor Tissue |
| Bld_T_8 | 42TCBAAXX.6 | Bld_T_8 | Bladder | Tumor Tissue |
| Bld_T_9 | 42TCBAAXX.7 | Bld_T_9 | Bladder | Tumor Tissue |
| Bld_T_10 | 42TCBAAXX.8 | Bld_T_10 | Bladder | Tumor Tissue |
| Bld_T_11 | 42TG0AAXX.2 | Bld_T_11 | Bladder | Tumor Tissue |
| Bld_T_12 | 42TG0AAXX.3 | Bld_T_12 | Bladder | Tumor Tissue |
| Bld_T_13 | 42TG0AAXX.4 | Bld_T_13 | Bladder | Tumor Tissue |
| Bld_T_14 | 42TG0AAXX.5 | Bld_T_14 | Bladder | Tumor Tissue |
| Bld_T_15 | 42TG0AAXX.6 | Bld_T_15 | Bladder | Tumor Tissue |
| Bld_T_16 | 42TG0AAXX.7 | Bld_T_16 | Bladder | Tumor Tissue |
| Bld_T_17 | 42TG0AAXX.8 | Bld_T_17 | Bladder | Tumor Tissue |
| Br_C_1 | 42PMUAAXX.5 | HCC38 | Breast | Tumor Cell Line |
| Br_C_2 | 42D3NAAXX.4, | HCC1008 | Breast | Tumor Cell Line |
| | 42Y39AAXX.8 | | | |
| Br_C_3 | 3035BAAXX.3 | HCC1143 | Breast | Tumor Cell Line |
| Br_C_4 | 314NNAAXX.3, | HCC1395 | Breast | Tumor Cell Line |
| | 314WAAAXX.3 | | | |
| Br_C_5 | 314WAAAXX.5, | HCC1599 | Breast | Tumor Cell Line |
| | 42Y39AAXX.1 | | | |
| Br_C_6 | 30WB6AAXX.7 | HCC1937 | Breast | Tumor Cell Line |
| Br_C_7 | 30LEJAAXX.7 | MCF7 | Breast | Tumor Cell Line |
| Br_C_8 | 315CUAAXX.3 | ZR-75-1 | Breast | Tumor Cell Line |
| Br_C_9 | 314WAAAXX.4 | HCC1954 | Breast | Tumor Cell Line |
| Br_C_10 | 42PMUAAXX.3 | HCC2157 | Breast | Tumor Cell Line |
| Br_C_11 | 6137LAAXX.2 | MDA-MB-231 | Breast | Tumor Cell Line |
| Br_C_12 | 30Y6GAAXX.1, | MDA-MB-361 | Breast | Tumor Cell Line |
| | 314NNAAXX.5 | | | |
| Br_C_13 | 30Y6GAAXX.3 | MDA-MB-435S | Breast | Tumor Cell Line |
| Br_C_14 | 30LUMAAXX.3, | T-47D | Breast | Tumor Cell Line |
| | 314WGAAXX.1 | | | |
| Br_C_15 | 302LVAAXX.2 | HCC1187 | Breast | Tumor Cell Line |
| Br_C_16 | 3035BAAXX.2 | ZR-75-30 | Breast | Tumor Cell Line |
| Br_C_17 | 3035BAAXX.5 | HCC1428 | Breast | Tumor Cell Line |
| Br_C_18 | 3035BAAXX.6 | HCC1419 | Breast | Tumor Cell Line |
| Br_C_19 | 30650AAXX.5 | UACC-812_T | Breast | Tumor Cell Line |
| Br_C_20 | 30650AAXX.6 | UACC-893_T | Breast | Tumor Cell Line |
| Br_C_21 | 30650AAXX.7 | MDA-MB-330 | Breast | Tumor Cell Line |
| Br_C_22 | 30WB6AAXX.5, | MDA-MB-134-VI | Breast | Tumor Cell Line |
| | 315CUAAXX.4 | | | |
| Br_C_23 | 30WB6AAXX.6 | HCC70 | Breast | Tumor Cell Line |
| Br_C_24 | 30Y6GAAXX.2 | CAMA-1 | Breast | Tumor Cell Line |
| Br_C_25 | 314NNAAXX.2 | MDA-MB-175-VII | Breast | Tumor Cell Line |
| Br_C_26 | 314RPAAXX.5 | HCC2218 | Breast | Tumor Cell Line |
| Br_C_27 | 314RPAAXX.7 | BT-483 | Breast | Tumor Cell Line |
| Br_C_28 | 314WAAAXX.1 | BT-20 | Breast | Tumor Cell Line |
| Br_C_29 | 314WAAAXX.2 | BT-549 | Breast | Tumor Cell Line |
| Br_C_30 | 314WAAAXX.7 | Hs-578T | Breast | Tumor Cell Line |
| Br_C_31 | 314WGAAXX.5 | HCC1569 | Breast | Tumor Cell Line |
| Br_C_32 | 314WGAAXX.7 | MDA-MB-157 | Breast | Tumor Cell Line |
| Br_C_33 | 315CUAAXX.1 | MDA-MB-453 | Breast | Tumor Cell Line |
| Br_C_34 | 315CUAAXX.2 | MDA-MB-468 | Breast | Tumor Cell Line |
| Br_C_35 | 429T4AAXX.1 | HCC202 | Breast | Tumor Cell Line |
| Br_C_36 | 42HAMAAXX.3 | DU4475 | Breast | Tumor Cell Line |
| Br_C_37 | 42HAMAAXX.5 | HCC1500 | Breast | Tumor Cell Line |
| Br_C_38 | 42PFAAAXX.7 | SUM_149PT | Breast | Tumor Cell Line |
| Br_C_39 | 42PFAAAXX.8 | SUM_190PT | Breast | Tumor Cell Line |
| Br_C_40 | 42PMUAAXX.1 | MDA-MB-436 | Breast | Tumor Cell Line |
| Br_C_41 | 42PMUAAXX.2 | MDA-MB-415 | Breast | Tumor Cell Line |
| Br_N_C_1 | 30Y6GAAXX.6 | MCF12A | Breast | Benign Cell Line |
| Br_N_C_2 | 30Y6GAAXX.7 | MCF10A | Breast | Benign Cell Line |
| Br_N_C_3 | 314NNAAXX.4 | MCF10F | Breast | Benign Cell Line |
| Br_N_C_4 | 314RPAAXX.6 | HMEC1 | Breast | Benign Cell Line |
| Br_N_C_5 | 314WAAAXX.6 | HBL-100 | Breast | Benign Cell Line |
| Br_N_C_6 | 42B3YAAXX.S | H16N2 | Breast | Benign Cell Line |
| Br_N_C_7 | 42B3YAAXX.6 | hTERT-HME1 | Breast | Benign Cell Line |
| Br_N_C_8 | 42B3YAAXX.7 | HMEC2 | Breast | Benign Cell Line |
| Br_N_T_1 | 30Y6GAAXX.4 | Br_N_T_1 | Breast | Benign Tissue |
| Br_N_T_2 | 30Y6GAAXX.5 | Br_N_T_2 | Breast | Benign Tissue |
| Br_N_T_3 | 42PMUAAXX.4 | Br_N_T_3 | Breast | Benign Tissue |
| Br_T_1 | 30U00AAXX.1 | Br_T_1 | Breast | Tumor Tissue |
| Br_T_2 | 30GC3AAXX.1 | Br_T_2 | Breast | Tumor Tissue |
| Br_T_3 | 30GC3AAXX.2 | Br_T_3 | Breast | Tumor Tissue |
| Br_T_4 | 30U00AAXX.2 | Br_T_4 | Breast | Metastatic Tissue |
| Br_T_5 | 30U00AAXX.3 | Br_T_5 | Breast | Metastatic Tissue |
| Br_T_6 | 314N5AAXX.1 | Br_T_6 | Breast | Tumor Tissue |
| Br_T_7 | 314N5AAXX.2 | Br_T_7 | Breast | Tumor Tissue |
| Br_T_8 | 314N5AAXX.3 | Br_T_8 | Breast | Tumor Tissue |
| Br_T_9 | 314N5AAXX.4 | Br_T_9 | Breast | Tumor Tissue |
| Br_T_10 | 315CUAAXX.5 | Br_T_10 | Breast | Tumor Tissue |
| Br_T_11 | 315CUAAXX.6 | Br_T_11 | Breast | Tumor Tissue |
| Br_T_12 | 315CUAAXX.7 | Br_T_12 | Breast | Tumor Tissue |
| Cer_C_1 | 30Y5NAAXX.2, | Caski | Cervical | Tumor Cell Line |
| | 42Y6NAAXX.3 | | | |
| Cer_C_2 | 42Y6NAAXX.1 | C-33_A | Cervical | Tumor Cell Line |
| Cer_C_3 | 42Y6NAAXX.2 | C-4_II | Cervical | Tumor Cell Line |
| Cer_C_4 | 42Y6NAAXX.4 | HeLa | Cervical | Tumor Cell Line |
| Cer_C_5 | 42Y6NAAXX.5 | MS751 | Cervical | Tumor Cell Line |
| Cer_C_6 | 42Y6NAAXX.6 | HT-3 | Cervical | Tumor Cell Line |
| Cer_C_7 | 42Y6NAAXX.7 | ME-180 | Cervical | Tumor Cell Line |
| Cer_C_8 | 42Y6NAAXX.8 | SiHa | Cervical | Tumor Cell Line |
| CL_C_1 | 61VFCAAXX.6 | HCT-15 | Colon | Tumor Cell Line |
| CL_C_2 | 6286PAAXX.3 | SW620 | Colon | Tumor Cell Line |
| CL_C_3 | 6286PAAXX.4 | HT-29 | Colon | Tumor Cell Line |
| CL_C_4 | 6286PAAXX.5 | HCT_116 | Colon | Tumor Cell Line |
| CL_C_5 | 6286PAAXX.6 | COLO_205 | Colon | Tumor Cell Line |
| CL_C_6 | 6286PAAXX.7 | SW837 | Colon | Tumor Cell Line |
| Gst_C_1 | 42RMPAAXX.7 | YCC-16 | Gastric | Tumor Cell Line |
| Gst_C_2 | 42RMPAAXX.8 | YCC-3 | Gastric | Tumor Cell Line |
| Gst_C_3 | 42Y6WAAXX.2, | AZ-521 | Gastric | Tumor Cell Line |
| | 42YMTAAXX.2 | | | |
| Gst_C_4 | 42YMMAAXX.1 | IM95 | Gastric | Tumor Cell Line |
| Gst_C_5 | 42YMMAAXX.2, | MKN-28 | Gastric | Tumor Cell Line |
| | 42YMMAAXX.8 | | | |
| Gst_C_6 | 42YMMAAXX.3 | AGS | Gastric | Tumor Cell Line |
| Gst_C_7 | 42YMMAAXX.4 | NCI-N87 | Gastric | Tumor Cell Line |
| Gst_C_8 | 42YMTAAXX.1 | SNU-16 | Gastric | Tumor Cell Line |
| Gst_N_T_1 | 42RYFAAXX.6 | Gst_N_T_1 | Gastric | Benign Tissue |
| Gst_N_T_2 | 42RYFAAXX.7 | Gst_N_T_2 | Gastric | Benign Tissue |
| Gst_N_T_3 | 42RYFAAXX.8 | Gst_N_T_3 | Gastric | Benign Tissue |
| Gst_N_T_4 | 42YMTAAXX.7 | Gst_N_T_4 | Gastric | Benign Tissue |
| Gst_T_1 | 42RYFAAXX.1 | Gst_T_1 | Gastric | Tumor Tissue |
| Gst_T_2 | 42RYFAAXX.2 | Gst_T_2 | Gastric | Tumor Tissue |
| Gst_T_3 | 42RYFAAXX.3 | Gst_T_3 | Gastric | Tumor Tissue |
| Gst_T_4 | 42RYFAAXX.4 | Gst_T_4 | Gastric | Tumor Tissue |
| Gst_T_5 | 42RYFAAXX.5 | Gst_T_5 | Gastric | Tumor Tissue |
| Gst_T_6 | 42Y6WAAXX.3, | Gst_T_6 | Gastric | Tumor Tissue |
| | 42YMTAAXX.4 | | | |
| Gst_T_7 | 42Y6WAAXX.4, | Gst_T_7 | Gastric | Benign Tissue |
| | 42YMTAAXX.8 | | | |
| Gst_T_8 | 42YMMAAXX.5 | Gst_T_8 | Gastric | Tumor Tissue |
| Gst_T_9 | 42YMMAAXX.6 | Gst_T_9 | Gastric | Tumor Tissue |
| Gst_T_10 | 42YMMAAXX.7 | Gst_T_10 | Gastric | Tumor Tissue |
| Gst_T_11 | 42YMTAAXX.3 | Gst_T_11 | Gastric | Tumor Tissue |
| Gst_T_12 | 42YMTAAXX.5 | Gst_T_12 | Gastric | Tumor Tissue |
| Gst_T_13 | 42YMTAAXX.6 | Gst_T_13 | Gastric | Tumor Tissue |
| Ky_T_1 | 42RMPAAXX.1 | Ky_T_1 | Renal | Tumor Tissue |
| Ky_T_2 | 42RMPAAXX.2 | Ky_T_2 | Renal | Tumor Tissue |
| Ky_T_3 | 42RMPAAXX.3 | Ky_T_3 | Renal | Tumor Tissue |
| Ky_T_4 | 42RMPAAXX.4 | Ky_T_4 | Renal | Tumor Tissue |
| Ky_T_5 | 42TBWAAXX.1 | Ky_T_5 | Renal | Tumor Tissue |
| Ky_T_6 | 42TBWAAXX.2 | Ky_T_6 | Renal | Tumor Tissue |
| Ky_T_7 | 42TBWAAXX.3 | Ky_T_7 | Renal | Tumor Tissue |
| Ky_T_8 | 42TBWAAXX.4 | Ky_T_8 | Renal | Tumor Tissue |
| Ky_T_9 | 42TBWAAXX.5 | Ky_T_9 | Renal | Tumor Tissue |
| Ky_T_10 | 42TBWAAXX.6, | Ky_T_10 | Renal | Tumor Tissue |
| | 42Y6WAAXX.1 | | | |
| Ky_T_11 | 42TBWAAXX.7 | Ky_T_11 | Renal | Tumor Tissue |
| Ky_T_12 | 42TBWAAXX.8 | Ky_T_12 | Renal | Tumor Tissue |
| Mel_C_1 | 42YRVAAXX.2 | SK-MEL-173 | Melanoma | Tumor Cell Line |
| Mel_C_2 | 42B20AAXX.2 | SK-MEL-94 | Melanoma | Tumor Cell Line |
| Mel_C_3 | 42HAMAAXX.1 | MM96L | Melanoma | Tumor Cell Line |
| Mel_C_4 | 42B20AAXX.1 | D14 | Melanoma | Tumor Cell Line |
| Mel_C_5 | 42YRVAAXX.3 | MelCelline_Z | Melanoma | Tumor Cell Line |
| Mel_C_6 | 42HAMAAXX.2 | SK-MEL-5 | Melanoma | Tumor Cell Line |
| Mel_C_7 | 42YRVAAXX.1 | SK-MEL-28 | Melanoma | Tumor Cell Line |
| Mel_N_T_1 | 3035BAAXX.1 | MelN_T_1 | Melanocyte | Benign Tissue |
| Mel_T_1 | 42PF0AAXX.6 | Mel_T_1 | Melanoma | Tumor Tissue |
| Mel_T_2 | 42PF0AAXX.7 | Mel_T_2 | Melanoma | Tumor Tissue |
| MPN_T_1 | 6137KAAXX.8 | MPN_T_1 | MPN | Tumor Tissue |
| MPN_T_2 | 62837AAXX.1 | MPN_T_2 | MPN | Tumor Tissue |
| MPN_T_3 | 62837AAXX.2 | MPN_T_3 | MPN | Tumor Tissue |
| MPN_T_4 | 62837AAXX.3 | MPN_T_4 | MPN | Tumor Tissue |
| MPN_T_5 | 62837AAXX.4 | MPN_T_5 | MPN | Tumor Tissue |
| MPN_T_6 | 62837AAXX.7 | MPN_T_6 | MPN | Tumor Tissue |
| MPN_T_7 | 62837AAXX.8 | MPN_T_7 | MPN | Tumor Tissue |
| MPN_T_8 | 6286PAAXX.1 | MPN_T_8 | MPN | Tumor Tissue |
| MPN_T_9 | 6286PAAXX.2 | MPN_T_9 | MPN | Tumor Tissue |
| OC_C_1 | 6137KAAXX.6 | CGHNK6 | Oral | Tumor Cell Line |
| OC_C_2 | 6137KAAXX.7 | TW2.6 | Oral | Tumor Cell Line |
| PAN_C_1 | 314N5AAXX.6 | BxPC-3 | Pancreas | Tumor Cell Line |
| PAN_C_2 | 314WGAAXX.4 | MIA-PaCa-2 | Pancreas | Tumor Cell Line |
| PAN_C_3 | 30Y5NAAXX.1, | PANC-1 | Pancreas | Tumor Cell Line |
| | 315C1AAXX.1 | | | |
| PAN_C_4 | 30LUMAAXX.2, | Panc-10.05 | Pancreas | Tumor Cell Line |
| | 42AV4AAXX.4 | | | |
| PAN_C_5 | 314N5AAXX.5 | HPAF-II | Pancreas | Tumor Cell Line |
| PAN_C_6 | 42HAMAAXX.7 | AsPC-1 | Pancreas | Tumor Cell Line |
| PAN_C_7 | 314N5AAXX.7 | Capan-2 | Pancreas | Tumor Cell Line |
| PAN_C_8 | 30650AAXX.1 | Panc-05.04 | Pancreas | Tumor Cell Line |
| PAN_C_9 | 30WG6AAXX.5 | Hs-766T | Pancreas | Tumor Cell Line |
| PAN_C_10 | 314WGAAXX.2 | Capan-1 | Pancreas | Tumor Cell Line |
| PAN_C_11 | 314WGAAXX.3 | HPAC | Pancreas | Tumor Cell Line |
| PAN_C_13 | 315C1AAXX.3 | CFPAC-1 | Pancreas | Tumor Cell Line |
| PAN_C_14 | 315C1AAXX.4 | SU.86.86 | Pancreas | Tumor Cell Line |
| PAN_C_16 | 42AV4AAXX.1 | Hs-700T | Pancreas | Tumor Cell Line |
| PAN_C_17 | 42AV4AAXX.2 | PL45 | Pancreas | Tumor Cell Line |
| PAN_C_18 | 42AV4AAXX.3 | L3.3 | Pancreas | Tumor Cell Line |
| PAN_C_19 | 42AV4AAXX.5 | Panc-08.13 | Pancreas | Tumor Cell Line |
| PAN_C_20 | 42AV4AAXX.6 | Panc-02.03 | Pancreas | Tumor Cell Line |
| PAN_C_21 | 42AV4AAXX.7 | Panc-02.13 | Pancreas | Tumor Cell Line |
| PAN_C_22 | 42HAMAAXX.6 | Panc-03.27 | Pancreas | Tumor Cell Line |
| PAN_C_23 | 42CJJAAXX.1 | Panc-04.03 | Pancreas | Tumor Cell Line |
| PAN_N_C_1 | 30WG6AAXX.6 | HPNE | Pancreas | Benign Cell Line |
| PAN_N_C_2 | 30WG6AAXX.7 | HPDE | Pancreas | Benign Cell Line |
| PAN_N_T_1 | 30WG6AAXX.4 | PAN_N_T_1 | Pancreas | Benign Tissue |
| PAN_T_1 | 42CJJAAXX.2 | PAN_T_1 | Pancreas | Tumor Tissue |
| PAN_T_2 | 42CJJAAXX.3 | PAN_T_2 | Pancreas | Tumor Tissue |
| PAN_T_3 | 42CJJAAXX.4 | PAN_T_3 | Pancreas | Tumor Tissue |
| PAN_T_4 | 42PMUAAXX.8 | PAN_T_4 | Pancreas | Tumor Tissue |
| PR_C_1 | 30TUEAAXX.3 | VCaP | Prostate | Tumor Cell Line |
| PR_C_2 | 314NNAAXX.6 | 22Rv1 | Prostate | Tumor Cell Line |
| PR_C_3 | 3064YAAXX.1, | PC3 | Prostate | Tumor Cell Line |
| | 30LF7AAXX.1 | | | |
| PR_C_4 | 429T4AAXX.2 | C4-2B | Prostate | Metastatic Cell Line |
| PR_C_5 | 429T4AAXX.3 | LAPC-4 | Prostate | Tumor Cell Line |
| PR_C_6 | 429T4AAXX.4 | NCI-H660 | Prostate | Metastatic Cell Line |
| PR_C_7 | 429T4AAXX.5 | CA-HPV.10 | Prostate | Tumor Cell Line |
| PR_C_8 | 429T4AAXX.6 | MDA-PCa-2B | Prostate | Metastatic Cell Line |
| PR_C_9 | 429T4AAXX.7 | CWR22 | Prostate | Tumor Cell Line |
| PR_C_10 | 42B08AAXX.2 | PWR-1E | Prostate | Benign Cell Line |
| PR_C_12 | 42B08AAXX.4 | WPE1-NB26 | Prostate | Metastatic Cell Line |
| PR_C_13 | 42PFAAAXX.5, | LNCaP | Prostate | Metastatic Cell Line |
| | 42PFAAAXX.6, | | | |
| | 42PMUAAXX.6, | | | |
| | 42PMUAAXX.7 | | | |
| PR_C_17 | 42TA8AAXX.1, | DU-145 | Prostate | Tumor Cell Line |
| | 42TA8AAXX.2, | | | |
| | 42TA8AAXX.3, | | | |
| | 42TA8AAXX.4, | | | |
| | 42TA8AAXX.5, | | | |
| | 42TA8AAXX.6, | | | |
| | 42TA8AAXX.7 | | | |
| PR_N_C_1 | 30351AAXX.7, | PrEC | Prostate | Benign Cell Line |
| | 314T1AAXX.1 | | | |
| PR_N_C_2 | 30LEJAAXX.2 | RWPE | Prostate | Benign Cell Line |
| PR_N_C_3 | 314T1AAXX.2 | PrSMC | Prostate | Benign Cell Line |
| PR_N_T_1 | 30LF7AAXX.2 | PR_N_T_1 | Prostate | Benign Tissue |
| PR_N_T_2 | 30LF7AAXX.3 | PR_N_T_2 | Prostate | Benign Tissue |
| PR_N_T_3 | 301YWAAXX.1, | PR_N_T_3 | Prostate | Benign Tissue |
| | 42P6UAAXX.4 | | | |
| PR_N_T_4 | 3064YAAXX.2, | PR_N_T_4 | Prostate | Benign Tissue |
| | 42P6UAAXX.1 | | | |
| PR_N_T_5 | 3064YAAXX.3, | PR_N_T_5 | Prostate | Benign Tissue |
| | 42P6UAAXX.2 | | | |
| PR_N_T_6 | 3064YAAXX.7, | PR_N_T_6 | Prostate | Benign Tissue |
| | 42P6UAAXX.3 | | | |
| PR_N_T_9 | 30TUEAAXX.2 | PR_N_T_9 | Prostate | Benign Tissue |
| PR_N_T_10 | 30TUEAAXX.7 | PR_N_T_10 | Prostate | Benign Tissue |
| PR_N_T_11 | 30TUEAAXX.1 | PR_N_T_11 | Prostate | Benign Tissue |
| PR_N_T_12 | 30TUEAAXX.8 | PR_N_T_12 | Prostate | Benign Tissue |
| PR_N_T_17 | 30WU2AAXX.5, | PR_N_T_17 | Prostate | Benign Tissue |
| | 42B48AAXX.4 | | | |
| PR_N_T_18 | 30WU2AAXX.6, | PR_N_T_18 | Prostate | Benign Tissue |
| | 42B48AAXX.8, | | | |
| | 42PFAAAXX.2 | | | |
| PR_N_T_19 | 42B20AAXX.5, | PR_N_T_19 | Prostate | Benign Tissue |
| | 42NY4AAXX.6 | | | |
| PR_N_T_20 | 42B48AAXX.1, | PR_N_T_20 | Prostate | Benign Tissue |
| | 42NY4AAXX.3 | | | |
| PR_N_T_21 | 42D3NAAXX.5 | PR_N_T_21 | Prostate | Benign Tissue |
| PR_T_1 | 30TYGAAXX.1 | PR_T_1 | Prostate | Metastatic Tissue |
| PR_T_2 | 30TYGAAXX.3 | PR_T_2 | Prostate | Metastatic Tissue |
| PR_T_3 | 42B20AAXX.6 | PR_T_3 | Prostate | Metastatic Tissue |
| PR_T_5 | 30U00AAXX.7 | PR_T_5 | Prostate | Metastatic Tissue |
| PR_T_7 | 30TYGAAXX.5 | PR_T_7 | Prostate | Metastatic Tissue |
| PR_T_10 | 301YWAAXX.2, | PR_T_10 | Prostate | Tumor Tissue |
| | 42P6UAAXX.5 | | | |
| PR_T_11 | 301YWAAXX.3 | PR_T_11 | Prostate | Tumor Tissue |
| PR_T_12 | 3064YAAXX.4, | PR_T_12 | Prostate | Tumor Tissue |
| | 30JD2AAXX.6 | | | |
| PR_T_13 | 3064YAAXX.5 | PR_T_13 | Prostate | Metastatic Tissue |
| PR_T_14 | 3064YAAXX.6 | PR_T_14 | Prostate | Metastatic Tissue |
| PR_T_15 | 30Y5NAAXX.3, | PR_T_15 | Prostate | Tumor Tissue |
| | 42B48AAXX.7 | | | |
| PR_T_16 | 30Y5NAAXX.4, | PR_T_16 | Prostate | Tumor Tissue |
| | 42CJFAAXX.2, | | | |
| | 42NY4AAXX.5 | | | |
| PR_T_17 | 30GC3AAXX.3, | PR_T_17 | Prostate | Tumor Tissue |
| | 30Y5NAAXX.6 | | | |
| PR_T_18 | 30Y5NAAXX.7 | PR_T_18 | Prostate | Tumor Tissue |
| PR_T_19 | 30TUEAAXX.5 | PR_T_19 | Prostate | Tumor Tissue |
| PR_T_20 | 30TUEAAXX.6 | PR_T_20 | Prostate | Tumor Tissue |
| PR_T_21 | 30GC3AAXX.5 | PR_T_21 | Prostate | Tumor Tissue |
| PR_T_22 | 30GC3AAXX.6, | PR_T_22 | Prostate | Tumor Tissue |
| | 30JD2AAXX.5 | | | |
| PR_T_23 | 30JD2AAXX.3 | PR_T_23 | Prostate | Tumor Tissue |
| PR_T_24 | 30JD2AAXX.7 | PR_T_24 | Prostate | Tumor Tissue |
| PR_T_25 | 30JD2AAXX.8 | PR_T_25 | Prostate | Tumor Tissue |
| PR_T_27 | 30U00AAXX.4 | PR_T_27 | Prostate | Tumor Tissue |
| PR_T_28 | 30U00AAXX.5, | PR_T_28 | Prostate | Metastatic Tissue |
| | 30U00AAXX.6, | | | |
| | 42B08AAXX.6 | | | |
| PR_T_29 | 30WU2AAXX.1, | PR_T_29 | Prostate | Tumor Tissue |
| | 42B08AAXX.1, | | | |
| | 42CJJAAXX.5 | | | |
| PR_T_30 | 42CJJAAXX.7, | PR_T_30 | Prostate | Benign Tissue |
| | 42NY4AAXX.2 | | | |
| PR_T_31 | 42B48AAXX.2, | PR_T_31 | Prostate | Tumor Tissue |
| | 42PFAAAXX.1 | | | |
| PR_T_32 | 30WU2AAXX.4, | PR_T_32 | Prostate | Tumor Tissue |
| | 42B48AAXX.3 | | | |
| PR_T_33 | 30WU2AAXX.7, | PR_T_33 | Prostate | Benign Tissue |
| | 42CJFAAXX.5 | | | |
| PR_T_34 | 30WU2AAXX.8, | PR_T_34 | Prostate | Tumor Tissue |
| | 42CJFAAXX.4, | | | |
| | 42PFAAAXX.3 | | | |
| PR_T_36 | 42B08AAXX.5 | PR_T_36 | Prostate | Tumor Tissue |
| PR_T_37 | 42B08AAXX.7 | PR_T_37 | Prostate | Tumor Tissue |
| PR_T_38 | 42B08AAXX.8 | PR_T_38 | Prostate | Tumor Tissue |
| PR_T_39 | 42B3YAAXX.4 | PR_T_39 | Prostate | Tumor Tissue |
| PR_T_40 | 42B48AAXX.5 | PR_T_40 | Prostate | Tumor Tissue |
| PR_T_41 | 42B48AAXX.6, | PR_T_41 | Prostate | Tumor Tissue |
| | 42NY4AAXX.4 | | | |
| PR_T_42 | 42CJFAAXX.1 | PR_T_42 | Prostate | Tumor Tissue |
| PR_T_44 | 42CJFAAXX.6 | PR_T_44 | Prostate | Metastatic Tissue |
| PR_T_45 | 42CJFAAXX.7, | PR_T_45 | Prostate | Tumor Tissue |
| | 42P6UAAXX.8, | | | |
| | 42PFAAAXX.4 | | | |
| PR_T_47 | 42CJJAAXX.6, | PR_T_47 | Prostate | Tumor Tissue |
| | 42PF0AAXX.3 | | | |
| PR_T_48 | 42CJJAAXX.8 | PR_T_48 | Prostate | Tumor Tissue |
| PR_T_49 | 42D3NAAXX.1 | PR_T_49 | Prostate | Tumor Tissue |
| PR_T_50 | 42D3NAAXX.2 | PR_T_50 | Prostate | Tumor Tissue |
| PR_T_51 | 42D3NAAXX.3 | PR_T_51 | Prostate | Tumor Tissue |
| PR_T_52 | 42D3NAAXX.6, | PR_T_52 | Prostate | Tumor Tissue |
| | 42P6UAAXX.6 | | | |
| PR_T_53 | 42D3NAAXX.7, | PR_T_53 | Prostate | Tumor Tissue |
| | 42P6UAAXX.7 | | | |
| PR_T_54 | 42TB9AAXX.1 | PR_T_54 | Prostate | Tumor Tissue |
| PR_T_55 | 42TB9AAXX.2 | PR_T_55 | Prostate | Tumor Tissue |
| PR_T_56 | 42TB9AAXX.3 | PR_T_56 | Prostate | Tumor Tissue |
| PR_T_57 | 42TB9AAXX.4, | PR_T_57 | Prostate | Tumor Tissue |
| | 42Y6WAAXX.6 | | | |
| PR_T_58 | 42TB9AAXX.6 | PR_T_58 | Prostate | Tumor Tissue |
| PR_T_59 | 42TB9AAXX.7 | PR_T_59 | Prostate | Tumor Tissue |
| PR_T_60 | 42Y2MAAXX.8 | PR_T_60 | Prostate | Tumor Tissue |
| PR_T_61 | 43336AAXX.1 | PR_T_61 | Prostate | Tumor Tissue |
| PR_T_62 | 43336AAXX.2 | PR_T_62 | Prostate | Tumor Tissue |
| PR_T_63 | 43336AAXX.3 | PR_T_63 | Prostate | Tumor Tissue |
| PR_T_64 | 6137KAAXX.3, | PR_T_64 | Prostate | Metastatic Tissue |
| | 6137KAAXX.4, | | | |
| | 6137LAAXX.6 | | | |
| PR_T_65 | 6137LAAXX.7 | PR_T_65 | Prostate | Tumor Tissue |
| PR_T_66 | 6137LAAXX.8 | PR_T_66 | Prostate | Tumor Tissue |
| SG_T_1 | 43336AAXX.4 | SG_T_1 | Salivary | Tumor Tissue |
| | | | Gland | |
| SG_T_2 | 43336AAXX.5 | SG_T_2 | Salivary | Tumor Tissue |
| | | | Gland | |
| SG_T_3 | 43336AAXX.6 | SG_T_3 | Salivary | Tumor Tissue |
| | | | Gland | |
| SG_T_4 | 43336AAXX.7 | SG_T_4 | Salivary | Tumor Tissue |
| | | | Gland | |

TABLE 2

| Flowcell_lane | Total_Count | PF_Count | hs_ref_chr1 | hs_ref_chr2 | hs_ref_chr3 | hs_ref_chr4 | hs_ref_chr5 |
|---|---|---|---|---|---|---|---|
| 314N5AAXX_6 | 10473424 | 8629317 | 891576 | 303708 | 315512 | 138671 | 224453 |
| 314WGAAXX_4 | 10220916 | 8510626 | 815415 | 383727 | 297329 | 123995 | 297034 |
| 30Y5NAAXX_1 | 21397810 | 10789719 | 1044813 | 501271 | 371407 | 168358 | 358977 |
| 315C1AAXX_1 | 20993016 | 8320198 | 777760 | 372380 | 285462 | 122081 | 274471 |
| 30LUMAAXX_2 | 11963090 | 7629386 | 694396 | 431203 | 326417 | 145063 | 264302 |
| 42AV4AAXX_4 | 23950519 | 18405648 | 1760521 | 831675 | 624394 | 339165 | 596817 |
| 314N5AAXX_5 | 10917232 | 9001619 | 771148 | 477219 | 392899 | 172329 | 307359 |
| 30TYGAAXX_1 | 10401208 | 7749518 | 202168 | 108883 | 89364 | 51464 | 79768 |
| 42PMUAAXX_5 | 10075786 | 8835013 | 814959 | 498501 | 320439 | 149215 | 281021 |
| 42D3NAAXX_4 | 11728387 | 9472843 | 889762 | 367340 | 322203 | 122027 | 227293 |
| 42Y39AAXX_8 | 22686263 | 16753241 | 1615986 | 684121 | 587253 | 243140 | 444007 |
| 42YRVAAXX_2 | 24910111 | 9952386 | 972856 | 462262 | 351827 | 204328 | 349844 |
| 42B20AAXX_2 | 15027956 | 13454580 | 1402697 | 485225 | 387638 | 173940 | 471147 |
| 30TYGAAXX_3 | 9461708 | 6778936 | 279229 | 149660 | 111711 | 85437 | 94044 |
| 42B20AAXX_6 | 15024331 | 13826315 | 1457854 | 486905 | 556446 | 224286 | 347700 |
| 42HAMAAXX_1 | 12153995 | 10924998 | 1210269 | 443524 | 368971 | 188525 | 449786 |
| 42B20AAXX_1 | 20012752 | 16653225 | 1742015 | 777175 | 720316 | 290880 | 512558 |
| 42YRVAAXX_3 | 15502449 | 13197913 | 1184945 | 558602 | 516507 | 209748 | 400480 |
| 42HAMAAXX_2 | 18573826 | 15911042 | 1242358 | 615536 | 555506 | 239122 | 489876 |
| 42YRVAAXX_1 | 24214865 | 8771902 | 873593 | 539427 | 323245 | 146812 | 274181 |
| 30TUEAAXX_3 | 8514234 | 6557403 | 428806 | 251831 | 260722 | 136415 | 180308 |
| 3035BAAXX_3 | 14593087 | 10308363 | 875982 | 386468 | 346520 | 140189 | 358437 |
| 314NNAAXX_3 | 8208384 | 6641235 | 601367 | 308758 | 229249 | 87188 | 207361 |
| 314WAAAXX_3 | 12230961 | 8474905 | 778636 | 392304 | 295037 | 121843 | 262822 |
| 314WAAAXX_5 | 10190342 | 6844572 | 761087 | 314352 | 302378 | 119767 | 214689 |
| 42Y39AAXX_1 | 23513773 | 16997748 | 1996837 | 845432 | 808437 | 331682 | 581896 |
| 30WB6AAXX_7 | 11819695 | 9295846 | 769796 | 316001 | 386746 | 142076 | 322589 |
| 30351AAXX_7 | 14162607 | 8883459 | 960562 | 470335 | 361236 | 216694 | 299455 |
| 314T1AAXX_1 | 12509198 | 7748627 | 818152 | 364685 | 284298 | 161372 | 233107 |
| 30U00AAXX_7 | 17567068 | 9138228 | 158370 | 56711 | 54006 | 25105 | 37817 |
| 30LEJAAXX_7 | 14448057 | 10580431 | 1039634 | 544881 | 517065 | 170280 | 323784 |
| 30TYGAAXX_5 | 10965999 | 6279578 | 340429 | 157019 | 140271 | 91261 | 139012 |
| 314NNAAXX_6 | 12721965 | 9214420 | 748120 | 311262 | 280228 | 144550 | 212740 |
| 3064YAAXX_1 | 13680801 | 10267396 | 1082363 | 464784 | 394853 | 146894 | 289100 |
| 30LF7AAXX_1 | 10074076 | 6427287 | 652955 | 362467 | 308101 | 107944 | 199068 |
| 315CUAAXX_3 | 10946793 | 8901027 | 833789 | 364348 | 238418 | 136556 | 215761 |
| 30LF7AAXX_2 | 10325885 | 7057252 | 708263 | 379082 | 297261 | 142093 | 224288 |
| 30LF7AAXX_3 | 8959909 | 6429978 | 603228 | 346857 | 307479 | 140724 | 211092 |
| 314WAAAXX_4 | 9936387 | 7095676 | 703162 | 286327 | 264486 | 123163 | 256150 |
| 42PMUAAXX_3 | 19054120 | 15939475 | 1597166 | 580652 | 468253 | 243090 | 457503 |
| 6137LAAXX_2 | 22630192 | 17712764 | 169653 | 91691 | 100521 | 61126 | 87522 |
| 30Y6GAAXX_1 | 8043771 | 6860609 | 467768 | 279284 | 229064 | 82281 | 193455 |
| 314NNAAXX_5 | 10823692 | 8336884 | 615226 | 366471 | 249684 | 153798 | 195543 |
| 30Y6GAAXX_3 | 10491542 | 8598099 | 604723 | 368526 | 229126 | 154385 | 201213 |
| 30LUMAAXX_3 | 10135038 | 7202481 | 720394 | 282040 | 379091 | 114522 | 276929 |
| 314WGAAXX_1 | 12754073 | 9789340 | 912346 | 307728 | 407574 | 152807 | 338065 |
| 42HAMAAXX_7 | 10819606 | 9632982 | 820121 | 433856 | 251257 | 147426 | 282843 |
| 314N5AAXX_7 | 10289131 | 8391763 | 857681 | 370661 | 344924 | 214015 | 305391 |
| 301YWAAXX_1 | 16784282 | 14755517 | 1254041 | 731559 | 550897 | 280867 | 424454 |
| 42P6UAAXX_4 | 19556063 | 16107801 | 1371541 | 794583 | 610961 | 309131 | 466080 |
| 301YWAAXX_2 | 15547938 | 14017921 | 1259951 | 711252 | 531236 | 291695 | 434508 |
| 42P6UAAXX_5 | 16096802 | 14031093 | 1254148 | 704316 | 531914 | 293325 | 434091 |
| 301YWAAXX_3 | 15995869 | 14290078 | 1284093 | 685587 | 501233 | 283207 | 384288 |
| 302LVAAXX_2 | 15811517 | 11110453 | 1245685 | 377497 | 312585 | 186718 | 301958 |
| 302LVAAXX_5 | 15661470 | 11158691 | 1055556 | 474996 | 391414 | 203743 | 413507 |
| 302LVAAXX_6 | 15468054 | 10988635 | 1185976 | 476401 | 436448 | 250860 | 436990 |
| 302LVAAXX_7 | 16094827 | 11172977 | 1085306 | 494116 | 415332 | 222285 | 410366 |
| 3035BAAXX_1 | 14440359 | 10322757 | 880960 | 438978 | 353826 | 183989 | 308704 |
| 3035BAAXX_2 | 17310015 | 11763778 | 1143851 | 457215 | 381332 | 227189 | 328779 |
| 3035BAAXX_5 | 18670237 | 11765186 | 1152166 | 558818 | 371277 | 159952 | 278184 |
| 3035BAAXX_6 | 20985359 | 9913182 | 789467 | 365239 | 334377 | 177886 | 240135 |
| 3064YAAXX_2 | 12526653 | 9792955 | 917410 | 445327 | 403053 | 198938 | 312297 |
| 42P6UAAXX_1 | 17158520 | 14658825 | 1385645 | 682127 | 626502 | 318411 | 482559 |
| 3064YAAXX_3 | 11198924 | 9282092 | 834038 | 440760 | 385247 | 189724 | 258161 |
| 42P6UAAXX_2 | 13515172 | 12317092 | 1102386 | 590936 | 519665 | 259519 | 347710 |
| 3064YAAXX_4 | 11599828 | 9642506 | 960275 | 396266 | 350242 | 198843 | 301378 |
| 30JD2AAXX_6 | 8009919 | 5423198 | 151991 | 112014 | 117072 | 60809 | 68853 |
| 3064YAAXX_5 | 12002880 | 9822270 | 805266 | 398736 | 366218 | 237516 | 283800 |
| 3064YAAXX_6 | 11774583 | 9663726 | 860019 | 425566 | 361082 | 211308 | 303153 |
| 3064YAAXX_7 | 10530065 | 8772663 | 794678 | 460881 | 319091 | 172022 | 268034 |
| 42P6UAAXX_3 | 15702879 | 14035929 | 1272427 | 740156 | 523410 | 287309 | 436019 |
| 30650AAXX_1 | 13497088 | 8903956 | 821036 | 453470 | 331926 | 249858 | 296224 |
| 30650AAXX_5 | 20054278 | 9272801 | 966989 | 373318 | 351056 | 130422 | 278925 |
| 30650AAXX_6 | 20008814 | 8795886 | 760539 | 322214 | 263045 | 117229 | 193909 |
| 30650AAXX_7 | 17519050 | 9187752 | 742680 | 340892 | 350967 | 168262 | 276685 |
| 30Y5NAAXX_2 | 13512323 | 9800244 | 974352 | 492091 | 329293 | 126263 | 303278 |
| 42Y6NAAXX_3 | 22968530 | 14883559 | 1027974 | 550009 | 481524 | 184661 | 430583 |
| 30Y5NAAXX_3 | 12542309 | 9148041 | 956907 | 458657 | 388067 | 232475 | 298194 |
| 42B48AAXX_7 | 17098482 | 14028075 | 1315195 | 701374 | 548547 | 315601 | 429237 |
| 30Y5NAAXX_4 | 11008484 | 8565125 | 921367 | 442543 | 404691 | 223552 | 284424 |
| 42CJFAAXX_2 | 13893986 | 12506692 | 1158430 | 603215 | 536013 | 276119 | 385579 |
| 42NY4AAXX_5 | 18282011 | 16107721 | 1539703 | 793992 | 721713 | 375998 | 515399 |
| 30GC3AAXX_3 | 8137365 | 6230727 | 544238 | 317069 | 293688 | 157048 | 231621 |
| 30Y5NAAXX_6 | 13272164 | 9555248 | 1034674 | 462120 | 408619 | 237626 | 339174 |
| 30Y5NAAXX_7 | 10734138 | 8372059 | 1003774 | 441641 | 406259 | 245672 | 288685 |
| 30U00AAXX_1 | 8065406 | 6928557 | 685805 | 423428 | 283985 | 141100 | 216675 |
| 30TUEAAXX_5 | 13187411 | 8126908 | 673665 | 380643 | 369409 | 198346 | 226176 |
| 30TUEAAXX_6 | 11745479 | 7561283 | 640583 | 370543 | 303645 | 159490 | 202695 |
| 30TUEAAXX_2 | 11402334 | 7512728 | 698092 | 388784 | 377375 | 179983 | 249311 |
| 30TUEAAXX_7 | 9234237 | 5434044 | 460773 | 279814 | 274071 | 134502 | 177644 |
| 30TUEAAXX_1 | 15001911 | 7811919 | 697998 | 419403 | 467074 | 200901 | 268534 |
| 30TUEAAXX_8 | 10957052 | 4101941 | 336084 | 198606 | 222053 | 105208 | 127601 |
| 30GC3AAXX_1 | 13060592 | 7190528 | 671121 | 303443 | 275922 | 154519 | 233339 |
| 30GC3AAXX_2 | 10791329 | 7138786 | 634508 | 389447 | 289395 | 102446 | 163582 |
| 30GC3AAXX_5 | 11550703 | 7428665 | 604199 | 367290 | 301939 | 139466 | 229210 |
| 30GC3AAXX_6 | 10332264 | 7000049 | 612422 | 343716 | 291190 | 152426 | 211237 |
| 30JD2AAXX_5 | 7815659 | 5890166 | 488459 | 285642 | 237893 | 118714 | 170698 |
| 30JD2AAXX_3 | 12182940 | 8226904 | 709813 | 411839 | 409623 | 180510 | 249013 |
| 30JD2AAXX_7 | 10466416 | 5811688 | 475953 | 298566 | 306195 | 148984 | 213773 |
| 30JD2AAXX_8 | 7196272 | 3468132 | 293154 | 193173 | 161245 | 96994 | 122015 |
| 30LEJAAXX_2 | 13926365 | 10500585 | 1130570 | 552261 | 400756 | 158876 | 439418 |
| 30U00AAXX_2 | 7480367 | 6313705 | 688748 | 318012 | 232080 | 138290 | 247471 |
| 30U00AAXX_3 | 11613062 | 9558816 | 1008478 | 440628 | 328333 | 240955 | 324088 |
| 30U00AAXX_4 | 11851329 | 9490697 | 780489 | 362583 | 329770 | 167153 | 235063 |
| 30U00AAXX_5 | 10201005 | 8380921 | 147623 | 62407 | 48447 | 29584 | 42648 |
| 30U00AAXX_6 | 12045910 | 9074361 | 136825 | 54617 | 50323 | 34559 | 36806 |
| 42B08AAXX_6 | 13508133 | 11415314 | 937374 | 432230 | 333672 | 208993 | 252573 |
| 30WB6AAXX_5 | 10970972 | 8650247 | 716594 | 388304 | 295593 | 176447 | 316131 |
| 315CUAAXX_4 | 11753778 | 9389202 | 789877 | 418228 | 328048 | 186288 | 333633 |
| 30WB6AAXX_6 | 10258297 | 8167896 | 1010772 | 300716 | 329123 | 118193 | 220171 |
| 30WG6AAXX_4 | 11737010 | 9704540 | 938694 | 323967 | 307603 | 140597 | 235431 |
| 30WG6AAXX_5 | 9610526 | 8208789 | 866851 | 304053 | 274840 | 130026 | 283871 |
| 30WG6AAXX_6 | 11573047 | 9503662 | 660186 | 726030 | 327275 | 143889 | 278469 |
| 30WG6AAXX_7 | 20177509 | 12540404 | 1388616 | 589607 | 377933 | 258825 | 508015 |
| 30WU2AAXX_1 | 15169755 | 13833345 | 1274723 | 676396 | 584363 | 336063 | 386335 |
| 42B08AAXX_1 | 11013257 | 9629325 | 737697 | 375578 | 300907 | 178996 | 214625 |
| 42CJJAAXX_5 | 18008710 | 15188424 | 1387181 | 751141 | 659659 | 366305 | 433529 |
| 42CJJAAXX_7 | 8191717 | 7584480 | 649794 | 377343 | 306254 | 137837 | 196484 |
| 42NY4AAXX_2 | 15839966 | 14331227 | 1269874 | 721430 | 603965 | 270712 | 380629 |
| 42B48AAXX_2 | 8191673 | 7583235 | 605136 | 397668 | 315594 | 159496 | 226487 |
| 42PFAAAXX_1 | 15545422 | 13554764 | 1116144 | 724890 | 585093 | 300196 | 416560 |
| 30WU2AAXX_4 | 9775907 | 9081533 | 859883 | 426875 | 394051 | 195865 | 258261 |
| 42B48AAXX_3 | 16385979 | 14322439 | 1345707 | 690842 | 652473 | 326620 | 427712 |
| 30WU2AAXX_5 | 10599137 | 9822744 | 943891 | 497993 | 463438 | 215538 | 302946 |
| 42B48AAXX_4 | 18364636 | 15313395 | 1462681 | 801286 | 765313 | 361983 | 496959 |
| 30WU2AAXX_6 | 4296772 | 3977109 | 360357 | 201670 | 148772 | 67078 | 99512 |
| 42B48AAXX_8 | 12898304 | 11242142 | 1011881 | 574798 | 435111 | 196802 | 289108 |
| 42PFAAAXX_2 | 7062367 | 6646551 | 605345 | 341407 | 253499 | 116252 | 169258 |
| 30WU2AAXX_7 | 2187981 | 1859890 | 162870 | 89992 | 68473 | 32165 | 50900 |
| 42CJFAAXX_5 | 15950426 | 14245213 | 1260654 | 709583 | 550501 | 263755 | 407352 |
| 30WU2AAXX_8 | 7535290 | 7094073 | 645799 | 327903 | 284731 | 146783 | 207061 |
| 42CJFAAXX_4 | 13074577 | 11855634 | 1062155 | 548869 | 475324 | 246110 | 346824 |
| 42PFAAAXX_3 | 14417900 | 13129950 | 1208846 | 613467 | 535071 | 277825 | 388507 |
| 30Y6GAAXX_2 | 7837225 | 6806921 | 559988 | 226196 | 140903 | 93280 | 208746 |
| 30Y6GAAXX_4 | 10519225 | 8903023 | 858780 | 418186 | 301080 | 153041 | 267808 |
| 30Y6GAAXX_5 | 9494877 | 8174421 | 734960 | 354917 | 300038 | 184316 | 289728 |
| 30Y6GAAXX_6 | 11601281 | 9479970 | 1024260 | 393584 | 351567 | 128541 | 368155 |
| 30Y6GAAXX_7 | 11309743 | 9257186 | 884524 | 371625 | 312238 | 163774 | 304335 |
| 314N5AAXX_1 | 15274660 | 9935485 | 838483 | 377919 | 305868 | 106254 | 214321 |
| 314N5AAXX_2 | 10498158 | 8468256 | 819390 | 399400 | 461360 | 209192 | 266499 |
| 314N5AAXX_3 | 14008114 | 10516608 | 975815 | 474319 | 395273 | 199576 | 298795 |
| 314N5AAXX_4 | 20018598 | 11984485 | 1225878 | 740523 | 439702 | 251948 | 418452 |
| 314NNAAXX_2 | 11352990 | 8504226 | 612711 | 258244 | 284219 | 100522 | 192393 |
| 314NNAAXX_4 | 11761525 | 8633746 | 705760 | 354986 | 308593 | 130094 | 233038 |
| 314RPAAXX_5 | 9434541 | 8108277 | 847130 | 287123 | 217142 | 124652 | 303901 |
| 314RPAAXX_6 | 9606020 | 8168925 | 892814 | 430456 | 310121 | 159273 | 229399 |
| 314RPAAXX_7 | 9030162 | 7798595 | 793837 | 307009 | 244126 | 171070 | 224143 |
| 314T1AAXX_2 | 10813833 | 8464529 | 686939 | 754756 | 371172 | 179098 | 362249 |
| 314WAAAXX_1 | 11881181 | 7879015 | 726139 | 418418 | 276903 | 123602 | 226094 |
| 314WAAAXX_2 | 11640006 | 8203409 | 807749 | 257641 | 314398 | 153194 | 326105 |
| 314WAAAXX_6 | 9932223 | 6246758 | 582081 | 289210 | 241235 | 91108 | 208770 |
| 314WAAAXX_7 | 11782173 | 6637857 | 530631 | 481163 | 301805 | 107355 | 280679 |
| 314WGAAXX_2 | 10115887 | 8436012 | 773699 | 394229 | 435257 | 123880 | 266465 |
| 314WGAAXX_3 | 10534103 | 8668663 | 913085 | 385752 | 371590 | 173533 | 293844 |
| 314WGAAXX_5 | 10280279 | 8664918 | 912614 | 452374 | 345114 | 204099 | 244867 |
| 314WGAAXX_7 | 10112388 | 8483903 | 893203 | 444846 | 315076 | 180678 | 333083 |
| 315C1AAXX_3 | 8656031 | 7039407 | 756252 | 322462 | 236206 | 158271 | 242139 |
| 315C1AAXX_4 | 11351441 | 8064164 | 748904 | 395348 | 283626 | 183601 | 240724 |
| 315CUAAXX_1 | 9779378 | 7798963 | 769486 | 263363 | 264290 | 91681 | 187258 |
| 315CUAAXX_2 | 13323321 | 10379933 | 1224672 | 492927 | 312093 | 155089 | 337578 |
| 315CUAAXX_5 | 16906901 | 11638086 | 1068478 | 601263 | 390493 | 193350 | 301648 |
| 315CUAAXX_6 | 9548547 | 7917384 | 710481 | 300668 | 269351 | 139609 | 242909 |
| 315CUAAXX_7 | 11281870 | 9132553 | 892121 | 452972 | 326344 | 164051 | 271569 |
| 429T4AAXX_1 | 15939075 | 14059890 | 1309531 | 607050 | 447331 | 207079 | 340330 |
| 429T4AAXX_2 | 13990667 | 12759909 | 1196365 | 417265 | 438941 | 161337 | 276014 |
| 429T4AAXX_3 | 16737622 | 14785826 | 1330242 | 516199 | 380405 | 292094 | 349603 |
| 429T4AAXX_4 | 13525603 | 12322606 | 1232750 | 473062 | 536972 | 225871 | 370266 |
| 429T4AAXX_5 | 15186850 | 13654861 | 1551199 | 630496 | 397625 | 239787 | 422647 |
| 429T4AAXX_6 | 14711120 | 13341323 | 1179785 | 700629 | 445884 | 298972 | 392510 |
| 429T4AAXX_7 | 15381935 | 13882984 | 1597386 | 532218 | 487069 | 264736 | 373791 |
| 42AV4AAXX_1 | 21861032 | 17580540 | 1822607 | 963303 | 647168 | 373372 | 584259 |
| 42AV4AAXX_2 | 23702565 | 18625543 | 1823996 | 833158 | 651442 | 376752 | 640555 |
| 42AV4AAXX_3 | 22754813 | 18504760 | 1791279 | 749272 | 710729 | 296998 | 546380 |
| 42AV4AAXX_5 | 23849133 | 18596628 | 1817977 | 990445 | 963362 | 360357 | 489249 |
| 42AV4AAXX_6 | 22506981 | 18367919 | 1925332 | 831857 | 744528 | 380874 | 582584 |
| 42AV4AAXX_7 | 22374610 | 18182294 | 2058632 | 905600 | 934726 | 360986 | 584334 |
| 42B08AAXX_2 | 8158385 | 7363048 | 669649 | 331988 | 214665 | 83674 | 243612 |
| 42B08AAXX_4 | 12412477 | 10593920 | 934590 | 394912 | 383742 | 162400 | 369608 |
| 42B08AAXX_5 | 13929047 | 11559996 | 1070689 | 550750 | 511850 | 279240 | 346186 |
| 42B08AAXX_7 | 14471615 | 12164542 | 1086345 | 577763 | 464233 | 254488 | 361538 |
| 42B08AAXX_8 | 11679239 | 10252280 | 953566 | 402128 | 366667 | 192719 | 337288 |
| 42B20AAXX_5 | 18772149 | 16616393 | 1499398 | 837945 | 744910 | 372394 | 552784 |
| 42NY4AAXX_6 | 17855189 | 15877894 | 1428829 | 802001 | 716209 | 361280 | 529816 |
| 42B3YAAXX_4 | 16090382 | 7806523 | 773858 | 371575 | 357127 | 202273 | 252636 |
| 42B3YAAXX_5 | 13638417 | 8313731 | 863467 | 359334 | 270346 | 150565 | 236363 |
| 42B3YAAXX_6 | 12991081 | 7844191 | 763166 | 395235 | 284009 | 152978 | 252556 |
| 42B3YAAXX_7 | 14131828 | 7884438 | 759833 | 306536 | 255559 | 142103 | 245490 |
| 42B48AAXX_1 | 13515240 | 12122294 | 1105988 | 619101 | 553907 | 274491 | 398241 |
| 42NY4AAXX_3 | 12785238 | 11809596 | 1091212 | 595453 | 528700 | 260108 | 380704 |
| 42B48AAXX_5 | 17474108 | 14583654 | 1360712 | 721888 | 686371 | 359946 | 456326 |
| 42B48AAXX_6 | 13434541 | 11883818 | 1120262 | 632036 | 482059 | 287759 | 367484 |
| 42NY4AAXX_4 | 12823878 | 11879138 | 1146548 | 633264 | 482284 | 289870 | 367396 |
| 42HAMAAXX_3 | 11686610 | 10676671 | 1147946 | 449262 | 340897 | 198137 | 360224 |
| 42HAMAAXX_5 | 14004607 | 12671255 | 983341 | 737891 | 365531 | 198535 | 369215 |
| 42HAMAAXX_6 | 12509418 | 11257837 | 956466 | 419541 | 477519 | 204899 | 401279 |
| 42CJFAAXX_1 | 10906620 | 9473417 | 863069 | 461978 | 357951 | 195126 | 287070 |
| 42CJFAAXX_6 | 9822464 | 9038499 | 837698 | 418641 | 282846 | 138916 | 253313 |
| 42CJFAAXX_7 | 10284465 | 9428987 | 813309 | 406280 | 311380 | 177889 | 267727 |
| 42P6UAAXX_8 | 1115689 | 936249 | 80533 | 39820 | 29652 | 16668 | 25849 |
| 42PFAAAXX_4 | 14600476 | 13242928 | 1186545 | 569340 | 436239 | 246695 | 375567 |
| 42CJJAAXX_1 | 18403563 | 15124338 | 1433081 | 678575 | 462464 | 377207 | 338370 |
| 42CJJAAXX_2 | 15464562 | 13813096 | 944585 | 483053 | 444698 | 352371 | 488218 |
| 42CJJAAXX_3 | 17817401 | 15322024 | 976323 | 542037 | 433830 | 454729 | 563106 |
| 42CJJAAXX_4 | 17729184 | 15191542 | 1116289 | 627812 | 488093 | 329655 | 473110 |
| 42CJJAAXX_6 | 5612280 | 5249645 | 476015 | 246037 | 222610 | 106891 | 146879 |
| 42PF0AAXX_3 | 8754238 | 8109134 | 758564 | 378964 | 344423 | 164030 | 225162 |
| 42CJJAAXX_8 | 16559593 | 14204491 | 1265888 | 666554 | 613521 | 314148 | 435995 |
| 42D3NAAXX_1 | 9656221 | 7781206 | 687419 | 328833 | 320199 | 184017 | 258760 |
| 42D3NAAXX_2 | 10804682 | 8614808 | 742875 | 330895 | 297992 | 169850 | 270717 |
| 42D3NAAXX_3 | 13239451 | 10305382 | 870309 | 437466 | 421349 | 216541 | 293122 |
| 42D3NAAXX_5 | 14413573 | 10102958 | 918533 | 488821 | 446197 | 210824 | 309550 |
| 42D3NAAXX_6 | 13276901 | 10323732 | 947868 | 474149 | 384611 | 210468 | 311543 |
| 42P6UAAXX_6 | 13975560 | 12591851 | 1140353 | 578112 | 467835 | 259940 | 378914 |
| 42D3NAAXX_7 | 13152559 | 9949944 | 899207 | 479861 | 400232 | 227324 | 283752 |
| 42P6UAAXX_7 | 14874983 | 13165443 | 1176272 | 625615 | 525627 | 299034 | 371458 |
| 42PF0AAXX_6 | 17138265 | 14777280 | 1418878 | 448287 | 363094 | 198359 | 575162 |
| 42PF0AAXX_7 | 10259719 | 9437980 | 919422 | 484822 | 340461 | 179479 | 316420 |
| 42PFAAAXX_5 | 16312993 | 14469668 | 1310683 | 438942 | 537294 | 344893 | 439485 |
| 42PFAAAXX_6 | 13802729 | 12368674 | 1049735 | 412077 | 422490 | 281936 | 394436 |
| 42PMUAAXX_6 | 12960276 | 10714939 | 1002883 | 328899 | 413668 | 249243 | 327093 |
| 42PMUAAXX_7 | 11527879 | 9643473 | 933929 | 297253 | 372202 | 235583 | 300690 |
| 42PFAAAXX_7 | 16812413 | 14565562 | 1548634 | 571000 | 458289 | 260402 | 320628 |
| 42PFAAAXX_8 | 12203177 | 10966281 | 1011379 | 482826 | 379777 | 216524 | 354063 |
| 42PMUAAXX_1 | 12941644 | 11722220 | 1236755 | 563809 | 438349 | 249205 | 475384 |
| 42PMUAAXX_2 | 10348488 | 9349822 | 782179 | 344890 | 247864 | 141994 | 386937 |
| 42PMUAAXX_4 | 8997114 | 8104333 | 744789 | 377994 | 323164 | 173304 | 308421 |
| 42PMUAAXX_8 | 8062434 | 6828931 | 490714 | 316631 | 229390 | 239851 | 224228 |
| 42RMPAAXX_1 | 20033861 | 16747158 | 1556530 | 773564 | 594964 | 358760 | 578347 |
| 42RMPAAXX_2 | 23510465 | 17757415 | 1772675 | 933603 | 640407 | 466324 | 700097 |
| 42RMPAAXX_3 | 23416048 | 17174723 | 1516250 | 743252 | 469269 | 323804 | 872020 |
| 42RMPAAXX_4 | 18907817 | 16067100 | 967331 | 418166 | 331287 | 172120 | 451421 |
| 42RMPAAXX_5 | 12587943 | 11512967 | 1134898 | 569225 | 410965 | 282471 | 366417 |
| 42RMPAAXX_6 | 10337171 | 9518597 | 1027957 | 406748 | 407610 | 153305 | 259925 |
| 42RMPAAXX_7 | 11314787 | 10294632 | 949415 | 635386 | 315153 | 215812 | 445715 |
| 42RMPAAXX_8 | 16118019 | 13865366 | 1368208 | 641133 | 567249 | 225113 | 457413 |
| 42RYFAAXX_1 | 6701387 | 6091407 | 541107 | 293672 | 238648 | 118329 | 191220 |
| 42RYFAAXX_2 | 8535025 | 7866350 | 682339 | 529480 | 262251 | 172022 | 256440 |
| 42RYFAAXX_3 | 9335393 | 8595830 | 789001 | 478405 | 249077 | 147088 | 289252 |
| 42RYFAAXX_4 | 7205785 | 6679446 | 754872 | 290028 | 194518 | 94252 | 162201 |
| 42RYFAAXX_5 | 8461912 | 7709883 | 734326 | 414432 | 261902 | 143368 | 181940 |
| 42RYFAAXX_6 | 16269578 | 13707045 | 1261556 | 868619 | 404518 | 242451 | 344173 |
| 42RYFAAXX_7 | 7776934 | 7089245 | 282746 | 129393 | 78958 | 38974 | 85200 |
| 42RYFAAXX_8 | 8657409 | 7754964 | 653480 | 398699 | 203431 | 129398 | 178850 |
| 42TA8AAXX_1 | 12473784 | 10904533 | 1017652 | 464271 | 387464 | 198216 | 412143 |
| 42TA8AAXX_2 | 14875828 | 12863744 | 1195481 | 544252 | 454581 | 231571 | 480735 |
| 42TA8AAXX_3 | 13701002 | 12150298 | 1129204 | 509613 | 426204 | 215722 | 450639 |
| 42TA8AAXX_4 | 520303 | 384462 | 34686 | 15155 | 12869 | 6333 | 13369 |
| 42TA8AAXX_5 | 16602477 | 14187743 | 1318484 | 611480 | 507468 | 260041 | 540095 |
| 42TA8AAXX_6 | 22272421 | 15765949 | 1474838 | 713316 | 581688 | 307987 | 628839 |
| 42TA8AAXX_7 | 14502277 | 12804352 | 1190867 | 538455 | 450340 | 227165 | 474658 |
| 42TB9AAXX_1 | 22151003 | 17881940 | 1699690 | 960954 | 884351 | 502755 | 639114 |
| 42TB9AAXX_2 | 11690336 | 10736010 | 978850 | 501275 | 385239 | 178334 | 287577 |
| 42TB9AAXX_3 | 20968245 | 17239720 | 1613626 | 863881 | 822572 | 428066 | 597692 |
| 42TB9AAXX_4 | 9260374 | 8674521 | 836000 | 440763 | 430637 | 226398 | 292154 |
| 42Y6WAAXX_6 | 16674284 | 13229893 | 1273136 | 664680 | 653155 | 338590 | 441620 |
| 42TB9AAXX_6 | 20238311 | 16892184 | 1561550 | 878083 | 756261 | 426556 | 557114 |
| 42TB9AAXX_7 | 19388639 | 16494766 | 1535474 | 846647 | 689021 | 401034 | 552658 |
| 42TBWAAXX_1 | 16584680 | 14517595 | 726201 | 321319 | 347256 | 149575 | 231412 |
| 42TBWAAXX_2 | 18974162 | 16027182 | 1554554 | 851116 | 437697 | 308159 | 739300 |
| 42TBWAAXX_3 | 20151660 | 16516878 | 1185446 | 567018 | 379973 | 215434 | 419872 |
| 42TBWAAXX_4 | 20228188 | 16739794 | 1403499 | 634072 | 450046 | 274738 | 598480 |
| 42TBWAAXX_5 | 17968130 | 15580317 | 870220 | 337559 | 224358 | 140758 | 511678 |
| 42TBWAAXX_6 | 9163107 | 8493696 | 435402 | 262769 | 220981 | 123200 | 208525 |
| 42Y6WAAXX_1 | 10658467 | 9753799 | 494391 | 298198 | 251661 | 139645 | 239657 |
| 42TBWAAXX_7 | 11090982 | 10171173 | 767953 | 347304 | 243158 | 141083 | 500496 |
| 42TBWAAXX_8 | 16715519 | 14772069 | 788617 | 273152 | 414265 | 220604 | 448624 |
| 42TCBAAXX_1 | 12389721 | 11278312 | 952126 | 338469 | 343188 | 153767 | 217768 |
| 42TCBAAXX_2 | 15562144 | 13752505 | 899997 | 419053 | 315061 | 115218 | 251296 |
| 42TCBAAXX_3 | 19115098 | 15793020 | 1073995 | 474565 | 341808 | 135870 | 298890 |
| 42TCBAAXX_4 | 16688619 | 14543000 | 1652962 | 482562 | 395835 | 214074 | 369564 |
| 42TCBAAXX_5 | 17240300 | 14941820 | 1577955 | 807155 | 522672 | 276990 | 427220 |
| 42TCBAAXX_6 | 10968754 | 9794031 | 579541 | 226658 | 198340 | 111846 | 185661 |
| 42TCBAAXX_7 | 11136557 | 8132925 | 889501 | 377617 | 316153 | 176420 | 261728 |
| 42TCBAAXX_8 | 14686845 | 8218867 | 883457 | 432825 | 332924 | 196097 | 304479 |
| 42TCLAAXX_1 | 2471336 | 2162651 | 220394 | 92889 | 81196 | 45924 | 81098 |
| 42Y39AAXX_2 | 22779225 | 17872799 | 1927753 | 821996 | 710057 | 415353 | 705322 |
| 42TCLAAXX_3 | 2222706 | 1986847 | 185319 | 98620 | 73584 | 40794 | 71347 |
| 42Y39AAXX_5 | 23078547 | 18126523 | 1739953 | 947445 | 716172 | 413628 | 684698 |
| 42TCLAAXX_4 | 5883815 | 5501174 | 547028 | 246681 | 201168 | 112288 | 222997 |
| 42Y39AAXX_6 | 25259413 | 16897050 | 1750498 | 823942 | 660557 | 400054 | 705044 |
| 42TCLAAXX_5 | 4729192 | 4440425 | 405425 | 185693 | 164125 | 79873 | 180211 |
| 42Y39AAXX_7 | 25099038 | 17028028 | 1597652 | 786652 | 673627 | 358122 | 704967 |
| 42TG0AAXX_2 | 18466720 | 5221623 | 397922 | 189757 | 145905 | 70083 | 131594 |
| 42TG0AAXX_3 | 22893676 | 14050183 | 1535569 | 454327 | 564018 | 325463 | 470999 |
| 42TG0AAXX_4 | 17976011 | 14928885 | 1872644 | 558444 | 645281 | 265094 | 472316 |
| 42TG0AAXX_5 | 11340090 | 10372700 | 996297 | 458948 | 284652 | 190856 | 286832 |
| 42TG0AAXX_6 | 15595089 | 13788146 | 1334309 | 502706 | 420009 | 232833 | 378000 |
| 42TG0AAXX_7 | 13268496 | 11972830 | 94942 | 37105 | 10177 | 6437 | 74747 |
| 42TG0AAXX_8 | 19950922 | 16033490 | 1352461 | 615512 | 488471 | 300931 | 406025 |
| 42Y2MAAXX_1 | 20654376 | 16879195 | 1696591 | 1013019 | 749705 | 456404 | 619353 |
| 42Y2MAAXX_2 | 21654676 | 16871003 | 1727483 | 1049221 | 785729 | 445061 | 689070 |
| 42Y2MAAXX_3 | 21951518 | 17575406 | 1897352 | 1099021 | 829304 | 484270 | 653253 |
| 42Y2MAAXX_4 | 21524788 | 17860772 | 1813365 | 1159726 | 813791 | 434011 | 751776 |
| 42Y2MAAXX_5 | 23084637 | 17934432 | 1913658 | 961820 | 796950 | 440431 | 667825 |
| 42Y2MAAXX_6 | 22865652 | 17511574 | 1716501 | 936793 | 779574 | 502509 | 686916 |
| 42Y2MAAXX_7 | 24399137 | 16610914 | 1681784 | 874618 | 765095 | 426173 | 667612 |
| 42Y2MAAXX_8 | 22139173 | 17564874 | 1709419 | 981684 | 931657 | 540364 | 659794 |
| 42Y39AAXX_3 | 23377121 | 17984167 | 1796870 | 1104424 | 673165 | 374514 | 695733 |
| 42Y39AAXX_4 | 24168890 | 17944184 | 1809641 | 1113916 | 677965 | 380425 | 696029 |
| 42Y6NAAXX_1 | 23389623 | 17275039 | 1259608 | 775079 | 550840 | 413550 | 536373 |
| 42Y6NAAXX_2 | 23264657 | 16085036 | 1191942 | 740770 | 604991 | 274437 | 648394 |
| 42Y6NAAXX_4 | 24444260 | 16752359 | 1209213 | 665726 | 632184 | 308495 | 714865 |
| 42Y6NAAXX_5 | 23721711 | 17004306 | 1257973 | 724474 | 502394 | 217832 | 537260 |
| 42Y6NAAXX_6 | 23626967 | 16833388 | 1147987 | 666556 | 490533 | 259158 | 632758 |
| 42Y6NAAXX_7 | 25247635 | 16094433 | 974839 | 685631 | 528842 | 283192 | 610160 |
| 42Y6NAAXX_8 | 24998330 | 15750336 | 1099139 | 705191 | 620226 | 247568 | 570458 |
| 42Y6WAAXX_2 | 12843319 | 11758026 | 1240403 | 636734 | 460547 | 305853 | 459691 |
| 42YMTAAXX_2 | 9117974 | 8307471 | 874869 | 456278 | 327315 | 218646 | 327761 |
| 42Y6WAAXX_3 | 17169930 | 14930941 | 1333028 | 851555 | 566657 | 291195 | 454512 |
| 42YMTAAXX_4 | 7277149 | 6738287 | 600082 | 386106 | 256396 | 130592 | 204929 |
| 42Y6WAAXX_4 | 16931356 | 14158319 | 801337 | 512630 | 282979 | 188851 | 309129 |
| 42YMTAAXX_8 | 8254726 | 7616608 | 425822 | 272243 | 152258 | 101555 | 165063 |
| 42YMMAAXX_1 | 27179622 | 15581828 | 1675836 | 880656 | 650217 | 490900 | 579750 |
| 42YMMAAXX_2 | 23960796 | 18522948 | 2089514 | 1129734 | 793599 | 431616 | 598650 |
| 42YMMAAXX_8 | 24421718 | 17527277 | 1932141 | 1015767 | 723755 | 385613 | 542821 |
| 42YMMAAXX_3 | 24240580 | 18639130 | 2143986 | 1004284 | 704081 | 444993 | 655547 |
| 42YMMAAXX_4 | 20218805 | 16951076 | 1760218 | 851693 | 610087 | 398848 | 567581 |
| 42YMMAAXX_5 | 25629955 | 17702630 | 1637730 | 1075193 | 696320 | 557477 | 942674 |
| 42YMMAAXX_6 | 19760882 | 16543416 | 1723093 | 1165268 | 605505 | 444307 | 562840 |
| 42YMMAAXX_7 | 22053981 | 17771472 | 1569047 | 990113 | 601965 | 420963 | 570341 |
| 42YMTAAXX_1 | 15974539 | 13197439 | 1474163 | 550133 | 351949 | 182001 | 309870 |
| 42YMTAAXX_3 | 12598585 | 11238842 | 1138764 | 552669 | 387041 | 219695 | 369448 |
| 42YMTAAXX_5 | 10122550 | 9282923 | 1065361 | 371202 | 378447 | 197088 | 264311 |
| 42YMTAAXX_6 | 20490262 | 16213794 | 1719054 | 1096986 | 632243 | 319200 | 507926 |
| 42YMTAAXX_7 | 9777093 | 8955815 | 818168 | 442925 | 347577 | 215036 | 338210 |
| 43336AAXX_1 | 41182928 | 29199424 | 3282485 | 2494181 | 2054429 | 1656621 | 1664805 |
| 43336AAXX_2 | 30427778 | 26000779 | 2942868 | 1848901 | 1646668 | 980298 | 1231858 |
| 43336AAXX_3 | 36695581 | 29692597 | 3180433 | 2459801 | 2038004 | 1643863 | 1680349 |
| 43336AAXX_4 | 33685866 | 28233404 | 3152312 | 2262058 | 1891706 | 1416895 | 1504660 |
| 43336AAXX_5 | 34123946 | 29037934 | 2841271 | 2369519 | 1902925 | 1703651 | 1611418 |
| 43336AAXX_6 | 36930819 | 29970635 | 3387754 | 2603541 | 2135972 | 1821332 | 1785740 |
| 43336AAXX_7 | 22592319 | 16434252 | 1363626 | 557602 | 432831 | 208094 | 440776 |
| 6137KAAXX_3 | 28993103 | 25436076 | 1679278 | 1016513 | 1276058 | 522583 | 788058 |
| 6137KAAXX_4 | 28637234 | 25227576 | 1678615 | 1015867 | 1275152 | 522414 | 787507 |
| 6137LAAXX_6 | 23039606 | 17612275 | 322328 | 246936 | 321464 | 119934 | 171343 |
| 6137KAAXX_6 | 37604910 | 28160358 | 2047799 | 1044930 | 693878 | 473279 | 706830 |
| 6137KAAXX_7 | 35375632 | 28709290 | 1703702 | 965900 | 692074 | 416504 | 1147051 |
| 6137KAAXX_8 | 26564268 | 23482778 | 2065015 | 1097025 | 894858 | 537824 | 1004032 |
| 6137LAAXX_7 | 24386843 | 18168576 | 1040190 | 1031400 | 857144 | 832554 | 757794 |
| 6137LAAXX_8 | 22519162 | 14829699 | 805927 | 718134 | 583657 | 550574 | 509443 |
| 61VFCAAXX_1 | 34016045 | 26772791 | 1805523 | 1137119 | 948378 | 625058 | 893217 |
| 61VFCAAXX_2 | 32848790 | 27315237 | 1863607 | 1181478 | 899219 | 539191 | 830998 |
| 61VFCAAXX_3 | 35034316 | 28213465 | 2248200 | 1408378 | 996794 | 678894 | 888057 |
| 61VFCAAXX_6 | 35584119 | 27477193 | 1976720 | 1242402 | 932723 | 651202 | 852967 |
| 62837AAXX_1 | 36322317 | 28954702 | 2455261 | 1112961 | 969389 | 554721 | 1145162 |
| 62837AAXX_2 | 36219937 | 29857852 | 2853516 | 1377353 | 1159360 | 708790 | 1178004 |
| 62837AAXX_3 | 38874310 | 29936016 | 2656683 | 1392016 | 1207580 | 658045 | 1144846 |
| 62837AAXX_4 | 41201556 | 25045035 | 2274218 | 1190562 | 970996 | 636507 | 1044171 |
| 62837AAXX_7 | 25030870 | 22199555 | 594091 | 423952 | 303604 | 221777 | 304797 |
| 62837AAXX_8 | 26730419 | 22967257 | 1958150 | 1043133 | 854667 | 506816 | 927617 |
| 6286PAAXX_1 | 28649741 | 25357315 | 2059856 | 1046117 | 868482 | 513959 | 936188 |
| 6286PAAXX_2 | 28358701 | 25406461 | 2225381 | 1093627 | 905384 | 538903 | 954925 |
| 6286PAAXX_3 | 30623502 | 26445894 | 1710145 | 1175390 | 819241 | 394364 | 777425 |
| 6286PAAXX_4 | 29595585 | 25749153 | 1858438 | 1073817 | 785363 | 477398 | 756183 |
| 6286PAAXX_5 | 28981686 | 25766181 | 1841538 | 1058166 | 868902 | 582438 | 744799 |
| 6286PAAXX_6 | 37228463 | 29351512 | 2257432 | 1251118 | 944465 | 555260 | 1074387 |
| 6286PAAXX_7 | 31279053 | 27035256 | 1862497 | 1152696 | 695624 | 526807 | 729312 |
| 6286PAAXX_8 | 31715233 | 26698158 | 1939233 | 1141192 | 953710 | 534387 | 901020 |

| Flowcell_lane | hs_ref_chr6 | hs_ref_chr7 | hs_ref_chr8 | hs_ref_chr9 | hs_ref_chr10 | hs_ref_chr11 | hs_ref_chr12 |
|---|---|---|---|---|---|---|---|
| 314N5AAXX_6 | 264975 | 321506 | 241835 | 248311 | 254544 | 564318 | 499437 |
| 314WGAAXX_4 | 282292 | 350053 | 163744 | 223864 | 314345 | 439785 | 486065 |
| 30Y5NAAXX_1 | 367214 | 366695 | 175575 | 203872 | 421652 | 700256 | 545133 |
| 315C1AAXX_1 | 296583 | 308238 | 138256 | 172248 | 325590 | 589946 | 450261 |
| 30LUMAAXX_2 | 260364 | 314083 | 307589 | 229131 | 25167 | 37696 | 423698 |
| 42AV4AAXX_4 | 625550 | 645612 | 511873 | 515508 | 541101 | 1095736 | 1317286 |
| 314N5AAXX_5 | 268207 | 341505 | 236600 | 202886 | 258494 | 536613 | 699681 |
| 30TYGAAXX_1 | 70720 | 77132 | 55781 | 58634 | 62504 | 181300 | 110012 |
| 42PMUAAXX_5 | 308256 | 260381 | 195290 | 217130 | 236110 | 463113 | 471461 |
| 42D3NAAXX_4 | 320274 | 279844 | 268952 | 182239 | 255978 | 569816 | 503637 |
| 42Y39AAXX_8 | 576951 | 468034 | 449982 | 310194 | 477928 | 963074 | 890638 |
| 42YRVAAXX_2 | 312636 | 319310 | 313758 | 224050 | 353283 | 432263 | 584077 |
| 42B20AAXX_2 | 317956 | 483593 | 251201 | 266768 | 327693 | 640468 | 904642 |
| 30TYGAAXX_3 | 109302 | 123748 | 67208 | 114189 | 74776 | 176534 | 123140 |
| 42B20AAXX_6 | 413963 | 464379 | 626530 | 442287 | 364328 | 881581 | 708353 |
| 42HAMAAXX_1 | 338298 | 398566 | 264551 | 302246 | 360651 | 594724 | 614349 |
| 42B20AAXX_1 | 871540 | 611610 | 450084 | 377730 | 488422 | 757497 | 662698 |
| 42YRVAAXX_3 | 357546 | 378963 | 363690 | 317401 | 271485 | 686573 | 833088 |
| 42HAMAAXX_2 | 463028 | 797212 | 467206 | 365731 | 467293 | 801463 | 855378 |
| 42YRVAAXX_1 | 263315 | 264060 | 188584 | 168153 | 232845 | 368123 | 462039 |
| 30TUEAAXX_3 | 171775 | 256122 | 190774 | 217501 | 21602 | 52235 | 265753 |
| 3035BAAXX_3 | 365804 | 288874 | 305701 | 199860 | 277424 | 507950 | 494303 |
| 314NNAAXX_3 | 212550 | 286794 | 197188 | 167958 | 182089 | 372361 | 313198 |
| 314WAAAXX_3 | 270056 | 375289 | 249590 | 205741 | 239452 | 475974 | 384771 |
| 314WAAAXX_5 | 236315 | 213935 | 264108 | 218225 | 184189 | 338770 | 356811 |
| 42Y39AAXX_1 | 605720 | 519210 | 649198 | 513136 | 501837 | 828660 | 876644 |
| 30WB6AAXX_7 | 271895 | 257163 | 335224 | 260003 | 197964 | 475979 | 424375 |
| 30351AAXX_7 | 310742 | 280146 | 223762 | 241207 | 329451 | 493113 | 510900 |
| 314T1AAXX_1 | 247371 | 227278 | 182542 | 204534 | 265793 | 419431 | 434031 |
| 30U00AAXX_7 | 47768 | 55843 | 42322 | 46912 | 53898 | 133930 | 68591 |
| 30LEJAAXX_7 | 298924 | 398876 | 346761 | 325513 | 36815 | 43897 | 505637 |
| 30TYGAAXX_5 | 136607 | 101860 | 100404 | 132992 | 89001 | 259365 | 149377 |
| 314NNAAXX_6 | 244973 | 341652 | 185424 | 243543 | 174684 | 417655 | 521831 |
| 3064YAAXX_1 | 291595 | 295445 | 251652 | 178341 | 418689 | 502820 | 575469 |
| 30LF7AAXX_1 | 172619 | 206216 | 224252 | 125489 | 58108 | 43641 | 280385 |
| 315CUAAXX_3 | 177663 | 308961 | 239945 | 235892 | 198356 | 672787 | 515163 |
| 30LF7AAXX_2 | 210113 | 260835 | 191786 | 194111 | 32710 | 54686 | 252507 |
| 30LF7AAXX_3 | 202367 | 249767 | 173140 | 167547 | 40741 | 58531 | 212755 |
| 314WAAAXX_4 | 256171 | 238988 | 189528 | 221558 | 169986 | 387680 | 361473 |
| 42PMUAAXX_3 | 410604 | 460208 | 528179 | 442031 | 657261 | 826793 | 601630 |
| 6137LAAXX_2 | 66939 | 62895 | 48703 | 63120 | 51804 | 86396 | 96126 |
| 30Y6GAAXX_1 | 230541 | 219908 | 181906 | 188641 | 164959 | 474273 | 277814 |
| 314NNAAXX_5 | 285692 | 245091 | 204696 | 244444 | 228396 | 450764 | 543358 |
| 30Y6GAAXX_3 | 272050 | 245692 | 200954 | 215397 | 202382 | 473227 | 502671 |
| 30LUMAAXX_3 | 204330 | 272815 | 233750 | 209946 | 27636 | 45785 | 351876 |
| 314WGAAXX_1 | 318803 | 344542 | 241743 | 291706 | 292956 | 685107 | 564684 |
| 42HAMAAXX_7 | 238387 | 448840 | 264500 | 195586 | 322180 | 543160 | 578018 |
| 314N5AAXX_7 | 201251 | 342687 | 208478 | 214485 | 325405 | 599559 | 500911 |
| 301YWAAXX_1 | 445588 | 495576 | 335975 | 415173 | 384449 | 759003 | 643076 |
| 42P6UAAXX_4 | 484824 | 527271 | 359791 | 439715 | 426878 | 816388 | 702261 |
| 301YWAAXX_2 | 478653 | 472813 | 317408 | 396153 | 439615 | 810545 | 689456 |
| 42P6UAAXX_5 | 473675 | 466083 | 314535 | 389971 | 438539 | 802298 | 683154 |
| 301YWAAXX_3 | 428675 | 490731 | 283888 | 436502 | 370537 | 820846 | 570078 |
| 302LVAAXX_2 | 443511 | 472528 | 292950 | 364310 | 366007 | 748232 | 528688 |
| 302LVAAXX_5 | 459133 | 401671 | 202911 | 253539 | 313491 | 487548 | 617593 |
| 302LVAAXX_6 | 503833 | 329551 | 231434 | 260230 | 358080 | 510659 | 681875 |
| 302LVAAXX_7 | 469624 | 385193 | 202237 | 273820 | 312055 | 541024 | 656601 |
| 3035BAAXX_1 | 327882 | 361924 | 217577 | 344418 | 342492 | 567754 | 618044 |
| 3035BAAXX_2 | 330242 | 307961 | 389238 | 229626 | 367016 | 751618 | 606554 |
| 3035BAAXX_5 | 283928 | 340202 | 294669 | 298182 | 410299 | 710242 | 742177 |
| 3035BAAXX_6 | 298446 | 247828 | 462192 | 247701 | 260466 | 516314 | 566127 |
| 3064YAAXX_2 | 309732 | 323042 | 216951 | 259188 | 313405 | 498013 | 450847 |
| 42P6UAAXX_1 | 473068 | 481219 | 328934 | 382067 | 482516 | 737601 | 670628 |
| 3064YAAXX_3 | 303827 | 302089 | 195521 | 252644 | 270589 | 490033 | 395158 |
| 42P6UAAXX_2 | 405908 | 400823 | 262842 | 333333 | 362140 | 647779 | 519236 |
| 3064YAAXX_4 | 292987 | 318652 | 205215 | 243541 | 224792 | 473949 | 415811 |
| 30JD2AAXX_6 | 62598 | 71790 | 64871 | 52519 | 37460 | 41413 | 61251 |
| 3064YAAXX_5 | 265964 | 277287 | 248816 | 252078 | 180054 | 483447 | 413565 |
| 3064YAAXX_6 | 330972 | 289805 | 186233 | 239125 | 236711 | 483646 | 442255 |
| 3064YAAXX_7 | 276697 | 305954 | 185137 | 256324 | 272059 | 500187 | 401526 |
| 42P6UAAXX_3 | 448983 | 490110 | 299556 | 404562 | 438999 | 788165 | 641300 |
| 30650AAXX_1 | 264597 | 265584 | 216393 | 160116 | 321589 | 467155 | 519468 |
| 30650AAXX_5 | 288151 | 263128 | 268647 | 170985 | 281014 | 599217 | 515936 |
| 30650AAXX_6 | 251739 | 248453 | 196553 | 176106 | 224377 | 490964 | 431677 |
| 30650AAXX_7 | 277741 | 251914 | 261314 | 234722 | 242580 | 566219 | 555313 |
| 30Y5NAAXX_2 | 347334 | 403034 | 255930 | 266400 | 281347 | 617565 | 499883 |
| 42Y6NAAXX_3 | 508716 | 607672 | 322689 | 328992 | 377142 | 607565 | 597670 |
| 30Y5NAAXX_3 | 315829 | 308358 | 198894 | 251165 | 291465 | 620228 | 420428 |
| 42B48AAXX_7 | 468654 | 484579 | 301524 | 414146 | 417449 | 840389 | 661094 |
| 30Y5NAAXX_4 | 305841 | 304739 | 200229 | 241731 | 283033 | 553249 | 403798 |
| 42CJFAAXX_2 | 433250 | 430419 | 276575 | 351255 | 361505 | 703194 | 594470 |
| 42NY4AAXX_5 | 564012 | 550489 | 360946 | 445909 | 487037 | 902731 | 774753 |
| 30GC3AAXX_3 | 212859 | 233668 | 156932 | 182872 | 71621 | 104303 | 220206 |
| 30Y5NAAXX_6 | 344889 | 331212 | 208658 | 279081 | 277027 | 620677 | 421243 |
| 30Y5NAAXX_7 | 248229 | 292112 | 178454 | 193511 | 313631 | 557734 | 330780 |
| 30U00AAXX_1 | 277592 | 250237 | 177572 | 189719 | 204194 | 324344 | 297766 |
| 30TUEAAXX_5 | 252724 | 383844 | 184080 | 228496 | 90866 | 132072 | 294065 |
| 30TUEAAXX_6 | 209617 | 277751 | 185704 | 212635 | 85552 | 118110 | 246549 |
| 30TUEAAXX_2 | 235001 | 293170 | 192652 | 197426 | 93413 | 96128 | 263132 |
| 30TUEAAXX_7 | 195835 | 223864 | 143099 | 147529 | 68304 | 76261 | 190662 |
| 30TUEAAXX_1 | 276009 | 303630 | 226767 | 212817 | 82313 | 99296 | 282370 |
| 30TUEAAXX_8 | 133988 | 147597 | 101579 | 103398 | 39650 | 48491 | 138071 |
| 30GC3AAXX_1 | 259946 | 253037 | 221524 | 137719 | 64848 | 88307 | 324084 |
| 30GC3AAXX_2 | 251463 | 248698 | 228695 | 183951 | 79007 | 96013 | 239489 |
| 30GC3AAXX_5 | 259762 | 259913 | 182021 | 196821 | 65739 | 97728 | 265393 |
| 30GC3AAXX_6 | 207679 | 227980 | 251606 | 167595 | 68739 | 91133 | 216685 |
| 30JD2AAXX_5 | 167987 | 183373 | 209140 | 135607 | 47667 | 63675 | 179627 |
| 30JD2AAXX_3 | 250708 | 320768 | 211871 | 233914 | 80860 | 100612 | 279067 |
| 30JD2AAXX_7 | 168371 | 215463 | 150382 | 138707 | 76904 | 90883 | 162154 |
| 30JD2AAXX_8 | 104100 | 123952 | 89025 | 82540 | 44299 | 52559 | 111423 |
| 30LEJAAXX_2 | 336630 | 358500 | 365015 | 383995 | 70246 | 72288 | 461037 |
| 30U00AAXX_2 | 335874 | 199501 | 124862 | 147489 | 206279 | 361455 | 366570 |
| 30U00AAXX_3 | 289757 | 323428 | 419765 | 234241 | 326349 | 603359 | 491045 |
| 30U00AAXX_4 | 327336 | 288153 | 176723 | 240365 | 231779 | 474561 | 371959 |
| 30U00AAXX_5 | 43408 | 62670 | 30944 | 41669 | 41394 | 136501 | 63564 |
| 30U00AAXX_6 | 62135 | 44396 | 38967 | 53309 | 44166 | 84222 | 58555 |
| 42B08AAXX_6 | 496058 | 279540 | 312055 | 412095 | 231227 | 525567 | 488577 |
| 30WB6AAXX_5 | 291973 | 260206 | 259023 | 223449 | 273804 | 591800 | 578643 |
| 315CUAAXX_4 | 321565 | 291159 | 296471 | 260609 | 284906 | 672868 | 649580 |
| 30WB6AAXX_6 | 234278 | 334187 | 155545 | 221289 | 211756 | 324991 | 328050 |
| 30WG6AAXX_4 | 274315 | 341038 | 267480 | 251382 | 259209 | 563210 | 557926 |
| 30WG6AAXX_5 | 233686 | 319169 | 213869 | 251835 | 260092 | 626974 | 388865 |
| 30WG6AAXX_6 | 251690 | 474232 | 226607 | 258402 | 277687 | 529644 | 472464 |
| 30WG6AAXX_7 | 455059 | 378764 | 271987 | 316854 | 377667 | 636835 | 738787 |
| 30WU2AAXX_1 | 457794 | 442280 | 273773 | 404886 | 391403 | 716906 | 600713 |
| 42B08AAXX_1 | 258342 | 321807 | 200085 | 283791 | 192930 | 522251 | 411291 |
| 42CJJAAXX_5 | 507579 | 487070 | 297097 | 440256 | 437374 | 783709 | 671904 |
| 42CJJAAXX_7 | 215438 | 247607 | 160436 | 230390 | 200428 | 391220 | 314190 |
| 42NY4AAXX_2 | 415602 | 463558 | 301663 | 424990 | 396008 | 741251 | 605626 |
| 42B48AAXX_2 | 242083 | 241669 | 166523 | 218783 | 219667 | 391154 | 314901 |
| 42PFAAAXX_1 | 439066 | 429401 | 298508 | 384644 | 406568 | 699917 | 570677 |
| 30WU2AAXX_4 | 275844 | 307352 | 184919 | 256465 | 269319 | 501869 | 394041 |
| 42B48AAXX_3 | 448718 | 491314 | 297034 | 400037 | 437776 | 782048 | 632609 |
| 30WU2AAXX_5 | 338955 | 359214 | 236944 | 292241 | 327646 | 554637 | 450769 |
| 42B48AAXX_4 | 544868 | 559268 | 369079 | 444826 | 532213 | 858736 | 722488 |
| 30WU2AAXX_6 | 112350 | 133568 | 83267 | 118555 | 116638 | 229524 | 171011 |
| 42B48AAXX_8 | 328392 | 381437 | 239043 | 332867 | 341800 | 647959 | 492590 |
| 42PFAAAXX_2 | 192653 | 224465 | 141821 | 198459 | 198843 | 384082 | 286449 |
| 30WU2AAXX_7 | 56381 | 60208 | 39619 | 51409 | 52525 | 98887 | 80307 |
| 42CJFAAXX_5 | 447003 | 470836 | 311538 | 387732 | 422233 | 753698 | 630823 |
| 30WU2AAXX_8 | 232604 | 238435 | 155899 | 196779 | 208731 | 399162 | 326258 |
| 42CJFAAXX_4 | 390379 | 397758 | 259676 | 327632 | 349445 | 662270 | 545561 |
| 42PFAAAXX_3 | 435827 | 438817 | 288565 | 363292 | 393925 | 737693 | 610012 |
| 30Y6GAAXX_2 | 205433 | 211180 | 174914 | 191708 | 115386 | 354329 | 248128 |
| 30Y6GAAXX_4 | 322389 | 289344 | 246556 | 238303 | 261620 | 516940 | 443501 |
| 30Y6GAAXX_5 | 294984 | 251602 | 254580 | 201529 | 267059 | 434289 | 418212 |
| 30Y6GAAXX_6 | 313434 | 401122 | 326653 | 310322 | 303326 | 384534 | 513721 |
| 30Y6GAAXX_7 | 264425 | 317931 | 300052 | 223830 | 259621 | 504147 | 648812 |
| 314N5AAXX_1 | 373275 | 371624 | 246483 | 239856 | 261321 | 512978 | 449886 |
| 314N5AAXX_2 | 301195 | 284165 | 208210 | 222120 | 278955 | 563168 | 400692 |
| 314N5AAXX_3 | 372857 | 350606 | 252890 | 299387 | 304925 | 637983 | 541642 |
| 314N5AAXX_4 | 492098 | 392958 | 279705 | 307863 | 389864 | 665552 | 584271 |
| 314NNAAXX_2 | 184360 | 311972 | 293671 | 233813 | 219561 | 537874 | 554933 |
| 314NNAAXX_4 | 243056 | 366269 | 252004 | 206180 | 208634 | 452700 | 643902 |
| 314RPAAXX_5 | 232250 | 231969 | 242899 | 199343 | 211365 | 469129 | 411525 |
| 314RPAAXX_6 | 266085 | 241241 | 198053 | 195648 | 270341 | 422788 | 567168 |
| 314RPAAXX_7 | 212444 | 298677 | 283912 | 210123 | 212234 | 361523 | 498287 |
| 314T1AAXX_2 | 272408 | 369367 | 189013 | 245396 | 242422 | 441080 | 405295 |
| 314WAAAXX_1 | 238171 | 279812 | 167183 | 251320 | 159014 | 376698 | 353028 |
| 314WAAAXX_2 | 294399 | 315726 | 297377 | 289486 | 277210 | 460242 | 380158 |
| 314WAAAXX_6 | 227285 | 200360 | 140738 | 176338 | 178003 | 316020 | 338304 |
| 314WAAAXX_7 | 230917 | 284448 | 227026 | 132607 | 186761 | 359756 | 261380 |
| 314WGAAXX_2 | 245992 | 362466 | 269324 | 233055 | 246155 | 481804 | 420996 |
| 314WGAAXX_3 | 231393 | 316653 | 263253 | 238650 | 261480 | 498075 | 552488 |
| 314WGAAXX_5 | 298042 | 373468 | 341765 | 195110 | 234123 | 500557 | 431127 |
| 314WGAAXX_7 | 251388 | 383462 | 234138 | 289674 | 261732 | 436505 | 438542 |
| 315C1AAXX_3 | 265227 | 248558 | 145140 | 120569 | 265770 | 380436 | 398784 |
| 315C1AAXX_4 | 316200 | 300440 | 208107 | 184575 | 178666 | 507666 | 495057 |
| 315CUAAXX_1 | 193958 | 213875 | 193595 | 280473 | 173309 | 475303 | 374482 |
| 315CUAAXX_2 | 483541 | 432169 | 181401 | 296232 | 254037 | 700716 | 427311 |
| 315CUAAXX_5 | 539174 | 400955 | 383542 | 298905 | 348624 | 587221 | 561018 |
| 315CUAAXX_6 | 222932 | 265779 | 154766 | 209655 | 239517 | 388127 | 508017 |
| 315CUAAXX_7 | 294603 | 341881 | 208224 | 231637 | 259160 | 497609 | 431053 |
| 429T4AAXX_1 | 382789 | 647126 | 318292 | 364134 | 361302 | 682844 | 658059 |
| 429T4AAXX_2 | 322427 | 409201 | 283419 | 383719 | 283098 | 783083 | 740227 |
| 429T4AAXX_3 | 359716 | 499370 | 310879 | 396351 | 375922 | 878319 | 697167 |
| 429T4AAXX_4 | 517622 | 424125 | 439739 | 411665 | 385470 | 591711 | 577947 |
| 429T4AAXX_5 | 432547 | 440917 | 320197 | 344669 | 389160 | 733196 | 947598 |
| 429T4AAXX_6 | 418111 | 401115 | 480055 | 378518 | 348099 | 643424 | 644298 |
| 429T4AAXX_7 | 454606 | 635828 | 474672 | 393267 | 342519 | 732451 | 725468 |
| 42AV4AAXX_1 | 542457 | 684031 | 347133 | 526033 | 544639 | 688634 | 980331 |
| 42AV4AAXX_2 | 648394 | 626834 | 417222 | 505364 | 613718 | 1064979 | 1345104 |
| 42AV4AAXX_3 | 536752 | 501492 | 499351 | 428004 | 559719 | 1116500 | 1061193 |
| 42AV4AAXX_5 | 681540 | 525730 | 667219 | 576483 | 535259 | 1357743 | 1203817 |
| 42AV4AAXX_6 | 489208 | 600355 | 442465 | 486968 | 545690 | 1096117 | 1236652 |
| 42AV4AAXX_7 | 659431 | 720553 | 417669 | 747257 | 460429 | 1151765 | 846070 |
| 42B08AAXX_2 | 179312 | 187761 | 194744 | 229523 | 141296 | 312203 | 394851 |
| 42B08AAXX_4 | 313180 | 307383 | 341882 | 342507 | 279211 | 644348 | 773484 |
| 42B08AAXX_5 | 394367 | 339090 | 236684 | 295508 | 370245 | 568235 | 522290 |
| 42B08AAXX_7 | 358586 | 404278 | 253866 | 327622 | 380880 | 645409 | 537088 |
| 42B08AAXX_8 | 395833 | 336030 | 206824 | 268868 | 272109 | 586744 | 502480 |
| 42B20AAXX_5 | 566170 | 565350 | 359826 | 444516 | 533654 | 892882 | 817867 |
| 42NY4AAXX_6 | 544295 | 538725 | 345261 | 422970 | 510191 | 846035 | 778309 |
| 42B3YAAXX_4 | 268051 | 273089 | 178698 | 204140 | 260649 | 433683 | 319837 |
| 42B3YAAXX_5 | 347328 | 296591 | 175729 | 231143 | 235330 | 402912 | 476894 |
| 42B3YAAXX_6 | 249391 | 256165 | 172196 | 174390 | 323646 | 401243 | 551721 |
| 42B3YAAXX_7 | 272955 | 182436 | 147358 | 146161 | 249762 | 367113 | 617958 |
| 42B48AAXX_1 | 426844 | 420714 | 281561 | 346592 | 375645 | 630810 | 540244 |
| 42NY4AAXX_3 | 411212 | 406848 | 273257 | 336344 | 363441 | 615797 | 525423 |
| 42B48AAXX_5 | 505144 | 510297 | 319792 | 402746 | 442457 | 788193 | 672512 |
| 42B48AAXX_6 | 489926 | 447908 | 269850 | 352757 | 390164 | 661827 | 569805 |
| 42NY4AAXX_4 | 491379 | 446120 | 270394 | 352649 | 392964 | 663123 | 567313 |
| 42HAMAAXX_3 | 273223 | 423007 | 298730 | 279073 | 290026 | 466668 | 466079 |
| 42HAMAAXX_5 | 313550 | 380432 | 326187 | 358189 | 262833 | 726483 | 620229 |
| 42HAMAAXX_6 | 417051 | 376494 | 279072 | 255638 | 329563 | 648834 | 776545 |
| 42CJFAAXX_1 | 319355 | 324402 | 199775 | 270465 | 282282 | 529757 | 441642 |
| 42CJFAAXX_6 | 404728 | 303566 | 178379 | 266911 | 160276 | 560277 | 355914 |
| 42CJFAAXX_7 | 247552 | 274577 | 182503 | 234049 | 268231 | 476016 | 411852 |
| 42P6UAAXX_8 | 23885 | 27093 | 17931 | 23845 | 25897 | 48224 | 40796 |
| 42PFAAAXX_4 | 345859 | 380883 | 253926 | 327361 | 385179 | 671278 | 580841 |
| 42CJJAAXX_1 | 400000 | 493794 | 363291 | 295629 | 486208 | 866729 | 1027733 |
| 42CJJAAXX_2 | 208539 | 423686 | 317813 | 400471 | 354756 | 336516 | 512097 |
| 42CJJAAXX_3 | 370843 | 537605 | 390944 | 342171 | 314395 | 553047 | 728442 |
| 42CJJAAXX_4 | 427184 | 404988 | 339986 | 336292 | 378608 | 896994 | 587996 |
| 42CJJAAXX_6 | 166823 | 182888 | 131687 | 159914 | 152639 | 304773 | 224612 |
| 42PF0AAXX_3 | 256193 | 277015 | 201812 | 243157 | 238564 | 469580 | 345399 |
| 42CJJAAXX_8 | 463080 | 473293 | 312551 | 413543 | 392959 | 727906 | 612507 |
| 42D3NAAXX_1 | 253129 | 253881 | 144411 | 206011 | 214512 | 382798 | 364444 |
| 42D3NAAXX_2 | 257838 | 318100 | 158358 | 226191 | 208794 | 429292 | 406360 |
| 42D3NAAXX_3 | 324872 | 324704 | 202305 | 257636 | 244187 | 528367 | 451118 |
| 42D3NAAXX_5 | 321704 | 356139 | 235205 | 303148 | 280248 | 549610 | 436979 |
| 42D3NAAXX_6 | 335291 | 331043 | 238375 | 348920 | 267023 | 536796 | 471344 |
| 42P6UAAXX_6 | 406459 | 404049 | 295503 | 426453 | 321575 | 652103 | 568790 |
| 42D3NAAXX_7 | 343180 | 333103 | 217989 | 297649 | 252905 | 504329 | 415447 |
| 42P6UAAXX_7 | 447330 | 437916 | 288741 | 393952 | 330752 | 663469 | 543509 |
| 42PF0AAXX_6 | 431745 | 596896 | 500730 | 243012 | 318887 | 619644 | 629689 |
| 42PF0AAXX_7 | 345830 | 369534 | 180031 | 141258 | 314043 | 573368 | 633038 |
| 42PFAAAXX_5 | 510117 | 409151 | 316893 | 360335 | 377021 | 727702 | 701089 |
| 42PFAAAXX_6 | 351970 | 315651 | 247163 | 266745 | 336842 | 549187 | 530696 |
| 42PMUAAXX_6 | 347637 | 308706 | 243632 | 248795 | 273149 | 498728 | 545016 |
| 42PMUAAXX_7 | 309746 | 276715 | 221268 | 249212 | 246423 | 496523 | 490566 |
| 42PFAAAXX_7 | 463349 | 378434 | 306458 | 362807 | 375355 | 808417 | 865367 |
| 42PFAAAXX_8 | 262025 | 404999 | 271012 | 239473 | 278245 | 507792 | 567257 |
| 42PMUAAXX_1 | 337092 | 511068 | 360437 | 408476 | 361467 | 646757 | 645450 |
| 42PMUAAXX_2 | 313539 | 224084 | 138599 | 147596 | 265593 | 608928 | 491305 |
| 42PMUAAXX_4 | 291164 | 251700 | 188771 | 226962 | 259024 | 432518 | 436355 |
| 42PMUAAXX_8 | 290107 | 270069 | 141306 | 188356 | 174749 | 251520 | 242922 |
| 42RMPAAXX_1 | 541190 | 578203 | 398818 | 474181 | 555241 | 914377 | 734648 |
| 42RMPAAXX_2 | 816560 | 608331 | 448538 | 461142 | 641759 | 948299 | 942350 |
| 42RMPAAXX_3 | 636506 | 500634 | 408695 | 448378 | 506507 | 898375 | 1001094 |
| 42RMPAAXX_4 | 438044 | 347672 | 255224 | 313602 | 300919 | 615181 | 614454 |
| 42RMPAAXX_5 | 450282 | 349526 | 254310 | 169252 | 374721 | 511935 | 547092 |
| 42RMPAAXX_6 | 338186 | 320702 | 315564 | 200243 | 280468 | 375965 | 467730 |
| 42RMPAAXX_7 | 314443 | 370017 | 197653 | 303188 | 373380 | 435495 | 637075 |
| 42RMPAAXX_8 | 366875 | 548182 | 371086 | 519333 | 372035 | 721384 | 884522 |
| 42RYFAAXX_1 | 239993 | 187237 | 133986 | 151228 | 158339 | 330659 | 304054 |
| 42RYFAAXX_2 | 225802 | 275201 | 144789 | 232845 | 245373 | 437856 | 370787 |
| 42RYFAAXX_3 | 388352 | 223605 | 182029 | 241253 | 228353 | 452196 | 525262 |
| 42RYFAAXX_4 | 239602 | 218825 | 168680 | 178161 | 222556 | 377571 | 405505 |
| 42RYFAAXX_5 | 336377 | 219426 | 208346 | 205693 | 229661 | 383066 | 413768 |
| 42RYFAAXX_6 | 476813 | 363827 | 235690 | 293247 | 373176 | 605111 | 574781 |
| 42RYFAAXX_7 | 368014 | 60474 | 56563 | 56793 | 223220 | 1926242 | 107417 |
| 42RYFAAXX_8 | 232078 | 187320 | 132595 | 161812 | 174637 | 338150 | 316673 |
| 42TA8AAXX_1 | 337395 | 349799 | 286005 | 324552 | 372232 | 558351 | 583496 |
| 42TA8AAXX_2 | 399592 | 412501 | 337022 | 383345 | 435290 | 656967 | 692204 |
| 42TA8AAXX_3 | 374872 | 391214 | 321009 | 362883 | 409268 | 623379 | 653436 |
| 42TA8AAXX_4 | 11614 | 12287 | 10124 | 11512 | 12444 | 19581 | 20076 |
| 42TA8AAXX_5 | 441252 | 455445 | 373109 | 420801 | 486847 | 717681 | 761975 |
| 42TA8AAXX_6 | 498209 | 485181 | 404807 | 448999 | 561407 | 775165 | 843694 |
| 42TA8AAXX_7 | 396240 | 413811 | 337777 | 383373 | 430495 | 659625 | 690794 |
| 42TB9AAXX_1 | 701493 | 614483 | 427909 | 486193 | 620489 | 925810 | 806405 |
| 42TB9AAXX_2 | 306511 | 313814 | 222612 | 251075 | 332821 | 559673 | 508985 |
| 42TB9AAXX_3 | 596637 | 554060 | 420399 | 440183 | 564395 | 920595 | 770777 |
| 42TB9AAXX_4 | 333090 | 298921 | 199591 | 248169 | 271231 | 494790 | 407025 |
| 42Y6WAAXX_6 | 503907 | 446688 | 299736 | 373967 | 413478 | 749102 | 621978 |
| 42TB9AAXX_6 | 578383 | 571612 | 387768 | 451846 | 596920 | 918552 | 773363 |
| 42TB9AAXX_7 | 549174 | 543847 | 369347 | 442765 | 511838 | 928405 | 774316 |
| 42TBWAAXX_1 | 213107 | 340508 | 223347 | 174593 | 203607 | 386949 | 277958 |
| 42TBWAAXX_2 | 715682 | 458230 | 336156 | 365276 | 452335 | 822350 | 751761 |
| 42TBWAAXX_3 | 402513 | 370287 | 261086 | 302832 | 346076 | 526045 | 470157 |
| 42TBWAAXX_4 | 608277 | 487185 | 292170 | 342064 | 462347 | 827438 | 753826 |
| 42TBWAAXX_5 | 321449 | 287044 | 212126 | 252221 | 246881 | 555223 | 662727 |
| 42TBWAAXX_6 | 206609 | 191093 | 159080 | 171116 | 190926 | 442304 | 339761 |
| 42Y6WAAXX_1 | 235190 | 217618 | 180852 | 195080 | 215268 | 505239 | 386580 |
| 42TBWAAXX_7 | 433703 | 291358 | 219416 | 241274 | 249903 | 571001 | 546028 |
| 42TBWAAXX_8 | 250244 | 342644 | 271515 | 285125 | 202059 | 703171 | 481618 |
| 42TCBAAXX_1 | 351373 | 419077 | 274630 | 191801 | 301973 | 522391 | 539437 |
| 42TCBAAXX_2 | 327078 | 272838 | 281904 | 147886 | 223767 | 572569 | 640449 |
| 42TCBAAXX_3 | 314543 | 376633 | 300688 | 237242 | 223720 | 513530 | 624677 |
| 42TCBAAXX_4 | 463711 | 374550 | 299267 | 276601 | 355816 | 718331 | 1016502 |
| 42TCBAAXX_5 | 482277 | 433517 | 287292 | 305925 | 452566 | 745880 | 661830 |
| 42TCBAAXX_6 | 156185 | 157769 | 133741 | 70544 | 164543 | 290917 | 222756 |
| 42TCBAAXX_7 | 255824 | 223430 | 179162 | 170008 | 289349 | 471425 | 406337 |
| 42TCBAAXX_8 | 290900 | 268471 | 194884 | 234782 | 253433 | 443328 | 431496 |
| 42TCLAAXX_1 | 92393 | 71995 | 40597 | 50592 | 66171 | 102815 | 129477 |
| 42Y39AAXX_2 | 758648 | 562779 | 346755 | 413984 | 582709 | 834120 | 1085509 |
| 42TCLAAXX_3 | 83018 | 72730 | 38196 | 45964 | 56367 | 88342 | 107587 |
| 42Y39AAXX_5 | 750945 | 622479 | 358399 | 417263 | 542184 | 787230 | 989219 |
| 42TCLAAXX_4 | 242176 | 198925 | 104431 | 132908 | 160615 | 260965 | 323697 |
| 42Y39AAXX_6 | 740344 | 559839 | 339626 | 413808 | 525743 | 781111 | 964689 |
| 42TCLAAXX_5 | 155185 | 160429 | 87422 | 111706 | 130476 | 193818 | 240306 |
| 42Y39AAXX_7 | 592188 | 553007 | 341967 | 414927 | 531617 | 707253 | 909000 |
| 42TG0AAXX_2 | 137889 | 124977 | 92412 | 105260 | 120943 | 243946 | 244088 |
| 42TG0AAXX_3 | 387703 | 421565 | 303166 | 190738 | 415709 | 1252234 | 716555 |
| 42TG0AAXX_4 | 522170 | 538998 | 369973 | 395422 | 422871 | 663592 | 648953 |
| 42TG0AAXX_5 | 335337 | 306012 | 242748 | 148524 | 310555 | 433428 | 531355 |
| 42TG0AAXX_6 | 360955 | 276081 | 288890 | 179373 | 406716 | 809648 | 619533 |
| 42TG0AAXX_7 | 7901 | 7399 | 41102 | 5235 | 11577 | 35462 | 21390 |
| 42TG0AAXX_8 | 399347 | 409339 | 401987 | 301174 | 470627 | 985831 | 669244 |
| 42Y2MAAXX_1 | 901665 | 576690 | 452125 | 429198 | 618887 | 854009 | 815845 |
| 42Y2MAAXX_2 | 887682 | 584101 | 472823 | 444541 | 656330 | 828428 | 812907 |
| 42Y2MAAXX_3 | 886319 | 609777 | 468895 | 459128 | 672298 | 814584 | 856084 |
| 42Y2MAAXX_4 | 937651 | 592311 | 482901 | 504684 | 683731 | 942624 | 904413 |
| 42Y2MAAXX_5 | 938914 | 611532 | 433685 | 457365 | 669186 | 880798 | 888033 |
| 42Y2MAAXX_6 | 841933 | 609966 | 486856 | 482064 | 628963 | 853937 | 792007 |
| 42Y2MAAXX_7 | 101933 | 554650 | 454616 | 495632 | 613003 | 886502 | 785581 |
| 42Y2MAAXX_8 | 646702 | 609730 | 436936 | 467558 | 618258 | 874836 | 812777 |
| 42Y39AAXX_3 | 673354 | 634281 | 333998 | 445712 | 539987 | 831377 | 992863 |
| 42Y39AAXX_4 | 675579 | 629181 | 335231 | 444632 | 544935 | 830766 | 999541 |
| 42Y6NAAXX_1 | 600356 | 533672 | 312069 | 368121 | 431534 | 619625 | 734096 |
| 42Y6NAAXX_2 | 462759 | 483740 | 358629 | 368516 | 460072 | 676315 | 732312 |
| 42Y6NAAXX_4 | 506551 | 547305 | 352262 | 481543 | 394449 | 700749 | 719183 |
| 42Y6NAAXX_5 | 600910 | 506646 | 357921 | 422683 | 490887 | 578122 | 822701 |
| 42Y6NAAXX_6 | 599608 | 445983 | 313741 | 497525 | 422316 | 718410 | 703833 |
| 42Y6NAAXX_7 | 427236 | 414690 | 316131 | 535220 | 446547 | 715324 | 811681 |
| 42Y6NAAXX_8 | 436779 | 498953 | 290765 | 387790 | 397160 | 671069 | 718987 |
| 42Y6WAAXX_2 | 427320 | 396370 | 250750 | 348263 | 427878 | 610387 | 701932 |
| 42YMTAAXX_2 | 303672 | 280851 | 179617 | 245775 | 304627 | 427337 | 493758 |
| 42Y6WAAXX_3 | 644069 | 465601 | 250875 | 378941 | 453762 | 747030 | 894556 |
| 42YMTAAXX_4 | 292698 | 212356 | 114642 | 173218 | 203598 | 341293 | 403235 |
| 42Y6WAAXX_4 | 558790 | 219674 | 183264 | 191628 | 326427 | 2303130 | 403530 |
| 42YMTAAXX_8 | 299176 | 118700 | 99539 | 104151 | 173108 | 1254430 | 214607 |
| 42YMMAAXX_1 | 549340 | 756994 | 528012 | 373234 | 600210 | 730055 | 920608 |
| 42YMMAAXX_2 | 634364 | 615276 | 492383 | 458444 | 632012 | 1471295 | 1129124 |
| 42YMMAAXX_8 | 591425 | 578478 | 457265 | 433784 | 570671 | 1454968 | 1084629 |
| 42YMMAAXX_3 | 625562 | 559133 | 394908 | 442943 | 629867 | 847814 | 992984 |
| 42YMMAAXX_4 | 609991 | 525836 | 398287 | 402853 | 588949 | 717072 | 947515 |
| 42YMMAAXX_5 | 699474 | 555822 | 425122 | 414372 | 556286 | 1021030 | 1010806 |
| 42YMMAAXX_6 | 614939 | 527430 | 367829 | 392187 | 542984 | 733697 | 880594 |
| 42YMMAAXX_7 | 601028 | 584552 | 380777 | 389541 | 521415 | 868310 | 865881 |
| 42YMTAAXX_1 | 434846 | 515864 | 290258 | 292322 | 442745 | 789722 | 796902 |
| 42YMTAAXX_3 | 403840 | 328910 | 278890 | 259904 | 333415 | 555821 | 599868 |
| 42YMTAAXX_5 | 375884 | 301372 | 245199 | 189895 | 267121 | 441538 | 612997 |
| 42YMTAAXX_6 | 720802 | 562078 | 407843 | 303370 | 532321 | 903866 | 1027834 |
| 42YMTAAXX_7 | 305418 | 277321 | 215272 | 241401 | 301194 | 505519 | 497449 |
| 43336AAXX_1 | 1712537 | 1322494 | 1102286 | 1189477 | 1429672 | 1441108 | 1577969 |
| 43336AAXX_2 | 1320676 | 1155227 | 858458 | 1112394 | 1197423 | 1462369 | 1316749 |
| 43336AAXX_3 | 1727497 | 1347935 | 1152498 | 1144008 | 1333119 | 1460548 | 1531510 |
| 43336AAXX_4 | 1571782 | 1254117 | 1012812 | 1148974 | 1333420 | 1422935 | 1476403 |
| 43336AAXX_5 | 1609521 | 1366735 | 1204238 | 1157876 | 1481372 | 1369016 | 1394935 |
| 43336AAXX_6 | 1814918 | 1380656 | 1250045 | 1198462 | 1382568 | 1511334 | 1607652 |
| 43336AAXX_7 | 460572 | 402430 | 320729 | 346164 | 435797 | 1216631 | 678482 |
| 6137KAAXX_3 | 828300 | 1006024 | 730135 | 406217 | 750881 | 1439489 | 1181757 |
| 6137KAAXX_4 | 828325 | 1007184 | 731332 | 407079 | 751374 | 1436803 | 1179209 |
| 6137LAAXX_6 | 148514 | 220001 | 152847 | 99100 | 166342 | 274867 | 198564 |
| 6137KAAXX_6 | 872920 | 796339 | 448299 | 736103 | 706989 | 1133142 | 1543977 |
| 6137KAAXX_7 | 1024242 | 928818 | 645784 | 687955 | 843777 | 1358258 | 1467999 |
| 6137KAAXX_8 | 1015056 | 888307 | 486127 | 589801 | 717160 | 1104910 | 1177943 |
| 6137LAAXX_7 | 740821 | 670949 | 614150 | 472038 | 617402 | 566485 | 570214 |
| 6137LAAXX_8 | 514717 | 491908 | 421410 | 334189 | 450649 | 401815 | 414097 |
| 61VFCAAXX_1 | 1067821 | 1074346 | 732192 | 550146 | 628846 | 970566 | 1523906 |
| 61VFCAAXX_2 | 915906 | 1113297 | 583047 | 561016 | 624447 | 920608 | 1380512 |
| 61VFCAAXX_3 | 1026827 | 1171452 | 951422 | 568151 | 689221 | 965464 | 1668906 |
| 61VFCAAXX_6 | 890190 | 857890 | 564774 | 654995 | 742342 | 1108949 | 1356889 |
| 62837AAXX_1 | 1502516 | 977756 | 561400 | 603758 | 875089 | 1258543 | 1405073 |
| 62837AAXX_2 | 1289278 | 814901 | 631190 | 694899 | 871395 | 1156158 | 1467138 |
| 62837AAXX_3 | 1228789 | 1030670 | 698400 | 731263 | 903572 | 1303910 | 1403485 |
| 62837AAXX_4 | 1081522 | 1013337 | 550037 | 603367 | 835872 | 1155843 | 1146485 |
| 62837AAXX_7 | 331944 | 279869 | 184291 | 216310 | 247144 | 329723 | 301481 |
| 62837AAXX_8 | 940905 | 792683 | 505733 | 578690 | 710415 | 1095507 | 1079212 |
| 6286PAAXX_1 | 1111411 | 893304 | 491026 | 641434 | 730107 | 1154407 | 1106115 |
| 6286PAAXX_2 | 1270939 | 866520 | 504949 | 582779 | 775955 | 1157554 | 1172921 |
| 6286PAAXX_3 | 811799 | 1082760 | 430290 | 547432 | 709247 | 1253463 | 1343195 |
| 6286PAAXX_4 | 714445 | 921894 | 734645 | 612984 | 656047 | 1313519 | 1242485 |
| 6286PAAXX_5 | 1011615 | 738873 | 679114 | 558441 | 718630 | 1011032 | 1185073 |
| 6286PAAXX_6 | 979056 | 801789 | 599104 | 800644 | 774714 | 1111793 | 1309382 |
| 6286PAAXX_7 | 744096 | 762662 | 569131 | 682524 | 725113 | 1338047 | 1273663 |
| 6286PAAXX_8 | 1179154 | 938183 | 523752 | 613977 | 679361 | 1043845 | 1287321 |
| Flowcell_lane | hs_ref_chr13 | hs_ref_chr14 | hs_ref_chr15 | hs_ref_chr16 | hs_ref_chr17 | hs_ref_chr18 | hs_ref_chr19 |
| 314N5AAXX_6 | 110454 | 228966 | 258831 | 349253 | 436308 | 64450 | 536009 |
| 314WGAAXX_4 | 65400 | 230062 | 164354 | 345896 | 518186 | 62970 | 569985 |
| 30Y5NAAXX_1 | 93922 | 188965 | 291865 | 318920 | 564040 | 80684 | 630204 |
| 315C1AAXX_1 | 62275 | 147983 | 241614 | 298705 | 486580 | 60665 | 558981 |
| 30LUMAAXX_2 | 8850 | 148530 | 203977 | 239514 | 470629 | 52246 | 385109 |
| 42AV4AAXX_4 | 145854 | 377392 | 507841 | 733419 | 1121589 | 112516 | 1076389 |
| 314N5AAXX_5 | 71947 | 323136 | 189669 | 326702 | 435753 | 69230 | 438817 |
| 30TYGAAXX_1 | 28966 | 50405 | 55974 | 80708 | 106332 | 21291 | 139848 |
| 42PMUAAXX_5 | 89640 | 188445 | 235643 | 369999 | 511144 | 91239 | 438849 |
| 42D3NAAXX_4 | 64179 | 209106 | 154846 | 433716 | 624989 | 48836 | 528562 |
| 42Y39AAXX_8 | 128547 | 380956 | 282242 | 668151 | 970481 | 99494 | 775578 |
| 42YRVAAXX_2 | 142709 | 217108 | 240971 | 256135 | 411665 | 94957 | 276838 |
| 42B20AAXX_2 | 97470 | 231670 | 282067 | 363245 | 708510 | 112361 | 656313 |
| 30TYGAAXX_3 | 26705 | 65430 | 74657 | 105391 | 178200 | 18598 | 354045 |
| 42B20AAXX_6 | 161797 | 305095 | 353884 | 582463 | 882583 | 78960 | 1099964 |
| 42HAMAAXX_1 | 163021 | 222622 | 315829 | 315287 | 595723 | 112549 | 507424 |
| 42B20AAXX_1 | 224409 | 331440 | 396897 | 514130 | 961863 | 130217 | 878318 |
| 42YRVAAXX_3 | 160447 | 260320 | 285349 | 434486 | 690923 | 96196 | 727185 |
| 42HAMAAXX_2 | 185619 | 334065 | 324542 | 442025 | 1145855 | 108643 | 837979 |
| 42YRVAAXX_1 | 110345 | 150910 | 204862 | 176377 | 340739 | 76301 | 222299 |
| 30TUEAAXX_3 | 10705 | 108166 | 156313 | 137070 | 357277 | 40507 | 456659 |
| 3035BAAXX_3 | 79755 | 284890 | 313514 | 386469 | 631149 | 97851 | 554870 |
| 314NNAAXX_3 | 51675 | 171828 | 184725 | 306420 | 376545 | 46100 | 370417 |
| 314WAAAXX_3 | 75544 | 218957 | 237410 | 381734 | 461083 | 63523 | 455388 |
| 314WAAAXX_5 | 59734 | 119613 | 207839 | 245405 | 394328 | 61271 | 431771 |
| 42Y39AAXX_1 | 168051 | 312030 | 532561 | 544248 | 896695 | 173698 | 905576 |
| 30WB6AAXX_7 | 101985 | 200861 | 178200 | 375324 | 439286 | 49854 | 562644 |
| 30351AAXX_7 | 121336 | 221612 | 264898 | 268312 | 456638 | 128622 | 343293 |
| 314T1AAXX_1 | 90470 | 179250 | 217638 | 243428 | 413846 | 100701 | 326059 |
| 30U00AAXX_7 | 18577 | 36787 | 31385 | 53139 | 99594 | 15268 | 88135 |
| 30LEJAAXX_7 | 14758 | 265732 | 214061 | 429011 | 660525 | 73933 | 539542 |
| 30TYGAAXX_5 | 40062 | 110141 | 59850 | 114808 | 188355 | 37858 | 235125 |
| 314NNAAXX_6 | 60032 | 178620 | 189267 | 353534 | 485599 | 47354 | 526962 |
| 3064YAAXX_1 | 92703 | 252288 | 280796 | 233049 | 561392 | 91805 | 461077 |
| 30LF7AAXX_1 | 13769 | 157626 | 164180 | 123771 | 357088 | 63774 | 268436 |
| 315CUAAXX_3 | 69353 | 204667 | 179183 | 484053 | 596470 | 77197 | 546949 |
| 30LF7AAXX_2 | 14606 | 148886 | 166255 | 241279 | 347871 | 48654 | 501228 |
| 30LF7AAXX_3 | 20120 | 139195 | 160932 | 229431 | 301901 | 48233 | 517710 |
| 314WAAAXX_4 | 64850 | 159907 | 153621 | 345091 | 591934 | 49040 | 389376 |
| 42PMUAAXX_3 | 95555 | 264173 | 304519 | 440244 | 746242 | 86763 | 672818 |
| 6137LAAXX_2 | 27800 | 56134 | 37120 | 46715 | 97923 | 22651 | 90047 |
| 30Y6GAAXX_1 | 52357 | 102283 | 160195 | 312267 | 333388 | 27281 | 419290 |
| 314NNAAXX_5 | 72945 | 174611 | 172954 | 560615 | 743143 | 41865 | 502940 |
| 30Y6GAAXX_3 | 85760 | 174195 | 176126 | 459496 | 634013 | 45498 | 476723 |
| 30LUMAAXX_3 | 8780 | 178551 | 149014 | 237664 | 473629 | 42476 | 267205 |
| 314WGAAXX_1 | 76226 | 288720 | 222157 | 403782 | 663472 | 52619 | 433314 |
| 42HAMAAXX_7 | 81390 | 147247 | 181428 | 340703 | 392982 | 58793 | 387011 |
| 314N5AAXX_7 | 120456 | 223323 | 229174 | 315249 | 438937 | 53363 | 413263 |
| 301YWAAXX_1 | 141792 | 397144 | 372323 | 611466 | 686750 | 119286 | 1180056 |
| 42P6UAAXX_4 | 154603 | 431923 | 408323 | 644780 | 730119 | 133315 | 1226049 |
| 301YWAAXX_2 | 154895 | 365092 | 364491 | 577523 | 684082 | 102088 | 915432 |
| 42P6UAAXX_5 | 156373 | 362105 | 363751 | 566520 | 672421 | 102948 | 891476 |
| 301YWAAXX_3 | 163849 | 332408 | 342585 | 600222 | 723729 | 72189 | 1390195 |
| 302LVAAXX_2 | 133046 | 252126 | 216459 | 337434 | 485170 | 107443 | 684484 |
| 302LVAAXX_5 | 140753 | 309536 | 293056 | 385346 | 536132 | 75519 | 582941 |
| 302LVAAXX_6 | 151494 | 279098 | 357278 | 343154 | 516346 | 96403 | 574709 |
| 302LVAAXX_7 | 139838 | 385372 | 297641 | 404122 | 579104 | 81782 | 626199 |
| 3035BAAXX_1 | 126588 | 218663 | 263204 | 415146 | 649139 | 85125 | 619621 |
| 3035BAAXX_2 | 89093 | 259990 | 265401 | 364267 | 987337 | 41578 | 617097 |
| 3035BAAXX_5 | 85914 | 264001 | 207437 | 510737 | 711380 | 55672 | 623367 |
| 3035BAAXX_6 | 75587 | 181821 | 176044 | 385162 | 849548 | 47199 | 546569 |
| 3064YAAXX_2 | 103178 | 267693 | 243369 | 361006 | 490831 | 70791 | 750012 |
| 42P6UAAXX_1 | 168532 | 402355 | 371153 | 518240 | 707387 | 113551 | 1044713 |
| 3064YAAXX_3 | 96279 | 239520 | 238781 | 369308 | 448819 | 63839 | 735945 |
| 42P6UAAXX_2 | 133325 | 320136 | 319600 | 479890 | 583158 | 87754 | 944975 |
| 3064YAAXX_4 | 117749 | 249209 | 215739 | 311841 | 453564 | 71918 | 732719 |
| 30JD2AAXX_6 | 18153 | 42673 | 49151 | 52705 | 90014 | 23121 | 98292 |
| 3064YAAXX_5 | 111240 | 225455 | 230853 | 337246 | 422121 | 45898 | 902886 |
| 3064YAAXX_6 | 104031 | 290397 | 256844 | 334055 | 510346 | 61459 | 758836 |
| 3064YAAXX_7 | 84785 | 214902 | 215536 | 391405 | 428370 | 53545 | 576268 |
| 42P6UAAXX_3 | 142874 | 343336 | 349752 | 609246 | 669868 | 89454 | 881812 |
| 30650AAXX_1 | 137860 | 264165 | 307997 | 201789 | 425756 | 72620 | 306464 |
| 30650AAXX_5 | 84918 | 198482 | 208289 | 324999 | 806904 | 69665 | 412263 |
| 30650AAXX_6 | 48253 | 180892 | 179298 | 343368 | 827309 | 42409 | 319893 |
| 30650AAXX_7 | 101573 | 270046 | 199035 | 242706 | 1036000 | 59674 | 388727 |
| 30Y5NAAXX_2 | 88762 | 310196 | 216976 | 343870 | 614293 | 67528 | 547869 |
| 42Y6NAAXX_3 | 104309 | 453364 | 271385 | 353107 | 586347 | 105013 | 460976 |
| 30Y5NAAXX_3 | 121510 | 284917 | 253511 | 320389 | 418443 | 80935 | 692498 |
| 42B48AAXX_7 | 168906 | 406638 | 390150 | 559615 | 697116 | 109464 | 1212908 |
| 30Y5NAAXX_4 | 121436 | 273376 | 246538 | 314778 | 409094 | 77039 | 600809 |
| 42CJFAAXX_2 | 150143 | 376859 | 343740 | 489005 | 614878 | 92795 | 995142 |
| 42NY4AAXX_5 | 208859 | 486918 | 452676 | 600323 | 770664 | 128459 | 1194032 |
| 30GC3AAXX_3 | 40673 | 141425 | 157501 | 200876 | 276371 | 58427 | 443436 |
| 30Y5NAAXX_6 | 132494 | 281641 | 258736 | 368926 | 455449 | 83815 | 853333 |
| 30Y5NAAXX_7 | 133619 | 224045 | 219900 | 235038 | 280459 | 102599 | 335486 |
| 30U00AAXX_1 | 68592 | 212318 | 181134 | 262216 | 335692 | 85836 | 362975 |
| 30TUEAAXX_5 | 50094 | 174184 | 234493 | 276475 | 403406 | 93683 | 673234 |
| 30TUEAAXX_6 | 43032 | 152181 | 191209 | 252127 | 375873 | 73884 | 579164 |
| 30TUEAAXX_2 | 39288 | 173144 | 188051 | 229382 | 319464 | 72571 | 448147 |
| 30TUEAAXX_7 | 30132 | 125591 | 145346 | 170485 | 234732 | 53865 | 314949 |
| 30TUEAAXX_1 | 40840 | 196471 | 222604 | 233891 | 342675 | 82330 | 490469 |
| 30TUEAAXX_8 | 20382 | 99366 | 109623 | 107661 | 161029 | 39487 | 229274 |
| 30GC3AAXX_1 | 19833 | 216767 | 155123 | 342100 | 475342 | 59841 | 430304 |
| 30GC3AAXX_2 | 31984 | 270786 | 162437 | 226892 | 467855 | 65932 | 361098 |
| 30GC3AAXX_5 | 32817 | 254669 | 166877 | 233117 | 360082 | 49002 | 478173 |
| 30GC3AAXX_6 | 29642 | 190541 | 168391 | 214841 | 296663 | 65504 | 448410 |
| 30JD2AAXX_5 | 20134 | 165598 | 136287 | 176325 | 260327 | 51442 | 394034 |
| 30JD2AAXX_3 | 43309 | 181130 | 198080 | 262218 | 395573 | 76142 | 584877 |
| 30JD2AAXX_7 | 54729 | 105273 | 132585 | 139113 | 222239 | 57504 | 213495 |
| 30JD2AAXX_8 | 26320 | 72150 | 94502 | 88283 | 136975 | 40380 | 172096 |
| 30LEJAAXX_2 | 22705 | 220240 | 236303 | 367802 | 636476 | 68057 | 544063 |
| 30U00AAXX_2 | 72747 | 205451 | 184506 | 254123 | 327484 | 51771 | 313619 |
| 30U00AAXX_3 | 85888 | 306359 | 182948 | 392337 | 583940 | 95732 | 417403 |
| 30U00AAXX_4 | 98769 | 229042 | 203727 | 337245 | 431258 | 64166 | 806722 |
| 30U00AAXX_5 | 22483 | 33631 | 37817 | 69397 | 67785 | 11923 | 81452 |
| 30U00AAXX_6 | 16565 | 40060 | 30478 | 59818 | 80342 | 17887 | 69870 |
| 42B08AAXX_6 | 84257 | 266310 | 192931 | 459460 | 589999 | 95449 | 560162 |
| 30WB6AAXX_5 | 58329 | 213761 | 169747 | 247801 | 359399 | 65928 | 476595 |
| 315CUAAXX_4 | 60763 | 233874 | 185706 | 304929 | 435331 | 67903 | 592772 |
| 30WB6AAXX_6 | 66511 | 189259 | 157032 | 335948 | 285659 | 69311 | 355089 |
| 30WG6AAXX_4 | 108458 | 230003 | 272366 | 333739 | 450299 | 66152 | 531590 |
| 30WG6AAXX_5 | 89504 | 158975 | 174990 | 325254 | 389800 | 52349 | 341750 |
| 30WG6AAXX_6 | 79145 | 175044 | 248897 | 351663 | 725813 | 52637 | 568939 |
| 30WG6AAXX_7 | 161914 | 308497 | 366115 | 476798 | 678489 | 132061 | 655427 |
| 30WU2AAXX_1 | 139422 | 359496 | 370975 | 544357 | 715207 | 106117 | 1516302 |
| 42B08AAXX_1 | 67560 | 214919 | 235922 | 437492 | 569886 | 50198 | 1229223 |
| 42CJJAAXX_5 | 153479 | 398307 | 417458 | 588271 | 781871 | 118994 | 1639961 |
| 42CJJAAXX_7 | 66856 | 191934 | 189737 | 344725 | 387388 | 52022 | 611569 |
| 42NY4AAXX_2 | 134820 | 363823 | 367529 | 640870 | 712870 | 104686 | 1121011 |
| 42B48AAXX_2 | 85318 | 238392 | 180357 | 333113 | 352026 | 52797 | 466631 |
| 42PFAAAXX_1 | 161486 | 426057 | 330987 | 583282 | 615810 | 99673 | 787985 |
| 30WU2AAXX_4 | 107158 | 257166 | 260585 | 364063 | 457824 | 72963 | 847236 |
| 42B48AAXX_3 | 178400 | 413501 | 424725 | 553732 | 703721 | 122437 | 1268896 |
| 30WU2AAXX_5 | 119764 | 303140 | 276466 | 412758 | 513580 | 86212 | 737230 |
| 42B48AAXX_4 | 198300 | 476293 | 446373 | 613859 | 771089 | 146200 | 1039091 |
| 30WU2AAXX_6 | 36012 | 96338 | 98866 | 176758 | 207249 | 25103 | 393633 |
| 42B48AAXX_8 | 105002 | 277871 | 288079 | 490249 | 577449 | 73888 | 1089918 |
| 42PFAAAXX_2 | 62296 | 163337 | 166976 | 291664 | 342768 | 43576 | 647016 |
| 30WU2AAXX_7 | 18684 | 60976 | 44539 | 79045 | 90466 | 12677 | 144897 |
| 42CJFAAXX_5 | 151457 | 468121 | 358115 | 592063 | 686200 | 102184 | 1069456 |
| 30WU2AAXX_8 | 84201 | 196939 | 188794 | 276939 | 355106 | 55170 | 589822 |
| 42CJFAAXX_4 | 139368 | 328458 | 315047 | 458915 | 586917 | 92973 | 974588 |
| 42PFAAAXX_3 | 159275 | 366709 | 350121 | 504811 | 647963 | 104827 | 1068917 |
| 30Y6GAAXX_2 | 63213 | 114350 | 140311 | 280078 | 319004 | 26300 | 470707 |
| 30Y6GAAXX_4 | 84046 | 312749 | 199117 | 307957 | 496654 | 56190 | 630057 |
| 30Y6GAAXX_5 | 116385 | 172875 | 189795 | 207068 | 354410 | 57781 | 385687 |
| 30Y6GAAXX_6 | 119195 | 206820 | 239749 | 388818 | 562880 | 60463 | 539480 |
| 30Y6GAAXX_7 | 87899 | 183739 | 267925 | 458514 | 554014 | 76639 | 594326 |
| 314N5AAXX_1 | 71107 | 182954 | 197882 | 285608 | 388935 | 74215 | 520942 |
| 314N5AAXX_2 | 61994 | 222959 | 230865 | 287419 | 398240 | 80603 | 504546 |
| 314N5AAXX_3 | 82523 | 287218 | 303276 | 483158 | 682054 | 79549 | 772357 |
| 314N5AAXX_4 | 122185 | 568836 | 307180 | 466198 | 624524 | 112907 | 608051 |
| 314NNAAXX_2 | 60994 | 220750 | 165823 | 296026 | 557343 | 25700 | 542157 |
| 314NNAAXX_4 | 71930 | 205485 | 298589 | 335591 | 645696 | 64334 | 528785 |
| 314RPAAXX_5 | 61650 | 188933 | 173020 | 236436 | 613383 | 57577 | 418295 |
| 314RPAAXX_6 | 74823 | 179578 | 218867 | 303579 | 564707 | 105618 | 374664 |
| 314RPAAXX_7 | 74875 | 197342 | 143288 | 323971 | 424811 | 45044 | 341804 |
| 314T1AAXX_2 | 104185 | 171544 | 240083 | 222891 | 474106 | 62985 | 318223 |
| 314WAAAXX_1 | 80358 | 172641 | 199079 | 383525 | 404039 | 39200 | 486359 |
| 314WAAAXX_2 | 94331 | 184282 | 154145 | 309634 | 394625 | 68341 | 446659 |
| 314WAAAXX_6 | 47552 | 171384 | 162695 | 289988 | 406986 | 34880 | 378767 |
| 314WAAAXX_7 | 80042 | 110445 | 170331 | 172179 | 363405 | 52490 | 351027 |
| 314WGAAXX_2 | 84421 | 220282 | 267707 | 355403 | 539265 | 56877 | 467736 |
| 314WGAAXX_3 | 80503 | 195567 | 195607 | 244213 | 526353 | 49670 | 577778 |
| 314WGAAXX_5 | 75563 | 119359 | 208828 | 234418 | 500216 | 79869 | 346894 |
| 314WGAAXX_7 | 97029 | 160675 | 241034 | 317884 | 355859 | 51874 | 396465 |
| 315C1AAXX_3 | 52106 | 210379 | 220216 | 247886 | 435208 | 43285 | 328369 |
| 315C1AAXX_4 | 88847 | 181459 | 226783 | 328769 | 432373 | 46965 | 456446 |
| 315CUAAXX_1 | 44676 | 201166 | 159345 | 493464 | 524838 | 29100 | 742682 |
| 315CUAAXX_2 | 113417 | 183698 | 185692 | 350114 | 482254 | 88101 | 726492 |
| 315CUAAXX_5 | 105492 | 597274 | 218232 | 385358 | 580263 | 62576 | 749043 |
| 315CUAAXX_6 | 75623 | 201907 | 186748 | 371729 | 506088 | 63512 | 741594 |
| 315CUAAXX_7 | 98023 | 237693 | 213868 | 327455 | 827048 | 55833 | 448263 |
| 429T4AAXX_1 | 87681 | 310286 | 203932 | 570413 | 1124589 | 75649 | 736188 |
| 429T4AAXX_2 | 94814 | 262330 | 278704 | 542068 | 858630 | 68126 | 1223716 |
| 429T4AAXX_3 | 93569 | 379415 | 306226 | 717944 | 1182984 | 84017 | 955212 |
| 429T4AAXX_4 | 135462 | 396095 | 230439 | 449738 | 662223 | 124059 | 805585 |
| 429T4AAXX_5 | 129981 | 328365 | 353647 | 542238 | 888647 | 119143 | 778172 |
| 429T4AAXX_6 | 125897 | 280370 | 299624 | 560182 | 836537 | 92190 | 994303 |
| 429T4AAXX_7 | 115015 | 292988 | 293608 | 550800 | 830020 | 95576 | 890576 |
| 42AV4AAXX_1 | 183664 | 464872 | 732513 | 659522 | 660194 | 150049 | 843567 |
| 42AV4AAXX_2 | 159657 | 366253 | 513960 | 667756 | 1108446 | 116987 | 989241 |
| 42AV4AAXX_3 | 239631 | 420607 | 535741 | 687861 | 1147108 | 109950 | 892661 |
| 42AV4AAXX_5 | 217624 | 589687 | 626171 | 526077 | 1103895 | 125369 | 797328 |
| 42AV4AAXX_6 | 144892 | 444177 | 536724 | 750227 | 1339842 | 153406 | 1044198 |
| 42AV4AAXX_7 | 240673 | 520321 | 587548 | 602459 | 816431 | 160655 | 885971 |
| 42B08AAXX_2 | 44985 | 181145 | 219314 | 449046 | 502897 | 52600 | 471005 |
| 42B08AAXX_4 | 87689 | 215252 | 294638 | 443632 | 648557 | 60423 | 710184 |
| 42B08AAXX_5 | 135398 | 337240 | 320597 | 354844 | 519585 | 95688 | 1051292 |
| 42B08AAXX_7 | 133321 | 301713 | 311821 | 488962 | 562428 | 87375 | 929170 |
| 42B08AAXX_8 | 107701 | 274115 | 235911 | 327694 | 511036 | 57126 | 659603 |
| 42B20AAXX_5 | 209190 | 467725 | 450993 | 694353 | 771903 | 128376 | 940873 |
| 42NY4AAXX_6 | 205342 | 444485 | 432596 | 656600 | 732221 | 124200 | 880094 |
| 42B3YAAXX_4 | 122911 | 217816 | 214838 | 259869 | 329743 | 89973 | 406654 |
| 42B3YAAXX_5 | 76991 | 238721 | 249871 | 444537 | 407853 | 48053 | 439647 |
| 42B3YAAXX_6 | 89415 | 173152 | 212637 | 254405 | 425387 | 67202 | 313961 |
| 42B3YAAXX_7 | 85187 | 147521 | 177033 | 199290 | 485369 | 84753 | 275899 |
| 42B48AAXX_1 | 157567 | 357096 | 324072 | 486779 | 588106 | 104340 | 855778 |
| 42NY4AAXX_3 | 149692 | 347162 | 308863 | 478696 | 576775 | 98369 | 851208 |
| 42B48AAXX_5 | 199068 | 419524 | 430967 | 546376 | 678342 | 141001 | 1099533 |
| 42B48AAXX_6 | 151211 | 337654 | 351131 | 501715 | 579271 | 95561 | 963573 |
| 42NY4AAXX_4 | 153712 | 337694 | 349846 | 502524 | 580799 | 95937 | 964750 |
| 42HAMAAXX_3 | 113762 | 203652 | 266405 | 396952 | 522081 | 87179 | 488568 |
| 42HAMAAXX_5 | 85058 | 279244 | 186740 | 523621 | 664276 | 136514 | 690685 |
| 42HAMAAXX_6 | 152712 | 268059 | 271697 | 264592 | 647029 | 87824 | 612018 |
| 42CJFAAXX_1 | 107210 | 253166 | 247695 | 371628 | 462859 | 66837 | 847172 |
| 42CJFAAXX_6 | 82791 | 225768 | 192873 | 399576 | 561510 | 39428 | 659569 |
| 42CJFAAXX_7 | 103297 | 227792 | 235351 | 325864 | 411291 | 58748 | 770142 |
| 42P6UAAXX_8 | 9434 | 22385 | 22468 | 34439 | 42540 | 5384 | 82838 |
| 42PFAAAXX_4 | 147237 | 321156 | 331444 | 454469 | 575947 | 82710 | 1075881 |
| 42CJJAAXX_1 | 191279 | 336934 | 450231 | 613173 | 966498 | 84967 | 706916 |
| 42CJJAAXX_2 | 124945 | 342249 | 321932 | 230558 | 562836 | 308232 | 622509 |
| 42CJJAAXX_3 | 153268 | 560000 | 303494 | 332662 | 741519 | 654827 | 396566 |
| 42CJJAAXX_4 | 163761 | 377701 | 341796 | 410706 | 616158 | 170117 | 535127 |
| 42CJJAAXX_6 | 57329 | 138102 | 137298 | 208204 | 273940 | 43922 | 398117 |
| 42PF0AAXX_3 | 89811 | 211165 | 211277 | 317068 | 420401 | 67987 | 609756 |
| 42CJJAAXX_8 | 180667 | 392391 | 376568 | 535904 | 674538 | 115827 | 1129041 |
| 42D3NAAXX_1 | 103626 | 210600 | 217406 | 281327 | 376340 | 64773 | 551192 |
| 42D3NAAXX_2 | 90017 | 192720 | 209294 | 327628 | 451149 | 57475 | 821143 |
| 42D3NAAXX_3 | 111605 | 270225 | 287060 | 395582 | 528421 | 74766 | 863473 |
| 42D3NAAXX_5 | 101033 | 258158 | 269703 | 424360 | 526339 | 78284 | 810150 |
| 42D3NAAXX_6 | 111949 | 275084 | 241921 | 395198 | 499482 | 72962 | 883991 |
| 42P6UAAXX_6 | 137492 | 334521 | 292827 | 479064 | 604936 | 90069 | 1075564 |
| 42D3NAAXX_7 | 119549 | 280565 | 246758 | 382287 | 512237 | 82333 | 984441 |
| 42P6UAAXX_7 | 157301 | 367949 | 321014 | 505824 | 676537 | 107458 | 1301604 |
| 42PF0AAXX_6 | 203757 | 276856 | 266202 | 289557 | 476877 | 79055 | 747840 |
| 42PF0AAXX_7 | 118782 | 205379 | 257383 | 235074 | 454991 | 77263 | 529297 |
| 42PFAAAXX_5 | 104288 | 332247 | 265231 | 554906 | 816378 | 116342 | 808975 |
| 42PFAAAXX_6 | 128537 | 274791 | 286314 | 350332 | 516328 | 115605 | 444542 |
| 42PMUAAXX_6 | 74306 | 246413 | 198971 | 441047 | 624707 | 85140 | 656551 |
| 42PMUAAXX_7 | 71528 | 223310 | 182823 | 397188 | 560509 | 70401 | 595361 |
| 42PFAAAXX_7 | 192472 | 290995 | 372479 | 432009 | 724848 | 117538 | 723638 |
| 42PFAAAXX_8 | 82184 | 296342 | 292370 | 465173 | 678357 | 59708 | 473132 |
| 42PMUAAXX_1 | 129056 | 216453 | 328197 | 436236 | 494706 | 68871 | 642534 |
| 42PMUAAXX_2 | 78874 | 165243 | 189323 | 297949 | 382713 | 42500 | 314179 |
| 42PMUAAXX_4 | 100035 | 210152 | 183766 | 279848 | 420999 | 57326 | 454008 |
| 42PMUAAXX_8 | 93102 | 242858 | 303405 | 192061 | 236039 | 64952 | 185788 |
| 42RMPAAXX_1 | 238255 | 397329 | 407617 | 751336 | 792457 | 163611 | 792703 |
| 42RMPAAXX_2 | 316884 | 465730 | 478955 | 534607 | 820007 | 174370 | 746104 |
| 42RMPAAXX_3 | 190174 | 345407 | 396993 | 621630 | 782162 | 142812 | 862010 |
| 42RMPAAXX_4 | 113098 | 247897 | 224218 | 575497 | 590309 | 82535 | 826440 |
| 42RMPAAXX_5 | 223687 | 299796 | 358355 | 309324 | 517339 | 125262 | 420903 |
| 42RMPAAXX_6 | 134288 | 211770 | 213619 | 213271 | 592476 | 128901 | 417823 |
| 42RMPAAXX_7 | 102052 | 307709 | 276830 | 279301 | 610360 | 91910 | 416047 |
| 42RMPAAXX_8 | 168652 | 397031 | 274988 | 439752 | 864037 | 80432 | 864558 |
| 42RYFAAXX_1 | 60954 | 215233 | 152194 | 212682 | 333975 | 38261 | 398124 |
| 42RYFAAXX_2 | 84209 | 188893 | 224859 | 375988 | 369090 | 42552 | 422784 |
| 42RYFAAXX_3 | 98139 | 445416 | 226564 | 227638 | 361383 | 43991 | 448483 |
| 42RYFAAXX_4 | 95431 | 205626 | 145190 | 214201 | 283889 | 38386 | 442908 |
| 42RYFAAXX_5 | 79966 | 314259 | 176886 | 203896 | 294773 | 50589 | 304253 |
| 42RYFAAXX_6 | 155410 | 371075 | 389499 | 349527 | 567297 | 92664 | 593761 |
| 42RYFAAXX_7 | 34764 | 75871 | 53317 | 80678 | 104595 | 15257 | 142358 |
| 42RYFAAXX_8 | 75724 | 293192 | 197216 | 199042 | 318750 | 46744 | 342216 |
| 42TA8AAXX_1 | 102803 | 316271 | 252989 | 371143 | 632994 | 104269 | 536160 |
| 42TA8AAXX_2 | 119709 | 370569 | 298626 | 438362 | 749774 | 122406 | 631885 |
| 42TA8AAXX_3 | 111053 | 348431 | 281692 | 419917 | 715548 | 114221 | 613093 |
| 42TA8AAXX_4 | 3240 | 10519 | 8670 | 13703 | 22909 | 3282 | 20952 |
| 42TA8AAXX_5 | 133410 | 413069 | 332652 | 470188 | 812571 | 138357 | 667935 |
| 42TA8AAXX_6 | 159910 | 477001 | 374295 | 461542 | 834394 | 165743 | 572009 |
| 42TA8AAXX_7 | 116306 | 367253 | 296708 | 445287 | 756406 | 119350 | 651243 |
| 42TB9AAXX_1 | 269012 | 519589 | 563046 | 588458 | 767793 | 194686 | 1186066 |
| 42TB9AAXX_2 | 111372 | 280365 | 269954 | 401921 | 468099 | 73869 | 856154 |
| 42TB9AAXX_3 | 249283 | 498473 | 485828 | 571011 | 741193 | 168650 | 1054156 |
| 42TB9AAXX_4 | 142274 | 264068 | 260072 | 299896 | 401818 | 83946 | 589019 |
| 42Y6WAAXX_6 | 213511 | 398844 | 397375 | 452581 | 606395 | 127677 | 887179 |
| 42TB9AAXX_6 | 226156 | 459793 | 497035 | 615896 | 753333 | 144199 | 1033291 |
| 42TB9AAXX_7 | 209280 | 445148 | 470696 | 584291 | 766432 | 132106 | 1087024 |
| 42TBWAAXX_1 | 90713 | 153577 | 143232 | 454342 | 400467 | 54907 | 355599 |
| 42TBWAAXX_2 | 190310 | 295823 | 377067 | 491265 | 688346 | 121515 | 739446 |
| 42TBWAAXX_3 | 138045 | 465226 | 317108 | 387097 | 581522 | 84087 | 575360 |
| 42TBWAAXX_4 | 207387 | 360421 | 366684 | 507993 | 676571 | 115219 | 798682 |
| 42TBWAAXX_5 | 71393 | 158732 | 197226 | 518476 | 456811 | 56643 | 691344 |
| 42TBWAAXX_6 | 59995 | 85395 | 147645 | 301800 | 372085 | 47095 | 463537 |
| 42Y6WAAXX_1 | 68832 | 97406 | 167908 | 343002 | 423787 | 53499 | 526991 |
| 42TBWAAXX_7 | 87998 | 207252 | 198292 | 471578 | 457264 | 61954 | 683259 |
| 42TBWAAXX_8 | 66505 | 236197 | 282722 | 449816 | 350419 | 85620 | 700960 |
| 42TCBAAXX_1 | 84856 | 199663 | 167372 | 382550 | 650452 | 61165 | 757773 |
| 42TCBAAXX_2 | 70643 | 261203 | 225729 | 366037 | 403955 | 41310 | 507333 |
| 42TCBAAXX_3 | 78757 | 170760 | 205826 | 489551 | 657392 | 68297 | 557364 |
| 42TCBAAXX_4 | 92377 | 270965 | 332339 | 380416 | 935060 | 107876 | 862216 |
| 42TCBAAXX_5 | 181775 | 478618 | 347656 | 418781 | 722648 | 112087 | 818399 |
| 42TCBAAXX_6 | 63472 | 141990 | 128695 | 185471 | 303696 | 40227 | 255344 |
| 42TCBAAXX_7 | 113459 | 208140 | 205107 | 243758 | 410627 | 77298 | 378240 |
| 42TCBAAXX_8 | 120100 | 233026 | 217898 | 262018 | 449792 | 90417 | 368525 |
| 42TCLAAXX_1 | 31294 | 72127 | 65745 | 68073 | 103428 | 15966 | 119141 |
| 42Y39AAXX_2 | 295412 | 565225 | 570491 | 503837 | 797180 | 146703 | 777303 |
| 42TCLAAXX_3 | 26408 | 76681 | 52640 | 62304 | 93057 | 14703 | 97166 |
| 42Y39AAXX_5 | 276531 | 610765 | 503292 | 504634 | 783040 | 153625 | 674447 |
| 42TCLAAXX_4 | 79280 | 173616 | 156022 | 186725 | 267954 | 43703 | 275560 |
| 42Y39AAXX_6 | 295081 | 484407 | 503591 | 487102 | 738030 | 157906 | 575911 |
| 42TCLAAXX_5 | 57284 | 105241 | 113242 | 176118 | 218812 | 34522 | 246277 |
| 42Y39AAXX_7 | 269077 | 404421 | 453634 | 563079 | 726716 | 156512 | 609086 |
| 42TG0AAXX_2 | 41743 | 134630 | 102861 | 158275 | 265361 | 24946 | 283270 |
| 42TG0AAXX_3 | 188885 | 227278 | 381770 | 322071 | 612352 | 97285 | 584511 |
| 42TG0AAXX_4 | 206182 | 326424 | 330167 | 493368 | 775245 | 150773 | 706215 |
| 42TG0AAXX_5 | 99836 | 228916 | 253742 | 248734 | 475330 | 90759 | 430398 |
| 42TG0AAXX_6 | 175839 | 285404 | 432567 | 314136 | 642173 | 98259 | 541707 |
| 42TG0AAXX_7 | 4593 | 7653 | 19042 | 13322 | 16109 | 2802 | 20993 |
| 42TGAAXX_8 | 206427 | 307061 | 334170 | 373589 | 705205 | 106244 | 678567 |
| 42Y2MAAXX_1 | 270534 | 484529 | 453173 | 616855 | 764725 | 204777 | 681284 |
| 42Y2MAAXX_2 | 273003 | 484835 | 479673 | 648395 | 751517 | 213746 | 664758 |
| 42Y2MAAXX_3 | 291368 | 494262 | 539380 | 623651 | 794378 | 231024 | 683412 |
| 42Y2MAAXX_4 | 278473 | 542426 | 499027 | 828490 | 875415 | 241471 | 798123 |
| 42Y2MAAXX_5 | 320283 | 621698 | 496634 | 629584 | 843018 | 198889 | 842852 |
| 42Y2MAAXX_6 | 288568 | 493990 | 492148 | 567998 | 713767 | 218092 | 652522 |
| 42Y2MAAXX_7 | 323956 | 499663 | 431385 | 617290 | 765655 | 177610 | 836956 |
| 42Y2MAAXX_8 | 278838 | 518161 | 541146 | 518374 | 693038 | 197100 | 807671 |
| 42Y39AAXX_3 | 271772 | 438482 | 465822 | 643374 | 881882 | 139761 | 831681 |
| 42Y39AAXX_4 | 278568 | 438735 | 467036 | 634647 | 874703 | 141720 | 801472 |
| 42Y6NAAXX_1 | 147165 | 351580 | 355956 | 421690 | 699986 | 169753 | 539676 |
| 42Y6NAAXX_2 | 177592 | 327524 | 386813 | 415661 | 609180 | 108153 | 550552 |
| 42Y6NAAXX_4 | 179971 | 299723 | 445732 | 397819 | 713063 | 130014 | 415320 |
| 42Y6NAAXX_5 | 128681 | 364378 | 467395 | 472436 | 724392 | 139509 | 375033 |
| 42Y6NAAXX_6 | 186750 | 405830 | 419444 | 405601 | 707452 | 117384 | 455500 |
| 42Y6NAAXX_7 | 158591 | 380982 | 364727 | 375161 | 819995 | 168414 | 430706 |
| 42Y6NAAXX_8 | 136345 | 355898 | 426104 | 632888 | 675552 | 127650 | 488296 |
| 42Y6WAAXX_2 | 179686 | 289551 | 304790 | 450183 | 709224 | 122166 | 690714 |
| 42YMTAAXX_2 | 129978 | 206485 | 216045 | 311371 | 492547 | 87948 | 475863 |
| 42Y6WAAXX_3 | 308213 | 593156 | 388950 | 366495 | 605803 | 86169 | 759055 |
| 42YMTAAXX_4 | 137863 | 272920 | 174411 | 169970 | 277948 | 38512 | 357263 |
| 42Y6WAAXX_4 | 116586 | 315063 | 205748 | 220942 | 348297 | 56923 | 372245 |
| 42YMTAAXX_8 | 62365 | 172185 | 109344 | 119953 | 187842 | 30500 | 206778 |
| 42YMMAAXX_1 | 206334 | 417554 | 434909 | 380359 | 667555 | 186948 | 416983 |
| 42YMMAAXX_2 | 255370 | 389854 | 687630 | 491737 | 882467 | 101665 | 694661 |
| 42YMMAAXX_8 | 223205 | 359462 | 633993 | 482187 | 860711 | 90103 | 708389 |
| 42YMMAAXX_3 | 289342 | 602271 | 475937 | 488053 | 831656 | 133494 | 897600 |
| 42YMMAAXX_4 | 232121 | 434887 | 446076 | 513033 | 1174870 | 121920 | 555755 |
| 42YMMAAXX_5 | 286426 | 630148 | 531084 | 397258 | 755888 | 132035 | 617896 |
| 42YMMAAXX_6 | 257279 | 579993 | 498907 | 428421 | 705012 | 132999 | 620498 |
| 42YMMAAXX_7 | 289351 | 413708 | 469439 | 428772 | 746295 | 140396 | 633067 |
| 42YMTAAXX_1 | 132472 | 414341 | 321691 | 491875 | 641655 | 86069 | 975114 |
| 42YMTAAXX_3 | 203277 | 376574 | 296507 | 322606 | 569455 | 88900 | 539572 |
| 42YMTAAXX_5 | 151436 | 201272 | 230075 | 320718 | 395592 | 110147 | 429042 |
| 42YMTAAXX_6 | 203005 | 544459 | 463162 | 377122 | 713350 | 110228 | 732114 |
| 42YMTAAXX_7 | 113731 | 209079 | 235116 | 311471 | 415222 | 57301 | 496528 |
| 43336AAXX_1 | 744888 | 890921 | 866567 | 737050 | 1097795 | 544417 | 661674 |
| 43336AAXX_2 | 486445 | 765152 | 797094 | 1010523 | 1372658 | 406991 | 1171495 |
| 43336AAXX_3 | 741071 | 878232 | 812387 | 735251 | 976647 | 572594 | 646032 |
| 43336AAXX_4 | 652793 | 836103 | 833781 | 814174 | 1167054 | 492746 | 814656 |
| 43336AAXX_5 | 769561 | 842001 | 832253 | 816255 | 952817 | 642681 | 620365 |
| 43336AAXX_6 | 813509 | 908603 | 890605 | 656326 | 941244 | 618115 | 513310 |
| 43336AAXX_7 | 146057 | 369815 | 297263 | 465849 | 945022 | 108262 | 735878 |
| 6137KAAXX_3 | 189071 | 649893 | 513012 | 802223 | 1185313 | 155758 | 1111030 |
| 6137KAAXX_4 | 187876 | 649534 | 514721 | 802108 | 1185621 | 155055 | 1111015 |
| 6137LAAXX_6 | 42289 | 127925 | 105348 | 176914 | 257226 | 44270 | 272519 |
| 6137KAAXX_6 | 235031 | 529845 | 635205 | 753290 | 1335989 | 232111 | 919193 |
| 6137KAAXX_7 | 211931 | 629023 | 672230 | 816612 | 1257774 | 141626 | 989410 |
| 6137KAAXX_8 | 363925 | 551408 | 591748 | 656046 | 959712 | 194438 | 761930 |
| 6137LAAXX_7 | 399414 | 374633 | 330883 | 320202 | 296130 | 333982 | 235103 |
| 6137LAAXX_8 | 265066 | 266204 | 241876 | 243873 | 246386 | 239609 | 238819 |
| 61VFCAAXX_1 | 263793 | 540753 | 617278 | 660685 | 939451 | 236516 | 1022661 |
| 61VFCAAXX_2 | 264858 | 502480 | 635239 | 662748 | 1006732 | 243369 | 945727 |
| 61VFCAAXX_3 | 349514 | 499480 | 798323 | 667540 | 1114518 | 230696 | 940702 |
| 61VFCAAXX_6 | 328964 | 611492 | 577030 | 849413 | 1257799 | 242381 | 976659 |
| 62837AAXX_1 | 341571 | 682004 | 676736 | 733062 | 1178080 | 209448 | 1112898 |
| 62837AAXX_2 | 428166 | 669624 | 793024 | 732727 | 1185800 | 268907 | 1030170 |
| 62837AAXX_3 | 441125 | 678942 | 772911 | 789617 | 1209917 | 252488 | 950079 |
| 62837AAXX_4 | 342498 | 624271 | 673985 | 589706 | 1031729 | 211420 | 810328 |
| 62837AAXX_7 | 111320 | 188931 | 177458 | 223203 | 331747 | 79936 | 283690 |
| 62837AAXX_8 | 317008 | 559922 | 592957 | 686832 | 1004832 | 191886 | 891973 |
| 6286PAAXX_1 | 316499 | 593025 | 596526 | 771301 | 1096203 | 182344 | 1075494 |
| 6286PAAXX_2 | 322378 | 662735 | 635231 | 713455 | 1080213 | 198049 | 1017599 |
| 6286PAAXX_3 | 361324 | 475785 | 511994 | 1029004 | 1251046 | 133584 | 841129 |
| 6286PAAXX_4 | 363630 | 380069 | 702809 | 727688 | 1128575 | 183817 | 892735 |
| 6286PAAXX_5 | 266943 | 481047 | 517230 | 746431 | 1233338 | 184982 | 923719 |
| 6286PAAXX_6 | 820287 | 542642 | 790078 | 750706 | 1451421 | 175697 | 829154 |
| 6286PAAXX_7 | 143150 | 669500 | 609770 | 921555 | 1234712 | 123521 | 930750 |
| 6286PAAXX_8 | 330126 | 735431 | 653599 | 744618 | 1161407 | 206579 | 1013607 |

| Flowcell_lane | hs_ref_chr20 | hs_ref_chr21 | hs_ref_chr22 | hs_ref_chrX | hs_ref_chrY | chrM | humRibosomal |
|---|---|---|---|---|---|---|---|
| 314N5AAXX_6 | 264760 | 48535 | 148277 | 187499 | 1578 | 490016 | 38426 |
| 314WGAAXX_4 | 208607 | 80798 | 153146 | 216088 | 673 | 346702 | 61513 |
| 30Y5NAAXX_1 | 206798 | 64850 | 139387 | 248069 | 1078 | 704786 | 55760 |
| 315C1AAXX_1 | 178511 | 56722 | 129297 | 209380 | 769 | 395222 | 17804 |
| 30LUMAAXX_2 | 147528 | 3870 | 136446 | 311228 | 10427 | 386156 | 59247 |
| 42AV4AAXX_4 | 397142 | 77346 | 280107 | 319781 | 1823 | 1614866 | 67488 |
| 314N5AAXX_5 | 281217 | 91492 | 148573 | 222501 | 1628 | 452571 | 31641 |
| 30TYGAAXX_1 | 41331 | 21922 | 45061 | 60598 | 2089 | 312334 | 107632 |
| 42PMUAAXX_5 | 271262 | 63408 | 176696 | 183360 | 2731 | 756960 | 15238 |
| 42D3NAAXX_4 | 246293 | 43473 | 193558 | 184299 | 5352 | 1099027 | 99879 |
| 42Y39AAXX_8 | 411418 | 78621 | 304239 | 319017 | 11795 | 2441835 | 142921 |
| 42YRVAAXX_2 | 131213 | 59404 | 126766 | 174844 | 7804 | 1295521 | 39776 |
| 42B20AAXX_2 | 295903 | 83566 | 245140 | 241371 | 10323 | 2430637 | 103065 |
| 30TYGAAXX_3 | 62167 | 31319 | 71037 | 89728 | 2289 | 183246 | 51935 |
| 42B20AAXX_6 | 288390 | 113523 | 181400 | 381290 | 20268 | 697945 | 103631 |
| 42HAMAAXX_1 | 270239 | 78308 | 179075 | 172573 | 972 | 820760 | 69893 |
| 42B20AAXX_1 | 542699 | 104767 | 377999 | 639599 | 1127 | 909271 | 42723 |
| 42YRVAAXX_3 | 349081 | 79500 | 288342 | 270650 | 3879 | 1222091 | 207520 |
| 42HAMAAXX_2 | 313482 | 109467 | 377034 | 321809 | 1294 | 1582009 | 102727 |
| 42YRVAAXX_1 | 198396 | 58227 | 160532 | 167110 | 2967 | 1279621 | 166910 |
| 30TUEAAXX_3 | 144238 | 19266 | 150868 | 188050 | 7571 | 143088 | 48138 |
| 3035BAAXX_3 | 270873 | 144633 | 391917 | 174556 | 3584 | 840891 | 102054 |
| 314NNAAXX_3 | 165232 | 61702 | 204862 | 169316 | 914 | 254577 | 141731 |
| 314WAAAXX_3 | 209569 | 75288 | 251538 | 209601 | 850 | 436895 | 39923 |
| 314WAAAXX_5 | 128799 | 61325 | 152127 | 93203 | 5039 | 338259 | 24734 |
| 42Y39AAXX_1 | 314279 | 148913 | 334422 | 239898 | 15772 | 1021588 | 44246 |
| 30WB6AAXX_7 | 238363 | 51390 | 143754 | 138258 | 2126 | 401820 | 32379 |
| 30351AAXX_7 | 177359 | 67345 | 133796 | 164677 | 12170 | 603105 | 108708 |
| 314T1AAXX_1 | 154062 | 54663 | 122891 | 135101 | 8916 | 510008 | 113368 |
| 30U00AAXX_7 | 27941 | 13937 | 26350 | 33910 | 1249 | 208058 | 145164 |
| 30LEJAAXX_7 | 290436 | 10613 | 220389 | 539069 | 20264 | 389604 | 35647 |
| 30TYGAAXX_5 | 80501 | 22687 | 49858 | 101447 | 4352 | 305815 | 78468 |
| 314NNAAXX_6 | 156308 | 50733 | 206291 | 167672 | 3918 | 505174 | 246036 |
| 3064YAAXX_1 | 258668 | 98825 | 143300 | 237962 | 1130 | 559937 | 71147 |
| 30LF7AAXX_1 | 145500 | 13855 | 105061 | 300722 | 8021 | 215405 | 243681 |
| 315CUAAXX_3 | 276168 | 48750 | 203555 | 187686 | 2420 | 525057 | 20386 |
| 30LF7AAXX_2 | 141515 | 15927 | 165401 | 272283 | 19581 | 503749 | 165698 |
| 30LF7AAXX_3 | 128787 | 15725 | 157166 | 253954 | 17654 | 372826 | 53181 |
| 314WAAAXX_4 | 169785 | 61317 | 134702 | 172398 | 2175 | 188945 | 16118 |
| 42PMUAAXX_3 | 362833 | 105473 | 288023 | 265635 | 13236 | 3017398 | 170765 |
| 6137LAAXX_2 | 64990 | 10613 | 22744 | 35566 | 129 | 110841 | 51820 |
| 30Y6GAAXX_1 | 205732 | 64524 | 185486 | 181507 | 1007 | 552801 | 260358 |
| 314NNAAXX_5 | 213831 | 64123 | 232681 | 205683 | 3705 | 261712 | 103346 |
| 30Y6GAAXX_3 | 193792 | 61419 | 202425 | 192551 | 8202 | 637496 | 240730 |
| 30LUMAAXX_3 | 161338 | 8064 | 140097 | 253648 | 12746 | 509672 | 92377 |
| 314WGAAXX_1 | 243329 | 76279 | 163901 | 158714 | 2518 | 679066 | 30546 |
| 42HAMAAXX_7 | 236770 | 55872 | 126158 | 147315 | 419 | 1474806 | 59654 |
| 314N5AAXX_7 | 247485 | 60251 | 120685 | 154217 | 708 | 300005 | 24985 |
| 301YWAAXX_1 | 296197 | 146038 | 320091 | 329034 | 13440 | 1535846 | 373796 |
| 42P6UAAXX_4 | 315238 | 155833 | 338091 | 352348 | 14812 | 1799099 | 357641 |
| 301YWAAXX_2 | 311218 | 146203 | 330331 | 345136 | 11668 | 1007857 | 349390 |
| 42P6UAAXX_5 | 306677 | 145095 | 324505 | 340798 | 11573 | 1026274 | 337389 |
| 301YWAAXX_3 | 316730 | 124188 | 308570 | 337868 | 11393 | 1072598 | 328031 |
| 302LVAAXX_2 | 220468 | 107965 | 198599 | 265735 | 2528 | 809655 | 95165 |
| 302LVAAXX_5 | 187009 | 70823 | 262823 | 251563 | 1284 | 1232115 | 17482 |
| 302LVAAXX_6 | 186199 | 92189 | 218576 | 213412 | 1340 | 782178 | 11930 |
| 302LVAAXX_7 | 201516 | 69168 | 224513 | 246003 | 919 | 824346 | 14462 |
| 3035BAAXX_1 | 195707 | 79568 | 239522 | 216757 | 5280 | 652672 | 46974 |
| 3035BAAXX_2 | 246460 | 65793 | 273653 | 224849 | 5632 | 1073783 | 149158 |
| 3035BAAXX_5 | 272144 | 117291 | 163485 | 323736 | 2035 | 1071459 | 144435 |
| 3035BAAXX_6 | 353701 | 88952 | 208121 | 149419 | 4697 | 786911 | 90961 |
| 3064YAAXX_2 | 204880 | 93200 | 221982 | 218346 | 6542 | 683142 | 63029 |
| 42P6UAAXX_1 | 300384 | 138967 | 316300 | 324423 | 11211 | 1075409 | 82822 |
| 3064YAAXX_3 | 196415 | 83900 | 198095 | 215114 | 5956 | 908011 | 136656 |
| 42P6UAAXX_2 | 255591 | 110250 | 255640 | 285539 | 8385 | 1227437 | 176274 |
| 3064YAAXX_4 | 190301 | 82067 | 193289 | 166213 | 9974 | 1459640 | 23445 |
| 30JD2AAXX_6 | 34103 | 12003 | 48672 | 100603 | 5604 | 41960 | 24852 |
| 3064YAAXX_5 | 200768 | 96170 | 204715 | 176561 | 9379 | 1482267 | 112734 |
| 3064YAAXX_6 | 196226 | 96151 | 219694 | 182302 | 6914 | 1056672 | 49998 |
| 3064YAAXX_7 | 219222 | 82234 | 206523 | 258158 | 4896 | 642516 | 86764 |
| 42P6UAAXX_3 | 343416 | 128957 | 320980 | 408774 | 8171 | 1091748 | 129297 |
| 30650AAXX_1 | 201415 | 63575 | 114582 | 168614 | 1863 | 1082790 | 15006 |
| 30650AAXX_5 | 261440 | 151217 | 169568 | 214353 | 4312 | 493577 | 34121 |
| 30650AAXX_6 | 171427 | 70889 | 121608 | 171219 | 4484 | 1434350 | 32251 |
| 30650AAXX_7 | 163188 | 99960 | 144578 | 204904 | 2555 | 604154 | 43781 |
| 30Y5NAAXX_2 | 285963 | 65682 | 204520 | 215990 | 2161 | 362641 | 102808 |
| 42Y6NAAXX_3 | 291369 | 71664 | 162992 | 288443 | 3359 | 1331820 | 69223 |
| 30Y5NAAXX_3 | 176009 | 92131 | 184616 | 183812 | 9216 | 562567 | 66893 |
| 42B48AAXX_7 | 296821 | 152337 | 325010 | 304731 | 13270 | 688006 | 167085 |
| 30Y5NAAXX_4 | 172648 | 83913 | 181686 | 186383 | 9215 | 306694 | 57853 |
| 42CJFAAXX_2 | 255774 | 130807 | 287063 | 268510 | 11223 | 725801 | 118010 |
| 42NY4AAXX_5 | 322689 | 166122 | 354478 | 344917 | 16237 | 1034324 | 132036 |
| 30GC3AAXX_3 | 114126 | 30175 | 166311 | 172603 | 12548 | 262440 | 144474 |
| 30Y5NAAXX_6 | 200256 | 99240 | 221610 | 182761 | 12177 | 391655 | 124451 |
| 30Y5NAAXX_7 | 125418 | 71583 | 108570 | 146078 | 14885 | 201067 | 225994 |
| 30U00AAXX_1 | 137685 | 78822 | 120894 | 126602 | 4047 | 847444 | 41986 |
| 30TUEAAXX_5 | 141139 | 37430 | 215138 | 212419 | 11024 | 361621 | 174618 |
| 30TUEAAXX_6 | 136657 | 37929 | 178390 | 191968 | 13170 | 450867 | 347304 |
| 30TUEAAXX_2 | 135949 | 27277 | 174035 | 219137 | 11407 | 612305 | 77664 |
| 30TUEAAXX_7 | 97573 | 19544 | 112730 | 161396 | 8386 | 260154 | 56389 |
| 30TUEAAXX_1 | 142800 | 30159 | 201038 | 215177 | 11680 | 343230 | 83842 |
| 30TUEAAXX_8 | 67701 | 12859 | 81410 | 102798 | 6000 | 164077 | 32855 |
| 30GC3AAXX_1 | 153592 | 16319 | 140228 | 192693 | 6910 | 393344 | 231851 |
| 30GC3AAXX_2 | 142383 | 24399 | 218087 | 217853 | 6852 | 386922 | 270139 |
| 30GC3AAXX_5 | 136700 | 22931 | 189379 | 198258 | 10317 | 271172 | 78527 |
| 30GC3AAXX_6 | 107449 | 27073 | 143112 | 185895 | 15326 | 495847 | 125728 |
| 30JD2AAXX_5 | 88417 | 19429 | 129429 | 176994 | 13967 | 319856 | 57004 |
| 30JD2AAXX_3 | 143814 | 30884 | 209733 | 257320 | 18377 | 355554 | 57692 |
| 30JD2AAXX_7 | 79913 | 33939 | 110679 | 236012 | 16534 | 414792 | 29319 |
| 30JD2AAXX_8 | 54420 | 15065 | 67681 | 110357 | 7558 | 171548 | 13018 |
| 30LEJAAXX_2 | 333048 | 18431 | 164598 | 419908 | 15719 | 519944 | 147483 |
| 30U00AAXX_2 | 120223 | 47129 | 147983 | 148878 | 1531 | 336810 | 38087 |
| 30U00AAXX_3 | 190758 | 108712 | 158320 | 181356 | 2146 | 701896 | 95366 |
| 30U00AAXX_4 | 165968 | 79518 | 194154 | 149740 | 7392 | 437843 | 285882 |
| 30U00AAXX_5 | 25101 | 16675 | 19583 | 21785 | 1196 | 187180 | 32847 |
| 30U00AAXX_6 | 22806 | 10083 | 28348 | 24156 | 1807 | 78677 | 17420 |
| 42B08AAXX_6 | 170695 | 68675 | 211136 | 224867 | 14378 | 1096607 | 1442668 |
| 30WB6AAXX_5 | 188638 | 60807 | 201312 | 169477 | 4296 | 622425 | 24766 |
| 315CUAAXX_4 | 224039 | 65022 | 249689 | 189366 | 3163 | 583042 | 26578 |
| 30WB6AAXX_6 | 131595 | 51520 | 158869 | 145576 | 2212 | 873721 | 27388 |
| 30WG6AAXX_4 | 266661 | 51297 | 152110 | 206493 | 1668 | 813621 | 298422 |
| 30WG6AAXX_5 | 349832 | 64972 | 233609 | 239297 | 1490 | 273293 | 23632 |
| 30WG6AAXX_6 | 161878 | 128314 | 257881 | 225580 | 3158 | 184512 | 53031 |
| 30WG6AAXX_7 | 437929 | 91782 | 141228 | 251518 | 1080 | 386527 | 68483 |
| 30WU2AAXX_1 | 312253 | 155290 | 328914 | 278912 | 9854 | 782351 | 129310 |
| 42B08AAXX_1 | 193982 | 145172 | 281632 | 189479 | 5819 | 682795 | 125957 |
| 42CJJAAXX_5 | 341889 | 170559 | 356621 | 309704 | 11655 | 924420 | 128762 |
| 42CJJAAXX_7 | 161774 | 70769 | 184820 | 164714 | 5914 | 233249 | 701465 |
| 42NY4AAXX_2 | 302275 | 133039 | 340638 | 312375 | 11867 | 475777 | 1196691 |
| 42B48AAXX_2 | 161654 | 72305 | 172124 | 188707 | 5337 | 566307 | 369749 |
| 42PFAAAXX_1 | 283982 | 126838 | 297888 | 333979 | 10473 | 1100375 | 570993 |
| 30WU2AAXX_4 | 195717 | 91417 | 216943 | 185577 | 8177 | 589225 | 126634 |
| 42B48AAXX_3 | 302309 | 143542 | 333352 | 294920 | 13995 | 989980 | 165254 |
| 30WU2AAXX_5 | 219775 | 106116 | 248463 | 219857 | 8298 | 291302 | 84988 |
| 42B48AAXX_4 | 335096 | 161063 | 367054 | 343368 | 14912 | 505577 | 101449 |
| 30WU2AAXX_6 | 94768 | 42084 | 97565 | 94181 | 2449 | 98226 | 187473 |
| 42B48AAXX_8 | 268942 | 116313 | 273270 | 265753 | 7503 | 297034 | 475827 |
| 42PFAAAXX_2 | 158006 | 69591 | 160822 | 157965 | 4413 | 166497 | 301230 |
| 30WU2AAXX_7 | 42888 | 17142 | 44540 | 37818 | 1563 | 102589 | 92820 |
| 42CJFAAXX_5 | 339178 | 132395 | 336641 | 290700 | 12924 | 880044 | 628447 |
| 30WU2AAXX_8 | 148087 | 74741 | 165864 | 154097 | 5417 | 476257 | 84960 |
| 42CJFAAXX_4 | 245983 | 124383 | 275706 | 256860 | 9107 | 810157 | 139181 |
| 42PFAAAXX_3 | 271859 | 137906 | 303397 | 286171 | 10228 | 919188 | 149340 |
| 30Y6GAAXX_2 | 131464 | 35468 | 175135 | 99105 | 2914 | 789026 | 562092 |
| 30Y6GAAXX_4 | 203015 | 81606 | 214998 | 169551 | 2989 | 648118 | 44199 |
| 30Y6GAAXX_5 | 127772 | 61942 | 160068 | 150870 | 778 | 1202754 | 61994 |
| 30Y6GAAXX_6 | 208508 | 64280 | 159650 | 142266 | 1353 | 531223 | 35653 |
| 30Y6GAAXX_7 | 251335 | 61594 | 182225 | 157814 | 3497 | 413938 | 31628 |
| 314N5AAXX_1 | 218643 | 69753 | 190267 | 181481 | 3851 | 844157 | 154187 |
| 314N5AAXX_2 | 187625 | 79233 | 195934 | 176220 | 953 | 414995 | 49478 |
| 314N5AAXX_3 | 218432 | 99142 | 310802 | 209616 | 1865 | 382975 | 93211 |
| 314N5AAXX_4 | 234844 | 93572 | 423264 | 257699 | 1799 | 513125 | 82212 |
| 314NNAAXX_2 | 306721 | 117621 | 250823 | 138066 | 1691 | 486708 | 289616 |
| 314NNAAXX_4 | 177690 | 63011 | 232450 | 174438 | 6562 | 319708 | 36583 |
| 314RPAAXX_5 | 179894 | 66247 | 168984 | 141012 | 4156 | 876841 | 89271 |
| 314RPAAXX_6 | 164691 | 55104 | 172065 | 160579 | 1481 | 327059 | 33900 |
| 314RPAAXX_7 | 241895 | 60428 | 147960 | 143518 | 2043 | 848335 | 54529 |
| 314T1AAXX_2 | 142629 | 89302 | 140127 | 181644 | 4884 | 399767 | 136140 |
| 314WAAAXX_1 | 228939 | 86247 | 150865 | 172110 | 2959 | 574871 | 64155 |
| 314WAAAXX_2 | 189688 | 63167 | 179307 | 253460 | 1149 | 344223 | 28611 |
| 314WAAAXX_6 | 165701 | 49018 | 183487 | 147114 | 1281 | 130114 | 12196 |
| 314WAAAXX_7 | 139081 | 73244 | 228107 | 139921 | 452 | 173025 | 18078 |
| 314WGAAXX_2 | 210203 | 63487 | 171429 | 99698 | 2523 | 373740 | 21854 |
| 314WGAAXX_3 | 204644 | 70034 | 240010 | 103007 | 855 | 381926 | 43066 |
| 314WGAAXX_5 | 370359 | 103175 | 225164 | 197402 | 1134 | 410446 | 47381 |
| 314WGAAXX_7 | 169076 | 62192 | 118793 | 278103 | 871 | 483810 | 92377 |
| 315C1AAXX_3 | 216424 | 69241 | 167846 | 148894 | 3090 | 214493 | 15292 |
| 315C1AAXX_4 | 239677 | 66296 | 176426 | 109906 | 1000 | 338711 | 28474 |
| 315CUAAXX_1 | 170575 | 48443 | 327916 | 213699 | 534 | 163300 | 37807 |
| 315CUAAXX_2 | 214352 | 110465 | 231279 | 291690 | 4468 | 512214 | 30544 |
| 315CUAAXX_5 | 247759 | 101772 | 352628 | 232916 | 3556 | 673610 | 111769 |
| 315CUAAXX_6 | 202014 | 74793 | 185387 | 135719 | 1264 | 551234 | 98533 |
| 315CUAAXX_7 | 172658 | 80746 | 199194 | 189783 | 962 | 777467 | 77675 |
| 429T4AAXX_1 | 249551 | 119718 | 281242 | 419753 | 4830 | 1601258 | 126735 |
| 429T4AAXX_2 | 267925 | 72106 | 351351 | 252611 | 1330 | 730772 | 199496 |
| 429T4AAXX_3 | 388148 | 137093 | 303818 | 321912 | 19998 | 1169947 | 193719 |
| 429T4AAXX_4 | 329878 | 105710 | 216856 | 282643 | 3086 | 742024 | 116066 |
| 429T4AAXX_5 | 404052 | 90265 | 183739 | 267473 | 6151 | 605693 | 50507 |
| 429T4AAXX_6 | 262807 | 114283 | 315127 | 242597 | 10509 | 485941 | 127036 |
| 429T4AAXX_7 | 264053 | 94533 | 359557 | 239140 | 11195 | 623870 | 194650 |
| 42AV4AAXX_1 | 420874 | 119406 | 236308 | 324944 | 13005 | 2120354 | 54167 |
| 42AV4AAXX_2 | 400046 | 78473 | 205630 | 301395 | 2445 | 2048491 | 47456 |
| 42AV4AAXX_3 | 463306 | 97799 | 324461 | 325154 | 1737 | 2062037 | 34842 |
| 42AV4AAXX_5 | 377952 | 115991 | 338960 | 270580 | 4824 | 1058598 | 61186 |
| 42AV4AAXX_6 | 482705 | 131023 | 434882 | 276641 | 1707 | 1227106 | 56910 |
| 42AV4AAXX_7 | 498381 | 112400 | 291280 | 410671 | 3349 | 869650 | 83496 |
| 42B08AAXX_2 | 271150 | 63237 | 220901 | 148004 | 2634 | 215196 | 26720 |
| 42B08AAXX_4 | 300359 | 66867 | 135530 | 185060 | 437 | 392084 | 54559 |
| 42B08AAXX_5 | 210323 | 98698 | 214852 | 224405 | 10427 | 917319 | 251404 |
| 42B08AAXX_7 | 265481 | 117534 | 255708 | 290859 | 9411 | 1051916 | 311425 |
| 42B08AAXX_8 | 205319 | 92302 | 223358 | 202092 | 9962 | 1180031 | 340986 |
| 42B20AAXX_5 | 362137 | 148783 | 357877 | 394729 | 12389 | 1424912 | 68003 |
| 42NY4AAXX_6 | 341976 | 141231 | 337090 | 375487 | 12052 | 1378888 | 64209 |
| 42B3YAAXX_4 | 138246 | 70875 | 136382 | 138391 | 12304 | 616691 | 49451 |
| 42B3YAAXX_5 | 170648 | 70467 | 168072 | 135388 | 974 | 566518 | 42326 |
| 42B3YAAXX_6 | 132475 | 44905 | 136459 | 160395 | 389 | 667026 | 26299 |
| 42B3YAAXX_7 | 113408 | 43048 | 107718 | 141494 | 2593 | 1283890 | 22330 |
| 42B48AAXX_1 | 256567 | 124172 | 269332 | 265243 | 10027 | 983648 | 157164 |
| 42NY4AAXX_3 | 251137 | 121415 | 265584 | 257036 | 9430 | 928636 | 161751 |
| 42B48AAXX_5 | 289925 | 151138 | 324297 | 301082 | 16442 | 839180 | 118722 |
| 42B48AAXX_6 | 268154 | 131026 | 276605 | 289938 | 13242 | 350851 | 117843 |
| 42NY4AAXX_4 | 266872 | 131493 | 274396 | 290415 | 12943 | 342377 | 123414 |
| 42HAMAAXX_3 | 209625 | 75012 | 179009 | 178200 | 1219 | 1534882 | 129974 |
| 42HAMAAXX_5 | 298877 | 111530 | 239274 | 229981 | 3278 | 2034501 | 56439 |
| 42HAMAAXX_6 | 269900 | 74133 | 186667 | 225872 | 2058 | 893981 | 50924 |
| 42CJFAAXX_1 | 199515 | 94642 | 214310 | 212679 | 8137 | 619494 | 95565 |
| 42CJFAAXX_6 | 213143 | 65160 | 209320 | 243647 | 2492 | 888964 | 192749 |
| 42CJFAAXX_7 | 167097 | 81067 | 176950 | 172649 | 8986 | 1424677 | 227067 |
| 42P6UAAXX_8 | 17215 | 8261 | 18561 | 17295 | 838 | 127745 | 27005 |
| 42PFAAAXX_4 | 234903 | 114839 | 247425 | 241570 | 13148 | 2053928 | 301739 |
| 42CJJAAXX_1 | 609310 | 96684 | 339568 | 295849 | 2164 | 989128 | 66148 |
| 42CJJAAXX_2 | 737464 | 63851 | 124388 | 275901 | 7126 | 3216582 | 130981 |
| 42CJJAAXX_3 | 620338 | 114045 | 191753 | 270642 | 1229 | 2838056 | 84081 |
| 42CJJAAXX_4 | 354431 | 85810 | 232698 | 336192 | 2110 | 3523049 | 100011 |
| 42CJJAAXX_6 | 297926 | 49000 | 122836 | 111915 | 5030 | 158056 | 119060 |
| 42PF0AAXX_3 | 474196 | 75648 | 186927 | 171612 | 7865 | 243691 | 175886 |
| 42CJJAAXX_8 | 276176 | 156128 | 314108 | 276910 | 12822 | 1482316 | 145473 |
| 42D3NAAXX_1 | 149531 | 76670 | 149834 | 155396 | 6781 | 909739 | 6467 |
| 42D3NAAXX_2 | 174357 | 81452 | 187633 | 155803 | 7382 | 986739 | 16225 |
| 42D3NAAXX_3 | 203877 | 99595 | 228529 | 197506 | 7263 | 1180391 | 24387 |
| 42D3NAAXX_5 | 216461 | 104278 | 243974 | 224609 | 10383 | 575251 | 183145 |
| 42D3NAAXX_6 | 216570 | 94026 | 248425 | 210652 | 6989 | 968227 | 101922 |
| 42P6UAAXX_6 | 262878 | 115240 | 301391 | 255754 | 8489 | 1184336 | 125429 |
| 42D3NAAXX_7 | 209014 | 111163 | 245063 | 178246 | 9808 | 748009 | 134024 |
| 42P6UAAXX_7 | 275545 | 147193 | 323736 | 233642 | 12781 | 997498 | 178517 |
| 42PF0AAXX_6 | 166272 | 77755 | 189860 | 252159 | 1035 | 3676417 | 226269 |
| 42PF0AAXX_7 | 172728 | 77054 | 166483 | 207201 | 697 | 747677 | 138139 |
| 42PFAAAXX_5 | 284380 | 85891 | 227994 | 270434 | 1701 | 2327751 | 49276 |
| 42PFAAAXX_6 | 208760 | 92853 | 146553 | 220040 | 15640 | 3028379 | 15541 |
| 42PMUAAXX_6 | 219362 | 83814 | 183818 | 207903 | 1308 | 1459593 | 42587 |
| 42PMUAAXX_7 | 197489 | 65724 | 166683 | 189731 | 6249 | 1195127 | 34780 |
| 42PFAAAXX_7 | 358890 | 109693 | 196656 | 272956 | 2409 | 2148064 | 93843 |
| 42PFAAAXX_8 | 163790 | 97732 | 299455 | 202865 | 2498 | 1402728 | 49005 |
| 42PMUAAXX_1 | 249100 | 88440 | 184288 | 420975 | 1051 | 485068 | 75760 |
| 42PMUAAXX_2 | 215351 | 49403 | 185381 | 197459 | 5227 | 2014100 | 33040 |
| 42PMUAAXX_4 | 153323 | 80214 | 178254 | 154882 | 996 | 789484 | 30244 |
| 42PMUAAXX_8 | 333209 | 72183 | 47727 | 152540 | 649 | 1083625 | 10980 |
| 42RMPAAXX_1 | 352483 | 146277 | 315190 | 373134 | 1534 | 2434765 | 85843 |
| 42RMPAAXX_2 | 326773 | 158346 | 290994 | 408029 | 26840 | 1939964 | 58283 |
| 42RMPAAXX_3 | 283991 | 129844 | 372065 | 360638 | 7922 | 2100050 | 80609 |
| 42RMPAAXX_4 | 232371 | 84290 | 280856 | 229378 | 13327 | 6164636 | 110174 |
| 42RMPAAXX_5 | 229712 | 79564 | 173748 | 249338 | 2193 | 1843288 | 13102 |
| 42RMPAAXX_6 | 198544 | 65203 | 101217 | 195516 | 4462 | 1440663 | 57432 |
| 42RMPAAXX_7 | 220849 | 55137 | 123864 | 283485 | 10462 | 532142 | 35666 |
| 42RMPAAXX_8 | 405746 | 80304 | 269425 | 281905 | 1106 | 482264 | 82124 |
| 42RYFAAXX_1 | 126325 | 58501 | 139344 | 108355 | 2024 | 287668 | 126960 |
| 42RYFAAXX_2 | 199112 | 73119 | 140234 | 224647 | 2859 | 482728 | 154222 |
| 42RYFAAXX_3 | 199754 | 54814 | 229473 | 167783 | 966 | 328480 | 106565 |
| 42RYFAAXX_4 | 153972 | 48697 | 117009 | 108928 | 4524 | 459772 | 82452 |
| 42RYFAAXX_5 | 213663 | 81749 | 171561 | 125466 | 23494 | 638285 | 111861 |
| 42RYFAAXX_6 | 223904 | 158701 | 216016 | 182990 | 15088 | 2352728 | 304136 |
| 42RYFAAXX_7 | 43960 | 20390 | 48778 | 50633 | 2990 | 1601294 | 358798 |
| 42RYFAAXX_8 | 114184 | 76460 | 161766 | 112155 | 3963 | 1418636 | 331269 |
| 42TA8AAXX_1 | 209504 | 110403 | 202939 | 170848 | 9397 | 1172234 | 29654 |
| 42TA8AAXX_2 | 247169 | 129395 | 241152 | 199778 | 11081 | 1370834 | 35377 |
| 42TA8AAXX_3 | 234874 | 122669 | 229713 | 188259 | 10421 | 1276791 | 34617 |
| 42TA8AAXX_4 | 7536 | 3825 | 7491 | 5993 | 286 | 35775 | 1186 |
| 42TA8AAXX_5 | 268255 | 142184 | 256195 | 219089 | 12736 | 1578967 | 36864 |
| 42TA8AAXX_6 | 276851 | 152735 | 241299 | 233912 | 16134 | 2055548 | 28646 |
| 42TA8AAXX_7 | 248149 | 130404 | 245678 | 200219 | 10514 | 1323804 | 36707 |
| 42TB9AAXX_1 | 396319 | 179265 | 325335 | 371466 | 24609 | 1225522 | 117300 |
| 42TB9AAXX_2 | 208997 | 80218 | 203743 | 193321 | 10447 | 581403 | 1227507 |
| 42TB9AAXX_3 | 322074 | 172149 | 328517 | 353218 | 18128 | 1714080 | 194007 |
| 42TB9AAXX_4 | 169764 | 92303 | 176972 | 176781 | 9923 | 522691 | 75863 |
| 42Y6WAAXX_6 | 258018 | 139150 | 266010 | 266081 | 14942 | 798382 | 108805 |
| 42TB9AAXX_6 | 481760 | 165996 | 328618 | 392708 | 17353 | 1309491 | 110620 |
| 42TB9AAXX_7 | 318764 | 180795 | 338405 | 380984 | 18276 | 1466092 | 139352 |
| 42TBWAAXX_1 | 174843 | 41078 | 122740 | 134525 | 5348 | 7476669 | 463671 |
| 42TBWAAXX_2 | 251776 | 117276 | 290676 | 297507 | 5228 | 2656337 | 96529 |
| 42TBWAAXX_3 | 263428 | 56732 | 374444 | 239685 | 2803 | 6108846 | 246493 |
| 42TBWAAXX_4 | 258983 | 109397 | 266951 | 288764 | 7738 | 4248023 | 71767 |
| 42TBWAAXX_5 | 194563 | 79854 | 242619 | 178498 | 3211 | 6974108 | 144065 |
| 42TBWAAXX_6 | 134492 | 69385 | 154278 | 141167 | 2070 | 2600381 | 110271 |
| 42Y6WAAXX_1 | 152163 | 78823 | 176127 | 160536 | 2273 | 2905347 | 126052 |
| 42TBWAAXX_7 | 183667 | 85704 | 237702 | 189840 | 2334 | 1577196 | 40273 |
| 42TBWAAXX_8 | 247824 | 59145 | 247057 | 213437 | 9842 | 5931445 | 161764 |
| 42TCBAAXX_1 | 238697 | 66651 | 213395 | 159981 | 2406 | 2465923 | 53836 |
| 42TCBAAXX_2 | 343158 | 44343 | 224613 | 244119 | 2275 | 4991631 | 186525 |
| 42TCBAAXX_3 | 202888 | 61467 | 175334 | 175424 | 7260 | 6344389 | 150794 |
| 42TCBAAXX_4 | 250931 | 89260 | 262811 | 265846 | 7913 | 1979162 | 115526 |
| 42TCBAAXX_5 | 346214 | 109441 | 303047 | 252941 | 62244 | 2027256 | 73368 |
| 42TCBAAXX_6 | 102369 | 37626 | 89193 | 96184 | 926 | 5156736 | 64094 |
| 42TCBAAXX_7 | 162829 | 55426 | 137376 | 144250 | 11463 | 950266 | 24830 |
| 42TCBAAXX_8 | 169038 | 66814 | 156278 | 172536 | 2439 | 600552 | 40208 |
| 42TCLAAXX_1 | 35214 | 14555 | 47234 | 49727 | 194 | 151194 | 3617 |
| 42Y39AAXX_2 | 286920 | 123459 | 340597 | 420782 | 1578 | 1504861 | 20725 |
| 42TCLAAXX_3 | 33503 | 13123 | 36969 | 46820 | 204 | 205823 | 4730 |
| 42Y39AAXX_5 | 300086 | 121717 | 288755 | 432597 | 1947 | 2200704 | 28989 |
| 42TCLAAXX_4 | 95968 | 38823 | 98256 | 121607 | 429 | 475967 | 9926 |
| 42Y39AAXX_6 | 278583 | 122949 | 247475 | 390709 | 1757 | 1761838 | 17979 |
| 42TCLAAXX_5 | 87059 | 32563 | 86535 | 93487 | 508 | 525589 | 12162 |
| 42Y39AAXX_7 | 309586 | 121273 | 259073 | 359794 | 2006 | 2539693 | 26110 |
| 42TG0AAXX_2 | 89555 | 39363 | 121935 | 94006 | 555 | 822062 | 45624 |
| 42TG0AAXX_3 | 337006 | 102567 | 224466 | 275071 | 4184 | 1968681 | 76644 |
| 42TG0AAXX_4 | 317211 | 132199 | 201538 | 285796 | 11463 | 1865626 | 107016 |
| 42TG0AAXX_5 | 160310 | 58797 | 163245 | 166896 | 2742 | 2133218 | 42712 |
| 42TG0AAXX_6 | 180374 | 66758 | 178252 | 198820 | 11046 | 3361874 | 59543 |
| 42TG0AAXX_7 | 4622 | 1745 | 4114 | 14065 | 732 | 10887925 | 239684 |
| 42TG0AAXX_8 | 238090 | 81509 | 210813 | 249715 | 17286 | 4144713 | 114865 |
| 42Y2MAAXX_1 | 314788 | 122954 | 286915 | 411080 | 1449 | 1119573 | 72575 |
| 42Y2MAAXX_2 | 342536 | 150475 | 257493 | 385614 | 852 | 1070738 | 57116 |
| 42Y2MAAXX_3 | 336717 | 131263 | 272622 | 423931 | 1480 | 1222218 | 53357 |
| 42Y2MAAXX_4 | 385161 | 188089 | 303151 | 438286 | 836 | 780280 | 32347 |
| 42Y2MAAXX_5 | 315167 | 129257 | 368062 | 427431 | 45084 | 967259 | 44119 |
| 42Y2MAAXX_6 | 311769 | 141127 | 295019 | 361097 | 32843 | 1385788 | 80079 |
| 42Y2MAAXX_7 | 297947 | 150482 | 347648 | 415776 | 39710 | 501461 | 44323 |
| 42Y2MAAXX_8 | 353037 | 175982 | 319235 | 377274 | 27713 | 1816186 | 107616 |
| 42Y39AAXX_3 | 318630 | 124611 | 345702 | 389291 | 1366 | 1497457 | 38382 |
| 42Y39AAXX_4 | 317502 | 125039 | 339426 | 391111 | 1354 | 1524817 | 36345 |
| 42Y6NAAXX_1 | 310148 | 105905 | 225357 | 351762 | 2037 | 1989445 | 65003 |
| 42Y6NAAXX_2 | 255610 | 112697 | 236814 | 282913 | 3002 | 1205222 | 43781 |
| 42Y6NAAXX_4 | 241813 | 107323 | 198062 | 345580 | 1755 | 1333802 | 52963 |
| 42Y6NAAXX_5 | 263562 | 110352 | 188811 | 314962 | 1741 | 2063447 | 47860 |
| 42Y6NAAXX_6 | 391444 | 81453 | 238663 | 274634 | 3171 | 1604541 | 60637 |
| 42Y6NAAXX_7 | 240630 | 79129 | 154442 | 394201 | 8730 | 1388814 | 45287 |
| 42Y6NAAXX_8 | 372036 | 153750 | 247142 | 373102 | 1871 | 1078869 | 48294 |
| 42Y6WAAXX_2 | 249661 | 80034 | 234127 | 218570 | 9870 | 225673 | 48769 |
| 42YMTAAXX_2 | 174158 | 56541 | 160917 | 154591 | 7244 | 163951 | 33809 |
| 42Y6WAAXX_3 | 310337 | 127874 | 349747 | 230248 | 15457 | 987906 | 91555 |
| 42YMTAAXX_4 | 141522 | 57992 | 160871 | 104253 | 6950 | 448005 | 44533 |
| 42Y6WAAXX_4 | 141372 | 86675 | 181038 | 139530 | 8252 | 3813971 | 171286 |
| 42YMTAAXX_8 | 76869 | 47101 | 99366 | 74503 | 4367 | 2048530 | 98007 |
| 42YMMAAXX_1 | 255209 | 118536 | 189888 | 318321 | 9170 | 1095200 | 41874 |
| 42YMMAAXX_2 | 456618 | 122574 | 352985 | 281578 | 8191 | 483556 | 46089 |
| 42YMMAAXX_8 | 436524 | 115533 | 356775 | 263380 | 6689 | 466182 | 43423 |
| 42YMMAAXX_3 | 460613 | 126702 | 253002 | 349039 | 3808 | 849766 | 39777 |
| 42YMMAAXX_4 | 300088 | 84040 | 266106 | 280756 | 11015 | 1013275 | 260335 |
| 42YMMAAXX_5 | 302870 | 138589 | 333931 | 388333 | 2888 | 1074957 | 99908 |
| 42YMMAAXX_6 | 282431 | 142583 | 314868 | 295993 | 20586 | 779333 | 526104 |
| 42YMMAAXX_7 | 320296 | 182451 | 248118 | 314541 | 23197 | 2797580 | 253615 |
| 42YMTAAXX_1 | 311197 | 76479 | 251236 | 288520 | 3717 | 282116 | 93455 |
| 42YMTAAXX_3 | 183338 | 85984 | 194383 | 171929 | 3546 | 1021747 | 107333 |
| 42YMTAAXX_5 | 261792 | 58340 | 101786 | 145663 | 3582 | 685778 | 106127 |
| 42YMTAAXX_6 | 377757 | 112265 | 280575 | 286452 | 1733 | 765014 | 258043 |
| 42YMTAAXX_7 | 169189 | 91632 | 147810 | 192700 | 604 | 621748 | 145760 |
| 43336AAXX_1 | 618869 | 283080 | 362368 | 885436 | 90951 | 890 | 4047 |
| 43336AAXX_2 | 779708 | 288848 | 629839 | 748113 | 63187 | 1269 | 9501 |
| 43336AAXX_3 | 615815 | 281066 | 323519 | 1898801 | 6057 | 1002 | 3566 |
| 43336AAXX_4 | 654494 | 283095 | 430670 | 1571822 | 6153 | 1817 | 2583 |
| 43336AAXX_5 | 613247 | 308861 | 362738 | 830697 | 204269 | 6366 | 19808 |
| 43336AAXX_6 | 592488 | 285012 | 272413 | 1052620 | 96495 | 1212 | 4063 |
| 43336AAXX_7 | 260886 | 122702 | 261558 | 278060 | 2193 | 3328173 | 427183 |
| 6137KAAXX_3 | 467505 | 175038 | 280504 | 545058 | 13898 | 1223990 | 788992 |
| 6137KAAXX_4 | 467309 | 175095 | 279192 | 543194 | 13939 | 1221633 | 803870 |
| 6137LAAXX_6 | 113573 | 49086 | 68793 | 113658 | 5050 | 182463 | 567284 |
| 6137KAAXX_6 | 495606 | 204129 | 294744 | 433045 | 5770 | 2679545 | 68438 |
| 6137KAAXX_7 | 644920 | 142191 | 489232 | 465857 | 18150 | 1630996 | 92026 |
| 6137KAAXX_8 | 413516 | 176516 | 361589 | 579628 | 1343 | 1827394 | 15836 |
| 6137LAAXX_7 | 248771 | 149999 | 118895 | 340837 | 65823 | 28396 | 65860 |
| 6137LAAXX_8 | 183526 | 105456 | 100667 | 232851 | 49008 | 18636 | 56496 |
| 61VFCAAXX_1 | 344817 | 184698 | 355025 | 473833 | 6429 | 2379159 | 420074 |
| 61VFCAAXX_2 | 343380 | 172365 | 349709 | 524423 | 7125 | 3636767 | 161470 |
| 61VFCAAXX_3 | 367218 | 179235 | 380369 | 481946 | 5388 | 2110933 | 84819 |
| 61VFCAAXX_6 | 407732 | 188260 | 412884 | 447411 | 13931 | 2327370 | 160521 |
| 62837AAXX_1 | 503729 | 269743 | 473051 | 583119 | 16923 | 1736659 | 107376 |
| 62837AAXX_2 | 461685 | 240696 | 435487 | 716066 | 1675 | 2233592 | 91061 |
| 62837AAXX_3 | 518636 | 220242 | 465628 | 674984 | 32060 | 1858618 | 27720 |
| 62837AAXX_4 | 454738 | 274093 | 405522 | 535531 | 19610 | 1480970 | 121512 |
| 62837AAXX_7 | 157455 | 71272 | 150237 | 162020 | 8868 | 226572 | 148401 |
| 62837AAXX_8 | 430193 | 184469 | 400194 | 520796 | 618 | 1262058 | 86580 |
| 6286PAAXX_1 | 466938 | 193877 | 468828 | 554183 | 20912 | 1576246 | 64497 |
| 6286PAAXX_2 | 431045 | 215647 | 431944 | 581655 | 1070 | 989915 | 75219 |
| 6286PAAXX_3 | 552869 | 176266 | 372616 | 641057 | 4679 | 2595818 | 196313 |
| 6286PAAXX_4 | 592530 | 133490 | 315300 | 510413 | 3370 | 2214274 | 206418 |
| 6286PAAXX_5 | 431870 | 140016 | 389672 | 427996 | 2534 | 3039154 | 291790 |
| 6286PAAXX_6 | 682128 | 187201 | 443613 | 497200 | 14922 | 2392482 | 162800 |
| 6286PAAXX_7 | 573423 | 200417 | 404063 | 754742 | 3864 | 2295990 | 241257 |
| 6286PAAXX_8 | 390526 | 177412 | 604225 | 529005 | 21529 | 1366406 | 187409 |

TABLE 3

| Ψ-Chromosomal | Ψ- | | | Bladder | Breast | Cervical | Colon | Gastric | Kidney | Lymphoblastoid | Melanoma |
|---|---|---|---|---|---|---|---|---|---|---|---|
| Region | Name | Encode | Yale | (17) | (64) | (8) | (6) | (25) | (12) | (19) | (10) |
| chr21: 10202689-10203575 | EIF3F | manual | proc | 2 | | | | | | | |
| chr10: 37636369-37640031 | ATP8A2 | manual | | | 6 | | | | | | |
| chr10: 37640002-37644728 | ATP8A2 | manual | | | 5 | | | | | | |
| chr4: 141781899-141782720 | KIAA1856 | | dup | | 4 | | | | | | |
| chr10: 37666189-37668498 | TMEM161B | manual | proc | | 4 | | | | | | |
| chr19: 12531356-12532856 | PGK1 | manual | dup | | 3 | | | | | | |
| chr22: 23438167-23438592 | CRIP1 | manual | proc | | 3 | | | | | | |
| chr9: 75280000-75280934 | DPP3 | manual | proc | | 3 | | | | | | |
| chr10: 37619903-37620019 | ATP8A2 | manual | | | 2 | | | | | | |
| chr10: 37580608-37581341 | ATP8A2 | manual | dup | | 2 | | | | | | |
| chr10: 37577033-37577556 | ATP8A2 | manual | dup | | 8 | | | | | | |
| chr10: 37590732-37594328 | ATP8A2 | manual | dup | | 6 | | | | | | |
| chr1: 109447342-109448872 | PCBP1 | manual | dup | | 2 | | | | | | |
| chr2: 131895157-131898651 | GNAQ | manual | proc | | 2 | | | | | | |
| chr8: 130810585-130813055 | MTRF1L | | dup | | | 2 | | | | | |
| chr5: 92250781-92251791 | CCT7 | auto | proc | | | | 2 | | | | |
| chr7: 138533692-138534485 | FCF1 | manual | proc | | | | 2 | | | | |
| chr17: 44331639-44331679 | RHOB | | dup | | | | | 2 | | | |
| chr12: 12243007-12245031 | MORF4L1 | | dup | | | | | 2 | | | |
| chr1: 234516058-234516726 | FKBP3 | manual | dup | | | | | 2 | | | |
| chr14: 53527463-53528434 | ATP5C1 | manual | proc | | | | | 2 | | | |
| chr10: 69180083-69181213 | AKR1B10 | manual | proc | | | | | 2 | | | |
| chr8: 26461161-26461703 | PSME2 | | proc | | | | | | 4 | | |
| chr2: 152955000-152955599 | NUDC | manual | proc | | | | | | 3 | | |
| chr7: 134463620-134464973 | RNF14 | manual | proc | | | | | | 2 | | |
| chr10: 121746618-121747198 | NACA | manual | proc | | | | | | 2 | | |
| chr1: 218507681-218508028 | AURKA | manual | proc | | | | | | | 7 | |
| chr3: 115431891-115434722 | KIAA1539 | | proc | | | | | | | 4 | |
| chr3: 120732546-120733113 | CSRP2 | | proc | | | | | | | 4 | |
| chr2: 201985004-201986130 | SCYL2 | manual | proc | | | | | | | 4 | |
| chr2: 231530573-231530995 | FAM36A | manual | proc | | | | | | | 4 | |
| chrX: 110747689-110749928 | EIF4B | manual | proc | | | | | | | 4 | |
| chr10: 75852293-75853672 | RAB5C | manual | proc | | | | | | | 4 | |
| chr15: 81278450-81278844 | BC117556 | auto | | | | | | | | 3 | |
| chr19: 40565000-40566763 | EEF1A1 | | dup | | | | | | | 3 | |
| chr4: 135468269-135469814 | PES1 | | proc | | | | | | | 3 | |
| chr10: 31689689-31689904 | SPTLC1 | manual | frag | | | | | | | 3 | |
| chr1: 202765009-202765499 | PEF1 | manual | proc | | | | | | | 3 | |
| chr10: 75957261-75958035 | MRPL35 | manual | proc | | | | | | | 3 | |
| chr8: 48152971-48153133 | A26A1 | | dup | | | | | | | 2 | |
| chr15: 81288859-81289520 | BC117556 | auto | | | | | | | | 2 | |
| chr12: 31355668-31356398 | SLC25A13 | | dup | | | | | | | 2 | |
| chr11: 85683591-85684437 | SET | | proc | | | | | | | 2 | |
| chr4: 185717093-185717964 | NOLA1 | | proc | | | | | | | 2 | |
| chr4: 100049539-100049960 | TBCA | | proc | | | | | | | 2 | |
| chr5: 126875001-126876506 | HNRNPK | | proc | | | | | | | 2 | |
| chr12: 92041871-92042457 | NACA | | proc | | | | | | | 2 | |
| chr2: 149346124-149347002 | USP8 | manual | frag | | | | | | | 2 | |
| chr7: 53822238-53822358 | ATM | manual | frag | | | | | | | 2 | |
| chr4: 93322413-93324234 | PMPCA | auto | proc | | | | | | | 2 | |
| chrX: 119227888-119229412 | EEF1A1 | manual | dup | | | | | | | 2 | |
| chr1: 117985009-117985810 | VDAC2 | manual | proc | | | | | | | 2 | |
| chr17: 59917896-59918632 | ATP5J2 | manual | proc | | | | | | | 2 | |
| chr21: 15051843-15053508 | POLR2C | manual | proc | | | | | | | 2 | |
| chr9: 139655582-139655864 | SET | manual | proc | | | | | | | 2 | |
| chr18: 41862332-41864360 | UTP18 | | proc | | | | | | | | |
| chr1: 54763083-54763321 | TALDO1 | manual | frag | | | | | | | | |
| chr22: 28770926-28773344 | CNN2 | manual | proc | | | | | | | | |
| chr3: 37151088-37151663 | UBE2D3 | manual | proc | | | | | | | | |
| chr15: 69242617-69244956 | HMGB1 | | proc | | | | | | | | |
| chr12: 31798916-31799483 | IFITM2 | | proc | | | | | | | | |
| chr12: 8555270-8556510 | SUPT4H1 | | proc | | | | | | | | |
| chr12: 64915002-64915315 | RBMS1 | auto | proc | | | | | | | | |
| chr19: 59424557-59425022 | LSM14A | manual | dup | | | | | | | | |
| chr1: 144988906-144990026 | IGF2BP2 | manual | proc | | | | | | | | |
| chr14: 68403267-68404827 | BLZF1 | | dup | | | | | | | | |
| chr19: 56731589-56731634 | FAM107B | | dup | | | | | | | | |
| chr12: 21491749-21494398 | TCEB1 | | dup | | | | | | | | |
| chr1: 222429477-222430009 | ZNF706 | manual | | | | | | | | | |
| chr8: 55597596-55598332 | SEC11A | auto | proc | | | | | | | | |
| chrX: 136350864-136351728 | RAC1 | manual | proc | | | | | | | | |
| chr1: 84482546-84483186 | NEDD8 | manual | proc | | | | | | | | |
| chrX: 152525161-152526553 | MRFAP1 | manual | proc | | | | | | | | |
| chr19: 59469095-59469457 | ORAOV1 | | dup | | | | | | | | |
| chr14: 68405160-68406719 | BLZF1 | | dup | | | | | | | | |
| chr11: 124182827-124183139 | RNF181 | | proc | | | | | | | | |
| chr12: 114514705-114515025 | UBA52 | | proc | | | | | | | | |
| chr10: 22554510-22555015 | PSME2 | manual | frag | | | | | | | | |
| chr13: 97437263-97437450 | FTL | manual | frag | | | | | | | | |
| chr2: 26225408-26227645 | SMARCE1 | manual | dup | | | | | | | | |
| chr4: 84387420-84388630 | AMT | auto | proc | | | | | | | | |
| chr13: 42405938-42406759 | ZDHHC4 | manual | proc | | | | | | | | |
| chr9: 96088678-96089876 | VDAC1 | manual | proc | | | | | | | | |
| chr6: 149475018-149475620 | SNRPB2 | manual | proc | | | | | | | | |
| chr1: 245420770-245420911 | ZNF430 | manual | proc | | | | | | | | |
| chr22: 22040076-22042678 | CES7 | manual | | | | | | | | | |
| chr15: 19267708-19269952 | CXADR | auto | proc | | | | | | | | |
| chr22: 22031558-22032127 | CES7 | manual | | | | | | | | | |
| chr18: 54508763-54509828 | MRPL37 | | proc | | | | | | | | |
| chr4: 166544280-166544941 | HADHA | | proc | | | | | | | | |
| chr11: 18573250-18574450 | MTCH1 | | proc | | | | | | | | |
| chr10: 104925212-104926869 | MARCKSL1 | manual | proc | | | | | | | | |
| chr15: 33377487-33379076 | RBM17 | | proc | | | | | | | | |
| chr2: 132075527-132076801 | AY458019 | manual | | | | | | | | | |
| chr12: 118116932-118118276 | PDAP1 | auto | proc | | | | | | | | |
| chr7: 63665694-63668112 | LOC402665 | manual | proc | | | | | | | | |
| chr15: 19391669-19394059 | NF1 | | dup | | | | | | | | |
| chr15: 19396218-19399993 | NF1 | | dup | | | | | | | | |
| chr4: 72385029-72386725 | SLC35A4 | | proc | | | | | | | | |
| chr4: 140255185-140256207 | PPP1R14B | | proc | | | | | | | | |
| chr11: 125442528-125444160 | NAP1L1 | | proc | | | | | | | | |
| chr12: 60911988-60914886 | KLF17 | | proc | | | | | | | | |
| chr8: 48666667-48667924 | UBE2N | | proc | | | | | | | | |
| chr5: 75410408-75411560 | SMS | | proc | | | | | | | | |
| chr10: 93965940-93966789 | NOLA2 | manual | | | | | | | | | |
| chr12: 17033298-17034633 | SKP1 | | proc | | | | | | | | |
| chr19: 63029371-63030025 | FKBP1A | | proc | | | | | | | | |
| chr3: 121508215-121509682 | PTOV1 | | proc | | | | | | | | |
| chr3: 52741566-52743049 | ZNF610 | | proc | | | | | | | | |
| chr2: 94890365-94894064 | LOC389834 | | proc | | | | | | | | |
| chr1: 245430003-245432118 | ZNF720 | manual | frag | | | | | | | | |
| chr2: 69678006-69679772 | B3GALNT1 | manual | dup | | | | | | | | |
| chr22: 16277647-16279389 | TERF2IP | manual | dup | | | | | | | | |
| chr3: 132029176-132030005 | GSTO1 | auto | proc | | | | | | | | |
| chr14: 19216101-19218960 | USP10 | auto | proc | | | | | | | | |
| chr2: 54131611-54132922 | HMGB1 | manual | proc | | | | | | | | |
| chrX: 18793802-18794774 | CCDC5 | manual | proc | | | | | | | | |
| chr20: 25027736-25029870 | VTCN1 | manual | proc | | | | | | | | |
| chr10: 104595191-104596503 | PTGES3 | manual | proc | | | | | | | | |
| chr6: 101085019-101087991 | DCUN1D1 | manual | proc | | | | | | | | |
| chr20: 44525241-44526750 | MKRN1 | manual | proc | | | | | | | | |
| chr10: 46552660-46554269 | HNRNPA1 | manual | frag | | | 5 | | 1 | | | |
| chr1: 174850424-174852903 | PTP4A1 | manual | proc | | | 2 | | 1 | | | |
| chr9: 33592667-33595028 | KIAA2015 | | proc | | | 1 | | | | | |
| chr22: 22386358-22389509 | GUSB | manual | | | | 1 | | | | | |
| chr1: 47081250-47081689 | CYP4Z1 | manual | proc | 4 | 6 | | | | | | |
| chr20: 32295708-32298037 | DENR | manual | proc | | | | 2 | | | | |
| chr8: 30225003-30227051 | TAGLN2 | auto | proc | | 2 | | | 5 | | | |
| chr1: 145020394-145021949 | SSB | manual | proc | | | | | 2 | | | |
| chr13: 44066841-44068205 | SMARCE1 | manual | proc | | | | | | 2 | | |
| chr1: 176981907-176983662 | PTPN2 | manual | proc | | 1 | | | | | 6 | |
| chr4: 113966768-113968338 | TAF4B | auto | frag | | | | | | | 4 | |
| chr13: 76456888-76460017 | DHX9 | manual | proc | | | | | | | 4 | |
| chr12: 8177240-8178828 | POU5F1 | auto | proc | | | | | | | 1 | |
| chr9: 5103911-5104767 | IGHE | manual | proc | | | | | | | 1 | |
| chr17: 30840954-30844887 | E2F3 | | proc | | | | | | | 2 | |
| chr5: 31945012-31947315 | TPT1 | auto | dup | | | | | | | 1 | |
| chr5: 102395954-102396670 | EIF3K | auto | proc | | | | | | | 1 | |
| chr9: 5075006-5076284 | PDSS1 | manual | proc | | | | | | | 2 | |
| chr15: 87332965-87334484 | CAPZA2 | | proc | | | | | | | | |
| chr12: 4613400-4615020 | RAB5A | | dup | | | | | | | | |
| chr12: 9199537-9200023 | TPT1 | | proc | | | | | | | | |
| chr1: 70157624-70158591 | PIN1 | manual | proc | | | | | | | | |
| chr3: 12344333-12344751 | PFDN1 | manual | proc | | | | | | | | |
| chr11: 77772536-77773641 | ZNF75D | | proc | | | | | | | 1 | |
| chr5: 172121628-172124841 | FAM58A | auto | proc | | | | | | | 1 | |
| chrX: 64605779-64607158 | AP1M2 | manual | dup | | 2 | | | | | | |
| chrX: 91255170-91255864 | EIF4A1 | manual | proc | | | | | | | 2 | |
| chr2: 162640152-162642586 | TIMM8A | manual | proc | | | | | | | | |
| chr4: 166545018-166547102 | HADHA | | proc | | | | | | | | |
| chr22: 34623087-34624393 | NDUFA9 | manual | proc | | 1 | | | | | | |
| chr1: 177822521-177824924 | CENTG2 | manual | proc | | 3 | | | | | | |
| chr9: 75278688-75279979 | DPP3 | manual | proc | | 3 | | | | | 1 | |
| chr9: 37625488-37627344 | RAB1B | manual | | | 3 | | | | | | |
| chr15: 98905757-98909660 | PRKY | auto | | | 2 | | | | | | |
| chr13: 29768191-29769131 | PRDX2 | manual | proc | | 2 | | | | | 1 | |
| chr17: 6771934-6772425 | SPIC | | proc | | | 3 | 1 | | | | |
| chr7: 55294166-55295027 | CALM1 | manual | proc | | | 2 | | | | | |
| chr14: 20065201-20066879 | SET | | proc | | | 2 | | | | | 1 |
| chrX: 61915436-61916582 | SSB | manual | proc | | | 2 | | | | | 1 |
| chr14: 57820991-57822278 | HMGB1 | auto | proc | | | 2 | | | | | |
| chr11: 47882316-47883972 | SEC61B | | proc | | 2 | 1 | | | | | |
| chr3: 198845497-198847337 | PPP4R2 | | dup | | | 1 | | | | | |
| chr1: 38947029-38949502 | HSPA5 | manual | proc | | | 1 | | | | | |
| chr6: 38663042-38664706 | TFG | manual | proc | | | | 2 | 2 | | | |
| chr5: 144588471-144590025 | ASS1 | auto | proc | | | | 1 | | 2 | | |
| chr12: 6865020-6866674 | DSTN | manual | | | | | 1 | | | 2 | |
| chr1: 81740357-81744083 | ARID3B | manual | proc | | | | 1 | | | 2 | |
| chr8: 50815164-50816385 | PSAT1 | | proc | | | | 1 | 2 | | | |
| chr2: 172056105-172057010 | DAP3 | manual | frag | | | | 1 | 2 | | | |
| chr11: 118208607-118209718 | SET | auto | proc | | | | 1 | 2 | | | |
| chr10: 120393399-120394061 | TOMM22 | manual | proc | | | | 1 | 2 | | | |
| chr11: 86208209-86209928 | MOBKL3 | | proc | | | | | 2 | | 2 | |
| chr3: 73060699-73061349 | FTH1 | | proc | | | | | 1 | | | |
| chr22: 22325000-22325556 | ASL | manual | dup | | | | 1 | | 4 | | |
| chr3: 173800619-173804689 | SLC31A1 | manual | proc | | | | | | 3 | | |
| chr13: 19035415-19037296 | CASC4 | manual | proc | | | | | | 1 | 3 | |
| chr4: 40482518-40483890 | ARL4A | | proc | | | | | | 1 | | |
| chr18: 53581850-53582535 | MRPS17 | | proc | | | | | | 1 | | |
| chr6: 1458119-1459994 | ELF2 | manual | proc | | | | | | 1 | | |
| chr21: 32790823-32791600 | EXOSC3 | manual | proc | | | | | | 1 | | |
| chr7: 46003545-46004373 | FTL | manual | proc | | | | | 2 | 1 | | |
| chr13: 40464767-40464951 | RGS17 | manual | proc | | | | | | | 6 | |
| chrX: 46468751-46470028 | CHMP5 | manual | proc | | | | | | | 6 | |
| chr6: 90581057-90582594 | FAM64A | manual | proc | | | | | | | 4 | |
| chr22: 37448047-37449602 | PRDX3 | manual | proc | | | | | | | 3 | |
| chr19: 62378395-62379706 | PTCD3 | | dup | | | | | | | 2 | |
| chr5: 42501936-42504461 | ZNF35 | | proc | | | | | | | 2 | |
| chr15: 93200012-93201180 | GLTSCR2 | auto | proc | | | | | | | 2 | |
| chr11: 18643045-18644377 | SFRS3 | auto | proc | | | | | | | 2 | |
| chr2: 43761451-43762368 | ZFP106 | manual | frag | | | | | | | 2 | |
| chr6: 158577413-158579430 | SRP72 | manual | dup | | | | | | | 2 | |
| chr14: 60316280-60317538 | MAD2L1 | | proc | | | | | | | 1 | |
| chr3: 31470003-31472686 | THRAP3 | manual | dup | | | | | | | 1 | |
| chr9: 83890554-83893228 | DDX10 | manual | proc | | | | | | | 1 | |
| chr1: 246951416-246952125 | AHCY | manual | frag | | | | | | | 2 | 1 |
| chrY: 5265643-5266973 | EIF4A1 | manual | proc | | | | | 2 | | | 1 |
| chr12: 17035000-17036276 | EEF1A1 | | proc | | | | | | | 3 | 1 |
| chr12: 6457164-6459964 | PKP2 | | proc | | | | | | | 8 | |
| chr2: 197835918-197836621 | ATPSG2 | manual | proc | | | | | | | 8 | |
| chr10: 118771731-118773667 | DDX3X | manual | | | | 1 | | | | | |
| chr13: 40465037-40465571 | RGS17 | manual | proc | | | | | | | 4 | |
| chr4: 103870768-103875031 | MANBAL | | proc | | | | | | | 3 | |
| chr13: 30025729-30027622 | PTPN2 | manual | proc | | | | | | | 3 | |
| chr10: 51695144-51697065 | DYNC1I2 | manual | proc | | | 1 | | | | | |
| chr5: 139941169-139942270 | CCDC5 | manual | proc | | | 1 | | | | | |
| chr2: 201982582-201984995 | SCYL2 | manual | proc | | | | | | | 6 | |
| chr2: 182267396-182268139 | SAP18 | manual | proc | | | | 1 | | | | |
| chr3: 133588073-133590018 | KIAA1333 | | dup | | | | | | | | |
| chr1: 202041181-202041985 | CHMP5 | manual | proc | | | | | | | 6 | |
| chr4: 83415253-83417203 | BIN2 | | proc | | | | | 1 | | | |
| chr18: 70208031-70208617 | FAU | | proc | | 3 | | | | | | |
| chr14: 30221784-30222571 | UBE2C | | proc | | | | | | | 2 | |
| chr7: 23625349-23630025 | FCF1 | manual | dup | | | | | | | 3 | |
| chr4: 24268370-24269026 | ATP5L | | proc | | | 1 | | | | | |
| chr17: 18752228-18753057 | RANBP1 | manual | proc | | | 1 | | | | | |
| chr6: 139701047-139702459 | DNAJC7 | manual | proc | | | | | | | 5 | |
| chr1: 2454300040-245431986 | ZNF430 | manual | frag | | | | 1 | | | | |
| chr3: 31197239-31199007 | CNN2 | manual | proc | | | | 1 | | | | |
| chr3: 138100000-138101718 | RAD51AP1 | auto | proc | | | | | | | | |
| chrX: 17026484-17028444 | CBX1 | manual | proc | | | | | | | | |
| chr3: 194779703-194779896 | EEF1A1 | manual | frag | | | | | | | 2 | |
| chr3: 32769304-32770026 | SUGT1 | manual | proc | | | | | | | 2 | |
| chr7: 25695625-25696086 | UBA52 | | proc | | | | | | | | |
| chr12: 110503660-110503886 | IFITM1 | | proc | | | | | 5 | | | |
| chr17: 18510983-18512998 | FOXO3 | manual | | | | | | | | | |
| chr22: 22070000-22074479 | ZDHHC8 | manual | frag | | | 1 | | | | | |
| chr17: 60205003-60209312 | LOC201175 | | proc | | 6 | | | | | | |
| chr3: 49329045-49329924 | SSNA1 | | proc | | 3 | | | | | | |
| chr22: 22045609-22049946 | CES7 | manual | | | | | | | 1 | | |
| chr2: 20355007-20355544 | RP9 | | dup | | 1 | | | | | | |
| chr15: 33171848-33172885 | U2AF1 | | proc | | 1 | | | | | | |
| chr7: 75540406-75544538 | LOC285908 | manual | proc | | 1 | | | | | | |
| chr5: 110312099-110314335 | BCLAF1 | | dup | | 1 | | | | | | |
| chr4: 119333317-119335028 | FKBP4 | | proc | | 1 | | | | | | |
| chr3: 171145011-171147501 | FHL1 | | proc | | | | | | | 4 | |
| chr2: 132196348-132197314 | BC016035 | manual | proc | | | 2 | | 1 | | | |
| chrX: 70180396-70182077 | SOCS5 | manual | dup | | | | 3 | | | 1 | |
| chr15: 36537765-36539186 | CTDSP2 | | proc | | | | 2 | | | 1 | |
| chr1: 92311703-92313396 | SET | manual | proc | | | | 2 | 1 | | 1 | |
| chr15: 34696499-34699553 | LARP4 | | proc | | 1 | | 1 | | | | |
| chr14: 74019321-74020027 | RAP1A | | proc | | | 1 | | | | 1 | |
| chr14: 99902477-99903076 | NDUFB3 | | proc | | | | | | | 1 | |
| chr1: 97783716-97784986 | SEC63 | manual | frag | | | | | | | 1 | |
| chrX: 154170506-154173981 | TWF1 | manual | proc | | | | | 1 | | 2 | |
| chr15: 57756054-57757383 | PIGH | | proc | | | | | | | 3 | |
| chr6: 145070172-145071408 | NT5DC1 | manual | frag | | | | | | | 1 | |
| chr5: 97575000-97575656 | PSME2 | | proc | | | | | 1 | | 1 | |
| chr20: 5220061-5221727 | UBE2D3 | manual | | | | | | 1 | | 1 | |
| chr9: 34906998-34908341 | GLUL | manual | proc | | 1 | | | | | 1 | |
| chr7: 63005787-63008547 | NMD3 | manual | proc | | 1 | | | | | 1 | |
| chr12: 46765022-46765808 | ATP6V1F | | proc | | | | | | | 1 | |
| chr5: 157345785-157347510 | CDC37 | auto | proc | | 1 | | | | | | 1 |
| chr15: 32573790-32575001 | FSCN1 | | proc | | | | | | | 1 | |
| chr2: 139375038-139376849 | AHCY | manual | proc | | 2 | | | | | | |
| chr1: 64080787-64082020 | CFL1 | manual | proc | | 1 | | | | | 3 | |
| chr19: 32986379-32989682 | BC040831 | | proc | | | | | 1 | | 2 | |
| chr1: 238372430-238375019 | PSMD2 | manual | proc | | | | | | | | |
| chr10: 21357922-21359678 | EIF4B | manual | proc | | | | | | | 2 | |
| chr14: 57677677-57678048 | UBA52 | | proc | | 3 | | | | | 1 | |
| chr16: 4552399-4553233 | SUB1 | | proc | | | 4 | | | | 4 | |
| chrX: 36139141-36140020 | HMGB1 | manual | proc | | | 3 | 1 | 2 | | | |
| chr16: 51246583-51248855 | TPM3 | auto | | | | 3 | | | | 2 | |
| chr3: 97550893-97552589 | HNRNPK | | proc | | | 2 | | | | 1 | |
| chr6: 151297464-151299652 | ARL4A | manual | proc | | | 2 | | | | 1 | |
| chrX: 62435062-62436548 | CBX1 | manual | proc | | 2 | 2 | 1 | | | | |
| chr7: 138506992-138509638 | MSL3L1 | manual | dup | | | 1 | | | | 2 | |
| chr2: 204206408-204208995 | ZNF207 | manual | proc | | | 1 | | | | 2 | |
| chr15: 49024038-49025027 | WDSOF1 | | proc | | | 1 | | | | 2 | |
| chr7: 84495121-84497088 | HSPA8 | manual | proc | | | 1 | | | | 2 | 1 |
| chr17: 42855745-42858503 | NFE2L3 | | dup | | 2 | 1 | | | | | |
| chr16: 17095569-17097803 | LYRM2 | | dup | | | 1 | | 3 | | 1 | |
| chr18: 22090016-22090564 | FAM60A | | proc | | | 1 | | 3 | | | |
| chr9: 37875565-37875969 | TXNDC14 | manual | proc | | | 5 | 4 | | | | |
| chr11: 17030098-17030554 | MGC72080 | auto | proc | | | | 3 | | 3 | | |
| chr4: 68265004-68266433 | ST3GAL1 | auto | proc | | | | 2 | | | 1 | |
| chr1: 16890145-16890316 | PDE4DIP | manual | | | | | 2 | | 1 | | |
| chr5: 108257676-108258248 | LSM2 | | proc | | | | 1 | 1 | | | |
| chr4: 174790868-174792152 | RAN | manual | proc | | | | 1 | 1 | | 2 | |
| chr1: 87282633-87284966 | UBA2 | manual | frag | | | 1 | 1 | | | 2 | |
| chr12: 40966857-40969856 | IGF2BP2 | auto | proc | | | 1 | 1 | | | 3 | |
| chr3: 187961542-187964137 | PSMD10 | manual | proc | | | 1 | 1 | 5 | | | |
| chr7: 16870050-16872687 | RAD17 | manual | proc | | 3 | | | 3 | | | |
| chr11: 82597892-82598941 | CKS1B | | proc | | 1 | | | 3 | | 2 | |
| chrX: 138942285-138943083 | HNRNPA3 | manual | proc | 1 | | | | 3 | 1 | | |
| chr8: 120561397-120562903 | ZNF532 | auto | frag | | 1 | | | 1 | | | |
| chr15: 100135383-100137014 | FLJ00278 | | dup | | | | | | 3 | 1 | |
| chr11: 16461350-16462832 | AKR1B1 | auto | proc | | 1 | 1 | | | 2 | | |
| chr11: 29253864-29254659 | EEF1A1 | | proc | | | | | | 1 | 3 | |
| chrX: 73296169-73299018 | MKRN1 | manual | proc | | | | | | 1 | 3 | |
| chr2: 25932706-25933414 | ENO3 | manual | frag | | 1 | | | | 1 | | |
| chr6: 143853283-143854931 | VDAC1 | manual | proc | | | | | | 1 | 7 | |
| chr1: 42738096-42738591 | TMSB4X | manual | | | 1 | | | 1 | | 5 | |
| chr13: 98665103-98666182 | H2AFZ | manual | proc | | | | | | | 4 | |
| chr10: 13238600-13239995 | BTBD7 | manual | dup | | 2 | | | | | 3 | |
| chr1: 104417120-104419481 | THAP3 | manual | proc | | 1 | | | | | 2 | |
| chr14: 91136604-91139705 | POLR3G | | proc | | 1 | | | | | 1 | |
| chr3: 73314742-73314982 | CCDC75 | auto | proc | | 2 | | | | | 1 | 1 |
| chrX: 40578772-40580026 | MKRN1 | | dup | | | | | 1 | | 2 | 1 |
| chr4: 136186068-136187470 | EEF1A1 | | dup | | | | | | | 3 | 1 |
| chr9: 5100700-5103470 | TCF3 | manual | proc | | | | | | 1 | 7 | |
| chr18: 27796204-27798261 | PGD | auto | proc | | | | | | | | |
| chr14: 70866169-70867395 | PDZD11 | | proc | | | | | | | 7 | |
| chr19: 21725455-21727418 | CCNYL1 | | proc | 1 | | | | | | 8 | |
| chr1: 218865561-218868015 | HDAC1 | manual | dup | | | | | | | 3 | |
| chr17: 55317138-55317916 | NDUFB8 | | proc | | | | 1 | | | | |
| chr7: 7986381-7988620 | PPIL4 | manual | dup | | | | | | | 12 | |
| chr20: 31962213-31964566 | TPM3 | manual | dup | | | | | 2 | | | 1 |
| chr2: 32280035-32282728 | DDX50 | manual | dup | | | | | | | 5 | |
| chr13: 21002739-21003960 | FNTA | manual | proc | | | | 1 | | | 2 | |
| chr7: 156680057-156683908 | UBXN2B | manual | dup | 1 | 1 | | | | | | |
| chr5: 67490784-67491620 | EEF1B2 | auto | proc | | | | | 1 | | | 1 |
| chr10: 64650560-64652770 | TATDN1 | manual | proc | | | | | | | 4 | |
| chr19: 23100389-23102504 | SNX6 | | proc | | 1 | 1 | | | | | |
| chr5: 72327995-72329739 | DULLARD | | proc | | | | | | | 6 | |
| chr13: 19542116-19543366 | KRR1 | manual | dup | | | | 1 | | | 2 | |
| chr9: 107325071-107329784 | GARNL1 | manual | dup | | | | 1 | | | 2 | |
| chr10: 65061132-65064798 | MRPL35 | manual | proc | | 1 | | | | | | |
| chrX: 72660041-72662733 | MAP2K4 | manual | proc | | | | | | | 1 | |
| chr6: 139012261-139013805 | MTCH1 | manual | frag | | 2 | | | | | | |
| chr1: 40371010-40373921 | OAZ1 | manual | proc | | 3 | | | | | | |
| chr10: 121370445-121373045 | RAD1 | manual | proc | | | | | 1 | | 2 | |
| chr20: 32452814-32454340 | CDC42 | manual | proc | | | | | | | 2 | |
| chr12: 100881924-100882389 | NENF | | proc | | | | | | | | |
| chr14: 81452361-81454170 | EIF3EIP | | proc | | | | | 2 | | 2 | |
| chr12: 70530011-70530466 | MRS2 | | proc | | | | | | 1 | 3 | |
| chr2: 231145010-231146919 | TPM3 | manual | proc | | | | | | | 3 | |
| chr7: 56602283-56604066 | YWHAE | manual | dup | | | 2 | | 1 | | | |
| chr5: 92628780-92629995 | POLD2 | auto | proc | | | | 2 | | | 2 | |
| chr10: 66255316-66256651 | ANXA2 | manual | proc | | 2 | | | | | | |
| chr1: 16882259-16884855 | EIF1AX | manual | | | 1 | | | | | | |
| chr15: 19186009-19190031 | PAK2 | auto | proc | | 4 | | | | | | |
| chr4: 129295826-129297562 | FOSL1 | | proc | | 3 | | | | | | |
| chr17: 60204622-60205031 | LOC201175 | | proc | | 2 | | | | | | |
| chr1: 201002645-201003292 | COX7C | manual | proc | | 2 | | | | | | |
| chr8: 87563050-87564438 | NTAN1 | auto | dup | | 1 | | | | | 1 | |
| chr13: 18425000-18427904 | PHF2 | manual | dup | 1 | | | | | | 2 | |
| chr7: 63832277-63834664 | MPZL1 | manual | proc | | 1 | | | | | 3 | |
| chr15: 19270058-19270354 | CXADR | auto | proc | | | | | | 1 | | |
| chr7: 98435001-98435457 | RNF14 | manual | proc | | | | | | | 5 | |
| chr9: 96300008-96304864 | LMLN | manual | dup | | | | | | 2 | | |
| chr16: 21692-25029 | IL9R | manual | dup | | | 1 | 4 | | | 4 | |
| chr21: 36588933-36589981 | SFRS9 | manual | proc | | 1 | | 3 | 1 | | | |
| chr4: 68263271-68265012 | ST3GAL1 | auto | proc | | 1 | | 2 | | | 1 | |
| chr5: 108413260-108415011 | GJA1 | | | | 6 | 1 | | 1 | | | |
| chr7: 80128544-80129812 | TFDP1 | manual | frag | | | | | 1 | 1 | | |
| chrX: 149149690-149150027 | XRCC6 | manual | proc | | 2 | | | | | 1 | |
| chr17: 71733295-71734754 | ATF4 | manual | | | | | | 2 | | 1 | |
| chr5: 108147697-108149170 | GNB2L1 | | dup | | 1 | | | | | | |
| chr4: 46420373-46421380 | RAC1 | auto | proc | | 1 | | | 1 | | | |
| chr17: 73665057-73666771 | EIF5A | | proc | | | 1 | | | | 1 | 1 |
| chr7: 104957816-104958449 | RNF181 | | proc | | 3 | | | 1 | | 1 | |
| chr11: 122335815-122337882 | ATP5F1 | | proc | 1 | | | | 1 | | 1 | |
| chr22: 23903543-23904988 | STMN3 | manual | proc | | 11 | | | | | | 1 |
| chr17: 22433702-22435031 | TUFM | | proc | | 3 | | | | | 1 | |
| chrX: 114805474-114806874 | ASS1 | manual | proc | | 1 | 3 | | 2 | | 1 | |
| chr17: 26359119-26359614 | YWHAE | | dup | | | 2 | | 1 | | 3 | |
| chr15: 7919666 | ANP32B | auto | proc | | | 2 | | | | 2 | 1 |
| chr13: 28070998-28072283 | EIF4A1 | manual | proc | | | 2 | | 1 | | 1 | 1 |
| chr6: 31351298-31355026 | USP8 | manual | proc | 1 | | 1 | | | | 3 | |
| chrX: 70176246-70179958 | SOCS5 | manual | dup | | | 1 | | | | 1 | |
| chr3: 25771373-25774090 | TAF9B | manual | proc | | | 1 | | | | 3 | |
| chr19: 14042134-14043294 | EEF1D | auto | frag | | 7 | 1 | | | | | |
| chrX: 49283282-49284875 | VDAC1 | manual | proc | | | 1 | | 3 | | 1 | |
| chr22: 39511857-39515009 | RFK | manual | proc | | | 2 | 4 | | | 3 | |
| chr6: 138067402-138069986 | PTPN11 | manual | proc | | | 1 | 3 | | | 1 | |
| chr3: 197148302-197149669 | PPP4R2 | manual | dup | | | | 3 | | 2 | 2 | |
| chr17: 72157677-72159762 | PTMA | auto | | | | 3 | 3 | 5 | | 4 | |
| chr5: 95130415-95132592 | HSPD1 | | proc | | | 3 | 3 | | | 2 | |
| chr2: 91318683-91319881 | IGSF3 | manual | | | | 1 | 2 | 1 | 1 | | |
| chr6: 30707737-30709987 | PTMA | manual | | | | 1 | 2 | | | 2 | |
| chrX: 135716676-135717846 | RAN | manual | proc | 1 | | 1 | 2 | | | | |
| chr17: 25951505-25952090 | SMURF2 | manual | | | | | 2 | 1 | | 1 | |
| chr6: 30107367-30109699 | ETF1 | manual | dup | | | 1 | 2 | | | | |
| chr16: 16366198-16368408 | PKD1 | | dup | | | | 1 | 2 | 2 | 1 | |
| chr13: 76460074-76462820 | DHX9 | manual | proc | 1 | | | 1 | | | 6 | |
| chr22: 40726971-40728762 | SLC25A5 | manual | proc | | | | 1 | | | 1 | |
| chr3: 123862123-123865031 | EIF4B | | proc | | | | 1 | | | 2 | |
| chr6: 128942962-128943901 | EEF1D | manual | proc | | 2 | | 1 | | | | 1 |
| chr2: 202908139-202909954 | FAM64A | manual | proc | | | 1 | 1 | | | 3 | |
| chr13: 88810056-88813657 | SP3 | manual | proc | | 1 | 1 | 1 | | | 4 | |
| chr13: 36410025-36411827 | EIF4A1 | manual | proc | | | | 1 | 4 | 1 | | 1 |
| chrX: 120164368-120165004 | HSPA8 | manual | | | | | | 3 | | 2 | 1 |
| chrX: 70451371-70452584 | ZCRB1 | manual | dup | 1 | | | | 2 | | 2 | |
| chr7: 98432519-98435031 | RNF14 | manual | proc | | | | 1 | | 3 | 6 | |
| chr1: 78085216-78088009 | CCDC55 | manual | proc | | 2 | | | | 3 | 5 | |
| chr1: 152090703-152092464 | ADFP | manual | proc | | 1 | | | | 2 | 1 | |
| chr7: 137395348-137395688 | TPT1 | manual | frag | | | | | | 2 | | 1 |
| chr11: 66285096-66286445 | C1QBP | auto | proc | 1 | 2 | | | | 2 | | |
| chr7: 127889632-127889961 | MLL3 | manual | | | | 1 | | | 2 | 1 | |
| chr1: 241495850-241498178 | FCF1 | manual | proc | | | | | | 2 | 5 | |
| chr12: 74606217-74606813 | TMEM141 | | proc | | | | | 1 | 1 | | |
| chr12: 78225570-78226943 | REST | | dup | | 1 | | | | 1 | 1 | |
| chr1: 169906030-169906835 | PFN1 | manual | proc | | 1 | | | | | 6 | |
| chr2: 224277513-224278767 | MLX | manual | proc | | | 1 | 1 | 2 | | | 5 |
| chr12: 64912853-64915025 | RBM51 | auto | proc | | | 1 | | | | 2 | |
| chr19: 59425000-59426807 | LSM14A | manual | dup | | | 1 | 1 | 1 | | | |
| chr6: 36167532-36168612 | NUDT5 | manual | proc | | | | 2 | | | 7 | |
| chr9: 70415029-70417781 | DSE | manual | proc | | | 1 | 1 | | | 4 | |
| chr15: 63850100-63851589 | DNAJB14 | auto | proc | | 1 | | | | | 8 | |
| chr1: 203861013-203862993 | GABARAP | manual | dup | | | | | 1 | | 1 | |
| chr6: 111285905-111288987 | CNN2 | manual | proc | | | | | | | 5 | |
| chr6: 36750047-36751612 | LAP3 | manual | proc | | | 1 | | | | 1 | |
| chr3: 133590336-133594125 | KIAA1333 | | dup | | | 1 | | 1 | | | |
| chr5: 172445309-172447866 | CDC42 | | proc | | | | | | | 4 | |
| chr7: 79844651-79844976 | SNRPB | manual | frag | | | 2 | | | | 1 | |
| chr3: 76565834-76568498 | CAP1 | | proc | | | 1 | | | | 1 | 1 |
| chr12: 70528835-70530029 | MRS2 | | proc | | | | | | 2 | 7 | |
| chr14: 69770604-69771489 | FLJ00248 | | proc | | | | | 1 | | | 1 |
| chrX: 18269212-18269601 | TMSB10 | manual | frag | | 1 | | | 1 | | | 1 |
| chr3: 23895319-23895949 | ARL4A | manual | proc | | 1 | 1 | | | | 6 | |
| chr3: 155553099-155554758 | DDX50 | | proc | | 1 | | | 2 | | 2 | |
| chr2: 48712985-48713883 | TPT1 | manual | proc | | | | | 1 | 1 | 2 | |
| chr17: 78125786-78128688 | ARL2BP | | dup | | 2 | | | | | 2 | |
| chr8: 124318219-124318608 | UBA52 | | proc | | 3 | | | | | | |
| chr14: 63130269-63131835 | GCAT | auto | proc | | | | 3 | | | 6 | |
| chr9: 89871235-89875006 | PNRC2 | manual | | | | | | | 2 | 2 | |
| chrX: 63570009-63572264 | SHC1 | manual | proc | | | 1 | 1 | | | | |
| chr1: 245420253-245420689 | ZNF720 | manual | proc | | | | | | | 3 | |
| chr5: 21918407-21920011 | HSPD1 | | proc | | | | 2 | 2 | | | 1 |
| chr5: 31959147-31959652 | TOMM7 | | proc | | | | | | | 1 | |
| chr1: 164511305-164513491 | EWSR1 | manual | dup | | | | | | | 2 | |
| chr4: 66642565-66643009 | IFITM3 | | proc | | 1 | | | | | | |
| chr9: 33009522-33010029 | LAGE3 | manual | proc | | 1 | | | | | 1 | |
| chr17: 10560021-10560742 | MAGOH | | proc | 1 | | | | | 2 | | |
| chrX: 41420046-41420852 | YWHAZ | manual | | | 1 | | | 1 | | 7 | |
| chr2: 135985004-135986146 | G3BP1 | manual | proc | | 1 | | 3 | 1 | | 2 | |
| chr16: 31486617-31488548 | CSDA | | proc | | | | | 3 | 1 | | |
| chr12: 17034921-17035029 | EEF1A1 | | proc | | | | | 1 | | 2 | |
| chr7: 143975043-143976509 | EEF1A1 | manual | proc | | | | | 2 | 1 | 2 | 1 |
| chr7: 88106317-88107757 | EEF1A1 | manual | proc | | 1 | | | | | 2 | 1 |
| chr10: 21952707-21955029 | HNRNPR | manual | proc | | | 7 | 1 | | | 3 | |
| chr7: 117310724-117311343 | SFRS3 | manual | | | | 7 | 3 | 3 | | 3 | |
| chr2: 41900352-41902242 | LDHA | manual | proc | | 1 | 5 | | 1 | | | 1 |
| chr4: 4946239-4947833 | LDHA | auto | proc | | | 3 | | 1 | 1 | 3 | 1 |
| chr5: 114752657-114755023 | CTNNA1 | | proc | | 1 | 3 | 1 | 5 | | | 1 |
| chr4: 146516051-146518502 | RTN3 | | proc | | 1 | 3 | | 3 | | | |
| chr3: 174029034-174029700 | ATP5G1 | manual | proc | 1 | | 2 | | 1 | 1 | 1 | |
| chr4: 358246-359943 | ZNF43 | | dup | | | 2 | | 1 | | 2 | |
| chr5: 107088630-107089995 | USP7 | | proc | | | 2 | 1 | | | 6 | |
| chr4: 131043706-131044827 | EEF1G | | proc | | 1 | 2 | 1 | 1 | | 2 | |
| chr13: 28191258-28194994 | CYP51A1 | manual | dup | | | 2 | 1 | 2 | 1 | 2 | |
| chr2: 179027517-179028802 | NUDC | manual | proc | | | 1 | | | 1 | 2 | 1 |
| chr15: 91140001-91141446 | ASB9 | | proc | | | 4 | 4 | | | 2 | |
| chr16: 46156547-46158361 | EIF4B | | proc | | | | 3 | 1 | | 2 | |
| chr3: 39375064-39376459 | EEF1A1 | manual | proc | | | | 3 | | | 5 | 1 |
| chr19: 1153449-1154923 | HMGB2 | auto | proc | | | 3 | 3 | | | 1 | |
| chr4: 110693368-110694731 | SET | | proc | | | 1 | 2 | | | 1 | |
| chrX: 115981067-115982444 | SET | manual | proc | | | 1 | 2 | 1 | 1 | 1 | |
| chr5: 37245024-37248385 | OFD1 | auto | proc | | 1 | 1 | 2 | | | 7 | |
| chr4: 79320311-79322415 | SERBP1 | | proc | | | 1 | 2 | 1 | | 3 | |
| chr22: 22600024-22604137 | KLHL5 | manual | dup | | | 2 | 2 | | | 1 | |
| chr5: 177330758-177333479 | SUDS3 | auto | proc | | 2 | | 2 | | | | |
| chr8: 82692562-82693415 | NIPA2 | | proc | | | | 1 | 1 | | 1 | |
| chrY: 11781394-11785025 | LOC389834 | | proc | | | | 1 | | 1 | 3 | |
| chrX: 73266994-73269840 | DDX3Y | manual | dup | | 1 | | 1 | | | 2 | |
| chr16: 15137027-15139197 | PKD1 | manual | dup | | 1 | | 1 | | 1 | | |
| chr12: 1843255-1844894 | LAMP1 | auto | proc | | | 1 | | 4 | | | 1 |
| chr11: 119196989-119198204 | BAT1 | | proc | | 6 | | | 6 | 8 | 6 | |
| chr6: 25131126-25132842 | ASS1 | manual | proc | | 1 | | | | 8 | 5 | |
| chr2: 152954216-152955031 | NUDC | manual | proc | | 1 | | 1 | | 5 | | |
| chr2: 25898653-25900030 | PTGES3 | manual | proc | | | | 1 | | 4 | 8 | |
| chr3: 97977193-97979955 | CDV3 | auto | proc | | 1 | | | 2 | | 5 | |
| chr3: 52382585-52384174 | PPP2R5C | | proc | | | | | 1 | 1 | 7 | |
| chr1: 245420126-245420978 | ZNF682 | manual | proc | | | 4 | 1 | | | 6 | |
| chrX: 73719066-73719697 | PABPC1 | manual | frag | 3 | | | 2 | | | 5 | |
| chr1: 212722108-212723497 | AP3S1 | manual | proc | | | 3 | 1 | 1 | | 2 | |
| chr11: 92908039-92909143 | SRP14 | | proc | | 2 | | | | | 3 | |
| chr21: 44290157-44291481 | H2AFZ | manual | dup | | | 1 | 2 | | | 8 | 1 |
| chr1: 158906538-158909903 | SET | manual | proc | | | 1 | 2 | 2 | | 7 | |
| chr12: 3820029-3825029 | OTUD4 | | proc | | | 1 | 1 | | | 5 | |
| chr14: 54835005-54835966 | CHMP4B | | proc | | 3 | | | | 2 | 4 | |
| chr15: 91077671-91078734 | H2AFV | auto | proc | | | 1 | | | 1 | | |
| chr6: 147770007-147774161 | YAP1 | manual | proc | | | 2 | | | | 2 | |
| chr8: 75677664-75679288 | PCBP2 | | proc | | 1 | | | | 1 | 1 | |
| chr6: 57002238-57004469 | MRPL30 | | proc | | | | | 1 | | 1 | 1 |
| chr19: 3821749-3823145 | FTL | auto | proc | 1 | | 1 | | 3 | | 3 | |
| chr1: 245410530-245415024 | PEX5 | manual | proc | | 1 | | | | 2 | 2 | |
| chr17: 63602780-63604389 | FBXO36 | | dup | | | 2 | 1 | | | | |
| chr5: 60223151-60223641 | GNL3L | manual | | | | 1 | 2 | | | 2 | |
| chr17: 18515359-18517127 | FOXO3 | manual | proc | | 1 | 1 | 2 | | | | |
| chr11: 129407907-129408375 | TCEB2 | | proc | | | 1 | | 1 | | | |
| chr5: 68645003-68645660 | CFL1 | | proc | | | | 3 | | | 2 | |
| chr5: 43530817-43532401 | EEF1A1 | | proc | | 1 | | | | | 4 | 1 |
| chr5: 14850010-14851722 | RBBP4 | | proc | | | 1 | | | | 1 | |
| chr9: 130067782-130069317 | FLJ00278 | | dup | | | 1 | 1 | | | 1 | |
| chr9: 21685064-21689997 | KHSRP | manual | proc | 1 | | | 1 | 3 | | | |
| chr1: 226368927-226369874 | FAM96A | manual | proc | | | 2 | 1 | 1 | | 2 | |
| chr13: 78381046-78382898 | CCT5 | manual | proc | | | 2 | | | | | 1 |
| chrX: 75471475-75472671 | LDHB | manual | proc | | | 2 | 1 | | | 3 | 1 |
| chr2: 236455494-236455892 | TMSB10 | manual | proc | | 1 | | | 2 | | | |
| chr10: 76705756-76706390 | HMGA1 | manual | | | 1 | | 1 | 10 | | 5 | |
| chr10: 79160515-79164753 | ZNF532 | manual | dup | | 1 | 2 | 2 | | | | |
| chr1: 97785042-97785538 | SEC63 | manual | frag | | | 1 | | 1 | | | |
| chr7: 6647990-6650019 | GZF1 | manual | | | | | | 3 | | 6 | |
| chr13: 18423767-18425026 | PHF2 | manual | dup | 1 | 1 | | | | | 2 | |
| chr19: 9746149-9746855 | UBE2L3 | | proc | | | 1 | | | 1 | | |
| chr17: 20628146-20629946 | SCD | manual | proc | | 9 | | | 1 | | | 2 |
| chr1: 76938038-76939270 | TPI1 | manual | proc | | 2 | | | 2 | | 2 | |
| chr2: 162065731-162069847 | AHCTF1 | manual | proc | | 1 | | | 2 | 1 | 1 | |
| chr1: 245429261-245429363 | ZNF720 | manual | | | 2 | | | | 1 | 5 | |
| chr22: 21935039-21937130 | FBXW4 | manual | proc | 1 | | | | | 1 | 3 | |
| chr19: 15881767-15883707 | IPMK | | proc | 3 | 1 | | 1 | 2 | | | |
| chr2: 158630084-158632092 | PTP4A1 | manual | proc | | 1 | 6 | 1 | 1 | | | |
| chr17: 24552608-24554860 | TWF1 | auto | proc | 1 | | 6 | 3 | 2 | | 1 | |
| chr7: 158107538-158109026 | THAP5 | manual | frag | | 1 | 5 | 3 | 2 | | 2 | |
| chr15: 88619130-88620026 | PERP | | proc | | 15 | 4 | 2 | 2 | | | |
| chr18: 55835065-55835698 | FAM60A | auto | proc | | | 4 | 1 | | | 3 | |
| chr2: 84856616-84858887 | LDHA | manual | proc | | 1 | 4 | | 1 | | 5 | 1 |
| chr8: 62011957-62014951 | NASP | | proc | | 1 | 3 | | | | 6 | 1 |
| chr9: 6656022-6660031 | RNF2 | manual | proc | | 1 | 2 | | | 3 | 9 | |
| chr2: 91130114-91132611 | TMEM128 | manual | frag | | | 2 | 1 | 3 | 1 | | |
| chr9: 6660006-6664836 | RNF2 | manual | proc | | | 2 | 1 | 1 | 2 | 8 | |
| chr11: 232267-233719 | SRP19 | | dup | | | 4 | 6 | | | 8 | |
| chr12: 74737397-74739842 | DENR | | proc | | | 7 | 6 | 1 | | 15 | |
| chr6: 127717472-127720031 | YWHAZ | manual | dup | 2 | | 1 | 5 | | 3 | 1 | |
| chr16: 25046-28284 | IL9R | manual | dup | 1 | | 1 | 5 | | | 3 | |
| chr8: 55046759-55049918 | ESF1 | | proc | 1 | 1 | | 4 | | | 11 | |
| chr7: 56261370-56264649 | CCNJ | manual | proc | | | 1 | 3 | 1 | | 4 | |
| chr15: 73400772-73402108 | ANP32B | | proc | | | 3 | 3 | 5 | | 6 | |
| chr5: 175667341-175669888 | BRCC3 | | dup | | | 1 | 3 | | | 7 | |
| chr21: 36345013-36346248 | FAM80B | manual | proc | 1 | | 1 | 2 | | | 2 | |
| chr2: 222535085-222537390 | HSPA9 | manual | proc | | | 1 | 2 | 2 | | 1 | 2 |
| chr7: 5566916-5567375 | S100A11 | manual | proc | 1 | 4 | | 1 | 1 | | | |
| chrX: 130608331-130609932 | HDGF | manual | frag | | 3 | 1 | 1 | 1 | | 1 | |
| chrX: 113915399-113917620 | HSPA8 | manual | proc | 2 | | 1 | 2 | 9 | | 6 | 2 |
| chr13: 27686701-27688179 | EEF1A1 | manual | proc | | 1 | | 1 | | 3 | 4 | |
| chr8: 59500000-59502148 | PTPN11 | | dup | | | 1 | 3 | | 1 | 9 | |
| chr17: 27578894-27579439 | UBL5 | | proc | | | 2 | | | | 6 | |
| chr7: 30528821-30529744 | FLJ00278 | manual | | 1 | | | | | 1 | 8 | |
| chr11: 91708751-91709133 | NDUFB11 | manual | proc | | | 1 | | | | 2 | |
| chr17: 20456347-20460008 | DEF8 | manual | proc | | | 1 | | 1 | | 6 | |
| chr11: 133498167-133500014 | PTP4A2 | | proc | | | 1 | 1 | 4 | 3 | 1 | |
| chr1: 207507608-207508364 | ATP5G2 | manual | proc | | 1 | | | | | 5 | |
| chr16: 18386391-18389697 | PKD1 | | dup | | | | 2 | | 1 | 3 | |
| chr17: 27461314-27463910 | WDR45L | auto | proc | | 1 | | | 1 | | 4 | |
| chr10: 59642034-59642720 | TPT1 | manual | proc | | | | 1 | 1 | 2 | 4 | |
| chr10: 94417431-94419676 | EIF2S2 | manual | proc | | | | 1 | | 2 | 6 | 1 |
| chrX: 48519005-48520014 | ACAA2 | manual | proc | | | | 1 | | 3 | 4 | |
| chr9: 126952546-126954985 | PRPS1 | manual | proc | | 6 | 1 | 1 | | | 4 | |
| chr9: 34308269-34310028 | SERPINH1 | manual | proc | | 2 | 1 | 2 | 1 | | 1 | |
| chr13: 48243346-48243963 | PSME2 | manual | proc | | | 2 | | 3 | | 5 | |
| chrX: 85225950-85227916 | STIP1 | manual | proc | | 4 | 1 | | 1 | | | |
| chr14: 95758990-95760012 | CKS1B | | proc | | | 2 | | 4 | | 2 | |
| chr4: 145986182-145988340 | HSPD1 | auto | proc | | | 2 | | 3 | | 3 | 1 |
| chr7: 22516700-22518121 | EEF1A1 | manual | dup | | | 1 | 1 | | | 2 | |
| chr10: 43420007-43421764 | CAP1 | manual | proc | | | | 1 | 2 | | | 1 |
| chr17: 63634210-63634432 | DQ587552 | | dup | | 2 | | 2 | | | | 1 |
| chr12: 48067082-48070015 | FGFR1OP2 | | proc | | 1 | | 2 | | 1 | 6 | |
| chr9: 138826841-138829981 | NCL | manual | dup | | | 2 | 2 | 1 | | | |
| chr10: 79210011-79211784 | IMPDH1 | manual | proc | | | 1 | 2 | | 1 | | |
| chr6: 109335327-109335840 | ATP5J2 | manual | proc | | | 1 | 2 | 2 | | 3 | |
| chr1: 229223694-229225016 | RNF4 | manual | proc | | | | 2 | | 1 | 2 | |
| chr15: 34692846-34694996 | TPM3 | | proc | | 1 | | | 2 | | 1 | 1 |
| chrX: 67920024-67922986 | SERBP1 | manual | dup | | | 3 | 1 | 1 | | 4 | |
| chrX: 51683397-51683809 | UCRC | manual | proc | | 1 | | | | | 3 | |
| chr6: 131545798-131548372 | DPF2 | manual | proc | | 1 | 2 | 2 | 1 | | 4 | |
| chr10: 135007719-135008625 | BANF1 | manual | frag | | 1 | | 1 | 2 | 1 | 2 | |
| chr10: 127345241-127346621 | ALDOA | manual | proc | | 7 | | 1 | 1 | | 1 | 2 |
| chr22: 19134094-19135031 | KLHL12 | manual | proc | | 1 | | 1 | | 2 | 6 | |
| chr15: 43132056-43133113 | YWHAZ | | proc | | | | 1 | 2 | | 1 | 1 |
| chr2: 106680143-106681581 | EEF1A1 | manual | dup | | | 1 | 1 | | 1 | 4 | 1 |
| chr12: 119766955-119768000 | ARF1 | auto | proc | | 7 | | | 2 | | 1 | 1 |
| chr6: 53207834-53209186 | HMGB1 | manual | proc | | | 8 | 5 | 6 | 2 | 8 | |
| chr9: 30678968-30680030 | RBMX | manual | proc | | | 5 | 3 | 2 | | 3 | 1 |
| chr17: 64826385-64826952 | SNRPG | | proc | | | 4 | 3 | 1 | | 13 | |
| chr7: 150830163-150832517 | ETF1 | | proc | 1 | | 3 | 6 | | | 9 | |
| chr21: 38796179-38796358 | SNRPG | manual | proc | 1 | | 2 | 5 | 7 | 1 | 3 | |
| chr1: 16885318-16888034 | PDE4DIP | manual | | | 5 | 1 | 5 | | 4 | 4 | |
| chr12: 99496386-99499915 | PIGA | | proc | | 1 | 3 | 5 | 2 | | 4 | |
| chr13: 44861259-44863071 | RCN1 | manual | | | 1 | | 3 | 3 | | 2 | |
| chr9: 124488119-124489981 | TLK1 | manual | dup | | 1 | | 2 | 3 | | 12 | |
| chrX: 65210128-65212872 | EIF4B | manual | proc | | 1 | 1 | 2 | 1 | | 5 | |
| chr15: 91139708-91140030 | ASB9 | | proc | | | 1 | 2 | 2 | | 2 | 3 |
| chr1: 166055742-166056259 | FAM177A1 | manual | dup | | 3 | 1 | 2 | 8 | | | 1 |
| chr9: 16765715-16765767 | LSM1 | manual | proc | | 2 | 1 | 1 | 3 | | | |
| chr2: 171165069-171166985 | HMGB1 | manual | proc | 1 | 1 | | 1 | 4 | 1 | 1 | 1 |
| chr3: 157187740-157189549 | SET | | proc | 1 | | | 3 | 14 | | 7 | 1 |
| chr12: 19985423-19989724 | TCP1 | | proc | 1 | | 1 | | 6 | | 2 | 2 |
| chr9: 98527969-98529661 | MKRN1 | manual | dup | | 1 | 1 | | 1 | 2 | 3 | |
| chr7: 46005000-46007016 | TTC4 | manual | proc | 1 | 1 | | | 1 | 1 | 2 | |
| chr2: 85421131-85422577 | PEBP1 | | proc | 2 | 7 | | | | | 1 | 2 |
| chr12: 86700634-86702602 | MKRN1 | auto | proc | 1 | 1 | 1 | | | 1 | 4 | 2 |
| chr15: 97615266-97617895 | HSP90B1 | | proc | | 1 | | | 2 | | 1 | 1 |
| chr13: 40090629-40094438 | RNF6 | manual | dup | | 2 | | 3 | 2 | | 7 | |
| chr17: 30756783-30758835 | DTWD2 | | proc | | 1 | 2 | | | | 1 | 1 |
| chr17: 4072185-4075029 | RYK | | proc | | 2 | 1 | 1 | | 1 | 7 | |
| chr19: 45140233-45141668 | PRR13 | auto | | | 1 | | 1 | 1 | | 5 | 1 |
| chr17: 30850001-30851322 | TAF5L | | proc | 1 | 2 | | | 2 | 4 | 8 | |
| chr2: 202772424-202775011 | DAZAP2 | manual | proc | | 1 | 1 | | 1 | | 1 | 1 |
| chr12: 109351042-109353797 | HMGA1 | | proc | | 1 | | 2 | 1 | | 1 | |
| chr10: 75161319-75163908 | DUSP8 | manual | proc | | 7 | 1 | | | | 2 | |
| chr21: 46897231-46898445 | DSTN | manual | frag | | 1 | 1 | 3 | | | 5 | |
| chr15: 54085179-54086686 | CNOT6L | auto | proc | 1 | 2 | 4 | | 1 | | 3 | |
| chr5: 25945144-25949126 | MSN | | proc | | 1 | 4 | | | | | 1 |
| chr3: 185226809-185228146 | EEF1A1 | manual | dup | | 2 | | | 1 | | 5 | 1 |
| chr7: 20588278-20589374 | EEF1A1 | manual | proc | | | | | 1 | 1 | 3 | 1 |
| chr3: 44595235-44597668 | MPRIP | manual | dup | | 1 | 1 | | | 1 | 1 | |
| chr7: 39575135-39576400 | TXNDC1 | manual | proc | | 4 | 2 | | | 3 | 6 | |
| chr1: 51395688-51396740 | CFL1 | manual | proc | | 3 | 1 | | | | 2 | |
| chr14: 64815693-64817730 | PTBP1 | auto | dup | | 2 | 1 | 2 | | | 5 | |
| chr1: 16006265-16007067 | UQCRH | manual | | | | | 1 | | 4 | 2 | |
| chr5: 177525251-177526202 | APOA1BP | | proc | | 5 | | 2 | 1 | | | 3 |
| chr1: 144990000-144992583 | IGF2BP2 | manual | proc | | | 2 | 2 | 3 | | 1 | 1 |
| chr7: 55672416-55674246 | CDC42 | manual | proc | | | 1 | 1 | 4 | | 1 | |
| chr5: 114875647-114877498 | CCT5 | | proc | | 1 | 3 | | | | 1 | 1 |
| chr2: 101256951-101257613 | PPIA | | dup | | 2 | | | 1 | 3 | 8 | |
| chr7: 27462505-27465027 | EIF4H | manual | proc | | | 1 | 1 | 1 | | 1 | |
| chrX: 100676203-100679915 | PRKCI | manual | proc | | | 1 | 1 | | 3 | 2 | |
| chr3: 171262714-171264755 | SETD8 | | proc | | 1 | | 1 | 3 | | 1 | |
| chr3: 52202197-52204001 | ALDOA | | dup | | 10 | 1 | | | | 2 | 1 |
| chrX: 25573624-25574445 | RANBP1 | manual | | | 3 | 8 | 5 | 4 | | 9 | 1 |
| chr2: 191565081-191567610 | RAB1A | manual | dup | | | 7 | 3 | 3 | | 4 | 1 |
| chrX: 53210000-53210656 | SUV39H2 | manual | frag | 2 | 1 | 7 | 5 | | 2 | 10 | |
| chrX: 41417881-41420027 | YWHAZ | manual | | 1 | 1 | 6 | 2 | 3 | | 5 | 1 |
| chr5: 43622114-43624741 | AMD1 | auto | proc | | 1 | 5 | 3 | 1 | | 10 | 1 |
| chr1: 218493241-218494999 | MORF4L1 | manual | proc | | | 4 | 3 | 5 | 1 | 6 | |
| chr3: 95276627-95278752 | RBBP4 | | proc | 4 | 1 | 7 | 6 | 2 | | 13 | |
| chr6: 160051470-160055015 | HNRNPH2 | manual | proc | | 2 | 4 | 5 | | 10 | 7 | |
| chr12: 55190981-55193352 | HSPD1 | | proc | | | 4 | 5 | 3 | 4 | 7 | 2 |
| chr3: 48272166-48273216 | MRPS18A | manual | proc | 1 | 1 | 1 | 5 | | | 12 | |
| chr11: 6465021-6467394 | VDAC1 | | dup | | 2 | 2 | 5 | | 1 | 12 | |
| chrX: 81638952-81640007 | EIF3J | manual | frag | 1 | | 6 | 5 | 7 | | 4 | |
| chr4: 118715123-118716642 | NT5C3 | manual | proc | | | 6 | 5 | 2 | | 5 | 1 |
| chr12: 110590068-110592654 | PCNP | auto | proc | 1 | | 4 | 4 | 1 | 1 | 8 | |
| chr20: 48963622-48964977 | PSMD10 | manual | proc | 1 | 2 | | 4 | | 1 | 11 | |
| chr4: 22337134-22338823 | CDC42 | auto | | | 1 | 2 | 4 | 2 | 1 | 6 | 1 |
| chr11: 61895019-61899450 | RCC2 | | proc | | | 2 | 3 | 2 | 4 | 3 | 1 |
| chr1: 52606731-52607671 | PLA2G12A | manual | proc | 1 | 5 | | 3 | 4 | | | |
| chr19: 58005048-58009278 | PABPN1 | | proc | 1 | 1 | 1 | 2 | 3 | 3 | 6 | |
| chr4: 88053138-88055031 | ARPC1A | | dup | 1 | | | 3 | | 12 | 6 | 1 |
| chr17: 30848980-30850029 | TAF5L | | proc | 1 | 2 | | | 4 | 9 | 10 | |
| chr6: 30262288-30264297 | PAIP1 | manual | dup | | | 2 | 3 | 1 | 2 | 11 | |
| chr9: 119885003-119885562 | TPT1 | manual | proc | | | | 1 | 2 | 1 | 5 | |
| chr12: 54660003-54663591 | RAB13 | auto | | | 1 | 1 | 2 | | | 6 | 1 |
| chr10: 70062143-70062538 | FAM36A | manual | | 1 | 1 | | 1 | 3 | 2 | 2 | |
| chr12: 19500335-19502091 | EEF1A1 | | proc | | 1 | | 1 | 4 | 1 | 4 | 1 |
| chr17: 39660007-39663513 | SHC1 | | proc | | 3 | | 1 | | | 2 | |
| chr12: 16442198-16445014 | SUPT16H | | proc | | 1 | 2 | 2 | 1 | | | 1 |
| chrX: 120165030-120166529 | HSPA8 | manual | proc | | 2 | 2 | 2 | 1 | | 4 | 1 |
| chr15: 51016593-51017949 | EEF1A1 | | proc | | | | 2 | 8 | 2 | 8 | 1 |
| chr22: 40833082-40834984 | OLA1 | manual | proc | | | 2 | 3 | 1 | | 3 | |
| chr12: 3818860-3819919 | OTUD4 | | proc | | 3 | 5 | 5 | 3 | | 5 | |
| chr2: 74498320-74499480 | HMGA1 | manual | | 1 | 1 | 1 | 5 | 2 | | 6 | |
| chr2: 138761156-138762932 | YWHAE | manual | proc | | | 3 | 5 | 4 | | 6 | |
| chr8: 30215043-30218410 | HSPA8 | | dup | | | | 1 | 2 | | 5 | 1 |
| chr13: 64430032-64431456 | LGMN | manual | dup | | 2 | | 1 | 3 | 2 | 2 | |
| chr1: 96685035-96686396 | EEF1A1 | manual | proc | | | 1 | | 2 | 1 | 3 | 1 |
| chr10: 32840157-32840929 | C1D | manual | proc | | | 1 | 2 | 1 | 1 | 5 | |
| chrX: 65092271-65093777 | RBMX | manual | proc | | | 2 | 2 | 2 | 1 | 2 | |
| chr17: 2436284-2437919 | EIF4A1 | | dup | | 1 | 3 | 1 | 1 | | 3 | 1 |
| chr2: 127030021-127032314 | YWHAZ | manual | proc | | 1 | 3 | 1 | 3 | | 2 | 1 |
| chr15: 19190000-19191609 | PAK2 | auto | proc | 1 | 4 | 1 | | | 1 | 3 | |
| chr9: 22001477-22002779 | UBA52 | manual | proc | | 4 | 3 | 2 | | 2 | 5 | 1 |
| chr1: 177437318-177437893 | EIF4A1 | manual | frag | 2 | | | 2 | 6 | 2 | 3 | 2 |
| chr4: 98042943-98043372 | COX7A2 | auto | proc | | 2 | 7 | 1 | 1 | | 5 | |
| chr10: 107436038-107439382 | YWHAZ | manual | proc | | 1 | 7 | 5 | 4 | | 4 | 1 |
| chr3: 15895007-15896926 | IMPDH1 | manual | dup | | 1 | 4 | 3 | | 1 | 2 | 1 |
| chr7: 144911241-144912979 | DPY19L4 | manual | proc | 1 | 2 | 4 | 3 | 4 | | 3 | |
| chrX: 44377591-44379921 | CTF8 | manual | proc | | 3 | 1 | 6 | 6 | | 8 | 1 |
| chrX: 73340516-73344880 | RAB11FIP1 | manual | proc | 2 | 4 | 5 | 6 | | 1 | 13 | |
| chr12: 67322298-67323260 | ATP5H | | proc | | 1 | 6 | 6 | 1 | 1 | 16 | |
| chr7: 72033364-72034127 | POMZP3 | | dup | | 1 | 1 | 6 | 4 | | 4 | 1 |
| chr1: 89067086-89067800 | TCEB1 | manual | proc | 2 | 3 | 6 | 6 | 3 | | 16 | |
| chrX: 135755159-135758921 | RAB28 | manual | proc | | 4 | 3 | 5 | 2 | 5 | 9 | |
| chr5: 68644250-68645023 | CFL1 | | proc | 1 | 1 | 1 | 5 | | 1 | 5 | |
| chr21: 31920001-31923002 | FBXW11 | manual | proc | 2 | 3 | 3 | 3 | 2 | 4 | 5 | |
| chr22: 24373154-24375027 | YES1 | manual | dup | 2 | 3 | 1 | 1 | | 9 | 13 | 1 |
| chr22: 22320853-22325031 | ASL | manual | dup | 1 | 2 | | 3 | | 8 | 2 | 1 |
| chr3: 53070978-53074165 | SERBP1 | | proc | 1 | | | 1 | 2 | 6 | 7 | |
| chr1: 89341252-89343268 | PTGES3 | manual | | 1 | | 2 | 1 | 11 | 5 | 11 | |
| chr6: 86193656-86194482 | TPT1 | manual | proc | | | 2 | 2 | 4 | 2 | 8 | 2 |
| chr4: 39650588-39652334 | PABPC1 | | dup | 1 | 2 | 1 | 1 | 3 | | 5 | 1 |
| chr3: 139081781-139083983 | HSPA8 | | proc | | 2 | | 2 | 1 | | 6 | 1 |
| chrX: 65635098-65636615 | PKM2 | manual | proc | | 1 | 3 | 1 | | 1 | 1 | |
| chr7: 10457157-10459773 | HSPA8 | manual | proc | | 4 | 2 | 3 | 4 | 1 | 11 | 2 |
| chr7: 151694812-151694991 | PSPH | | proc | | 2 | 2 | 1 | 13 | | 3 | |
| chr12: 4076871-4079190 | HSPA8 | | dup | | | 2 | 4 | 2 | | 7 | 1 |
| chrX: 13306389-13307499 | GPX1 | manual | proc | | 1 | | 4 | 3 | 1 | 1 | 2 |
| chr14: 61368169-61368862 | COX4I1 | | proc | 1 | 8 | 6 | | 1 | | 3 | |
| chr10: 23465825-23468573 | YWHAZ | manual | | 1 | | 6 | 4 | 6 | | 3 | 1 |
| chr3: 182442769-182443381 | FAU | | proc | | 4 | 6 | 2 | 1 | | 7 | |
| chr8: 107777994-107779579 | TAGLN2 | | proc | | 1 | 3 | 2 | 1 | 1 | 5 | |
| chr1: 72512203-72513454 | GDI2 | manual | frag | | 1 | 2 | 1 | 3 | | 5 | 1 |
| chr14: 35420023-35420673 | EWSR1 | | proc | | 1 | 7 | 1 | 3 | 1 | 9 | 2 |
| chrX: 79711705-79715004 | HK2 | manual | proc | | 1 | 3 | 1 | 1 | | 4 | |
| chr1: 245460000-245461944 | HSPA8 | manual | proc | | 1 | 3 | 1 | 2 | | 7 | 2 |
| chr3: 27649231-27650012 | TPM4 | manual | frag | | 3 | 1 | 1 | 10 | 5 | | 1 |
| chr16: 14920001-14923862 | PKD1 | | dup | | 2 | | 1 | 3 | 1 | 3 | 1 |
| chr6: 158950293-158952897 | TATDN2 | manual | proc | | 4 | 7 | 6 | 6 | | 14 | 1 |
| chrX: 70555627-70557712 | PABPN1 | manual | proc | | 3 | 7 | 6 | | 4 | 16 | |
| chr7: 32762364-32763505 | DPY19L2P2 | manual | dup | | 2 | 7 | 6 | 13 | | 7 | 1 |
| chr19: 35220001-35221268 | TAF9 | auto | proc | 1 | 5 | 3 | 5 | 5 | 8 | 7 | |
| chr3: 122007401-122009653 | NAP1L1 | auto | proc | | 1 | 8 | 5 | 5 | | 11 | 1 |
| chr3: 40612582-40613978 | EEF1G | manual | proc | | 3 | 2 | | 14 | 4 | 8 | 2 |
| chr12: 41110881-41113477 | FXR1 | | proc | 1 | 1 | 8 | 6 | 2 | 1 | 13 | |
| chr1: 233967900-233969581 | LDHA | manual | proc | | 1 | 5 | 1 | 2 | 2 | 13 | 4 |
| chr9: 32935789-32937584 | ASS1 | manual | proc | | 1 | 4 | 5 | 2 | 2 | 14 | |
| chr1: 106235172-106236952 | MATR3 | manual | proc | 4 | 4 | 5 | 2 | 4 | | 10 | 1 |
| chr20: 60947228-60948154 | DPH3 | manual | proc | | 4 | 7 | 6 | 4 | | 5 | 3 |
| chr12: 51833223-51834834 | EIF4A1 | auto | proc | | | 3 | 4 | 7 | 2 | 8 | 3 |
| chr12: 13046376-13049181 | HTR7 | | proc | 2 | 2 | 5 | 2 | 5 | 2 | 3 | |
| chr19: 14461030-14461871 | SNRPG | | proc | 1 | 3 | 7 | 5 | | 4 | 1 | |
| chr7: 65525025-65526430 | SKP1 | manual | proc | 2 | 3 | 5 | 2 | 1 | 2 | 9 | 1 |
| chr9: 88816231-88818084 | CDC20 | manual | proc | | 1 | 4 | 3 | 1 | 1 | 3 | |
| chr3: 115086410-115088196 | DULLARD | auto | proc | | | 2 | 3 | 4 | 9 | 7 | 1 |
| chr8: 48585762-48587345 | NSFL1C | | proc | 1 | 3 | 2 | 3 | 1 | 4 | 6 | |
| chr1: 240223113-240224419 | CFL1 | manual | proc | 1 | 3 | 2 | | 2 | 1 | 6 | 1 |
| chr3: 36783146-36785030 | HSPD1 | manual | proc | 1 | 1 | 1 | 3 | 2 | 1 | 1 | 1 |
| chr1: 145141125-145143358 | CCT8 | manual | proc | 2 | 7 | 4 | 5 | 16 | 6 | 13 | |
| chr10: 71593210-71594625 | CALM2 | manual | proc | 3 | 10 | 8 | 6 | 8 | | 6 | 4 |
| chr7: 137738291-137739988 | PTMA | manual | frag | | 3 | 7 | 6 | 18 | 5 | 14 | 3 |
| chr22: 24375001-24378128 | YES1 | manual | dup | 2 | 9 | 8 | 4 | 12 | 1 | 9 | |
| chr9: 97948095-97950029 | EIF4B | manual | proc | | 3 | 8 | 6 | 15 | 3 | 15 | 3 |
| chrX: 115766164-115766603 | PRPF4B | manual | frag | 4 | 5 | 2 | 6 | 10 | 5 | 9 | 2 |
| chr7: 104095351-104099270 | EIF4B | manual | proc | 1 | 1 | 8 | 6 | 9 | 2 | 13 | 3 |
| chr12: 12155020-12156368 | PTMA | auto | frag | 1 | 1 | 8 | 6 | 12 | 4 | 13 | 2 |
| chrX: 74880003-74883639 | TTC3 | manual | proc | 4 | 11 | 6 | 5 | 9 | 4 | 4 | |
| chr2: 61016549-61018625 | NONO | manual | dup | 2 | 2 | 7 | 6 | 5 | 1 | 14 | |
| chr6: 143705076-143706473 | PARL | manual | proc | 2 | 3 | 5 | 5 | 13 | 9 | 3 | |
| chr3: 48306896-48308964 | FCF1 | manual | proc | 1 | 3 | 2 | 5 | 11 | 6 | 10 | |
| chr17: 37935489-37939966 | PTP4A2 | | proc | 1 | 3 | 7 | 6 | 9 | 3 | 9 | 1 |
| chr5: 96416174-96418578 | SET | auto | proc | 2 | 3 | 8 | 6 | 10 | | 14 | 3 |
| chr22: 29627969-29630029 | EIF4H | manual | proc | 2 | 3 | 6 | 6 | 3 | 10 | 6 | 1 |
| chr1: 116875720-116878445 | NAP1L4 | manual | proc | | 5 | 3 | 4 | 1 | 2 | 15 | 2 |
| chr10: 120681594-120682842 | LDHA | manual | frag | | 3 | 7 | 3 | 5 | 6 | 9 | 2 |
| chr11: 18465871-18469227 | YWHAB | | proc | 1 | 1 | 3 | 4 | 4 | 3 | 12 | 2 |
| chr9: 12962422-12963578 | PRDX1 | manual | proc | 2 | 3 | 8 | 5 | 4 | 1 | 2 | |
| chr9: 34911199-34912896 | YWHAZ | manual | proc | 2 | 1 | 8 | 5 | 10 | 2 | 8 | 2 |
| chr17: 11959531-11959908 | DYNLT1 | | proc | 2 | 12 | 2 | 5 | 3 | 4 | | |
| chr19: 18002173-18005024 | CS | | proc | | 1 | 3 | 5 | 5 | 4 | 8 | 3 |
| chr20: 17940242-17942547 | PTMA | manual | | 2 | 2 | 6 | 4 | 6 | | 5 | 1 |
| chr4: 74183155-74184968 | HMGA1 | | dup | | 1 | 2 | 6 | 1 | 2 | 9 | |
| chr6: 144563059-144563906 | TPT1 | manual | proc | | 1 | 2 | 1 | 6 | 3 | 10 | |
| chr7: 124460752-124462651 | EEF1G | manual | dup | | 4 | 4 | 3 | 2 | 1 | 10 | 2 |
| chr9: 14910713-14912555 | LDHA | manual | proc | | 2 | 5 | 1 | 1 | 3 | 8 | 2 |
| chr22: 30995341-30999218 | CPSF1 | manual | proc | | 4 | 2 | 4 | 1 | | 1 | 4 |
| chr22: 24075369-24076698 | PHF10 | manual | proc | 1 | 1 | 1 | 5 | 1 | | 8 | |
| chrY: 11770408-11775026 | FLJ00219 | | dup | 12 | 37 | 3 | 5 | 2 | 9 | 10 | |
| chr11: 43875453-43878110 | SEC14L1 | auto | proc | | 11 | 7 | 6 | 8 | 5 | 16 | 3 |
| chrX: 23003631-23004877 | FAM3C | manual | | 7 | 10 | 1 | 4 | 13 | 1 | 6 | 3 |
| chr12: 32770077-32771796 | NAP1L1 | | proc | 2 | 4 | 8 | 6 | 7 | 9 | 16 | 1 |
| chr1: 75980428-75983188 | DLST | manual | dup | 2 | 6 | 6 | 4 | | 6 | 17 | 2 |
| chr6: 74058239-74059970 | EIF3E | manual | proc | 2 | 2 | 5 | 2 | 9 | 7 | 13 | 1 |
| chr18: 1350751-1353998 | ATAD1 | auto | dup | 3 | 4 | 6 | 5 | 12 | | 6 | 3 |
| chr17: 25420266-25421375 | STX18 | | proc | 4 | 9 | 1 | 4 | 5 | 2 | 5 | |
| chrX: 114843363-114844764 | EEF1G | manual | proc | | 3 | 5 | 5 | 5 | 6 | 11 | 2 |
| chr16: 11905084-11905547 | COX6C | | proc | 1 | 14 | 1 | 4 | 1 | 3 | 11 | 1 |
| chr4: 10982554-10984464 | RNPS1 | | proc | 2 | 5 | 3 | 1 | 10 | 5 | 7 | |
| chrX: 30558147-30559009 | FTL | manual | proc | 1 | 1 | 3 | 3 | 9 | 4 | 8 | 1 |
| chr12: 9283162-9284461 | PTMA | | dup | 2 | 1 | 8 | 6 | 10 | 2 | 11 | 1 |
| chr16: 68346815-68349469 | NONO | | proc | 1 | 1 | 7 | 4 | 7 | 1 | 8 | 2 |
| chr6: 5917621-5919864 | PKM2 | manual | proc | | 8 | 5 | 1 | 3 | 1 | 4 | 2 |
| chr5: 75708184-75708770 | SNRPC | auto | proc | 8 | 55 | 8 | 6 | 19 | 6 | 19 | 10 |
| chrX: 74875425-74880031 | TTC3 | manual | proc | 4 | 28 | 7 | 6 | 19 | 12 | 12 | 6 |
| chr17: 60176781-60178361 | ASCC3L1 | | dup | 11 | 30 | 6 | 6 | 18 | 12 | 13 | 2 |
| chr11: 113145499-113147696 | COX7A2L | | proc | 3 | 37 | 7 | 6 | 15 | 7 | 12 | 5 |
| chr2: 234438687-234440030 | MSL3L1 | manual | proc | 5 | 21 | 7 | 6 | 16 | 4 | 9 | 2 |
| chr17: 63636429-63636974 | DQ587552 | | dup | 12 | 49 | 1 | 6 | 11 | 12 | 10 | 5 |
| chr3: 117845959-117848342 | BZW1 | auto | frag | 12 | 20 | 7 | 6 | 23 | 2 | 15 | 7 |
| chrX: 122077782-122079346 | MRRF | | proc | 3 | 27 | 8 | 6 | 18 | 9 | 11 | 5 |
| chr2: 234440001-234441782 | MSL3L1 | manual | proc | 5 | 17 | 7 | 6 | 19 | 3 | 9 | 2 |
| chr11: 33051914-33054815 | PIGC | auto | proc | 1 | 7 | 8 | 6 | 19 | 8 | 16 | 3 |
| chr10: 42335001-42336262 | ZNF37A | manual | dup | 2 | 6 | 8 | 6 | 16 | 9 | 16 | 2 |
| chr18: 12056629-12058570 | SSBP4 | | dup | 2 | 21 | 1 | 3 | 16 | 7 | 11 | 1 |
| chr3: 180460196-180461897 | LRRFIP1 | | proc | 1 | 12 | 6 | 5 | 8 | 1 | 15 | 2 |
| chr22: 19135000-19137681 | KLHL12 | manual | proc | 2 | 11 | 7 | 6 | 2 | 8 | 14 | 3 |
| chr17: 60380103-60384663 | SLC16A6 | | dup | 1 | 5 | 7 | 6 | 4 | 8 | 13 | 2 |
| chr20: 3952190-3953301 | FTL | manual | | 6 | 5 | 5 | 4 | 19 | 6 | 9 | 1 |
| chr12: 6864001-6865025 | DSTN | manual | proc | 2 | 2 | 6 | 6 | 19 | 7 | 11 | 2 |
| chr12: 62501627-62504537 | PABPC1 | | proc | 3 | 9 | 4 | 3 | 6 | 2 | 7 | 1 |
| chr2: 132196980-132198589 | TOMM40 | manual | proc | 1 | 7 | 7 | 5 | 9 | 2 | 11 | 1 |
| chr1: 114880845-114882611 | PKM2 | manual | proc | 1 | 12 | 5 | 5 | 5 | 6 | 10 | 4 |

| Ψ-Chromosomal | MPN | Oral | Pancreas | Prostate | Salivary | | Sub | Tissue |
|---|---|---|---|---|---|---|---|---|
| Region | (9) | (2) | (28) | (89) | (4) | Sample # | type # | Expression |
| chr21: 10202689-10203575 | | | | | | 2 | 1 | Bladder |
| chr10: 37636369-37640031 | | | | | | 6 | 1 | Breast |
| chr10: 37640002-37644728 | | | | | | 5 | 1 | Breast |
| chr4: 141781899-141782720 | | | | | | 4 | 1 | Breast |
| chr10: 37666189-37668498 | | | | | | 4 | 1 | Breast |
| chr19: 12531356-12532856 | | | | | | 3 | 1 | Breast |
| chr22: 23438167-23438592 | | | | | | 3 | 1 | Breast |
| chr9: 75280000-75280934 | | | | | | 3 | 1 | Breast |
| chr10: 37619903-37620019 | | | | | | 2 | 1 | Breast |
| chr10: 37580608-37581341 | | | | | | 2 | 1 | Breast |
| chr10: 37577033-37577556 | | | | | | 8 | 1 | Breast |
| chr10: 37590732-37594328 | | | | | | 6 | 1 | Breast |
| chr1: 109447342-109448872 | | | | | | 2 | 1 | Breast |
| chr2: 131895157-131898651 | | | | | | 2 | 1 | Breast |
| chr8: 130810585-130813055 | | | | | | 2 | 1 | Cervical |
| chr5: 92250781-92251791 | | | | | | 2 | 1 | Colon |
| chr7: 138533692-138534485 | | | | | | 2 | 1 | Colon |
| chr17: 44331639-44331679 | | | | | | 2 | 1 | Gastric |
| chr12: 12243007-12245031 | | | | | | 2 | 1 | Gastric |
| chr1: 234516058-234516726 | | | | | | 2 | 1 | Gastric |
| chr14: 53527463-53528434 | | | | | | 2 | 1 | Gastric |
| chr10: 69180083-69181213 | | | | | | 2 | 1 | Gastric |
| chr8: 26461161-26461703 | | | | | | 4 | 1 | Kidney |
| chr2: 152955000-152955599 | | | | | | 3 | 1 | Kidney |
| chr7: 134463620-134464973 | | | | | | 2 | 1 | Kidney |
| chr10: 121746618-121747198 | | | | | | 2 | 1 | Kidney |
| chr1: 218507681-218508028 | | | | | | 7 | 1 | Lymphoma |
| chr3: 115431891-115434722 | | | | | | 4 | 1 | Lymphoma |
| chr3: 120732546-120733113 | | | | | | 4 | 1 | Lymphoma |
| chr2: 201985004-201986130 | | | | | | 4 | 1 | Lymphoma |
| chr2: 231530573-231530995 | | | | | | 4 | 1 | Lymphoma |
| chrX: 110747689-110749928 | | | | | | 4 | 1 | Lymphoma |
| chr10: 75852293-75853672 | | | | | | 4 | 1 | Lymphoma |
| chr15: 81278450-81278844 | | | | | | 3 | 1 | Lymphoma |
| chr19: 40565000-40566763 | | | | | | 3 | 1 | Lymphoma |
| chr4: 135468269-135469814 | | | | | | 3 | 1 | Lymphoma |
| chr10: 31689689-31689904 | | | | | | 3 | 1 | Lymphoma |
| chr1: 202765009-202765499 | | | | | | 3 | 1 | Lymphoma |
| chr10: 75957261-75958035 | | | | | | 3 | 1 | Lymphoma |
| chr8: 48152971-48153133 | | | | | | 2 | 1 | Lymphoma |
| chr15: 81288859-81289520 | | | | | | 2 | 1 | Lymphoma |
| chr12: 31355668-31356398 | | | | | | 2 | 1 | Lymphoma |
| chr11: 85683591-85684437 | | | | | | 2 | 1 | Lymphoma |
| chr4: 185717093-185717964 | | | | | | 2 | 1 | Lymphoma |
| chr4: 100049539-100049960 | | | | | | 2 | 1 | Lymphoma |
| chr5: 126875001-126876506 | | | | | | 2 | 1 | Lymphoma |
| chr12: 92041871-92042457 | | | | | | 2 | 1 | Lymphoma |
| chr2: 149346124-149347002 | | | | | | 2 | 1 | Lymphoma |
| chr7: 53822238-53822358 | | | | | | 2 | 1 | Lymphoma |
| chr4: 93322413-93324234 | | | | | | 2 | 1 | Lymphoma |
| chrX: 119227888-119229412 | | | | | | 2 | 1 | Lymphoma |
| chr1: 117985009-117985810 | | | | | | 2 | 1 | Lymphoma |
| chr17: 59917896-59918632 | | | | | | 2 | 1 | Lymphoma |
| chr21: 15051843-15053508 | | | | | | 2 | 1 | Lymphoma |
| chr9: 139655582-139655864 | | | | | | 2 | 1 | Lymphoma |
| chr18: 41862332-41864360 | 7 | | | | | 7 | 1 | MPN |
| chr1: 54763083-54763321 | 5 | | | | | 5 | 1 | MPN |
| chr22: 28770926-28773344 | 5 | | | | | 5 | 1 | MPN |
| chr3: 37151088-37151663 | 5 | | | | | 5 | 1 | MPN |
| chr15: 69242617-69244956 | 4 | | | | | 4 | 1 | MPN |
| chr12: 31798916-31799483 | 4 | | | | | 4 | 1 | MPN |
| chr12: 8555270-8556510 | 4 | | | | | 4 | 1 | MPN |
| chr12: 64915002-64915315 | 4 | | | | | 4 | 1 | MPN |
| chr19: 59424557-59425022 | 4 | | | | | 4 | 1 | MPN |
| chr1: 144988906-144990026 | 4 | | | | | 4 | 1 | MPN |
| chr14: 68403267-68404827 | 3 | | | | | 3 | 1 | MPN |
| chr19: 56731589-56731634 | 3 | | | | | 3 | 1 | MPN |
| chr12: 21491749-21494398 | 3 | | | | | 3 | 1 | MPN |
| chr1: 222429477-222430009 | 3 | | | | | 3 | 1 | MPN |
| chr8: 55597596-55598332 | 3 | | | | | 3 | 1 | MPN |
| chrX: 136350864-136351728 | 3 | | | | | 3 | 1 | MPN |
| chr1: 84482546-84483186 | 3 | | | | | 3 | 1 | MPN |
| chrX: 152525161-152526553 | 3 | | | | | 3 | 1 | MPN |
| chr19: 59469095-59469457 | 2 | | | | | 2 | 1 | MPN |
| chr14: 68405160-68406719 | 2 | | | | | 2 | 1 | MPN |
| chr11: 124182827-124183139 | 2 | | | | | 2 | 1 | MPN |
| chr12: 114514705-114515025 | 2 | | | | | 2 | 1 | MPN |
| chr10: 22554510-22555015 | 2 | | | | | 2 | 1 | MPN |
| chr13: 97437263-97437450 | 2 | | | | | 2 | 1 | MPN |
| chr2: 26225408-26227645 | 2 | | | | | 2 | 1 | MPN |
| chr4: 84387420-84388630 | 2 | | | | | 2 | 1 | MPN |
| chr13: 42405938-42406759 | 2 | | | | | 2 | 1 | MPN |
| chr9: 96088678-96089876 | 2 | | | | | 2 | 1 | MPN |
| chr6: 149475018-149475620 | 2 | | | | | 2 | 1 | MPN |
| chr1: 245420770-245420911 | 2 | | | | | 2 | 1 | MPN |
| chr22: 22040076-22042678 | | | | 9 | | 9 | 1 | Prostate |
| chr15: 19267708-19269952 | | | | 9 | | 9 | 1 | Prostate |
| chr22: 22031558-22032127 | | | | 6 | | 6 | 1 | Prostate |
| chr18: 54508763-54509828 | | | | 6 | | 6 | 1 | Prostate |
| chr4: 166544280-166544941 | | | | 4 | | 4 | 1 | Prostate |
| chr11: 18573250-18574450 | | | | 4 | | 4 | 1 | Prostate |
| chr10: 104925212-104926869 | | | | 4 | | 4 | 1 | Prostate |
| chr15: 33377487-33379076 | | | | 3 | | 3 | 1 | Prostate |
| chr2: 132075527-132076801 | | | | 3 | | 3 | 1 | Prostate |
| chr12: 118116932-118118276 | | | | 3 | | 3 | 1 | Prostate |
| chr7: 63665694-63668112 | | | | 3 | | 3 | 1 | Prostate |
| chr15: 19391669-19394059 | | | | 2 | | 2 | 1 | Prostate |
| chr15: 19396218-19399993 | | | | 2 | | 2 | 1 | Prostate |
| chr4: 72385029-72386725 | | | | 2 | | 2 | 1 | Prostate |
| chr4: 140255185-140256207 | | | | 2 | | 2 | 1 | Prostate |
| chr11: 125442528-125444160 | | | | 2 | | 2 | 1 | Prostate |
| chr12: 60911988-60914886 | | | | 2 | | 2 | 1 | Prostate |
| chr8: 48666667-48667924 | | | | 2 | | 2 | 1 | Prostate |
| chr5: 75410408-75411560 | | | | 2 | | 2 | 1 | Prostate |
| chr10: 93965940-93966789 | | | | 2 | | 2 | 1 | Prostate |
| chr12: 17033298-17034633 | | | | 2 | | 2 | 1 | Prostate |
| chr19: 63029371-63030025 | | | | 2 | | 2 | 1 | Prostate |
| chr3: 121508215-121509682 | | | | 2 | | 2 | 1 | Prostate |
| chr3: 52741566-52743049 | | | | 2 | | 2 | 1 | Prostate |
| chr2: 94890365-94894064 | | | | 2 | | 2 | 1 | Prostate |
| chr1: 245430003-245432118 | | | | 2 | | 2 | 1 | Prostate |
| chr2: 69678006-69679772 | | | | 2 | | 2 | 1 | Prostate |
| chr22: 16277647-16279389 | | | | 2 | | 2 | 1 | Prostate |
| chr3: 132029176-132030005 | | | | 2 | | 2 | 1 | Prostate |
| chr14: 19216101-19218960 | | | | 2 | | 2 | 1 | Prostate |
| chr2: 54131611-54132922 | | | | 2 | | 2 | 1 | Prostate |
| chrX: 18793802-18794774 | | | | 2 | | 2 | 1 | Prostate |
| chr20: 25027736-25029870 | | | | 2 | | 2 | 1 | Prostate |
| chr10: 104595191-104596503 | | | | 2 | | 2 | 1 | Prostate |
| chr6: 101085019-101087991 | | | | 2 | | 2 | 1 | Prostate |
| chr20: 44525241-44526750 | | | | 2 | | 2 | 1 | Prostate |
| chr10: 46552660-46554269 | | | | | | 6 | 2 | Cervical, Gastric |
| chr1: 174850424-174852903 | | | | | | 3 | 2 | Cervical, Gastric |
| chr9: 33592667-33595028 | | | | 2 | | 3 | 2 | Prostate, Cervical |
| chr22: 22386358-22389509 | | | | 2 | | 3 | 2 | Prostate, Cervical |
| chr1: 47081250-47081689 | | | | | | 10 | 2 | Breast, Bladder |
| chr20: 32295708-32298037 | | | | 1 | | 3 | 2 | Colon, Prostate |
| chr8: 30225003-30227051 | | | | | | 7 | 2 | Gastric, Breast |
| chr1: 145020394-145021949 | | | | 1 | | 3 | 2 | Gastric, Prostate |
| chr13: 44066841-44068205 | | | | 1 | | 3 | 2 | Kidney, Prostate |
| chr1: 176981907-176983662 | | | | | | 7 | 2 | Lymphoma, |
| | | | | | | | | Breast |
| chr4: 113966768-113968338 | | | | 1 | | 5 | 2 | Lymphoma, |
| | | | | | | | | Prostate |
| chr13: 76456888-76460017 | | | | 1 | | 5 | 2 | Lymphoma, |
| | | | | | | | | Prostate |
| chr12: 8177240-8178828 | 9 | | | | | 10 | 2 | MPN, Lymphoma |
| chr9: 5103911-5104767 | 5 | | | | | 6 | 2 | MPN, Lymphoma |
| chr17: 30840954-30844887 | 5 | | | | | 7 | 2 | MPN, Lymphoma |
| chr5: 31945012-31947315 | 4 | | | | | 5 | 2 | MPN, Lymphoma |
| chr5: 102395954-102396670 | 4 | | | | | 5 | 2 | MPN, Lymphoma |
| chr9: 5075006-5076284 | 4 | | | | | 6 | 2 | MPN, Lymphoma |
| chr15: 87332965-87334484 | 3 | | | 1 | | 4 | 2 | MPN, Prostate |
| chr12: 4613400-4615020 | 2 | | | 1 | | 3 | 2 | MPN, Prostate |
| chr12: 9199537-9200023 | 2 | | | 1 | | 3 | 2 | MPN, Prostate |
| chr1: 70157624-70158591 | 2 | | | 1 | | 3 | 2 | MPN, Prostate |
| chr3: 12344333-12344751 | 2 | | | 1 | | 3 | 2 | MPN, Prostate |
| chr11: 77772536-77773641 | 2 | | | | | 3 | 2 | MPN, Lymphoma |
| chr5: 172121628-172124841 | 2 | | | | | 3 | 2 | MPN, Lymphoma |
| chrX: 64605779-64607158 | | 1 | | | | 3 | 2 | Breast, Oral |
| chrX: 91255170-91255864 | | 1 | | | | 3 | 2 | Lymphoma, |
| | | | | | | | | Oral |
| chr2: 162640152-162642586 | | | 1 | 45 | | 46 | 2 | Prostate, Pancreas |
| chr4: 166545018-166547102 | | | 1 | 21 | | 22 | 2 | Prostate, Pancreas |
| chr22: 34623087-34624393 | | | | 13 | | 14 | 2 | Prostate, Breast |
| chr1: 177822521-177824924 | | | | 1 | | 4 | 2 | Breast, Prostate |
| chr9: 75278688-75279979 | | | | | | 4 | 2 | Breast, Lymphoma |
| chr9: 37625488-37627344 | | | 1 | | | 4 | 2 | Breast, Pancreas |
| chr15: 98905757-98909660 | | | | 1 | | 3 | 2 | Breast, Prostate |
| chr13: 29768191-29769131 | | | | | | 3 | 2 | Breast, Lymphoma |
| chr17: 6771934-6772425 | | | | | | 4 | 2 | Cervical, Colon |
| chr7: 55294166-55295027 | 1 | | | | | 3 | 2 | Cervical, MPN |
| chr14: 20065201-20066879 | | | | | | 3 | 2 | Cervical, Melanoma |
| chrX: 61915436-61916582 | | | | | | 3 | 2 | Cervical, Melanoma |
| chr14: 57820991-57822278 | 2 | | | | | 4 | 2 | Cervical, MPN |
| chr11: 47882316-47883972 | | | | | | 3 | 2 | Breast, Cervical |
| chr3: 198845497-198847337 | | | | 3 | | 4 | 2 | Prostate, Cervical |
| chr1: 38947029-38949502 | | | | 3 | | 4 | 2 | Prostate, Cervical |
| chr6: 38663042-38664706 | | | | | | 4 | 2 | Colon, Gastric |
| chr5: 144588471-144590025 | | | | | | 3 | 2 | Kidney, Colon |
| chr12: 6865020-6866674 | | | | | | 3 | 2 | Lymphoma, |
| | | | | | | | | Colon |
| chr1: 81740357-81744083 | | | | | | 3 | 2 | Lymphoma, |
| | | | | | | | | Colon |
| chr8: 50815164-50816385 | | | | | | 3 | 2 | Gastric, Colon |
| chr2: 172056105-172057010 | | | | | | 3 | 2 | Gastric, Colon |
| chr11: 118208607-118209718 | | | | | | 3 | 2 | Gastric, Colon |
| chr10: 120393399-120394061 | | | | | | 3 | 2 | Gastric, Colon |
| chr11: 86208209-86209928 | | | | | | 4 | 2 | Gastric, Lymphoma |
| chr3: 73060699-73061349 | | | | 2 | | 3 | 2 | Prostate, Gastric |
| chr22: 22325000-22325556 | | | | | | 5 | 2 | Kidney, Colon |
| chr3: 173800619-173804689 | 2 | | | | | 5 | 2 | Kidney, MPN |
| chr13: 19035415-19037296 | | | | | | 4 | 2 | Lymphoma, |
| | | | | | | | | Kidney |
| chr4: 40482518-40483890 | | | | 2 | | 3 | 2 | Prostate, Kidney |
| chr18: 53581850-53582535 | | | | 3 | | 4 | 2 | Prostate, Kidney |
| chr6: 1458119-1459994 | | | | 3 | | 4 | 2 | Prostate, Kidney |
| chr21: 32790823-32791600 | | | | 3 | | 4 | 2 | Prostate, Kidney |
| chr7: 46003545-46004373 | | | | | | 3 | 2 | Gastric, Kidney |
| chr13: 40464767-40464951 | 1 | | | | | 7 | 2 | Lymphoma, |
| | | | | | | | | MPN |
| chrX: 46468751-46470028 | 1 | | | | | 7 | 2 | Lymphoma, |
| | | | | | | | | MPN |
| chr6: 90581057-90582594 | 1 | | | | | 5 | 2 | Lymphoma, |
| | | | | | | | | MPN |
| chr22: 37448047-37449602 | | | | 3 | | 6 | 2 | Lymphoma, |
| | | | | | | | | Prostate |
| chr19: 62378395-62379706 | | | | 1 | | 3 | 2 | Lymphoma, |
| | | | | | | | | Prostate |
| chr5: 42501936-42504461 | | | | 1 | | 3 | 2 | Lymphoma, |
| | | | | | | | | Prostate |
| chr15: 93200012-93201180 | | | | 1 | | 3 | 2 | Lymphoma, |
| | | | | | | | | Prostate |
| chr11: 18643045-18644377 | | | | 1 | | 3 | 2 | Lymphoma, |
| | | | | | | | | Prostate |
| chr2: 43761451-43762368 | | | | 2 | | 4 | 2 | Lymphoma, |
| | | | | | | | | Prostate |
| chr6: 158577413-158579430 | | | | 2 | | 4 | 2 | Lymphoma, |
| | | | | | | | | Prostate |
| chr14: 60316280-60317538 | | | | 2 | | 3 | 2 | Prostate, Lymphoma |
| chr3: 31470003-31472686 | | | | 2 | | 3 | 2 | Prostate, Lymphoma |
| chr9: 83890554-83893228 | | | | 2 | | 3 | 2 | Prostate, Lymphoma |
| chr1: 246951416-246952125 | | | | | | 3 | 2 | Lymphoma, |
| | | | | | | | | Melanoma |
| chrY: 5265643-5266973 | | | | | | 3 | 2 | Gastric, Melanoma |
| chr12: 17035000-17036276 | | | | | | 4 | 2 | Lymphoma, |
| | | | | | | | | Melanoma |
| chr12: 6457164-6459964 | 7 | | | | | 15 | 2 | Lymphoma, |
| | | | | | | | | MPN |
| chr2: 197835918-197836621 | 7 | | | | | 15 | 2 | Lymphoma, |
| | | | | | | | | MPN |
| chr10: 118771731-118773667 | 6 | | | | | 7 | 2 | MPN, Cervical |
| chr13: 40465037-40465571 | 5 | | | | | 9 | 2 | MPN, Lymphoma |
| chr4: 103870768-103875031 | 4 | | | | | 7 | 2 | MPN, Lymphoma |
| chr13: 30025729-30027622 | 4 | | | | | 7 | 2 | MPN, Lymphoma |
| chr10: 51695144-51697065 | 4 | | | | | 5 | 2 | MPN, Cervical |
| chr5: 139941169-139942270 | 4 | | | | | 5 | 2 | MPN, Cervical |
| chr2: 201982582-201984995 | 4 | | | | | 10 | 2 | Lymphoma, |
| | | | | | | | | MPN |
| chr2: 182267396-182268139 | 4 | | | | | 5 | 2 | MPN, Colon |
| chr3: 133588073-133590018 | 3 | | | 6 | | 9 | 2 | Prostate, MPN |
| chr1: 202041181-202041985 | 3 | | | | | 9 | 2 | Lymphoma, |
| | | | | | | | | MPN |
| chr4: 83415253-83417203 | 2 | | | | | 3 | 2 | MPN, Gastris |
| chr18: 70208031-70208617 | 2 | | | | | 5 | 2 | Breast, MPN |
| chr14: 30221784-30222571 | 2 | | | | | 4 | 2 | Lymphoma, |
| | | | | | | | | MPN |
| chr7: 23625349-23630025 | 2 | | | | | 5 | 2 | Lymphoma, |
| | | | | | | | | MPN |
| chr4: 24268370-24269026 | 2 | | | | | 3 | 2 | MPN, Cervical |
| chr17: 18752228-18753057 | 2 | | | | | 3 | 2 | MPN, Cervical |
| chr6: 139701047-139702459 | 2 | | | | | 7 | 2 | Lymphoma, |
| | | | | | | | | MPN |
| chr1: 2454300040-245431986 | 2 | | | | | 3 | 2 | MPN, Colon |
| chr3: 31197239-31199007 | 2 | | | | | 3 | 2 | MPN, Colon |
| chr3: 138100000-138101718 | 1 | | | 2 | | 3 | 2 | Prostate, MPN |
| chrX: 17026484-17028444 | 1 | | | 2 | | 3 | 2 | Prostate, MPN |
| chr3: 194779703-194779896 | 1 | | | | | 3 | 2 | Lymphoma, |
| | | | | | | | | MPN |
| chr3: 32769304-32770026 | 1 | | | | | 3 | 2 | Lymphoma, |
| | | | | | | | | MPN |
| chr7: 25695625-25696086 | 2 | 1 | | | | 3 | 2 | MPN, Oral |
| chr12: 110503660-110503886 | | 1 | | | | 6 | 2 | Gastric, Oral |
| chr17: 18510983-18512998 | | | 1 | 2 | | 3 | 2 | Prostate, Pancreas |
| chr22: 22070000-22074479 | | | | 14 | | 15 | 2 | Prostate, Cervical |
| chr17: 60205003-60209312 | | | | 13 | | 19 | 2 | Breast, Prostate |
| chr3: 49329045-49329924 | | | | 9 | | 12 | 2 | Prostate, Breast |
| chr22: 22045609-22049946 | | | | 8 | | 9 | 2 | Prostate, Kidney |
| chr2: 20355007-20355544 | | | | 5 | | 6 | 2 | Prostate, Breast |
| chr15: 33171848-33172885 | | | | 5 | | 6 | 2 | Prostate, Breast |
| chr7: 75540406-75544538 | | | | 4 | | 5 | 2 | Prostate, Breast |
| chr5: 110312099-110314335 | | | | 2 | | 3 | 2 | Prostate, Breast |
| chr4: 119333317-119335028 | | | | 2 | | 3 | 2 | Prostate, Breast |
| chr3: 171145011-171147501 | | | | | 1 | 5 | 2 | Lymphoma |
| chr2: 132196348-132197314 | | | | 2 | | 5 | 3 | Cervical, Prostate |
| chrX: 70180396-70182077 | | | | 2 | | 6 | 3 | Colon, Prostate |
| chr15: 36537765-36539186 | | | | 1 | | 4 | 3 | Colon, Lymphoma |
| chr1: 92311703-92313396 | | | | | | 4 | 3 | Colon, Gastric |
| chr15: 34696499-34699553 | | | | 2 | | 4 | 3 | Prostate, Breast |
| chr14: 74019321-74020027 | 7 | | | | | 9 | 3 | MPN, Cervical |
| chr14: 99902477-99903076 | 6 | | | 1 | | 8 | 3 | MPN, Lymphoma |
| chr1: 97783716-97784986 | 5 | | | 1 | | 7 | 3 | MPN, Lymphoma |
| chrX: 154170506-154173981 | 5 | | | | | 8 | 3 | MPN, Lymphoma |
| chr15: 57756054-57757383 | 5 | | | 3 | | 11 | 3 | MPN, Lymphoma |
| chr6: 145070172-145071408 | 3 | | | 1 | | 5 | 3 | MPN, Lymphoma |
| chr5: 97575000-97575656 | 3 | | | | | 5 | 3 | MPN, Gastric |
| chr20: 5220061-5221727 | 3 | | | | | 5 | 3 | MPN, Gastric |
| chr9: 34906998-34908341 | 2 | | | | | 4 | 3 | MPN, Breast |
| chr7: 63005787-63008547 | 2 | | | | | 4 | 3 | MPN, Breast |
| chr12: 46765022-46765808 | 2 | | | 2 | | 5 | 3 | MPN, Prostate |
| chr5: 157345785-157347510 | | 2 | | | | 4 | 3 | Oral, Breast |
| chr15: 32573790-32575001 | | 2 | 1 | | | 4 | 3 | Oral, Lymphoma |
| chr2: 139375038-139376849 | | 1 | 1 | | | 4 | 3 | Breast, Oral |
| chr1: 64080787-64082020 | | 1 | | | | 5 | 3 | Lymphoma, |
| | | | | | | | | Breast |
| chr19: 32986379-32989682 | | 1 | | | | 4 | 3 | Lymphoma, |
| | | | | | | | | Gastric |
| chr1: 238372430-238375019 | | 1 | 2 | 1 | | 4 | 3 | Pancreas, Oral |
| chr10: 21357922-21359678 | 1 | 1 | | | | 4 | 3 | Lymphoma, |
| | | | | | | | | MPN |
| chr14: 57677677-57678048 | | | | 1 | | 5 | 3 | Breast, Lymphoma |
| chr16: 4552399-4553233 | 3 | | | | | 11 | 3 | Cervical, Lymphoma |
| chrX: 36139141-36140020 | | | | | | 6 | 3 | Cervical, Gastric |
| chr16: 51246583-51248855 | 2 | | | | | 7 | 3 | Cervical, Lymphoma |
| chr3: 97550893-97552589 | 1 | | | | | 4 | 3 | Cervical, Lymphoma |
| chr6: 151297464-151299652 | 1 | | | | | 4 | 3 | Cervical, Lymphoma |
| chrX: 62435062-62436548 | | | | | | 5 | 3 | Breast, Cervical |
| chr7: 138506992-138509638 | | | | 1 | | 4 | 3 | Lymphoma, |
| | | | | | | | | Cervical |
| chr2: 204206408-204208995 | | | | 1 | | 4 | 3 | Lymphoma, |
| | | | | | | | | Cervical |
| chr15: 49024038-49025027 | 1 | | | | | 4 | 3 | Lymphoma, |
| | | | | | | | | Cervical |
| chr7: 84495121-84497088 | | | | | | 4 | 3 | Lymphoma, |
| | | | | | | | | Cervical |
| chr17: 42855745-42858503 | | | | 10 | | 13 | 3 | Breast, Prostate |
| chr16: 17095569-17097803 | | | | | | 5 | 3 | Gastric, Cervical |
| chr18: 22090016-22090564 | | | | 2 | | 6 | 3 | Gastric, Prostate |
| chr9: 37875565-37875969 | | 1 | | | | 10 | 3 | Cervical, Colon |
| chr11: 17030098-17030554 | | | | 1 | | 7 | 3 | Colon, Kidney |
| chr4: 68265004-68266433 | 1 | | | | | 4 | 3 | Colon, Lymphoma |
| chr1: 16890145-16890316 | | | | | 1 | 4 | 3 | Colon, Kidney |
| chr5: 108257676-108258248 | | | | 3 | | 5 | 3 | Prostate, Colon |
| chr4: 174790868-174792152 | | | | | | 4 | 3 | Lymphoma, |
| | | | | | | | | Colon |
| chr1: 87282633-87284966 | | | | | | 4 | 3 | Lymphoma, |
| | | | | | | | | Cervical |
| chr12: 40966857-40969856 | | | | | | 5 | 3 | Lymphoma, |
| | | | | | | | | Cervical |
| chr3: 187961542-187964137 | | | | | | 7 | 3 | Gastric, Cervical |
| chr7: 16870050-16872687 | | | | 3 | | 9 | 3 | Breast, Gastric |
| chr11: 82597892-82598941 | | | | | | 6 | 3 | Gastric, Lymphoma |
| chrX: 138942285-138943083 | | | | | | 5 | 3 | Gastric, Bladder |
| chr8: 120561397-120562903 | | | | 2 | | 4 | 3 | Prostate, Breast |
| chr15: 100135383-100137014 | 1 | | | | | 5 | 3 | Kidney, Lymphoma |
| chr11: 16461350-16462832 | | | | | | 4 | 3 | Kidney, Breast |
| chr11: 29253864-29254659 | | | | 1 | | 5 | 3 | Lymphoma, |
| | | | | | | | | Kidney |
| chrX: 73296169-73299018 | | | | 2 | | 6 | 3 | Lymphoma, |
| | | | | | | | | Prostate |
| chr2: 25932706-25933414 | | | | 2 | | 4 | 3 | Prostate, Breast |
| chr6: 143853283-143854931 | 1 | | | | | 9 | 3 | Lymphoma, |
| | | | | | | | | Kidney |
| chr1: 42738096-42738591 | | | | | | 7 | 3 | Lymphoma, |
| | | | | | | | | Breast |
| chr13: 98665103-98666182 | 1 | | | 1 | | 6 | 3 | Lymphoma, |
| | | | | | | | | MPN |
| chr10: 13238600-13239995 | | | | 1 | | 6 | 3 | Lymphoma, |
| | | | | | | | | Breast |
| chr1: 104417120-104419481 | | | | 2 | | 5 | 3 | Lymphoma, |
| | | | | | | | | Prostate |
| chr14: 91136604-91139705 | | | | 2 | | 4 | 3 | Prostate, Breast |
| chr3: 73314742-73314982 | | | | | | 4 | 3 | Breast, Lymphoma |
| chrX: 40578772-40580026 | | | | | | 4 | 3 | Lymphoma, |
| | | | | | | | | Gastric |
| chr4: 136186068-136187470 | | | | 1 | | 5 | 3 | Lymphoma, |
| | | | | | | | | Melanoma |
| chr9: 5100700-5103470 | 9 | | | | | 17 | 3 | MPN, Lymphoma |
| chr18: 27796204-27798261 | 8 | 1 | | 1 | | 10 | 3 | MPN, Oral |
| chr14: 70866169-70867395 | 6 | | | 1 | | 14 | 3 | Lymphoma, |
| | | | | | | | | MPN |
| chr19: 21725455-21727418 | 6 | | | | | 15 | 3 | Lymphoma, |
| | | | | | | | | MPN |
| chr1: 218865561-218868015 | 4 | | | 4 | | 11 | 3 | MPN, Prostate |
| chr17: 55317138-55317916 | 4 | | | 3 | | 8 | 3 | MPN, Prostate |
| chr7: 7986381-7988620 | 4 | | | 1 | | 17 | 3 | MPN, Lymphoma |
| chr20: 31962213-31964566 | 3 | | | | | 6 | 3 | MPN, Gastric |
| chr2: 32280035-32282728 | 3 | | | 2 | | 10 | 3 | Lymphoma, |
| | | | | | | | | MPN |
| chr13: 21002739-21003960 | 3 | | | | | 6 | 3 | MPN, Lymphoma |
| chr7: 156680057-156683908 | 2 | | | | | 4 | 3 | MPN, Bladder |
| chr5: 67490784-67491620 | 2 | | | | | 4 | 3 | MPN, Gastric |
| chr10: 64650560-64652770 | 2 | | | 1 | | 7 | 3 | Lymphoma, |
| | | | | | | | | MPN |
| chr19: 23100389-23102504 | 2 | | | | | 4 | 3 | MPN, Breast |
| chr5: 72327995-72329739 | 2 | | | 2 | | 10 | 3 | Lymphoma, |
| | | | | | | | | MPN |
| chr13: 19542116-19543366 | 2 | | | | | 5 | 3 | Lymphoma, |
| | | | | | | | | MPN |
| chr9: 107325071-107329784 | 2 | | | | | 5 | 3 | Lymphoma, |
| | | | | | | | | MPN |
| chr10: 65061132-65064798 | 1 | | | 2 | | 4 | 3 | Prostate, Breast |
| chrX: 72660041-72662733 | 1 | | | 2 | | 4 | 3 | Prostate, Lymphoma |
| chr6: 139012261-139013805 | 1 | | | 1 | | 4 | 3 | Breast, MPN |
| chr1: 40371010-40373921 | 1 | | | 4 | | 8 | 3 | Prostate, Breast |
| chr10: 121370445-121373045 | 1 | | | | | 4 | 3 | Lymphoma, |
| | | | | | | | | Gastric |
| chr20: 32452814-32454340 | 1 | | | 2 | | 5 | 3 | Lymphoma, |
| | | | | | | | | Prostate |
| chr12: 100881924-100882389 | 1 | | 2 | 2 | | 5 | 3 | Pancreas, Prostate |
| chr14: 81452361-81454170 | 1 | | | | | 5 | 3 | Gastric, Lymphoma |
| chr12: 70530011-70530466 | 1 | | | | | 5 | 3 | Lymphoma, |
| | | | | | | | | Kidney |
| chr2: 231145010-231146919 | 2 | 1 | | | | 6 | 3 | Lymphoma, |
| | | | | | | | | MPN |
| chr7: 56602283-56604066 | | 1 | | | | 4 | 3 | Cervical, Gastric |
| chr5: 92628780-92629995 | | 1 | | | | 5 | 3 | Colon, Lymphoma |
| chr10: 66255316-66256651 | 1 | | 4 | | | 7 | 3 | Pancreas, Breast |
| chr1: 16882259-16884855 | | | 1 | 2 | | 4 | 3 | Prostate, Breast |
| chr15: 19186009-19190031 | 2 | | | 21 | | 27 | 3 | Breast, Prostate |
| chr4: 129295826-129297562 | | | 1 | 14 | | 18 | 3 | Breast, Prostate |
| chr17: 60204622-60205031 | 1 | | | 9 | | 12 | 3 | Prostate, Breast |
| chr1: 201002645-201003292 | | | 1 | 7 | | 10 | 3 | Prostate, Breast |
| chr8: 87563050-87564438 | | | | 6 | | 8 | 3 | Prostate, Breast |
| chr13: 18425000-18427904 | | | | | 1 | 4 | 3 | Lymphoma, |
| | | | | | | | | Bladder |
| chr7: 63832277-63834664 | | | | | 1 | 5 | 3 | Lymphoma, |
| | | | | | | | | Breast |
| chr15: 19270058-19270354 | | | | 8 | 1 | 10 | 3 | Prostate, Kidney |
| chr7: 98435001-98435457 | 1 | | | | 1 | 7 | 3 | Lymphoma, |
| | | | | | | | | MPN |
| chr9: 96300008-96304864 | | | | 2 | 1 | 5 | 3 | Kidney, Prostate |
| chr16: 21692-25029 | 1 | | | | | 10 | 4 | Colon, Lymphoma |
| chr21: 36588933-36589981 | | | | 5 | | 10 | 4 | Prostate, Colon |
| chr4: 68263271-68265012 | | | | 1 | | 5 | 4 | Colon, Breast |
| chr5: 108413260-108415011 | | | | 5 | | 13 | 4 | Breast, Prostate |
| chr7: 80128544-80129812 | 9 | | | 1 | | 12 | 4 | MPN, Gastric |
| chrX: 149149690-149150027 | 5 | | 1 | | | 9 | 4 | MPN, Breast |
| chr17: 71733295-71734754 | 5 | | | 2 | | 10 | 4 | MPN, Gastric |
| chr5: 108147697-108149170 | 1 | 2 | 1 | | | 5 | 4 | Oral, Breast |
| chr4: 46420373-46421380 | 1 | 2 | | | | 5 | 4 | Oral, Breast |
| chr17: 73665057-73666771 | | 2 | | | | 5 | 4 | Oral, Cervical |
| chr7: 104957816-104958449 | | 1 | | | | 6 | 4 | Breast, Gastric |
| chr11: 122335815-122337882 | | | | 4 | | 7 | 4 | Prostate, Bladder |
| chr22: 23903543-23904988 | | | 4 | 9 | | 25 | 4 | Prostate, Pancreas |
| chr17: 22433702-22435031 | | | 1 | 1 | | 6 | 4 | Breast, Lymphoma |
| chrX: 114805474-114806874 | | | | | | 7 | 4 | Cervical, Gastric |
| chr17: 26359119-26359614 | | | | 2 | | 8 | 4 | Lymphoma, |
| | | | | | | | | Cervical |
| chr15: 7919666 | 1 | | | | | 6 | 4 | Cervical, Lymphoma |
| chr13: 28070998-28072283 | | | | | | 5 | 4 | Cervical, Gastric |
| chr6: 31351298-31355026 | | | | 2 | | 7 | 4 | Lymphoma, |
| | | | | | | | | Prostate |
| chrX: 70176246-70179958 | 1 | | | 2 | | 5 | 4 | Prostate, Cervical |
| chr3: 25771373-25774090 | 1 | | | 1 | | 6 | 4 | Lymphoma, |
| | | | | | | | | Cervical |
| chr19: 14042134-14043294 | | | 1 | 2 | | 11 | 4 | Breast, Prostate |
| chrX: 49283282-49284875 | | | | 1 | | 6 | 4 | Gastric, Cervical |
| chr22: 39511857-39515009 | 1 | | | | | 10 | 4 | Colon, Lymphoma |
| chr6: 138067402-138069986 | | | | 1 | | 6 | 4 | Colon, Cervical |
| chr3: 197148302-197149669 | | | | 1 | | 8 | 4 | Colon, Kidney |
| chr17: 72157677-72159762 | | | | | | 15 | 4 | Gastric, Lymphoma |
| chr5: 95130415-95132592 | | | | 1 | | 9 | 4 | Cervical, Colon |
| chr2: 91318683-91319881 | | | | | | 5 | 4 | Colon, Cervical |
| chr6: 30707737-30709987 | | | | 1 | | 6 | 4 | Colon, Lymphoma |
| chrX: 135716676-135717846 | | | | 5 | | 9 | 4 | Prostate, Colon |
| chr17: 25951505-25952090 | 2 | | | | | 6 | 4 | Colon, MPN |
| chr6: 30107367-30109699 | 2 | | | 1 | | 6 | 4 | Colon, MPN |
| chr16: 16366198-16368408 | | | | | | 6 | 4 | Gastric, Kidney |
| chr13: 76460074-76462820 | | | | 1 | | 9 | 4 | Lymphoma, |
| | | | | | | | | Bladder |
| chr22: 40726971-40728762 | | | 1 | 2 | | 5 | 4 | Prostate, Colon |
| chr3: 123862123-123865031 | 1 | | | 1 | | 5 | 4 | Lymphoma, |
| | | | | | | | | Colon |
| chr6: 128942962-128943901 | | | | 1 | | 5 | 4 | Breast, Colon |
| chr2: 202908139-202909954 | | | | 1 | | 6 | 4 | Lymphoma, |
| | | | | | | | | Cervical |
| chr13: 88810056-88813657 | | | | | | 7 | 4 | Lymphoma, |
| | | | | | | | | Breast |
| chr13: 36410025-36411827 | | | | | | 7 | 4 | Gastric, Colon |
| chrX: 120164368-120165004 | | | | 1 | | 7 | 4 | Gastric, Lymphoma |
| chrX: 70451371-70452584 | | | | 3 | | 8 | 4 | Prostate, Gastric |
| chr7: 98432519-98435031 | | | | 4 | | 14 | 4 | Lymphoma, |
| | | | | | | | | Prostate |
| chr1: 78085216-78088009 | | | | 22 | | 32 | 4 | Lymphoma, |
| | | | | | | | | Kidney |
| chr1: 152090703-152092464 | 1 | | | | | 5 | 4 | Kidney, Breast |
| chr7: 137395348-137395688 | 1 | | | 1 | | 5 | 4 | Kidney, Melanoma |
| chr11: 66285096-66286445 | | | | 10 | | 15 | 4 | Breast, Kidney |
| chr7: 127889632-127889961 | | | | 1 | | 5 | 4 | Kidney, Cervical |
| chr1: 241495850-241498178 | 1 | | | 1 | | 9 | 4 | Lymphoma, |
| | | | | | | | | Kidney |
| chr12: 74606217-74606813 | | | 1 | 3 | | 6 | 4 | Prostate, Gastric |
| chr12: 78225570-78226943 | | | | 4 | | 7 | 4 | Prostate, Breast |
| chr1: 169906030-169906835 | 1 | | | 1 | | 9 | 4 | Lymphoma, |
| | | | | | | | | Breast |
| chr2: 224277513-224278767 | | | | | | 9 | 4 | Melanoma, |
| | | | | | | | | Gastric |
| chr12: 64912853-64915025 | 9 | 1 | | | | 13 | 4 | MPN, Lymphoma |
| chr19: 59425000-59426807 | 8 | | | | | 11 | 4 | MPN, Cervical |
| chr6: 36167532-36168612 | 8 | 1 | | | | 18 | 4 | MPN, Lymphoma |
| chr9: 70415029-70417781 | 7 | | | | | 13 | 4 | MPN, Lymphoma |
| chr15: 63850100-63851589 | 6 | | | 4 | | 19 | 4 | Lymphoma, |
| | | | | | | | | MPN |
| chr1: 203861013-203862993 | 6 | | | 20 | | 28 | 4 | MPN, Prostate |
| chr6: 111285905-111288987 | 6 | 1 | | 1 | | 13 | 4 | MPN, Lymphoma |
| chr6: 36750047-36751612 | 6 | 1 | | | | 9 | 4 | MPN, Cervical |
| chr3: 133590336-133594125 | 5 | | | 7 | | 14 | 4 | Prostate, MPN |
| chr5: 172445309-172447866 | 5 | | | 2 | 1 | 12 | 4 | MPN, Lymphoma |
| chr7: 79844651-79844976 | 5 | | | 1 | | 9 | 4 | MPN, Cervical |
| chr3: 76565834-76568498 | 3 | | | | | 6 | 4 | MPN, Cervical |
| chr12: 70528835-70530029 | 3 | | | 4 | | 16 | 4 | Lymphoma, |
| | | | | | | | | Prostate |
| chr14: 69770604-69771489 | 2 | | | 2 | | 6 | 4 | MPN, Prostate |
| chrX: 18269212-18269601 | 2 | | | | | 5 | 4 | MPN, Breast |
| chr3: 23895319-23895949 | 2 | | | | | 10 | 4 | Lymphoma, |
| | | | | | | | | MPN |
| chr3: 155553099-155554758 | 1 | | | | | 6 | 4 | Gastric, Lymphoma |
| chr2: 48712985-48713883 | 1 | | | | | 5 | 4 | Lymphoma, |
| | | | | | | | | Gastric |
| chr17: 78125786-78128688 | 1 | | | 8 | | 13 | 4 | Prostate, Breast |
| chr8: 124318219-124318608 | 3 | 2 | | 1 | | 9 | 4 | Breast, MPN |
| chr14: 63130269-63131835 | | 2 | | 2 | | 13 | 4 | Lymphoma, |
| | | | | | | | | Colon |
| chr9: 89871235-89875006 | | 1 | | 1 | | 6 | 4 | Kidney, Lymphoma |
| chrX: 63570009-63572264 | | 1 | | 2 | | 5 | 4 | Prostate, Cervical |
| chr1: 245420253-245420689 | 3 | 1 | | 1 | | 8 | 4 | Lymphoma, |
| | | | | | | | | MPN |
| chr5: 21918407-21920011 | | 1 | | | | 6 | 4 | Colon, Gastric |
| chr5: 31959147-31959652 | 4 | 1 | | 1 | | 7 | 4 | MPN, Lymphoma |
| chr1: 164511305-164513491 | 4 | 1 | | 1 | | 8 | 4 | MPN, Lymphoma |
| chr4: 66642565-66643009 | 5 | 1 | 1 | | | 8 | 4 | MPN, Breast |
| chr9: 33009522-33010029 | | | 1 | 2 | | 5 | 4 | Prostate, Breast |
| chr17: 10560021-10560742 | 2 | | | 18 | | 23 | 4 | Kidney, MPN |
| chrX: 41420046-41420852 | | | | | 1 | 10 | 4 | Lymphoma, |
| | | | | | | | | Breast |
| chr2: 135985004-135986146 | | | | 1 | | 8 | 5 | Colon, Lymphoma |
| chr16: 31486617-31488548 | 1 | 2 | 1 | | | 8 | 5 | Gastric, Oral |
| chr12: 17034921-17035029 | 1 | 1 | | 1 | | 6 | 5 | Lymphoma, |
| | | | | | | | | Gastric |
| chr7: 143975043-143976509 | | 1 | | | | 7 | 5 | Gastric, Lymphoma |
| chr7: 88106317-88107757 | | | | 1 | 2 | 7 | 5 | Lymphoma, |
| | | | | | | | | Breast |
| chr10: 21952707-21955029 | 2 | | | 2 | | 15 | 5 | Cervical, Lymphoma |
| chr7: 117310724-117311343 | 2 | | | | | 18 | 5 | Cervical, Colon |
| chr2: 41900352-41902242 | | 1 | | | | 9 | 5 | Cervical, Breast |
| chr4: 4946239-4947833 | | | | | | 9 | 5 | Cervical, Lymphoma |
| chr5: 114752657-114755023 | | | | | | 11 | 5 | Gastric, Cervical |
| chr4: 146516051-146518502 | 3 | | | 2 | | 12 | 5 | Cervical, Gastric |
| chr3: 174029034-174029700 | | | | | | 6 | 5 | Cervical, Bladder |
| chr4: 358246-359943 | 1 | | | 1 | | 7 | 5 | Cervical, Lymphoma |
| chr5: 107088630-107089995 | 1 | | | 3 | | 13 | 5 | Lymphoma, |
| | | | | | | | | Prostate |
| chr4: 131043706-131044827 | | | | | | 7 | 5 | Cervical, Lymphoma |
| chr13: 28191258-28194994 | | | | | | 8 | 5 | Cervical, Gastric |
| chr2: 179027517-179028802 | | | | 1 | | 6 | 5 | Lymphoma, |
| | | | | | | | | Cervical |
| chr15: 91140001-91141446 | 1 | | | 8 | | 19 | 5 | Prostate, Cervical |
| chr16: 46156547-46158361 | 2 | | | 1 | | 9 | 5 | Colon, Lymphoma |
| chr3: 39375064-39376459 | 2 | | | 3 | | 14 | 5 | Lymphoma, |
| | | | | | | | | Colon |
| chr19: 1153449-1154923 | 3 | | 1 | | | 11 | 5 | Cervical, Colon |
| chr4: 110693368-110694731 | 1 | | | 1 | | 6 | 5 | Colon, Cervical |
| chrX: 115981067-115982444 | | | | | | 6 | 5 | Colon, Cervical |
| chr5: 37245024-37248385 | 1 | | | | | 12 | 5 | Lymphoma, |
| | | | | | | | | Colon |
| chr4: 79320311-79322415 | | | | | 1 | 8 | 5 | Lymphoma, |
| | | | | | | | | Colon |
| chr22: 22600024-22604137 | 3 | | | 4 | | 12 | 5 | Prostate, MPN |
| chr5: 177330758-177333479 | 1 | | 3 | 27 | | 35 | 5 | Pancreas, Prostate |
| chr8: 82692562-82693415 | | | 2 | 3 | | 8 | 5 | Prostate, Pancreas |
| chrY: 11781394-11785025 | | | 1 | 4 | | 10 | 5 | Prostate, Lymphoma |
| chrX: 73266994-73269840 | 1 | | | 4 | | 9 | 5 | Prostate, Lymphoma |
| chr16: 15137027-15139197 | 1 | | | 3 | | 7 | 5 | Prostate, Breast |
| chr12: 1843255-1844894 | 1 | | | 1 | | 8 | 5 | Gastric, Cervical |
| chr11: 119196989-119198204 | | | | 9 | | 35 | 5 | Prostate, Kidney |
| chr6: 25131126-25132842 | 3 | | | | 1 | 18 | 5 | Kidney, Lymphoma |
| chr2: 152954216-152955031 | | | | 1 | 1 | 9 | 5 | Kidney, Breast |
| chr2: 25898653-25900030 | 3 | | | 4 | | 20 | 5 | Lymphoma, |
| | | | | | | | | Kidney |
| chr3: 97977193-97979955 | 1 | | 1 | | | 10 | 5 | Lymphoma, |
| | | | | | | | | Gastric |
| chr3: 52382585-52384174 | 8 | | | 1 | | 18 | 5 | MPN, Lymphoma |
| chr1: 245420126-245420978 | 8 | | | 1 | | 20 | 5 | MPN, Lymphoma |
| chrX: 73719066-73719697 | 6 | | | 1 | | 17 | 5 | MPN, Lymphoma |
| chr1: 212722108-212723497 | 6 | | | | | 13 | 5 | MPN, Cervical |
| chr11: 92908039-92909143 | 5 | | | 2 | 1 | 13 | 5 | MPN, Lymphoma |
| chr21: 44290157-44291481 | 5 | | | | | 17 | 5 | Lymphoma, |
| | | | | | | | | MPN |
| chr1: 158906538-158909903 | 5 | | | | | 17 | 5 | Lymphoma, |
| | | | | | | | | MPN |
| chr12: 3820029-3825029 | 4 | | | 1 | | 12 | 5 | Lymphoma, |
| | | | | | | | | MPN |
| chr14: 54835005-54835966 | 4 | | | 18 | | 31 | 5 | Lymphoma, |
| | | | | | | | | MPN |
| chr15: 91077671-91078734 | 4 | | | 16 | 1 | 23 | 5 | MPN, Prostate |
| chr6: 147770007-147774161 | 4 | | | 4 | 1 | 13 | 5 | MPN, Prostate |
| chr8: 75677664-75679288 | 2 | | | 1 | | 6 | 5 | MPN, Breast |
| chr6: 57002238-57004469 | 2 | | | 1 | | 6 | 5 | MPN, Gastric |
| chr19: 3821749-3823145 | 2 | | | | | 10 | 5 | Gastric, Lymphoma |
| chr1: 245410530-245415024 | 2 | | | 1 | | 8 | 5 | Kidney, Lymphoma |
| chr17: 63602780-63604389 | | 2 | | 1 | 1 | 7 | 5 | Cervical, Oral |
| chr5: 60223151-60223641 | 1 | 2 | | | | 8 | 5 | Colon, Lymphoma |
| chr17: 18515359-18517127 | | 2 | | 1 | | 7 | 5 | Colon, Oral |
| chr11: 129407907-129408375 | 5 | 2 | | 2 | | 11 | 5 | MPN, Oral |
| chr5: 68645003-68645660 | 2 | 2 | | 11 | | 20 | 5 | Colon, Lymphoma, |
| chr5: 43530817-43532401 | | 1 | | 1 | | 8 | 5 | Lymphoma, |
| | | | | | | | | Breast |
| chr5: 14850010-14851722 | 1 | 1 | | 5 | | 9 | 5 | Prostate, Cervical |
| chr9: 130067782-130069317 | | 1 | | 3 | | 7 | 5 | Prostate, Cervical |
| chr9: 21685064-21689997 | | 1 | 1 | | | 7 | 5 | Gastric, Bladder |
| chr1: 226368927-226369874 | | 1 | | | | 7 | 5 | Cervical, Lymphoma |
| chr13: 78381046-78382898 | | 1 | 1 | 1 | | 6 | 5 | Cervical, Melanoma |
| chrX: 75471475-75472671 | | 1 | | | | 8 | 5 | Lymphoma, |
| | | | | | | | | Cervical |
| chr2: 236455494-236455892 | 3 | 1 | | 2 | | 9 | 5 | MPN, Gastric |
| chr10: 76705756-76706390 | | 1 | | | | 18 | 5 | Lymphoma, |
| | | | | | | | | Gastric |
| chr10: 79160515-79164753 | | | | 10 | 4 | 19 | 5 | Cervical, Colon |
| chr1: 97785042-97785538 | 1 | | | 1 | 2 | 6 | 5 | Cervical, Gastric |
| chr7: 6647990-6650019 | 1 | | | 1 | 2 | 13 | 5 | Lymphoma, |
| | | | | | | | | Gastric |
| chr13: 18423767-18425026 | | | | 2 | 1 | 7 | 5 | Lymphoma, |
| | | | | | | | | Prostate |
| chr19: 9746149-9746855 | 1 | | | 2 | 1 | 6 | 5 | Prostate, Cervical |
| chr17: 20628146-20629946 | | 2 | 3 | 2 | | 19 | 6 | Breast, Pancreas |
| chr1: 76938038-76939270 | 1 | 1 | 1 | | | 9 | 6 | Breast, Gastric |
| chr2: 162065731-162069847 | 1 | 1 | | | | 7 | 6 | Gastric, Breast |
| chr1: 245429261-245429363 | 1 | | | 2 | 4 | 15 | 6 | Lymphoma, |
| | | | | | | | | Breast |
| chr22: 21935039-21937130 | 1 | | | 6 | 2 | 14 | 6 | Prostate, Lymphoma |
| chr19: 15881767-15883707 | | | 4 | 12 | | 23 | 6 | Pancreas, Bladder |
| chr2: 158630084-158632092 | 2 | | | 1 | | 12 | 6 | Cervical, MPN |
| chr17: 24552608-24554860 | | | | 1 | | 14 | 6 | Cervical, Colon |
| chr7: 158107538-158109026 | | | | 1 | | 14 | 6 | Cervical, Colon |
| chr15: 88619130-88620026 | | | 7 | 20 | | 50 | 6 | Pancreas, Cervical |
| chr18: 55835065-55835698 | 1 | | | 3 | 2 | 14 | 6 | Cervical, Lymphoma |
| chr2: 84856616-84858887 | | 1 | | | | 13 | 6 | Lymphoma, |
| | | | | | | | | Cervical |
| chr8: 62011957-62014951 | 1 | | | 4 | | 16 | 6 | Lymphoma, |
| | | | | | | | | Prostate |
| chr9: 6656022-6660031 | 1 | | | 3 | | 19 | 6 | Lymphoma, |
| | | | | | | | | Kidney |
| chr2: 91130114-91132611 | 1 | | | 2 | | 10 | 6 | Gastric, Cervical |
| chr9: 6660006-6664836 | | | | 3 | | 17 | 6 | Lymphoma, |
| | | | | | | | | Prostate |
| chr11: 232267-233719 | 6 | 1 | | 2 | | 27 | 6 | Lymphoma, |
| | | | | | | | | Colon |
| chr12: 74737397-74739842 | 8 | | | 11 | | 48 | 6 | MPN, Cervical |
| chr6: 127717472-127720031 | | | | 3 | | 15 | 6 | Colon, Kidney |
| chr16: 25046-28284 | 1 | 1 | | | | 12 | 6 | Colon, Lymphoma |
| chr8: 55046759-55049918 | 6 | | | 4 | | 27 | 6 | MPN, Colon |
| chr7: 56261370-56264649 | 4 | | | 1 | | 14 | 6 | Lymphoma, |
| | | | | | | | | MPN |
| chr15: 73400772-73402108 | 4 | | | 1 | | 22 | 6 | Lymphoma, |
| | | | | | | | | Gastric |
| chr5: 175667341-175669888 | 2 | 1 | | 1 | | 15 | 6 | Lymphoma, |
| | | | | | | | | Colon |
| chr21: 36345013-36346248 | 2 | | | 2 | | 10 | 6 | Colon, Lymphoma |
| chr2: 222535085-222537390 | | | 1 | | | 9 | 6 | Colon, Gastric |
| chr7: 5566916-5567375 | 1 | | | 1 | | 9 | 6 | Breast, Bladder |
| chrX: 130608331-130609932 | | | | 4 | | 11 | 6 | Prostate, Breast |
| chrX: 113915399-113917620 | | | | | | 22 | 6 | Gastric, Lymphoma |
| chr13: 27686701-27688179 | 2 | | | 1 | | 12 | 6 | Lymphoma, |
| | | | | | | | | Kidney |
| chr8: 59500000-59502148 | 7 | | | 1 | | 22 | 6 | Lymphoma, |
| | | | | | | | | MPN |
| chr17: 27578894-27579439 | 6 | 1 | 3 | 3 | | 21 | 6 | Lymphoma, |
| | | | | | | | | MPN |
| chr7: 30528821-30529744 | 6 | | | 3 | 2 | 21 | 6 | Lymphoma, |
| | | | | | | | | MPN |
| chr11: 91708751-91709133 | 6 | 1 | 3 | 4 | | 17 | 6 | MPN, Prostate |
| chr17: 20456347-20460008 | 5 | | 1 | 1 | | 15 | 6 | Lymphoma, |
| | | | | | | | | MPN |
| chr11: 133498167-133500014 | 5 | | | | | 15 | 6 | MPN, Gastric |
| chr1: 207507608-207508364 | 5 | | 1 | 1 | 1 | 14 | 6 | Lymphoma, |
| | | | | | | | | MPN |
| chr16: 18386391-18389697 | 5 | | | 3 | 1 | 15 | 6 | MPN, Lymphoma |
| chr17: 27461314-27463910 | 4 | | 2 | 3 | | 15 | 6 | Lymphoma, |
| | | | | | | | | MPN |
| chr10: 59642034-59642720 | 4 | | | 1 | | 13 | 6 | Lymphoma, |
| | | | | | | | | MPN |
| chr10: 94417431-94419676 | 4 | | | 1 | | 15 | 6 | Lymphoma, |
| | | | | | | | | MPN |
| chrX: 48519005-48520014 | 4 | | | 4 | 1 | 17 | 6 | Lymphoma, |
| | | | | | | | | MPN |
| chr9: 126952546-126954985 | 3 | | | 13 | | 28 | 6 | Breast, Lymphoma |
| chr9: 34308269-34310028 | | 2 | | | | 9 | 6 | Breast, Colon |
| chr13: 48243346-48243963 | 4 | 2 | | 1 | | 17 | 6 | Lymphoma, |
| | | | | | | | | MPN |
| chrX: 85225950-85227916 | | 1 | 3 | 2 | | 12 | 6 | Breast, Pancreas |
| chr14: 95758990-95760012 | | 1 | | 2 | 1 | 12 | 6 | Gastric, Cervical |
| chr4: 145986182-145988340 | | 1 | | 1 | | 11 | 6 | Gastric, Lymphoma |
| chr7: 22516700-22518121 | 3 | 1 | | | 1 | 9 | 6 | MPN, Lymphoma |
| chr10: 43420007-43421764 | 3 | 1 | | 1 | | 9 | 6 | MPN, Gastric |
| chr17: 63634210-63634432 | | 1 | 1 | 1 | | 8 | 6 | Breast, Colon |
| chr12: 48067082-48070015 | 1 | 1 | | | | 12 | 6 | Lymphoma, |
| | | | | | | | | Colon |
| chr9: 138826841-138829981 | | 1 | 1 | 2 | | 9 | 6 | Cervical, Colon |
| chr10: 79210011-79211784 | | 1 | | 1 | 1 | 7 | 6 | Colon, Cervical |
| chr6: 109335327-109335840 | | 1 | | 2 | | 11 | 6 | Lymphoma, |
| | | | | | | | | Colon |
| chr1: 229223694-229225016 | 3 | 1 | | 1 | | 10 | 6 | MPN, Colon |
| chr15: 34692846-34694996 | 4 | 1 | | | | 10 | 6 | MPN, Gastric |
| chrX: 67920024-67922986 | | 1 | | | 1 | 11 | 6 | Lymphoma, |
| | | | | | | | | Cervical |
| chrX: 51683397-51683809 | 5 | 1 | 1 | 4 | | 15 | 6 | MPN, Prostate |
| chr6: 131545798-131548372 | | | | | 3 | 13 | 6 | Lymphoma, |
| | | | | | | | | Cervical |
| chr10: 135007719-135008625 | | | | | 1 | 8 | 6 | Gastric, Lymphoma |
| chr10: 127345241-127346621 | | 2 | 1 | | | 15 | 7 | Breast, Melanoma |
| chr22: 19134094-19135031 | 1 | 2 | | 14 | | 27 | 7 | Lymphoma, |
| | | | | | | | | Kidney |
| chr15: 43132056-43133113 | 2 | 2 | | 5 | | 14 | 7 | Prostate, Gastric |
| chr2: 106680143-106681581 | 2 | 2 | | | | 12 | 7 | Lymphoma, |
| | | | | | | | | MPN |
| chr12: 119766955-119768000 | 1 | | 1 | 5 | | 18 | 7 | Breast, Prostate |
| chr6: 53207834-53209186 | 1 | 1 | | | | 31 | 7 | Cervical, Lymphoma |
| chr9: 30678968-30680030 | 1 | | | 1 | | 16 | 7 | Cervical, Colon |
| chr17: 64826385-64826952 | 2 | 1 | | 1 | | 25 | 7 | Cervical, Colon |
| chr7: 150830163-150832517 | 7 | 1 | | 2 | | 29 | 7 | Lymphoma, |
| | | | | | | | | MPN |
| chr21: 38796179-38796358 | | | | 1 | | 20 | 7 | Gastric, Colon |
| chr1: 16885318-16888034 | | | | 1 | 1 | 21 | 7 | Breast, Colon |
| chr12: 99496386-99499915 | 5 | 1 | | | | 21 | 7 | Colon, MPN |
| chr13: 44861259-44863071 | | | 1 | 9 | 1 | 20 | 7 | Prostate, Colon |
| chr9: 124488119-124489981 | 1 | | 1 | 3 | | 23 | 7 | Gastric, Prostate |
| chrX: 65210128-65212872 | 1 | | | 2 | | 13 | 7 | Lymphoma, |
| | | | | | | | | Colon |
| chr15: 91139708-91140030 | | | | 4 | 1 | 15 | 7 | Prostate, Melanoma |
| chr1: 166055742-166056259 | | | 1 | 10 | | 26 | 7 | Gastric, Breast |
| chr9: 16765715-16765767 | 1 | | 1 | 2 | | 11 | 7 | Gastric, Breast |
| chr2: 171165069-171166985 | | | | | | 10 | 7 | Gastric, Bladder |
| chr3: 157187740-157189549 | 3 | | | 1 | | 30 | 7 | Lymphoma, |
| | | | | | | | | Colon |
| chr12: 19985423-19989724 | | | 1 | 4 | | 17 | 7 | Gastric, Prostate |
| chr9: 98527969-98529661 | 1 | | | 4 | | 13 | 7 | Prostate, Lymphoma |
| chr7: 46005000-46007016 | | | 1 | 3 | | 10 | 7 | Prostate, Lymphoma |
| chr2: 85421131-85422577 | 1 | | 1 | 4 | | 18 | 7 | Breast, Prostate |
| chr12: 86700634-86702602 | | | | 2 | | 12 | 7 | Lymphoma, |
| | | | | | | | | Melanoma |
| chr15: 97615266-97617895 | 1 | | 1 | 2 | | 9 | 7 | Gastric, Prostate |
| chr13: 40090629-40094438 | 8 | | | 3 | 1 | 26 | 7 | MPN, Lymphoma |
| chr17: 30756783-30758835 | 7 | | 2 | 4 | | 18 | 7 | MPN, Prostate |
| chr17: 4072185-4075029 | 6 | | | 10 | | 28 | 7 | Lymphoma, |
| | | | | | | | | MPN |
| chr19: 45140233-45141668 | 5 | | | 2 | | 16 | 7 | Lymphoma, |
| | | | | | | | | MPN |
| chr17: 30850001-30851322 | 5 | | | 2 | | 24 | 7 | Lymphoma, |
| | | | | | | | | MPN |
| chr2: 202772424-202775011 | 2 | | | 1 | | 8 | 7 | MPN, Breast |
| chr12: 109351042-109353797 | 1 | 2 | 2 | | | 10 | 7 | Colon, Oral |
| chr10: 75161319-75163908 | 3 | 2 | 5 | 30 | | 50 | 7 | Breast, Pancreas |
| chr21: 46897231-46898445 | 4 | 2 | | 2 | | 18 | 7 | Lymphoma, |
| | | | | | | | | MPN |
| chr15: 54085179-54086686 | | 2 | 4 | | | 17 | 7 | Cervical, Pancreas |
| chr5: 25945144-25949126 | 7 | 2 | 1 | 1 | | 17 | 7 | MPN, Cervical |
| chr3: 185226809-185228146 | 1 | 1 | | 1 | | 12 | 7 | Lymphoma, |
| | | | | | | | | Breast |
| chr7: 20588278-20589374 | 2 | 1 | | 1 | | 10 | 7 | Lymphoma, |
| | | | | | | | | MPN |
| chr3: 44595235-44597668 | | 1 | | 9 | 1 | 15 | 7 | Prostate, Breast |
| chr7: 39575135-39576400 | | 1 | 4 | 6 | | 26 | 7 | Lymphoma, |
| | | | | | | | | Prostate |
| chr1: 51395688-51396740 | 3 | 1 | 1 | | 1 | 12 | 7 | Breast, MPN |
| chr14: 64815693-64817730 | | 1 | 5 | 1 | | 17 | 7 | Lymphoma, |
| | | | | | | | | Pancreas |
| chr1: 16006265-16007067 | 2 | 1 | | 2 | 1 | 13 | 7 | Kidney, Lymphoma, |
| chr5: 177525251-177526202 | | 1 | 2 | 1 | | 15 | 7 | Breast, Melanoma |
| chr1: 144990000-144992583 | 3 | 1 | | | | 13 | 7 | Gastric, MPN |
| chr7: 55672416-55674246 | 4 | 1 | | 1 | | 13 | 7 | Gastric, MPN |
| chr5: 114875647-114877498 | | 1 | 1 | 1 | | 9 | 7 | Cervical, Breast |
| chr2: 101256951-101257613 | 5 | 1 | | 2 | | 22 | 7 | Lymphoma, |
| | | | | | | | | MPN |
| chr7: 27462505-27465027 | 5 | 1 | 1 | | | 11 | 7 | MPN, Cervical |
| chrX: 100676203-100679915 | 3 | | | 11 | 2 | 23 | 7 | Kidney, MPN |
| chr3: 171262714-171264755 | 1 | | | 2 | 1 | 10 | 7 | Gastric, Prostate |
| chr3: 52202197-52204001 | 2 | 2 | 2 | 2 | | 22 | 8 | Lymphoma, |
| | | | | | | | | MPN |
| chrX: 25573624-25574445 | 2 | 2 | | | | 34 | 8 | Lymphoma, |
| | | | | | | | | Cervical |
| chr2: 191565081-191567610 | 8 | | | 4 | 1 | 31 | 8 | MPN, Cervical |
| chrX: 53210000-53210656 | 5 | | | 4 | | 36 | 8 | Cervical, Colon |
| chrX: 41417881-41420027 | 4 | | | | | 23 | 8 | Cervical, Lymphoma, |
| chr5: 43622114-43624741 | 1 | | | 6 | | 28 | 8 | Prostate, Cervical |
| chr1: 218493241-218494999 | 5 | 1 | | 1 | | 26 | 8 | Lymphoma, |
| | | | | | | | | Gastric |
| chr3: 95276627-95278752 | 9 | | | 4 | | 46 | 8 | MPN, Cervical |
| chr6: 160051470-160055015 | 1 | | | 10 | 2 | 41 | 8 | Lymphoma, |
| | | | | | | | | Colon |
| chr12: 55190981-55193352 | 2 | | | 1 | | 28 | 8 | Lymphoma, |
| | | | | | | | | Colon |
| chr3: 48272166-48273216 | 6 | 1 | | 14 | | 41 | 8 | MPN, Colon |
| chr11: 6465021-6467394 | 7 | 1 | | 3 | | 33 | 8 | MPN, Colon |
| chrX: 81638952-81640007 | 1 | 1 | | 2 | | 27 | 8 | Gastric, Cervical |
| chr4: 118715123-118716642 | 5 | 1 | | 2 | | 27 | 8 | Cervical, Colon |
| chr12: 110590068-110592654 | 5 | | | 2 | | 26 | 8 | Lymphoma, |
| | | | | | | | | MPN |
| chr20: 48963622-48964977 | 4 | 1 | | 5 | | 29 | 8 | Prostate, Colon |
| chr4: 22337134-22338823 | 7 | | | | | 24 | 8 | MPN, Lymphoma |
| chr11: 61895019-61899450 | 4 | | | 15 | | 34 | 8 | Kidney, MPN |
| chr1: 52606731-52607671 | | 1 | 6 | 20 | 1 | 41 | 8 | Pancreas, Breast |
| chr19: 58005048-58009278 | | | | 11 | | 28 | 8 | Lymphoma, |
| | | | | | | | | Gastric |
| chr4: 88053138-88055031 | 2 | | | 11 | 2 | 38 | 8 | Lymphoma, |
| | | | | | | | | Colon |
| chr17: 30848980-30850029 | 6 | | | 3 | 1 | 36 | 8 | Kidney, MPN |
| chr6: 30262288-30264297 | 8 | 1 | | 5 | | 33 | 8 | MPN, Prostate |
| chr9: 119885003-119885562 | 6 | | 1 | 2 | 2 | 20 | 8 | MPN, Lymphoma |
| chr12: 54660003-54663591 | 5 | | | 6 | 1 | 23 | 8 | Lymphoma, |
| | | | | | | | | Prostate |
| chr10: 70062143-70062538 | 2 | | | 3 | | 15 | 8 | Gastric, Prostate |
| chr12: 19500335-19502091 | 2 | | | 3 | | 17 | 8 | Gastric, Lymphoma |
| chr17: 39660007-39663513 | 3 | 2 | 2 | 2 | 1 | 16 | 8 | Breast, MPN |
| chr12: 16442198-16445014 | 1 | 2 | | 1 | | 11 | 8 | Cervical, Colon |
| chrX: 120165030-120166529 | | 2 | | 1 | | 15 | 8 | Lymphoma, |
| | | | | | | | | Breast |
| chr15: 51016593-51017949 | 4 | 2 | | 3 | | 30 | 8 | Gastric, Lymphoma |
| chr22: 40833082-40834984 | 6 | 2 | 1 | 6 | | 24 | 8 | MPN, Prostate |
| chr12: 3818860-3819919 | 6 | 2 | | 1 | | 30 | 8 | MPN, Cervical |
| chr2: 74498320-74499480 | | 2 | | 1 | | 19 | 8 | Lymphoma, |
| | | | | | | | | Colon |
| chr2: 138761156-138762932 | 4 | 2 | 1 | 2 | | 27 | 8 | Lymphoma, |
| | | | | | | | | Colon |
| chr8: 30215043-30218410 | 1 | 1 | | 1 | 1 | 13 | 8 | Lymphoma, |
| | | | | | | | | Gastric |
| chr13: 64430032-64431456 | | 1 | 1 | | 1 | 13 | 8 | Gastric, Breast |
| chr1: 96685035-96686396 | | 1 | | 1 | 1 | 11 | 8 | Lymphoma, |
| | | | | | | | | Gastric |
| chr10: 32840157-32840929 | 1 | 1 | | 3 | | 15 | 8 | Lymphoma, |
| | | | | | | | | Prostate |
| chrX: 65092271-65093777 | 4 | 1 | | 1 | | 15 | 8 | MPN, Cervical |
| chr17: 2436284-2437919 | | 1 | | 1 | | 12 | 8 | Cervical, Lymphoma |
| chr2: 127030021-127032314 | 2 | 1 | | | | 14 | 8 | Cervical, Gastric |
| chr15: 19190000-19191609 | 2 | 1 | | 36 | | 49 | 8 | Breast, Prostate |
| chr9: 22001477-22002779 | 5 | 1 | | | | 23 | 8 | Lymphoma, |
| | | | | | | | | MPN |
| chr1: 177437318-177437893 | 1 | | | | 2 | 20 | 8 | Gastric, Lymphoma, |
| chr4: 98042943-98043372 | 8 | 1 | 1 | 4 | | 30 | 9 | MPN, Cervical |
| chr10: 107436038-107439382 | 2 | 1 | | 1 | | 26 | 9 | Cervical, Colon |
| chr3: 15895007-15896926 | 1 | 1 | | 1 | | 15 | 9 | Cervical, Colon |
| chr7: 144911241-144912979 | | 1 | 4 | 1 | | 23 | 9 | Cervical, Gastric |
| chrX: 44377591-44379921 | 2 | 1 | | 2 | | 30 | 9 | Lymphoma, |
| | | | | | | | | Colon |
| chrX: 73340516-73344880 | 7 | 1 | | 21 | | 60 | 9 | MPN, Colon |
| chr12: 67322298-67323260 | 9 | 1 | | 5 | | 46 | 9 | MPN, Cervical |
| chr7: 72033364-72034127 | 2 | 2 | | 2 | | 23 | 9 | Colon, Gastric |
| chr1: 89067086-89067800 | 9 | 2 | | 7 | | 54 | 9 | MPN, Prostate |
| chrX: 135755159-135758921 | 1 | | | 17 | 1 | 47 | 9 | Lymphoma, |
| | | | | | | | | Colon |
| chr5: 68644250-68645023 | 1 | 1 | | 6 | | 22 | 9 | Prostate, Colon |
| chr21: 31920001-31923002 | | 1 | | 4 | | 27 | 9 | Lymphoma, |
| | | | | | | | | Kidney |
| chr22: 24373154-24375027 | 7 | | | 28 | | 65 | 9 | Kidney, MPN |
| chr22: 22320853-22325031 | 2 | | | 7 | 2 | 28 | 9 | Kidney, Prostate |
| chr3: 53070978-53074165 | 2 | 1 | | 2 | 1 | 23 | 9 | Lymphoma, |
| | | | | | | | | Kidney |
| chr1: 89341252-89343268 | 9 | | | 14 | 2 | 56 | 9 | MPN, Kidney |
| chr6: 86193656-86194482 | 7 | 1 | | 2 | | 30 | 9 | Lymphoma, |
| | | | | | | | | MPN |
| chr4: 39650588-39652334 | 4 | | | 2 | | 20 | 9 | Lymphoma, |
| | | | | | | | | MPN |
| chr3: 139081781-139083983 | 1 | 2 | 1 | 1 | | 17 | 9 | Lymphoma, |
| | | | | | | | | Breast |
| chrX: 65635098-65636615 | 1 | 2 | 3 | | 1 | 14 | 9 | Cervical, Pancreas |
| chr7: 10457157-10459773 | | 2 | | 2 | | 31 | 9 | Breast, Gastric |
| chr7: 151694812-151694991 | 1 | 2 | | 1 | 2 | 27 | 9 | Lymphoma, |
| | | | | | | | | Breast |
| chr12: 4076871-4079190 | 2 | 2 | 1 | 1 | | 22 | 9 | Lymphoma, |
| | | | | | | | | Colon |
| chrX: 13306389-13307499 | 6 | 2 | | 2 | | 22 | 9 | MPN, Colon |
| chr14: 61368169-61368862 | 7 | 2 | 7 | 7 | | 42 | 9 | Breast, MPN |
| chr10: 23465825-23468573 | 4 | 2 | | 2 | | 29 | 9 | Cervical, Gastric |
| chr3: 182442769-182443381 | 8 | 2 | 6 | 5 | | 41 | 9 | MPN, Lymphoma |
| chr8: 107777994-107779579 | 9 | 2 | | 1 | | 25 | 9 | MPN, Lymphoma |
| chr1: 72512203-72513454 | 9 | 2 | | 3 | | 27 | 9 | MPN, Lymphoma |
| chr14: 35420023-35420673 | | 2 | | 1 | | 27 | 9 | Lymphoma, |
| | | | | | | | | Cervical |
| chrX: 79711705-79715004 | 4 | 1 | 1 | 1 | | 17 | 9 | Lymphoma, |
| | | | | | | | | MPN |
| chr1: 245460000-245461944 | | 1 | 1 | 1 | | 19 | 9 | Lymphoma, |
| | | | | | | | | Cervical |
| chr3: 27649231-27650012 | | 1 | 3 | 7 | | 32 | 9 | Prostate, Kidney |
| chr16: 14920001-14923862 | 2 | | | 7 | 1 | 21 | 9 | Prostate, Gastric |
| chr6: 158950293-158952897 | 7 | 2 | 1 | 7 | | 55 | 10 | Cervical, MPN |
| chrX: 70555627-70557712 | 8 | 2 | 1 | 20 | 4 | 71 | 10 | MPN, Cervical |
| chr7: 32762364-32763505 | 5 | 1 | | 7 | 3 | 52 | 10 | Cervical, Lymphoma |
| chr19: 35220001-35221268 | 4 | | | 13 | 1 | 52 | 10 | Kidney, Lymphoma |
| chr3: 122007401-122009653 | 5 | 1 | | 6 | 3 | 46 | 10 | Cervical, Prostate |
| chr3: 40612582-40613978 | 2 | 2 | | 7 | 2 | 46 | 10 | Lymphoma, |
| | | | | | | | | Prostate |
| chr12: 41110881-41113477 | 5 | 2 | | 4 | | 43 | 10 | Cervical, Colon |
| chr1: 233967900-233969581 | 9 | 2 | | 3 | | 42 | 10 | MPN, Cervical |
| chr9: 32935789-32937584 | 5 | 1 | | 4 | 2 | 40 | 10 | Colon, MPN |
| chr1: 106235172-106236952 | 8 | | 7 | 4 | | 49 | 10 | MPN, Pancreas |
| chr20: 60947228-60948154 | 2 | 2 | | 3 | 1 | 37 | 10 | Cervical, Colon |
| chr12: 51833223-51834834 | 5 | 2 | | 1 | 1 | 36 | 10 | Lymphoma, |
| | | | | | | | | Gastric |
| chr12: 13046376-13049181 | 7 | 1 | | 6 | | 35 | 10 | MPN, Prostate |
| chr19: 14461030-14461871 | 1 | 2 | | 8 | 2 | 34 | 10 | Prostate, Cervical |
| chr7: 65525025-65526430 | 3 | | | 6 | | 34 | 10 | Lymphoma, |
| | | | | | | | | Prostate |
| chr9: 88816231-88818084 | | 1 | 1 | 14 | 3 | 32 | 10 | Cervical, Colon |
| chr3: 115086410-115088196 | 1 | 1 | 1 | 1 | | 30 | 10 | Kidney, Lymphoma |
| chr8: 48585762-48587345 | 6 | 1 | | 2 | | 29 | 10 | Lymphoma, |
| | | | | | | | | MPN |
| chr1: 240223113-240224419 | 4 | 2 | 1 | | | 23 | 10 | Lymphoma, |
| | | | | | | | | MPN |
| chr3: 36783146-36785030 | | | | 1 | 1 | 13 | 10 | Colon, Gastric |
| chr1: 145141125-145143358 | 9 | 2 | 1 | 24 | | 89 | 11 | MPN, Breast |
| chr10: 71593210-71594625 | 8 | 2 | 6 | 24 | | 85 | 11 | Cervical, Gastric |
| chr7: 137738291-137739988 | 9 | 2 | | 4 | 4 | 75 | 11 | MPN, Cervical |
| chr22: 24375001-24378128 | 8 | 1 | 8 | 12 | | 74 | 11 | Breast, Lymphoma |
| chr9: 97948095-97950029 | 7 | 2 | | 9 | 2 | 73 | 11 | Prostate, Cervical |
| chrX: 115766164-115766603 | 8 | | 3 | 16 | | 70 | 11 | Lymphoma, |
| | | | | | | | | MPN |
| chr7: 104095351-104099270 | 8 | | | 5 | 2 | 58 | 11 | Gastric, Cervical |
| chr12: 12155020-12156368 | 6 | 2 | | 2 | | 57 | 11 | Cervical, Colon |
| chrX: 74880003-74883639 | 2 | | 5 | 30 | 1 | 81 | 11 | Gastric, Cervical |
| chr2: 61016549-61018625 | 9 | 2 | | 4 | 1 | 53 | 11 | MPN, Cervical |
| chr6: 143705076-143706473 | | 2 | 1 | 8 | 2 | 53 | 11 | Kidney, Prostate |
| chr3: 48306896-48308964 | 5 | 1 | | 14 | 3 | 61 | 11 | Kidney, Colon |
| chr17: 37935489-37939966 | 9 | | | 3 | 1 | 52 | 11 | Gastric, Lymphoma |
| chr5: 96416174-96418578 | 7 | 2 | | 3 | 1 | 59 | 11 | Cervical, MPN |
| chr22: 29627969-29630029 | 7 | | | 12 | 3 | 59 | 11 | MPN, Cervical |
| chr1: 116875720-116878445 | 9 | 2 | 3 | 2 | | 48 | 11 | MPN, Breast |
| chr10: 120681594-120682842 | 1 | 2 | 1 | 4 | | 43 | 11 | Lymphoma, |
| | | | | | | | | Cervical |
| chr11: 18465871-18469227 | 5 | 1 | | 7 | | 43 | 11 | Prostate, MPN |
| chr9: 12962422-12963578 | 8 | 2 | 1 | 6 | | 42 | 11 | Cervical, MPN |
| chr9: 34911199-34912896 | 6 | 2 | | 3 | | 49 | 11 | Cervical, Lymphoma |
| chr17: 11959531-11959908 | 2 | 2 | 5 | 10 | 2 | 49 | 11 | Colon, Pancreas |
| chr19: 18002173-18005024 | 3 | 1 | | 2 | 1 | 36 | 11 | Lymphoma, |
| | | | | | | | | Colon |
| chr20: 17940242-17942547 | 4 | 1 | | 4 | 1 | 36 | 11 | Cervical, Gastric |
| chr4: 74183155-74184968 | 1 | 2 | 1 | 7 | 3 | 35 | 11 | Lymphoma, |
| | | | | | | | | Prostate |
| chr6: 144563059-144563906 | 6 | 2 | 1 | 5 | 2 | 39 | 11 | Gastric, MPN |
| chr7: 124460752-124462651 | 4 | 1 | 1 | 6 | | 38 | 11 | Prostate, Breast |
| chr9: 14910713-14912555 | 2 | 2 | | 1 | 1 | 28 | 11 | Lymphoma, |
| | | | | | | | | Cervical |
| chr22: 30995341-30999218 | 1 | 1 | 2 | 50 | 1 | 71 | 11 | Prostate, Breast |
| chr22: 24075369-24076698 | 3 | 1 | 1 | 3 | 1 | 26 | 11 | Lymphoma, |
| | | | | | | | | Colon |
| chrY: 11770408-11775026 | 1 | 2 | 23 | 73 | 3 | 180 | 12 | Kidney, Prostate |
| chr11: 43875453-43878110 | 8 | 2 | 8 | 32 | 3 | 109 | 12 | Gastric, MPN |
| chrX: 23003631-23004877 | | 1 | 18 | 13 | 4 | 81 | 12 | Bladder, Lymphoma |
| chr12: 32770077-32771796 | 9 | 2 | | 20 | 3 | 87 | 12 | Kidney, MPN |
| chr1: 75980428-75983188 | 7 | 1 | 1 | 14 | 2 | 68 | 12 | MPN, Breast |
| chr6: 74058239-74059970 | 2 | 2 | 2 | 19 | | 66 | 12 | Gastric, Kidney |
| chr18: 1350751-1353998 | 1 | 2 | 8 | 9 | 1 | 60 | 12 | Prostate, Pancreas |
| chr17: 25420266-25421375 | 4 | 1 | 7 | 16 | 1 | 59 | 12 | Breast, Pancreas |
| chrX: 114843363-114844764 | 4 | 2 | 1 | 6 | 2 | 52 | 12 | Kidney, Prostate |
| chr16: 11905084-11905547 | 1 | | 5 | 4 | 1 | 47 | 12 | Pancreas, Colon |
| chr4: 10982554-10984464 | 6 | 2 | 1 | 12 | 1 | 55 | 12 | Lymphoma, |
| | | | | | | | | MPN |
| chrX: 30558147-30559009 | 7 | 2 | 4 | 2 | | 45 | 12 | Gastric, Lymphoma |
| chr12: 9283162-9284461 | 8 | 2 | | 1 | 1 | 53 | 12 | Cervical, MPN |
| chr16: 68346815-68349469 | 2 | 2 | | 4 | 1 | 40 | 12 | Lymphoma, |
| | | | | | | | | Cervical |
| chr6: 5917621-5919864 | 2 | 2 | 9 | 2 | 1 | 40 | 12 | Pancreas, Breast |
| chr5: 75708184-75708770 | 6 | 2 | 24 | 62 | 4 | 229 | 13 | Bladder, Cervical |
| chrX: 74875425-74880031 | 9 | 2 | 10 | 71 | 4 | 190 | 13 | MPN, Prostate |
| chr17: 60176781-60178361 | 9 | 2 | 14 | 67 | 4 | 194 | 13 | MPN, Prostate |
| chr11: 113145499-113147696 | 6 | 2 | 15 | 47 | 1 | 163 | 13 | Cervical, Kidney |
| chr2: 234438687-234440030 | 3 | 2 | 15 | 47 | 2 | 139 | 13 | Lymphoma, |
| | | | | | | | | Cervical |
| chr17: 63636429-63636974 | 5 | 2 | 21 | 60 | 3 | 197 | 13 | Prostate, Colon |
| chr3: 117845959-117848342 | 8 | 2 | 17 | 23 | 1 | 143 | 13 | MPN, Cervical |
| chrX: 122077782-122079346 | 4 | 2 | 5 | 23 | 4 | 125 | 13 | Kidney, Cervical |
| chr2: 234440001-234441782 | 3 | 2 | 13 | 33 | 1 | 120 | 13 | Lymphoma, |
| | | | | | | | | Cervical |
| chr11: 33051914-33054815 | 9 | 2 | 2 | 23 | 4 | 108 | 13 | MPN, Cervical |
| chr10: 42335001-42336262 | 9 | 2 | 1 | 27 | 3 | 107 | 13 | Kidney, MPN |
| chr18: 12056629-12058570 | 5 | 1 | 12 | 25 | 2 | 107 | 13 | Kidney, MPN |
| chr3: 180460196-180461897 | 7 | 2 | 4 | 36 | 3 | 102 | 13 | Gastric, MPN |
| chr22: 19135000-19137681 | 3 | 2 | 1 | 37 | 3 | 99 | 13 | Kidney, Cervical |
| chr17: 60380103-60384663 | 6 | 2 | 1 | 18 | 4 | 77 | 13 | Kidney, Cervical |
| chr20: 3952190-3953301 | 9 | 2 | 2 | 7 | 1 | 76 | 13 | Lymphoma, |
| | | | | | | | | MPN |
| chr12: 6864001-6865025 | 7 | 2 | 2 | 10 | 3 | 79 | 13 | Kidney, MPN |
| chr12: 62501627-62504537 | 4 | 2 | 2 | 17 | 1 | 61 | 13 | Breast, Lymphoma |
| chr2: 132196980-132198589 | 5 | 2 | 2 | 6 | 2 | 60 | 13 | Gastric, Breast |
| chr1: 114880845-114882611 | 2 | 2 | 4 | 3 | 1 | 60 | 13 | Kidney, Cervical |

TABLE 4

| | | | | CANCER | | | | | BENIGN | | | | |
|---|---|---|---|---|---|---|---|---|---|---|---|---|---|
| | | | | | | | Pan- | Pros- | | | Lympho- | Pan- | Pros- |
| Ψ-Chromosomal | | | | Breast | Gastric | CLL | creas | tate | Breast | Gastric | blast | creas | tate |
| Region | Ψ-Name | ENCODE | Yale | (53) | (21) | (11) | (25) | (71) | (18) | (4) | (8) | (3) | (18) |
| chr10: 37577033-37577556 | ATP8A2 | manual | dup | 8 | | | | | | | | | |
| chr10: 37636369-37640031 | ATP8A2 | manual | | 6 | | | | | | | | | |
| chr10: 37590732-37594328 | ATP8A2 | manual | dup | 6 | | | | | | | | | |
| chr10: 37640002-37644728 | ATP8A2 | manual | | 5 | | | | | | | | | |
| chr4: 141781899-141782720 | KIAA1856 | | dup | 4 | | | | | | | | | |
| chr10: 37666189-37668498 | TMEM161B | manual | proc | 4 | | | | | | | | | |
| chr18: 70208031-70208617 | FAU | | proc | 3 | | | | | | | | | |
| chr19: 12531356-12532856 | PGK1 | manual | dup | 3 | | | | | | | | | |
| chr22: 23438167-23438592 | CRIP1 | manual | proc | 3 | | | | | | | | | |
| chr9: 75280000-75280934 | DPP3 | manual | proc | 3 | | | | | | | | | |
| chr12: 110503660-110503886 | IFITM1 | | proc | | 5 | | | | | | | | |
| chr13: 36410025-36411827 | EIF4A1 | manual | proc | | 4 | | | | | | | | |
| chrX: 138942285-138943083 | HNRNPA3 | manual | proc | | 3 | | | | | | | | |
| chr19: 21725455-21727418 | CCNYL1 | | proc | | | 8 | | | | | | | |
| chr21: 44290157-44291481 | H2AFZ | manual | dup | | | 8 | | | | | | | |
| chr6: 143853283-143854931 | VDAC1 | manual | proc | | | 7 | | | | | | | |
| chr13: 40464767-40464951 | RGS17 | manual | proc | | | 6 | | | | | | | |
| chrX: 46468751-46470028 | CHMP5 | manual | proc | | | 6 | | | | | | | |
| chr1: 202041181-202041985 | CHMP5 | manual | proc | | | 6 | | | | | | | |
| chr2: 201982582-201984995 | SCYL2 | manual | proc | | | 6 | | | | | | | |
| chr6: 139701047-139702459 | DNAJC7 | manual | proc | | | 5 | | | | | | | |
| chr7: 98435001-98435457 | RNF14 | manual | proc | | | 5 | | | | | | | |
| chr3: 115431891-115434722 | KIAA1539 | | proc | | | 4 | | | | | | | |
| chr2: 106680143-106681581 | EEF1A1 | manual | dup | | | 4 | | | | | | | |
| chr2: 201985004-201986130 | SCYL2 | manual | proc | | | 4 | | | | | | | |
| chrX: 110747689-110749928 | EIF4B | manual | proc | | | 4 | | | | | | | |
| chr10: 75852293-75853672 | RAB5C | manual | proc | | | 4 | | | | | | | |
| chr6: 90581057-90582594 | FAM64A | manual | proc | | | 4 | | | | | | | |
| chr13: 40465037-40465571 | RGS17 | manual | proc | | | 4 | | | | | | | |
| chr15: 81278450-81278844 | BC117556 | auto | | | | 3 | | | | | | | |
| chr4: 135468269-135469814 | PES1 | | proc | | | 3 | | | | | | | |
| chr12: 70530011-70530466 | MRS2 | | proc | | | 3 | | | | | | | |
| chr4: 103870768-103875031 | MANBAL | | proc | | | 3 | | | | | | | |
| chr10: 31689689-31689904 | SPTLC1 | manual | frag | | | 3 | | | | | | | |
| chr7: 23625349-23630025 | FCF1 | manual | dup | | | 3 | | | | | | | |
| chr12: 40966857-40969856 | IGF2BP2 | auto | proc | | | 3 | | | | | | | |
| chr1: 202765009-202765499 | PEF1 | manual | proc | | | 3 | | | | | | | |
| chr10: 75957261-75958035 | MRPL35 | manual | proc | | | 3 | | | | | | | |
| chr15: 19267708-19269952 | CXADR | auto | proc | | | | | 9 | | | | | |
| chr15: 19270058-19270354 | CXADR | auto | proc | | | | | 8 | | | | | |
| chrX: 135716676-135717846 | RAN | manual | proc | | | | | 5 | | | | | |
| chr11: 18573250-18574450 | MTCH1 | | proc | | | | | 4 | | | | | |
| chr10: 104925212-104926869 | MARCKSL1 | manual | proc | | | | | 4 | | | | | |
| chr3: 198845497-198847337 | PPP4R2 | | dup | | | | | 3 | | | | | |
| chr2: 132075527-132076801 | AY458019 | manual | | | | | | 3 | | | | | |
| chr17: 55317138-55317916 | NDUFB8 | | proc | | | | | 3 | | | | | |
| chr6: 1458119-1459994 | ELF2 | manual | proc | | | | | 3 | | | | | |
| chr21: 32790823-32791600 | EXOSC3 | manual | proc | | | | | 3 | | | | | |
| chr5: 110312099-110314335 | BCLAF1 | | dup | 1 | | | | 2 | | | | | |
| chr4: 119333317-119335028 | FKBP4 | | proc | 1 | | | | 2 | | | | | |
| chr15: 34696499-34699553 | LARP4 | | proc | 1 | | | | 2 | | | | | |
| chr2: 25932706-25933414 | ENO3 | manual | frag | 1 | | | | 2 | | | | | |
| chr10: 65061132-65064798 | MRPL35 | manual | proc | 1 | | | | 2 | | | | | |
| chr6: 139012261-139013805 | MTCH1 | manual | frag | 2 | | | | 1 | | | | | |
| chr16: 15137027-15139197 | PKD1 | manual | dup | 1 | | | | 3 | | | | | |
| chr17: 18510983-18512998 | FOXO3 | manual | | | | | 1 | 2 | | | | | |
| chr8: 124318219-124318608 | UBA52 | | proc | 3 | | | | 1 | | | | | |
| chr1: 177822435-177824942 | CENTG2 | manual | proc | 3 | | | | 1 | | | | | |
| chr5: 25945144-25949126 | MSN | | proc | 1 | | | 1 | 1 | | | | | |
| chr7: 75540406-75544538 | LOC285908 | manual | proc | 1 | | | | 4 | | | | | |
| chr3: 73060699-73061349 | FTH1 | | proc | | 1 | | | 2 | | | | | |
| chr14: 69770604-69771489 | FLJ00248 | | proc | | 1 | | | 2 | | | | | |
| chr11: 129407907-129408375 | TCEB2 | | proc | | 1 | | | 2 | | | | | |
| chr2: 132196348-132197314 | BC016035 | manual | proc | | 1 | | | 2 | | | | | |
| chr2: 139375038-139376849 | AHCY | manual | proc | 2 | | | 1 | | | | | | |
| chr12: 16442198-16445014 | SUPT16H | | proc | 1 | 1 | | | 1 | | | | | |
| chr2: 158630084-158632092 | PTP4A1 | manual | proc | 1 | 1 | | | 1 | | | | | |
| chr1: 16882259-16884855 | EIF1AX | manual | | 1 | | | 1 | 2 | | | | | |
| chr5: 108257676-108258248 | LSM2 | | proc | | 1 | | | 3 | | | | | |
| chr17: 63634210-63634432 | DQ587552 | | dup | 2 | | | 1 | 1 | | | | | |
| chr8: 120561397-120562903 | ZNF532 | auto | frag | 1 | 1 | | | 2 | | | | | |
| chr12: 100881924-100882389 | NENF | | proc | | | | 2 | 2 | | | | | |
| chr1: 145020394-145021949 | SSB | manual | proc | | 2 | | | 1 | | | | | |
| chr10: 43420007-43421764 | CAP1 | manual | proc | | 2 | | | 1 | | | | | |
| chr1: 40371010-40373921 | OAZ1 | manual | proc | 3 | | | | 4 | | | | | |
| chr14: 60316280-60317538 | MAD2L1 | | proc | | | 1 | | 2 | | | | | |
| chr3: 31470003-31472686 | THRAP3 | manual | dup | | | 1 | | 2 | | | | | |
| chrX: 70176246-70179958 | SOCS5 | manual | dup | | | 1 | | 2 | | | | | |
| chrX: 70180396-70182077 | SOCS5 | manual | dup | | | 1 | | 2 | | | | | |
| chr9: 83890554-83893228 | DDX10 | manual | proc | | | 1 | | 2 | | | | | |
| chrX: 72660041-72662733 | MAP2K4 | manual | proc | | | 1 | | 2 | | | | | |
| chr8: 75677664-75679288 | PCBP2 | | proc | 1 | | 1 | | 1 | | | | | |
| chr4: 68263271-68265012 | ST3GAL1 | auto | proc | 1 | | 1 | | 1 | | | | | |
| chr3: 73314742-73314982 | CCDC75 | auto | proc | 2 | | 1 | | | | | | | |
| chr13: 29768191-29769131 | PRDX2 | manual | proc | 2 | | 1 | | | | | | | |
| chr12: 74606217-74606813 | TMEM141 | | proc | | 1 | | 1 | 3 | | | | | |
| chr6: 127717472-127720031 | YWHAZ | manual | dup | | | 1 | | 3 | | | | | |
| chr21: 36588933-36589981 | SFRS9 | manual | proc | 1 | 1 | | | 5 | | | | | |
| chr14: 91136604-91139705 | POLR3G | | proc | 1 | | 1 | | 2 | | | | | |
| chr3: 133590336-133594125 | KIAA1333 | | dup | | 1 | | | 7 | | | | | |
| chr9: 75278688-75279979 | DPP3 | manual | proc | 3 | | 1 | | | | | | | |
| chr22: 40726971-40728762 | SLC25A5 | manual | proc | | | 1 | 1 | 2 | | | | | |
| chr5: 114875647-114877498 | CCT5 | | proc | 1 | | 1 | 1 | 1 | | | | | |
| chr18: 22090016-22090564 | FAM60A | | proc | | 3 | | | 2 | | | | | |
| chr9: 34308269-34310028 | SERPINH1 | manual | proc | 2 | 1 | 1 | | | | | | | |
| chr7: 27462505-27465027 | EIF4H | manual | proc | | 1 | 1 | 1 | | | | | | |
| chr16: 31486617-31488548 | CSDA | | proc | | 3 | | 1 | | | | | | |
| chr9: 21685064-21689997 | KHSRP | manual | proc | | 3 | | 1 | | | | | | |
| chr3: 49329045-49329924 | SSNA1 | | proc | 3 | | | | 9 | | | | | |
| chr4: 146516051-146518502 | RTN3 | | proc | 1 | 3 | | | 2 | | | | | |
| chr8: 87563050-87564438 | NTAN1 | auto | dup | 1 | | 1 | | 6 | | | | | |
| chr7: 104957816-104958449 | RNF181 | | proc | 3 | 1 | 1 | | | | | | | |
| chr19: 62378395-62379706 | PTCD3 | | dup | | | 2 | | 1 | | | | | |
| chr9: 89871235-89875006 | PNRC2 | manual | | | | 2 | | 1 | | | | | |
| chr6: 30707737-30709987 | PTMA | manual | | | | 2 | | 1 | | | | | |
| chr11: 18643045-18644377 | SFRS3 | auto | proc | | | 2 | | 1 | | | | | |
| chr7: 138506992-138509638 | MSL3L1 | manual | dup | | | 2 | | 1 | | | | | |
| chr3: 197148302-197149669 | PPP4R2 | manual | dup | | | 2 | | 1 | | | | | |
| chr2: 204206408-204208995 | ZNF207 | manual | proc | | | 2 | | 1 | | | | | |
| chr2: 179027517-179028802 | NUDC | manual | proc | | | 2 | | 1 | | | | | |
| chr17: 24552608-24554860 | TWF1 | auto | proc | | 2 | 1 | | 1 | | | | | |
| chr17: 22433702-22435031 | TUFM | | proc | 3 | | 1 | 1 | 1 | | | | | |
| chr12: 1843255-1844894 | LAMP1 | auto | proc | | 4 | | | 1 | | | | | |
| chrX: 114805474-114806874 | ASS1 | manual | proc | 1 | 2 | 1 | | | | | | | |
| chr2: 162065731-162069847 | AHCTF1 | manual | proc | 1 | 2 | 1 | | | | | | | |
| chr1: 16006265-16007067 | UQCRH | manual | | | | 2 | | 2 | | | | | |
| chr2: 43761451-43762368 | ZFP106 | manual | frag | | | 2 | | 2 | | | | | |
| chr6: 158577413-158579430 | SRP72 | manual | dup | | | 2 | | 2 | | | | | |
| chr3: 15895007-15896926 | IMPDH1 | manual | dup | 1 | | 2 | | 1 | | | | | |
| chr1: 245410530-245415024 | PEX5 | manual | proc | 1 | | 2 | | 1 | | | | | |
| chr13: 18423767-18425026 | PHF2 | manual | dup | 1 | | 2 | | 2 | | | | | |
| chr1: 104417120-104419481 | THAP3 | manual | proc | 1 | | 2 | | 2 | | | | | |
| chrX: 40578772-40580026 | MKRN1 | | dup | | 1 | 2 | | | | | | | |
| chr19: 32986379-32989682 | BC040831 | | proc | | 1 | 2 | | | | | | | |
| chr10: 121370445-121373045 | RAD1 | manual | proc | | 1 | 2 | | | | | | | |
| chr1: 212722108-212723497 | AP3S1 | manual | proc | | 1 | 2 | | | | | | | |
| chr1: 226368927-226369874 | FAM96A | manual | proc | | 1 | 2 | | | | | | | |
| chr16: 17095569-17097803 | LYRM2 | | dup | | 3 | 1 | | | | | | | |
| chr1: 144990000-144992583 | IGF2BP2 | manual | proc | | 3 | 1 | | | | | | | |
| chr3: 44595235-44597668 | MPRIP | manual | dup | 1 | | 1 | | 9 | | | | | |
| chr12: 109351042-109353797 | HMGA1 | | proc | 1 | 1 | 1 | 2 | | | | | | |
| chr7: 16870050-16872687 | RAD17 | manual | proc | 3 | 3 | | | 3 | | | | | |
| chr4: 358246-359943 | ZNF43 | | dup | | 1 | 2 | | 1 | | | | | |
| chr16: 46156574-46158361 | EIF4B | | proc | | 1 | 2 | | 1 | | | | | |
| chr9: 16765715-16765767 | LSM1 | manual | proc | 2 | 3 | | 1 | 2 | | | | | |
| chr15: 43132056-43133113 | YWHAZ | | proc | | 2 | 1 | | 5 | | | | | |
| chr5: 114752657-114755023 | CTNNA1 | | proc | 1 | 5 | | | | | | | | |
| chrX: 73266994-73269840 | DDX3Y | manual | dup | 1 | | 2 | | 4 | | | | | |
| chr19: 14461030-14461871 | SNRPG | | proc | 3 | | 1 | | 8 | | | | | |
| chr2: 135985004-135986146 | G3BP1 | manual | proc | 1 | 1 | 2 | | 1 | | | | | |
| chrX: 85225950-85227916 | STIP1 | manual | proc | 4 | 1 | | 3 | 2 | | | | | |
| chr11: 86208209-86209928 | MOBKL3 | | proc | | 2 | 2 | | | | | | | |
| chr13: 28191258-28194994 | CYP51A1 | manual | dup | | 2 | 2 | | | | | | | |
| chr3: 171262714-171264755 | SETD8 | | proc | 1 | 3 | 1 | | 2 | | | | | |
| chrX: 13306389-13307499 | GPX1 | manual | proc | 1 | 3 | 1 | | 2 | | | | | |
| chr11: 133498167-133500014 | PTP4A2 | | proc | | 4 | 1 | | | | | | | |
| chr1: 245420253-245420689 | ZNF720 | manual | proc | | | 3 | | 1 | | | | | |
| chrX: 65092271-65093777 | RBMX | manual | proc | | 2 | 2 | | 1 | | | | | |
| chr7: 63832277-63834664 | MPZL1 | manual | proc | 1 | | 3 | | | | | | | |
| chr15: 91140001-91141446 | ASB9 | | proc | | | 2 | | 8 | | | | | |
| chr10: 135007719-135008625 | BANF1 | manual | frag | 1 | 2 | 2 | | | | | | | |
| chr2: 171165069-171166985 | HMGB1 | manual | proc | 1 | 4 | 1 | | | | | | | |
| chrX: 73296169-73299018 | MKRN1 | manual | proc | | | 3 | | 2 | | | | | |
| chr6: 31351298-31355026 | USP8 | manual | proc | | | 3 | | 2 | | | | | |
| chr10: 21952707-21955029 | HNRNPR | manual | proc | | | 3 | | 2 | | | | | |
| chr7: 158107538-158109026 | THAP5 | manual | frag | 1 | 2 | 2 | | 1 | | | | | |
| chr15: 57756054-57757383 | PIGH | | proc | | | 3 | | 3 | | | | | |
| chr18: 55835065-55835698 | FAM60A | auto | proc | | | 3 | | 3 | | | | | |
| chr22: 37448047-37449602 | PRDX3 | manual | proc | | | 3 | | 3 | | | | | |
| chr22: 22320853-22325031 | ASL | manual | dup | 2 | | 2 | | 7 | | | | | |
| chr10: 13238600-13239995 | BTBD7 | manual | dup | 2 | | 3 | | 1 | | | | | |
| chr7: 46005000-46007016 | TTC4 | manual | proc | 1 | 1 | 2 | 1 | 3 | | | | | |
| chr15: 91139708-91140030 | ASB9 | | proc | | 2 | 2 | | 4 | | | | | |
| chr5: 68645003-68645660 | CFL1 | | proc | | | 2 | | 11 | | | | | |
| chrX: 120164368-120165004 | HSPA8 | manual | | | 3 | 2 | | 1 | | | | | |
| chr17: 39660007-39663513 | SHC1 | | proc | 3 | | 2 | 2 | 2 | | | | | |
| chr17: 26359119-26359614 | YWHAE | | dup | | 1 | 3 | | 2 | | | | | |
| chr1: 76938038-76939270 | TPI1 | manual | proc | 2 | 2 | 2 | 1 | | | | | | |
| chr4: 113966768-113968338 | TAF4B | auto | frag | | | 4 | | 1 | | | | | |
| chr13: 76456888-76460017 | DHX9 | manual | proc | | | 4 | | 1 | | | | | |
| chr13: 98665103-98666182 | H2AFZ | manual | proc | | | 4 | | 1 | | | | | |
| chr10: 64650560-64652770 | TATDN1 | manual | proc | | | 4 | | 1 | | | | | |
| chr9: 30678968-30680030 | RBMX | manual | proc | | 2 | 3 | | 1 | | | | | |
| chr13: 88810056-88813657 | SP3 | manual | proc | 1 | | 4 | | | | | | | |
| chr10: 70062143-70062538 | FAM36A | manual | | 1 | 3 | 2 | | 3 | | | | | |
| chr5: 172445309-172447866 | CDC42 | | proc | | | 4 | | 2 | | | | | |
| chr9: 98527969-98529661 | MKRN1 | manual | dup | 1 | 1 | 3 | | 4 | | | | | |
| chr12: 86700634-86702602 | MKRN1 | auto | proc | 1 | | 4 | | 2 | | | | | |
| chr7: 117310724-117311343 | SFRS3 | manual | | | 3 | 3 | | | | | | | |
| chr7: 56261370-56264649 | CCNJ | manual | proc | | 1 | 4 | | 1 | | | | | |
| chr6: 131545798-131548372 | DPF2 | manual | proc | 1 | 1 | 4 | | | | | | | |
| chr12: 3820029-3825029 | OTUD4 | | proc | | | 5 | | 1 | | | | | |
| chrX: 73719066-73719697 | PABPC1 | manual | frag | | | 5 | | 1 | | | | | |
| chr1: 241495850-241498178 | FCF1 | manual | proc | | | 5 | | 1 | | | | | |
| chr1: 16885318-16888034 | PDE4DIP | manual | | 5 | | 4 | | 1 | | | | | |
| chr12: 99496386-99499915 | PIGA | | proc | 1 | 2 | 4 | | | | | | | |
| chrX: 79711705-79715004 | HK2 | manual | proc | 1 | 1 | 4 | 1 | 1 | | | | | |
| chr8: 107777994-107779579 | TAGLN2 | | proc | 1 | 1 | 5 | | 1 | | | | | |
| chr1: 177437318-177437893 | EIF4A1 | manual | frag | | 6 | 3 | | | | | | | |
| chr13: 76460074-76462820 | DHX9 | manual | proc | | | 6 | | 1 | | | | | |
| chr10: 94417431-94419676 | EIF2S2 | manual | proc | | | 6 | | 1 | | | | | |
| chr2: 191565081-191567610 | RAB1A | manual | dup | | 3 | 4 | | 4 | | | | | |
| chr1: 176981907-176983662 | PTPN2 | manual | proc | 1 | | 6 | | | | | | | |
| chr5: 72327995-72329739 | DULLARD | | proc | | | 6 | | 2 | | | | | |
| chr11: 61895019-61899450 | RCC2 | | proc | | 2 | 3 | | 15 | | | | | |
| chr10: 23465825-23468573 | YWHAZ | manual | | | 6 | 3 | | 2 | | | | | |
| chr10: 107436038-107439382 | YWHAZ | manual | proc | 1 | 4 | 4 | | 1 | | | | | |
| chr7: 98432519-98435031 | RNF14 | manual | proc | | | 6 | | 4 | | | | | |
| chr3: 97977193-97979955 | CDV3 | auto | proc | 1 | 2 | 5 | 1 | | | | | | |
| chrX: 41417881-41420027 | YWHAZ | manual | | 1 | 3 | 5 | | | | | | | |
| chr17: 20456347-20460008 | DEF8 | manual | proc | | 1 | 6 | 1 | 1 | | | | | |
| chr14: 70866169-70867395 | PDZD11 | | proc | | | 7 | | 1 | | | | | |
| chr5: 37245024-37248385 | OFD1 | auto | proc | 1 | | 7 | | | | | | | |
| chr12: 3818860-3819919 | OTUD4 | | proc | 3 | 3 | 5 | | 1 | | | | | |
| chr7: 6647990-6650019 | GZF1 | manual | | | 3 | 6 | | 1 | | | | | |
| chr12: 110590068-110592654 | PCNP | auto | proc | | 1 | 8 | | 2 | | | | | |
| chr20: 17940242-17942547 | PTMA | manual | | 2 | 6 | 5 | | 4 | | | | | |
| chr7: 150830163-150832517 | ETF1 | | proc | | | 9 | | 2 | | | | | |
| chr22: 24075369-24076698 | PHF10 | manual | proc | 1 | 1 | 8 | 1 | 3 | | | | | |
| chr6: 53207834-53209186 | HMGB1 | manual | proc | | 6 | 8 | | | | | | | |
| chr7: 32762364-32763505 | DPY19L2P2 | manual | dup | 2 | 13 | 7 | | 7 | | | | | |
| chr14: 54835005-54835966 | CHMP4B | | proc | 3 | | 4 | | 17 | | | | | 1 |
| chr22: 29627969-29630029 | EIF4H | manual | proc | 3 | 3 | 5 | | 12 | | | 1 | | |
| chr17: 4072185-4075029 | RYK | | proc | 2 | | 7 | | 9 | | | | | 1 |
| chr6: 160051470-160055015 | HNRNPH2 | manual | proc | 2 | | 6 | | 10 | | | 1 | | |
| chr4: 129295826-129297562 | FOSL1 | | proc | 3 | | | 1 | 13 | | | | | 1 |
| chr11: 6465021-6467394 | VDAC1 | | dup | 2 | | 11 | | 3 | | | 1 | | |
| chr12: 13046376-13049181 | HTR7 | | proc | 2 | 4 | 3 | | 6 | | 1 | | | |
| chr12: 51833223-51834834 | EIF4A1 | auto | proc | | 7 | 7 | | 1 | | | 1 | | |
| chr15: 91077671-91078734 | H2AFV | auto | proc | | | | | 15 | | | | | 1 |
| chr16: 14920001-14923862 | PKD1 | | dup | 2 | 2 | 3 | | 7 | | 1 | | | |
| chr13: 40090629-40094438 | RNF6 | manual | dup | 2 | 2 | 7 | | 2 | | | | | 1 |
| chr7: 144911241-144912979 | DPY19L4 | manual | proc | 1 | 4 | 3 | 4 | 1 | 1 | | | | |
| chr21: 31920001-31923002 | FBXW11 | manual | proc | 3 | 2 | 4 | | 4 | | | 1 | | |
| chr22: 34623087-34624393 | NDUFA9 | manual | proc | 1 | | | | 12 | | | | | 1 |
| chr2: 101256951-101257613 | PPIA | | dup | 2 | 1 | 7 | | 2 | | | 1 | | |
| chr12: 54660003-54663591 | RAB13 | auto | | 1 | | 5 | | 6 | | | 1 | | |
| chr2: 85421131-85422577 | PEBP1 | | proc | 7 | | 1 | 1 | 3 | | | | | 1 |
| chrX: 81638952-81640007 | EIF3J | manual | frag | | 7 | 3 | | 2 | | | 1 | | |
| chr15: 63850100-63851589 | DNAJB14 | auto | proc | 1 | | 7 | | 4 | | | 1 | | |
| chr17: 78125786-78128688 | ARL2BP | | dup | 2 | | 2 | | 7 | | | | | 1 |
| chr8: 48585762-48587345 | NSFL1C | | proc | 3 | 1 | 5 | | 2 | | | 1 | | |
| chr11: 66285096-66286445 | C1QBP | auto | proc | 2 | | | | 9 | | | | | 1 |
| chr7: 72033364-72034127 | POMZP3 | | dup | 1 | 4 | 3 | | 2 | | | 1 | | |
| chr7: 30528821-30529744 | FLJ00278 | manual | | | | 7 | | 3 | | | 1 | | |
| chr12: 70528835-70530029 | MRS2 | | proc | | | 7 | | 3 | | | | | 1 |
| chr17: 27461314-27463910 | WDR45L | auto | proc | 1 | 1 | 4 | 1 | 3 | | | | 1 | |
| chr10: 79160515-79164753 | ZNF532 | manual | dup | 1 | | | | 9 | | | | | 1 |
| chr11: 232267-233719 | SRP19 | | dup | | | 7 | | 2 | | | 1 | | |
| chr8: 59500000-59502148 | PTPN11 | | dup | | | 8 | | 1 | | | 1 | | |
| chr19: 14042134-14043294 | EEF1D | auto | frag | 7 | | | 1 | 1 | | | | | 1 |
| chr15: 54085179-54086686 | CNOT6L | auto | proc | 1 | 1 | 3 | 4 | | 1 | | | | |
| chr10: 127345241-127346621 | ALDOA | manual | proc | 7 | 1 | | 1 | | | | 1 | | |
| chr19: 45140233-45141668 | PRR13 | auto | | 1 | 1 | 4 | | 2 | | | 1 | | |
| chr17: 72157677-72159762 | PTMA | auto | | | 5 | 3 | | | | | 1 | | |
| chr5: 177525251-177526202 | APOA1BP | | proc | 5 | 1 | | 1 | 1 | | | | 1 | |
| chrX: 41420046-41420852 | YWHAZ | manual | | 1 | 1 | 6 | | | | | 1 | | |
| chr3: 52382585-52384174 | PPP2R5C | | proc | | 1 | 6 | | 1 | | | 1 | | |
| chr1: 245429261-245429363 | ZNF720 | manual | | 2 | | 4 | | 2 | | | 1 | | |
| chr5: 107088630-107089995 | USP7 | | proc | | | 5 | | 3 | | | 1 | | |
| chr4: 118715123-118716642 | NT5C3 | manual | proc | | 2 | 4 | | 2 | | | 1 | | |
| chr22: 21935039-21937130 | FBXW4 | manual | proc | | | 3 | | 5 | | | | | 1 |
| chr5: 175667341-175669888 | BRCC3 | | dup | | | 6 | | 1 | | | 1 | | |
| chr14: 95758990-95760012 | CKS1B | | proc | | 3 | 2 | | 2 | | 1 | | | |
| chrY: 11781394-11785025 | LOC389834 | | proc | | | 3 | 1 | 3 | | | | | 1 |
| chr12: 6457164-6459964 | PKP2 | | proc | | | 7 | | | | | 1 | | |
| chr21: 46897231-46898445 | DSTN | manual | frag | 1 | | 4 | | 2 | | | 1 | | |
| chrX: 48519005-48520014 | ACAA2 | manual | proc | | | 3 | | 4 | | | 1 | | |
| chr2: 197835918-197836621 | ATP5G2 | manual | proc | | | 7 | | | | | 1 | | |
| chr15: 97615266-97617895 | HSP90B1 | | proc | 1 | 2 | 1 | 1 | 1 | | | | | 1 |
| chr8: 82692562-82693415 | NIPA2 | | proc | | 1 | | 2 | 3 | | | 1 | | |
| chr4: 145986182-145988340 | HSPD1 | auto | proc | | 3 | 2 | | 1 | | | 1 | | |
| chrX: 70451371-70452584 | ZCRB1 | manual | dup | | 2 | 1 | | 3 | | | 1 | | |
| chr8: 30225003-30227051 | TAGLN2 | auto | proc | 2 | 4 | | | | | 1 | | | |
| chr2: 32280035-32282728 | DDX50 | manual | dup | | | 4 | | 2 | | | 1 | | |
| chr2: 84856616-84858887 | LDHA | manual | proc | 1 | 1 | 4 | | | | | 1 | | |
| chr1: 245420126-245420978 | ZNF682 | manual | proc | | | 5 | | 1 | | | 1 | | |
| chr6: 36167532-36168612 | NUDT5 | manual | proc | | | 6 | | | | | 1 | | |
| chr17: 2436284-2437919 | EIF4A1 | | dup | 1 | 1 | 2 | | 1 | | | 1 | | |
| chr12: 78225570-78226943 | REST | | dup | 1 | | 1 | | 3 | | | | | 1 |
| chr2: 20355007-20355544 | RP9 | | dup | 1 | | | | 4 | | | | | 1 |
| chr16: 18386391-18389697 | PKD1 | | dup | | | 2 | | 3 | | | 1 | | |
| chr3: 133588073-133590018 | KIAA1333 | | dup | | | | | 5 | | | | | 1 |
| chr11: 82597892-82598941 | CKS1B | | proc | 1 | 3 | 1 | | | | | 1 | | |
| chr11: 122335815-122337882 | ATP5F1 | | proc | | 1 | | | 4 | | | 1 | | |
| chr15: 33171848-33172885 | U2AF1 | | proc | 1 | | | | 4 | | | | | 1 |
| chr19: 3821749-3823145 | FTL | auto | proc | | 3 | 2 | | | | | 1 | | |
| chr1: 96685035-96686396 | EEF1A1 | manual | proc | | 2 | 2 | | 1 | | | 1 | | |
| chr10: 59642034-59642720 | TPT1 | manual | proc | | 1 | 3 | | 1 | | | 1 | | |
| chr7: 55672416-55674246 | CDC42 | manual | proc | | 4 | | | 1 | | | 1 | | |
| chr7: 5566916-5567375 | S100A11 | manual | proc | 4 | 1 | | | | | | | | 1 |
| chr6: 147770007-147774161 | YAP1 | manual | proc | | | 2 | | 3 | | | | | 1 |
| chr3: 155553099-155554758 | DDX50 | | proc | 1 | 2 | 1 | | | | | 1 | | |
| chr14: 57677677-57678048 | UBA52 | | proc | 3 | | | | 1 | | | 1 | | |
| chr2: 91130114-91132611 | TMEM128 | manual | frag | | 3 | | | 1 | | | | | 1 |
| chrX: 67920024-67922986 | SERBP1 | manual | dup | | 1 | 3 | | | | | 1 | | |
| chr22: 22600024-22604137 | KLHL5 | manual | dup | | | 1 | | 3 | | | | | 1 |
| chr3: 36783146-36785030 | HSPD1 | manual | proc | 1 | 2 | | | 1 | | | 1 | | |
| chrX: 49283282-49284875 | VDAC1 | manual | proc | | 3 | | | 1 | | | 1 | | |
| chrX: 65635098-65636615 | PKM2 | manual | proc | 1 | | | 3 | | | | 1 | | |
| chr9: 130067782-130069317 | FLJ00278 | | dup | | | | | 3 | | | 1 | | |
| chr15: 34692846-34694996 | TPM3 | | proc | 1 | 2 | | | | | | 1 | | |
| chr4: 79320311-79322415 | SERBP1 | | proc | | 1 | 2 | | | | | 1 | | |
| chr9: 37625488-37627344 | RAB1B | manual | | 3 | | | | | | | | 1 | |
| chr16: 4552399-4553233 | SUB1 | | proc | | | 3 | | | | | 1 | | |
| chr3: 171145011-171147501 | FHL1 | | proc | | | 3 | | | | | 1 | | |
| chr4: 166544280-166544941 | HADHA | | proc | | | | | 3 | | | | | 1 |
| chr9: 138826841-138829981 | NCL | manual | dup | | 1 | | 1 | 1 | | | | | 1 |
| chr16: 21692-25029 | IL9R | manual | dup | | | 3 | | | | | 1 | | |
| chr2: 202772424-202775011 | DAZAP2 | manual | proc | 1 | 1 | 1 | | | | | | | 1 |
| chr7: 88106317-88107757 | EEF1A1 | manual | proc | 1 | | 1 | | 1 | | | 1 | | |
| chr2: 222535085-222537390 | HSPA9 | manual | proc | | 2 | | 1 | | | | 1 | | |
| chr7: 143975043-143976509 | EEF1A1 | manual | proc | | 2 | 1 | | | | | 1 | | |
| chr1: 64080787-64082020 | CFL1 | manual | proc | 1 | | 2 | | | | | 1 | | |
| chr21: 36345013-36346248 | FAM80B | manual | proc | | | 1 | | 2 | | | 1 | | |
| chr20: 32452814-32454340 | CDC42 | manual | proc | | | 1 | | 2 | | | 1 | | |
| chr3: 25771373-25774090 | TAF9B | manual | proc | | | 3 | | | | | | | 1 |
| chr1: 78085216-78088009 | CCDC55 | manual | proc | 2 | | 5 | | 20 | | | | | 2 |
| chr6: 143705076-143706473 | PARL | manual | proc | 3 | 12 | 3 | 1 | 7 | | 1 | | | 1 |
| chr3: 122007401-122009653 | NAP1L1 | auto | proc | 1 | 5 | 9 | | 6 | | | 2 | | |
| chr9: 126952546-126954985 | PRPS1 | manual | proc | 6 | | 4 | | 11 | | | | | 2 |
| chr9: 34911199-34912896 | YWHAZ | manual | proc | 1 | 9 | 7 | | 3 | | 1 | 1 | | |
| chr19: 58005048-58009278 | PABPN1 | | proc | 1 | 3 | 5 | | 10 | | | 1 | | 1 |
| chr3: 95276627-95278752 | RBBP4 | | proc | 1 | 2 | 11 | | 4 | | | 2 | | |
| chr12: 41110881-41113477 | FXR1 | | proc | 1 | 2 | 11 | | 4 | | | 2 | | |
| chr15: 51016593-51017949 | EEF1A1 | | proc | | 8 | 6 | | 3 | | | 2 | | |
| chr4: 88053138-88055031 | ARPC1A | | dup | | | 5 | | 10 | | | 1 | | 1 |
| chr6: 30262288-30264297 | PAIP1 | manual | dup | | 1 | 9 | | 5 | | | 2 | | |
| chr3: 52202197-52204001 | ALDOA | | dup | 9 | | 1 | 2 | 2 | 1 | | 1 | | |
| chr9: 12962422-12963578 | PRDX1 | manual | proc | 3 | 4 | 2 | 1 | 4 | | | | | 2 |
| chr12: 19985423-19989724 | TCP1 | | proc | | 6 | | 1 | 4 | | | 2 | | |
| chr3: 115086410-115088196 | DULLARD | auto | proc | | 4 | 5 | 1 | 1 | | | 2 | | |
| chr7: 7986381-7988620 | PPIL4 | manual | dup | | | 10 | | 1 | | | 2 | | |
| chr17: 42855745-42858503 | NFE2L3 | | dup | 2 | | | | 8 | | | | | 2 |
| chr15: 73400772-73402108 | ANP32B | | proc | | 5 | 4 | | 1 | | | 2 | | |
| chr1: 218493241-218494999 | MORF4L1 | manual | proc | | 5 | 4 | | 1 | | | 2 | | |
| chr2: 25898653-25900030 | PTGES3 | manual | proc | | | 6 | | 4 | | | 2 | | |
| chr3: 53070978-53074165 | SERBP1 | | proc | | 2 | 5 | | 2 | | | 2 | | |
| chr8: 62011957-62014951 | NASP | | proc | 1 | | 4 | | 4 | | | 2 | | |
| chr17: 60204622-60205031 | LOC201175 | | proc | 2 | | | | 7 | | | | | 2 |
| chr22: 40833082-40834984 | OLA1 | manual | proc | | 1 | 1 | 1 | 6 | | | 2 | | |
| chr2: 74498320-74499480 | HMGA1 | manual | | 1 | 2 | 4 | | 1 | | | 2 | | |
| chr22: 22040076-22042678 | CES7 | manual | | | | | | 7 | | | | | 2 |
| chrX: 130608331-130609932 | HDGF | manual | frag | 3 | 1 | 1 | | 2 | | | | | 2 |
| chr1: 158906538-158909903 | SET | manual | proc | | 2 | 5 | | | | | 2 | | |
| chr9: 22001477-22002779 | UBA52 | manual | proc | 4 | | 3 | | | | | 2 | | |
| chr11: 91708751-91709133 | NDUFB11 | manual | proc | | | | 3 | 4 | | | 2 | | |
| chr22: 22045609-22049946 | CES7 | manual | | | | | | 6 | | | | | 2 |
| chr13: 64430032-64431456 | LGMN | manual | dup | 2 | 3 | | 1 | | | | 2 | | |
| chr14: 63130269-63131835 | GCAT | auto | proc | | | 4 | | 2 | | | 2 | | |
| chrX: 120165030-120166529 | HSPA8 | manual | proc | 2 | 1 | 2 | | 1 | | | 2 | | |
| chr1: 207507608-207508364 | ATP5G2 | manual | proc | 1 | | 3 | 1 | 1 | | | 2 | | |
| chr11: 92908039-92909143 | SRP14 | | proc | 2 | | 1 | | 2 | | | 2 | | |
| chr3: 23895319-23895949 | ARL4A | manual | proc | 1 | | 4 | | | | | 2 | | |
| chr9: 5100700-5103470 | TCF3 | manual | proc | | | 5 | | | | | 2 | | |
| chr1: 218507681-218508028 | AURKA | manual | proc | | | 5 | | | | | 2 | | |
| chr5: 43530817-43532401 | EEF1A1 | | proc | 1 | | 2 | | 1 | | | 2 | | |
| chr22: 22031558-22032127 | CES7 | manual | | | | | | 4 | | | | | 2 |
| chr18: 54508763-54509828 | MRPL37 | | proc | | | | | 4 | | | | | 2 |
| chr5: 14850010-14851722 | RBBP4 | | proc | | | | | 4 | | | 1 | | 1 |
| chr2: 127030021-127032314 | YWHAZ | manual | proc | 1 | 3 | | | | | | 2 | | |
| chr6: 25131126-25132842 | ASS1 | manual | proc | 1 | | 3 | | | | | 2 | | |
| chr6: 111285905-111288987 | CNN2 | manual | proc | | | 3 | | 1 | | | 2 | | |
| chr10: 66255316-66256651 | ANXA2 | manual | proc | | | | 4 | | 2 | | | | |
| chr1: 51395688-51396740 | CFL1 | manual | proc | 2 | | 1 | 1 | | 1 | | 1 | | |
| chr17: 71733295-71734754 | ATF4 | manual | | | 2 | | | 1 | | | 1 | | 1 |
| chr7: 20588278-20589374 | EEF1A1 | manual | proc | | 1 | 1 | | 1 | | | 2 | | |
| chr2: 236455494-236455892 | TMSB10 | manual | proc | 1 | 2 | | | | | | | | 2 |
| chr3: 187961542-187964137 | PSMD10 | manual | proc | | 3 | | | | | 2 | | | |
| chr4: 10982554-10984464 | RNPS1 | | proc | 5 | 10 | 5 | 1 | 11 | | | 2 | | 1 |
| chr17: 11959531-11959908 | DYNLT1 | | proc | 12 | 3 | | 5 | 7 | | | | | 3 |
| chr1: 106235172-106236952 | MATR3 | manual | proc | 3 | 4 | 8 | 7 | 4 | 1 | | 2 | | |
| chr11: 18465871-18469227 | YWHAB | | proc | 1 | 4 | 9 | | 7 | | | 3 | | |
| chr22: 23903543-23904988 | STMN3 | manual | proc | 10 | | | 4 | 7 | 1 | | | | 2 |
| chr1: 166055742-166056259 | FAM177A1 | manual | dup | 3 | 8 | | 1 | 7 | | | | | 3 |
| chr16: 68346815-68349469 | NONO | | proc | 1 | 6 | 6 | | 4 | | 1 | 2 | | |
| chr9: 88816231-88818084 | CDC20 | manual | proc | 1 | 1 | 3 | 1 | 11 | | | | | 3 |
| chr1: 233967900-233969581 | LDHA | manual | proc | 1 | 2 | 10 | | 3 | | | 3 | | |
| chr7: 65525025-65526430 | SKP1 | manual | proc | 3 | 1 | 6 | | 6 | | | 3 | | |
| chr8: 55046759-55049918 | ESF1 | | proc | 1 | | 8 | | 4 | | | 3 | | |
| chr13: 44861259-44863071 | RCN1 | manual | | 1 | 3 | 1 | 1 | 7 | | | 1 | | 2 |
| chr12: 119766955-119768000 | ARF1 | auto | proc | 6 | 2 | 1 | | 4 | 1 | | | 1 | 1 |
| chr20: 60947228-60948154 | DPH3 | manual | proc | 4 | 4 | 3 | | 2 | | | 2 | | 1 |
| chrX: 53210000-53210656 | SUV39H2 | manual | frag | 1 | | 8 | | 3 | | | 2 | | 1 |
| chr17: 20628146-20629946 | SCD | manual | proc | 9 | 1 | | 2 | | | | | 1 | 2 |
| chr5: 68644250-68645023 | CFL1 | | proc | 1 | | 2 | | 6 | | | 3 | | |
| chr17: 27578894-27579439 | UBL5 | | proc | | | 3 | 3 | 3 | | | 3 | | |
| chr1: 72512203-72513454 | GDI2 | manual | frag | 1 | 3 | 2 | | 3 | | | 3 | | |
| chr1: 240223113-240224419 | CFL1 | manual | proc | 3 | 2 | 3 | 1 | | | | 3 | | |
| chr3: 139081781-139083983 | HSPA8 | | proc | 2 | 1 | 3 | 1 | 1 | | | 3 | | |
| chr12: 55190981-55193352 | HSPD1 | | proc | | 3 | 4 | | 1 | | | 3 | | |
| chr21: 38796179-38796358 | SNRPG | manual | proc | | 6 | 1 | | 1 | | 1 | 2 | | |
| chrX: 65210128-65212872 | EIF4B | manual | proc | 1 | 1 | 2 | | 2 | | | 3 | | |
| chrX: 51683397-51683809 | UCRC | manual | proc | 1 | | | 1 | 4 | | | 3 | | |
| chr8: 30215043-30218410 | HSPA8 | | dup | | 2 | 2 | | 1 | | | 3 | | |
| chr17: 30756783-30758835 | DTWD2 | | proc | 1 | | 1 | 2 | 1 | | | | | 3 |
| chr12: 48067082-48070015 | FGFR1OP2 | | proc | 1 | | 3 | | | | | 3 | | |
| chr1: 218865561-218868015 | HDAC1 | manual | dup | | | | | 4 | | | 3 | | |
| chr4: 136186068-136187470 | EEF1A1 | | dup | | | | | 1 | | | 3 | | |
| chr11: 29253864-29254659 | EEF1A1 | | proc | | | | | 1 | | | 3 | | |
| chr4: 4946239-4947833 | LDHA | auto | proc | | 1 | | | | | | 3 | | |
| chr2: 231530573-231530995 | FAM36A | manual | proc | | | 1 | | | | | 3 | | |
| chr12: 17035000-17036276 | EEF1A1 | | proc | | | | | | | | 3 | | |
| chr2: 231145010-231146919 | TPM3 | manual | proc | | | | | | | | 3 | | |
| chr1: 38947029-38949502 | HSPA5 | manual | proc | | | | | | | | | | 3 |
| chr2: 48712985-48713883 | TPT1 | manual | proc | | | | | | | 1 | 2 | | |
| chrX: 115766164-115766603 | PRPF4B | manual | frag | 5 | 8 | 7 | 3 | 16 | | 2 | 2 | | |
| chr1: 89341252-89343268 | PTGES3 | manual | | | 10 | 10 | | 12 | | 1 | 1 | | 2 |
| chrX: 135755159-135758921 | RAB28 | manual | proc | 4 | 2 | 8 | | 14 | | | 1 | | 3 |
| chr19: 35220001-35221268 | TAF9 | auto | proc | 5 | 5 | 3 | | 13 | | | 4 | | |
| chr12: 74737397-74739842 | DENR | | proc | | 1 | 11 | | 11 | | | 4 | | |
| chr3: 48272166-48273216 | MRPS18A | manual | proc | 1 | | 8 | | 14 | | | 4 | | |
| chr12: 9283162-9284461 | PTMA | | dup | 1 | 10 | 7 | | 1 | | | 4 | | |
| chr3: 27649231-27650012 | TPM4 | manual | frag | 3 | 7 | | 3 | 6 | | 3 | | | 1 |
| chr7: 124460752-124462651 | EEF1G | manual | dup | 4 | 2 | 7 | 1 | 5 | | | 3 | | 1 |
| chr3: 157187740-157189549 | SET | | proc | | 14 | 3 | | 1 | | | 4 | | |
| chr1: 203861013-203862993 | GABARAP | manual | dup | | 1 | 1 | | 16 | | | | | 4 |
| chr22: 19134094-19135031 | KLHL12 | manual | proc | 1 | | 2 | | 14 | | | 4 | | |
| chr7: 39575135-39576400 | TXNDC1 | manual | proc | 4 | | 2 | 4 | 6 | | | 4 | | |
| chr4: 74183155-74184968 | HMGA1 | | dup | 1 | 1 | 6 | | 7 | | | 3 | 1 | |
| chr17: 30848980-30850029 | TAF5L | | proc | 2 | 4 | 6 | | 3 | | | 4 | | |
| chr19: 15881767-15883707 | IPMK | | proc | 1 | 2 | | 4 | 8 | | | | | 4 |
| chr7: 151694812-151694991 | PSPH | | proc | 2 | 11 | 1 | | 1 | | 2 | 2 | | |
| chr17: 10560021-10560742 | MAGOH | | proc | | | | | 14 | | | | | 4 |
| chr20: 48963622-48964977 | PSMD10 | manual | proc | 2 | | 8 | | 4 | | | 3 | | 1 |
| chr19: 18002173-18005024 | CS | | proc | 1 | 5 | 4 | | 2 | | | 4 | | |
| chrX: 113915399-113917620 | HSPA8 | manual | proc | | 9 | 2 | | | | | 4 | | |
| chr17: 30850001-30851322 | TAF5L | | proc | 2 | 2 | 4 | | 2 | | | 4 | | |
| chr6: 86193656-86194482 | TPT1 | manual | proc | | 4 | 4 | | 2 | | | 4 | | |
| chr14: 64815693-64817730 | PTBP1 | auto | dup | 2 | | 1 | 5 | 1 | | | 4 | | |
| chr4: 98042943-98043372 | COX7A2 | auto | proc | 1 | 1 | 2 | 1 | 4 | 1 | | 3 | | |
| chr2: 138761156-138762932 | YWHAE | manual | proc | | 4 | 2 | 1 | 2 | | | 4 | | |
| chrX: 100676203-100679915 | PRKCI | manual | proc | | | 2 | | 7 | | | | | 4 |
| chr4: 39650588-39652334 | PABPC1 | | dup | 2 | 3 | 1 | | 2 | | | 4 | | |
| chr12: 19500335-19502091 | EEF1A1 | | proc | 1 | 4 | | | 3 | | | 4 | | |
| chr12: 4076871-4079190 | HSPA8 | | dup | | 2 | 3 | 1 | 1 | | | 4 | | |
| chr1: 201002645-201003292 | COX7C | manual | proc | 2 | | | 1 | 3 | | | | | 4 |
| chr9: 119885003-119885562 | TPT1 | manual | proc | | 2 | 2 | | 2 | | | 3 | 1 | |
| chr4: 22337134-22338823 | CDC42 | auto | | 1 | 2 | 2 | | | | | 4 | | |
| chr13: 48243346-48243963 | PSME2 | manual | proc | | 3 | 1 | | 1 | | | 4 | | |
| chr10: 32840157-32840929 | C1D | manual | proc | | 1 | 1 | | 3 | | | 4 | | |
| chr3: 39375064-39376459 | EEF1A1 | manual | proc | | | 1 | | 3 | | | 4 | | |
| chr6: 109335327-109335840 | ATP5J2 | manual | proc | | 1 | 2 | | | | 1 | 1 | | 2 |
| chr13: 27686701-27688179 | EEF1A1 | manual | proc | 1 | | | | 1 | | | 4 | | |
| chr3: 120732546-120733113 | CSRP2 | | proc | | | | | | | | 4 | | |
| chrX: 74880003-74883639 | TTC3 | manual | proc | 11 | 8 | 4 | 5 | 26 | | 1 | | | 4 |
| chr16: 11905084-11905547 | COX6C | | proc | 14 | 1 | 7 | 4 | 4 | | | 4 | 1 | |
| chr1: 52606731-52607671 | PLA2G12A | manual | proc | 5 | 4 | | 6 | 15 | | | | | 5 |
| chr6: 158950293-158952897 | TATDN2 | manual | proc | 4 | 6 | 10 | 1 | 6 | | | 4 | | 1 |
| chr5: 96416174-96418578 | SET | auto | proc | 3 | 10 | 9 | | 3 | | | 5 | | |
| chr1: 89067086-89067800 | TCEB1 | manual | proc | 3 | 3 | 11 | | 7 | | | 5 | | |
| chr12: 12155020-12156368 | PTMA | auto | frag | 1 | 10 | 10 | | 2 | | 2 | 3 | | |
| chr7: 104095351-104099270 | EIF4B | manual | proc | 1 | 9 | 8 | | 5 | | | 5 | | |
| chrX: 114843363-114844764 | EEF1G | manual | proc | 3 | 5 | 6 | 1 | 6 | | | 5 | | |
| chr2: 61016549-61018625 | NONO | manual | dup | 2 | 5 | 9 | | 4 | | | 5 | | |
| chr17: 37935489-37939966 | PTP4A2 | | proc | 3 | 9 | 4 | | 3 | | | 5 | | |
| chr12: 67322298-67323260 | ATP5H | | proc | 1 | 1 | 11 | | 5 | | | 5 | | |
| chr9: 32935789-32937584 | ASS1 | manual | proc | 1 | 2 | 9 | | 4 | | | 5 | | |
| chr17: 60205003-60209312 | LOC201175 | | proc | 6 | | | | 8 | | | | | 5 |
| chrX: 25573624-25574445 | RANBP1 | manual | | 3 | 4 | 4 | | | | | 5 | | |
| chr17: 64826385-64826952 | SNRPG | | proc | | 1 | 8 | | 1 | | | 5 | | |
| chr14: 35420023-35420673 | EWSR1 | | proc | 1 | 3 | 4 | | 1 | | | 5 | | |
| chr22: 22070000-22074479 | ZDHHC8 | manual | frag | | | | | 9 | | | | | 5 |
| chr9: 6656022-6660031 | RNF2 | manual | proc | 1 | | 4 | | 3 | | | 5 | | |
| chr1: 245460000-245461944 | HSPA8 | manual | proc | 1 | 2 | 2 | 1 | 1 | | | 5 | | |
| chr9: 6660006-6664836 | RNF2 | manual | proc | | 1 | 3 | | 3 | | | 5 | | |
| chr9: 14910713-14912555 | LDHA | manual | proc | 1 | 1 | 4 | | 1 | 1 | | 4 | | |
| chr3: 185226809-185228146 | EEF1A1 | manual | dup | 2 | 1 | | | 1 | | | 5 | | |
| chr1: 42738096-42738591 | TMSB4X | manual | | 1 | 1 | | | | | | 5 | | |
| chrX: 23003631-23004877 | FAM3C | manual | | 10 | 12 | 6 | 17 | 9 | | 1 | | 1 | 4 |
| chr22: 24375001-24378128 | YES1 | manual | dup | 8 | 10 | 8 | 8 | 10 | 1 | 2 | 1 | | 2 |
| chr3: 48306896-48308964 | FCF1 | manual | proc | 3 | 10 | 6 | | 13 | | 1 | 4 | | 1 |
| chr2: 132196980-132198589 | TOMM40 | manual | proc | 7 | 8 | 6 | 2 | 6 | | 1 | 5 | | |
| chr1: 116875720-116878445 | NAP1L4 | manual | proc | 5 | 1 | 10 | 3 | 1 | | | 5 | | 1 |
| chr6: 5917621-5919864 | PKM2 | manual | proc | 8 | 3 | | 8 | 1 | | | 4 | 1 | 1 |
| chr15: 19186009-19190031 | PAK2 | auto | proc | 4 | | | | 15 | | | | | 6 |
| chrX: 30558147-30559009 | FTL | manual | proc | 1 | 8 | 3 | 4 | 2 | | 1 | 5 | | |
| chr3: 182442769-182443381 | FAU | | proc | 2 | 1 | 3 | 6 | 5 | 2 | | 4 | | |
| chr6: 144563059-144563906 | TPT1 | manual | proc | 1 | 6 | 5 | 1 | 4 | | | 5 | | 1 |
| chrX: 44377591-44379921 | CTF8 | manual | proc | 3 | 5 | 3 | | 2 | | 1 | 5 | | |
| chr1: 169906030-169906835 | PFN1 | manual | proc | 1 | | | | 1 | | | 6 | | |
| chr12: 32770077-32771796 | NAP1L1 | | proc | 4 | 7 | 11 | | 18 | | | 5 | | 2 |
| chr10: 75161319-75163908 | DUSP8 | manual | proc | 7 | | 1 | 5 | 24 | | | 1 | | 6 |
| chr15: 19190000-19191609 | PAK2 | auto | proc | 3 | | 2 | | 31 | 1 | | 1 | | 5 |
| chr17: 25420266-25421375 | STX18 | | proc | 8 | 4 | 2 | 7 | 14 | 1 | 1 | 3 | | 2 |
| chr9: 97948095-97950029 | EIF4B | manual | proc | 3 | 13 | 10 | | 9 | | 2 | 5 | | |
| chr17: 60380103-60384663 | SLC16A6 | | dup | 5 | 3 | 10 | 1 | 15 | | 1 | 3 | | 3 |
| chrX: 70555627-70557712 | PABPN1 | manual | proc | 3 | | 11 | 1 | 18 | | | 5 | | 2 |
| chr18: 1350751-1353998 | ATAD1 | auto | dup | 3 | 11 | 4 | 7 | 7 | 1 | 1 | 2 | 1 | 2 |
| chr7: 137738291-137739988 | PTMA | manual | frag | 3 | 16 | 9 | | 4 | | 2 | 5 | | |
| chrX: 73340516-73344880 | RAB11FIP1 | manual | proc | 4 | | 9 | | 18 | | | 4 | | 3 |
| chr3: 40612582-40613978 | EEF1G | manual | proc | 3 | 13 | 3 | | 6 | | 1 | 5 | | 1 |
| chr4: 166545018-166547102 | HADHA | | proc | | | | 1 | 14 | | | | | 7 |
| chr7: 10457157-10459773 | HSPA8 | manual | proc | 4 | 4 | 5 | | 1 | | | 6 | | 1 |
| chr5: 43622114-43624741 | AMD1 | auto | proc | 1 | 1 | 5 | | 4 | | | 5 | | 2 |
| chr10: 76705756-76706390 | HMGA1 | manual | | 1 | 8 | | | | | 2 | 5 | | |
| chr6: 74058239-74059970 | EIF3E | manual | proc | 2 | 9 | 9 | 1 | 16 | | | 4 | 1 | 3 |
| chr15: 88619130-88620026 | PERP | | proc | 14 | 1 | | 7 | 14 | 1 | 1 | | | 6 |
| chr22: 24373154-24375027 | YES1 | manual | dup | 3 | | 9 | | 24 | | | 4 | | 4 |
| chr12: 6864001-6865025 | DSTN | manual | proc | 2 | 17 | 6 | 2 | 9 | | 2 | 5 | | 1 |
| chr12: 62501627-62504537 | PABPC1 | | proc | 9 | 6 | 3 | 2 | 13 | | | 4 | | 4 |
| chr1: 75980428-75983188 | DLST | manual | dup | 6 | | 11 | 1 | 12 | | | 6 | | 2 |
| chr14: 61368169-61368862 | COX4I1 | | proc | 4 | 1 | | 7 | 6 | 4 | | 3 | | 1 |
| chr10: 42335001-42336262 | ZNF37A | manual | dup | 6 | 15 | 11 | 1 | 24 | | 1 | 5 | | 3 |
| chr1: 145141125-145143358 | CCT8 | manual | proc | 7 | 13 | 9 | 1 | 22 | | 3 | 4 | | 2 |
| chr5: 177330758-177333479 | SUDS3 | auto | proc | 2 | | | 3 | 18 | | | | | 9 |
| chr11: 119196989-119198204 | BAT1 | | proc | 6 | 6 | 1 | | 5 | | | 5 | | 4 |
| chr10: 120681594-120682842 | LDHA | manual | frag | 2 | 4 | 4 | | 3 | 1 | 1 | 5 | 1 | 1 |
| chr9: 124488119-124489981 | TLK1 | manual | dup | 1 | 3 | 4 | 1 | 2 | | | 8 | | 1 |
| chr2: 234438687-234440030 | MSL3L1 | manual | proc | 19 | 16 | 4 | 14 | 45 | 2 | | 5 | 1 | 2 |
| chr3: 180460196-180461897 | LRRFIP1 | | proc | 12 | 8 | 10 | 4 | 31 | | | 5 | | 5 |
| chr11: 33051914-33054815 | PIGC | auto | proc | 7 | 18 | 11 | 2 | 19 | | 1 | 5 | | 4 |
| chr20: 3952190-3953301 | FTL | manual | | 4 | 16 | 4 | 1 | 7 | 1 | 3 | 5 | 1 | |
| chr2: 234440001-234441782 | MSL3L1 | manual | proc | 16 | 18 | 4 | 12 | 30 | 1 | 1 | 5 | 1 | 3 |
| chr2: 162640152-162642586 | TIMM8A | manual | proc | | | | 1 | 34 | | | | | 11 |
| chr1: 114880845-114882611 | PKM2 | manual | proc | 9 | 5 | 5 | 3 | 1 | 3 | | 5 | 1 | 2 |
| chr11: 43875453-43878110 | SEC14L1 | auto | proc | 11 | 7 | 11 | 8 | 26 | | 1 | 5 | | 6 |
| chr22: 19135000-19137681 | KLHL12 | manual | proc | 10 | 2 | 9 | 1 | 31 | 1 | | 5 | | 6 |
| chr22: 30995341-30999218 | CPSF1 | manual | proc | 4 | 1 | 1 | 2 | 38 | | | | | 12 |
| chr10: 71593210-71594625 | CALM2 | manual | proc | 9 | 8 | 5 | 6 | 13 | 1 | | 1 | | 11 |
| chr3: 117845959-117848342 | BZW1 | auto | frag | 18 | 20 | 10 | 14 | 20 | 2 | 3 | 5 | 3 | 3 |
| chr18: 12056629-12058570 | SSBP4 | | dup | 18 | 15 | 5 | 12 | 18 | 3 | 1 | 6 | | 7 |
| chrX: 122077782-122079346 | MRRF | | proc | 20 | 17 | 4 | 5 | 19 | 7 | 1 | 7 | | 4 |
| chr17: 60176781-60178361 | ASCC3L1 | | dup | 28 | 15 | 10 | 14 | 54 | 2 | 3 | 3 | | 13 |
| chrX: 74875425-74880031 | TTC3 | manual | proc | 28 | 15 | 8 | 10 | 55 | | 4 | 4 | | 16 |
| chr17: 63636429-63636974 | DQ587552 | | dup | 39 | 9 | 6 | 19 | 50 | 10 | 2 | 4 | 2 | 10 |
| chrY: 11770408-11775026 | FLJ00219 | | dup | 29 | 2 | 4 | 20 | 62 | 8 | | 6 | 3 | 11 |
| chr11: 113145499-113147696 | COX7A2L | | proc | 31 | 13 | 6 | 14 | 33 | 6 | 2 | 6 | 1 | 14 |
| chr5: 75708184-75708770 | SNRPC | auto | proc | 48 | 17 | 11 | 21 | 51 | 7 | 2 | 8 | 3 | 11 |

All publications, patents, patent applications and accession numbers mentioned in the above specification are herein incorporated by reference in their entirety. Although the invention has been described in connection with specific embodiments, it should be understood that the invention as claimed should not be unduly limited to such specific embodiments. Indeed, various modifications and variations of the described compositions and methods of the invention will be apparent to those of ordinary skill in the art and are intended to be within the scope of the following claims.

We claim:

1. A method of screening for the presence of breast cancer in a subject, comprising
(a) contacting a biological sample from a subject with a reagent for detecting the level of expression of ATPase, aminophospholipid transporter, class I, type 8A, member 2 pseudogene (ATP8A2-Ψ); and
(b) detecting the level of expression of said ATP8A2-Ψ in said sample using an in vitro assay,
wherein an increased level of expression of said ATP8A2-Ψ in said sample relative to the level in normal breast cells is indicative of breast cancer in said subject.

2. The method of claim 1, wherein the sample is selected from the group consisting of tissue, blood, plasma, serum and breast cells.

3. The method of claim 1, wherein detection is carried out utilizing a method selected from the group consisting of a sequencing technique, a nucleic acid hybridization technique, a nucleic acid amplification technique, and an immunoassay.

4. The method of claim 3, wherein the nucleic acid amplification technique is selected from the group consisting of polymerase chain reaction, reverse transcription polymerase chain reaction, transcription-mediated amplification, ligase chain reaction, strand displacement amplification, and nucleic acid sequence based amplification.

5. The method of claim 1, wherein said breast cancer is luminal breast cancer.

6. The method of claim 1, wherein said reagent is selected from the group consisting of a pair of amplification oligonucleotides and an oligonucleotide probe.

7. A method of screening for the presence of prostate cancer in a subject, comprising
(a) contacting a biological sample from a subject with a reagent for detecting the level of expression of coxsackie virus and adenovirus receptor pseudogene (CXADR-Ψ); and
(b) detecting the level of expression of said CXADR-Ψ in said sample using an in vitro assay,
wherein an increased level of expression of said CXADR-Ψ in said sample relative to the level in normal prostate cells is indicative of prostate cancer in said subject.

8. The method of claim 7, wherein the sample is selected from the group consisting of tissue, blood, plasma, serum, urine, urine supernatant, urine cell pellet, semen, prostatic secretions and prostate cells.

9. The method of claim 7, wherein detection is carried out utilizing a method selected from the group consisting of a sequencing technique, a nucleic acid hybridization technique, a nucleic acid amplification technique, and an immunoassay.

10. The method of claim 9, wherein the nucleic acid amplification technique is selected from the group consisting of polymerase chain reaction, reverse transcription polymerase chain reaction, transcription-mediated amplification, ligase chain reaction, strand displacement amplification, and nucleic acid sequence based amplification.

11. The method of claim 7, wherein said cancer is selected from the group consisting of localized prostate cancer and metastatic prostate cancer.

12. The method of claim 7, wherein said reagent is selected from the group consisting of a pair of amplification oligonucleotides and an oligonucleotide probe.

13. The method of claim 7, wherein said biological sample lacks and ETS gene fusion.

14. A method of screening for the presence of prostate cancer in a subject, comprising
(a) contacting a biological sample from a subject with a reagent for detecting the presence of a kallikrein-related peptidase 4-kallikrein pseudogene 1 (KLK4-KLKP1) gene fusion; and
(b) detecting the presence or absence of said KLK4-KLKP1 gene fusion in said sample using an in vitro assay,
wherein the presence of said KLK4-KLKP1 gene fusion in said sample is indicative of prostate cancer in said subject.

15. The method of claim 14, wherein the sample is selected from the group consisting of tissue, blood, plasma, serum, urine, urine supernatant, urine cell pellet, semen, prostatic secretions and prostate cells.

16. The method of claim 14, wherein detection is carried out utilizing a method selected from the group consisting of a sequencing technique, a nucleic acid hybridization technique, a nucleic acid amplification technique, and an immunoassay.

17. The method of claim 16, wherein the nucleic acid amplification technique is selected from the group consisting of polymerase chain reaction, reverse transcription polymerase chain reaction, transcription-mediated amplification, ligase chain reaction, strand displacement amplification, and nucleic acid sequence based amplification.

18. The method of claim 14, wherein said cancer is selected from the group consisting of localized prostate cancer and metastatic prostate cancer.

19. The method of claim 14, wherein said reagent is selected from the group consisting of a pair of amplification oligonucleotides and an oligonucleotide probe.

20. The method of claim 14, wherein said KLK4-KLKP1 gene fusion is an mRNA.


SEQUENCE LISTING (60 sequences)

SEQ ID NO: 1 - DNA, 25 bp, Artificial Sequence
  Synthetic
cggtttcagtgctctatgttgtttg

SEQ ID NO: 2 - DNA, 29 bp, Artificial Sequence
  Synthetic
taaatttaggattacatgtttctagaaca

SEQ ID NO: 3 - DNA, 16 bp, Artificial Sequence
  Synthetic
atgccatccaaaacca

SEQ ID NO: 4 - DNA, 24 bp, Artificial Sequence
  Synthetic
ctggtgttctttggcatctactca

SEQ ID NO: 5 - DNA, 21 bp, Artificial Sequence
  Synthetic
cagctcaggatcacagttgct

SEQ ID NO: 6 - DNA, 16 bp, Artificial Sequence
  Synthetic
ctggtccaccattctc

SEQ ID NO: 7 - DNA, 26 bp, Artificial Sequence
  Synthetic
atcctattgaaggaggactctttgga

SEQ ID NO: 8 - DNA, 22 bp, Artificial Sequence
  Synthetic
ccagcaaattcccaaggtcagt

SEQ ID NO: 9 - DNA, 16 bp, Artificial Sequence
  Synthetic
aagggcagccattact

SEQ ID NO: 10 - DNA, 20 bp, Artificial Sequence
  Synthetic
atggaaaacgaattgttctg

SEQ ID NO: 11 - DNA, 20 bp, Artificial Sequence
  Synthetic
cagtgttccgggtgatgcag

SEQ ID NO: 12 - DNA, 2476 bp, Homo sapiens
gagtgcgccaggcgcggggagcctaggacctggagcgagagccgcctacctgcagccgccgcccacggcacggcagccaccgtggcgctcctgctgcgcttcgtgctcctgtgcagagtcgcggatttcatcagaggttggagtatcactactcctgagcagatgattgaaaaagccaaaggggaaactgcctatctgccatgcaaatttacgcttagtcctgaagaccagggaccactggacatcgagtggctgatatcaccagctgataatcagaaggtggatcaagtgattattttatattctggagacaaaaattatgatgactactatccagatctgaaaggccgagtacattttaagagtaatgatctcaaatctggtgatgcatcaataaatgtaacgaattttcagctgtcagatattggcacagatcagtgcaaagtgaaaagagctcctggtgttgcaaataggaagattcagctggtagttcttggtaagccttcaggtacaagatgttacgttgatggatcagaagaaattggaagtgactttaaattaaaatgtgaaccaaaagaaggttcacttccattacagtatgagtggcaaaaattgtctgactcacagaaaatgcccacttcatggttagcagaaatgacttcatctgttatatctgtaaaaatgcttcttctgagtactctgggacatacagctgtacatcagaaacagagtgggctctgatcagtgcctgttgcgtgtaaacgttgtccctccttcaaataaagctggactaattgcaggagccattataggaactttgcttgctctagtgctcattggtcttatcatcttttgctgtcgtaaaaagcgcagagaagaaaaatatgaaaaggaagttcatcacgatatcaaggaagatgtgccgcctccaaagagccacacgtccactgccagaagctacataggcagtaatcattcatccctgggatccatatctccttccaacatggaaggatattccaagactcagtataaacaagtaccaagtgaagactttgaacgcactcctcagagtccgactctcccacctgctaaggtagctgcccctaatctaagtcgaatgggcgcgattcctgtgatgattcccgcacagagcaaggatgggtctatagtatagagcctccatacatctcatctgtgctctccgtgttcctttcctttttttgatatatgaaaacctattctggtctaaattttgttactagcctcagaatgtatcagaaaataagttaatcaggagctgtaaggaatatatttttaaaaatttttgtttggttatatcgaaatagttacgggcattaaagttagtaaagacaagtttaccatctgaaaaggctggattttctttaagaggctgattataaaggtttctaaatgttatcagtacctaagtaagatgtagcactttgagtatgaaatcataggtgaagaaatcggtgaacttacttgcataccaagttgatacttgagtaaccatctgaaagtggtacttgataatttttaccattatttttaggatgtgtatctcatttatttatggcccacaagtctcccccaaattagtacagaaacatccctgacaaaattacttatgtacgtttgtacttgttttcacagctcctcggaaaactctgtgttaggaatatctctaaaaacatagaaaacactacagtgatttagaaattactaattttacttctaagtcattcataaaccttgcctatgaaatgacttcttaaatatttagttgatagactgctacaggtaatagggacttagcaagctcttttatatgctaaaggagcatctatcagattaagttagaacatttgctgtctgccacatattgagatggcactaggtgcaatagcagggatagattttgttggtgagaggtctcatgccttgagatctgtggtggtctttaaaatggtggccagccagaccaaggatgtagtatctcatagtccccaactaaatgctggctttccactttaggtgatatttttctaattagaaaaatattataactcacttattgtttgacaattatagattgaaatttcctaattctaaattttaagtggctcttcggtttcagtgctctatgttgtttgttgttggttttggatggcattacatattatatgttctagaaacatgtaatcctaaatttaccctcttgaatatgatccctggatgatatttttatcataaatgcagaataatcaaatacattttaagcaagtaagtgtcctccatcaattccgtattccagacttgggaggatgtacagttgctgttgtgtgatcaaacatgtctctgtgtagttccagcaaatcaagctgagcttcaaaaaagtttgagtctcagttttgtgaaagtgatttattcttaaaaaaaaaaaaaaaagaaa

SEQ ID NO: 13 - DNA, 540 bp, Homo sapiens
attttcaccgctatgcctcctttcactctgggagtcttccagaggtcttgcactcgggagagcatgctcaggtttccccagctctacaaaatcacccagaatgccaaagacttcaacacaaggatactgtgttgaccagtggtcatgccactgcctgttgatttgttgaaaatattgtttacacgtatgttcttgttactgattgtcagaaagctggttttgagactgcagcttggactaaattcagtcatctggctgtctggggaagcatgctgaccagtctggtgttctttggcatctactcagccatctggtccaccattctcattgccccaaatatgagaggacagcaactgtgatcctgagctgtgcacacttctggttgggattatttctggtttctacttcctgtttgaagatgtggcatggagagtgccaagcacatctgcaaaaaagacattgctggaggaggtgcaaagagctggaaaccaagtctccagtcctgggaaaagcagtggtatggaaaagcaatggaaagag

SEQ ID NO: 14 - DNA, 1135 bp, Homo sapiens
atggcggccgctgcacaatcccgggttgtctgggccctgccaatgtcacattctgccattactgcaatagccacatccgtgtgtcacagcccactccattgccagcttcatcatgccctcatacctcatgggaaaggtgaatgttcctcaagtcagtgggattgtggccactgggtttggagcaacaggattcctgggccgatgtgttgtcaaccaccttggacgcgtggggtcacaggtgatcataccctatcggtgtgatacatgtgacatcatgcaccttcgtcccatgggagacctgggctagcttctgtttctggaatgggacgcgagagataaagattctatctgacgagtagtggaacacagcaatgtggtcataaatcttgttggacgagactgggaaaccaaaaactttgattttgaggatgtttttgtgaagattccccaagcaattgctcaggtgtccaaggaagctgaagttggaaaattcattcatgttgcacatctgaatgtgaatattaaaagctcttctagatatttgagaaagaaggctgttggagagaaagcagtgagagacacatttccagaagccattatcgtaaagctgttggacatctttggaagagaggatagattccttaattattttgcaaatatgtgttggtttggtgctataccccttgtttccttgggctggaaggcagttaaacaaccagtatacgtcgtagatgtatccaaaggaattgttaatgcagttaaggatcctgatgccaatgggaaaacctttgctttcgttgggccaaatcgatatctcctctttgacctggtgaagtacatctttgctgtggctcacagcaaaggggtacttcctcccgtaccccttgccaccttttgcctatcagtgggtagcaagagactttgaaataagcccatttgagtcctggacaacaagggataaagtggagcgtattcacatcacagacatgaccttgtctcacctgcccagcttagaagaccttggtattcaggcaacaccactggaactcaaggccattgaggtgctgcggcttcatcacacttactgctggctgtctgctgaaattgaggatgtgaagctggccaagaccgtcaa

SEQ ID NO: 15 - DNA, 1696 bp, Homo sapiens
caccacctatttctttggtaattgtaccatggaagatggcaaactggcccaagactttctggactcacagaacctcagtgcgtgtacaacacctggctcttcaaagaggtcgatggagaagggaagccccactacgaggtgcagctggcttctgtgctcggcacagagccttccctggactccgaggtgattccaagctgaagagctaggaattccggggaagccctttccaggtgacttgggggaactacatgcccatcctccagaaggtggtggagcagctggagaaagccaagacgtatgcagccaacagccaccaggagcaaatgctggcccagtacatagagagcttcacccagggctccacagaggcccacaagaaggggtcccgcttctggatctaggacaaaggccccatcgtggagagttacatcgagttcatccagagctaccgtgactcctttggttcccgaggagtatgtgaaggcttcgtagccgtggtgaacaaggccatgagtgccaagtttgagtggctggtggtgagcgcagagcagctgctgaaggagttgccctggtccccagcctttgagaaagacaagttcctcacccctgacttcacctccgtggatgttcttaccttcgctggctctggcatcgctgccggcatcaacatctccaactacaatgacctgaagcagacggaaggctttaagaacgtgtcactggggaacgtcctggctgtggtctaagccacgcagtgggagaagctcaccgttctggaggagagtgacaaggacttgtacatcgtcctgatggggccctccttcgatgtacaggtgggcctgcacgaactgctgggctatggcagcggcaagctcatcgagcaggatgaaaaaggagcattcaactttgaccaggagacccatcaatccagagatgagagagcagattcagagctggtatcggagcatgaagacctgggacagcaagttcagcaccattgtctccagctacgaagagtgccgggctgagagcgtgggcctctacctctgtctccacccgcaagtgctggagacctttggctttgagggggctgatgcagaggaggtgatctccgtgaactggctcaacatggttggggctgggctgctcgctctggagttctacacacctgaggcctccaactggcagcaggcccatatacgggcccggattgtgatcctgagagtcttaccagaggctggcgagggactcggtaccatcactcccaccgcaggctccgatgggcgcccggaagcccaggtccgtctcgaccgcagcaagatccagtctgtgggcaaccctgccctagagcgcttcctgcggaggtgctggtccacaggggatgtggccggagggtggaccctgtacgagaggtatgcagcagtcgctgatgcgccccctgagggctttctcaccctcagggacagggtgctgctgcgtaaggaatcttggaagctcatcgttcagcccaacattcgccttgaaggctcagacgtgcagcttctggaatacgaggtctcagctgctggcctcatcagatccttctctgagcatttcccagaggatggactcgagttggaggacatcctaacacagctggcaacagccgatgcccaattctgaaagggccccagtgaggccccatctggccagg

SEQ ID NO: 16 - DNA, 859 bp, Homo sapiens
gttttgttgtggttcccaggcggtgccttcgagactggctcagcctccatcttcgatgggtcctccctggctgcctatgaggacatgctggttgtgatcgtccagtaccggctgggaatatttggcttcttcaccacatgggatcagcacgctcccgggaactgggccttcaaggaccaggtggctgctctatcctgggtccagaagaacatcgagttcttcggtggggaccccagctctgtgaccatctttggtgagtccgtgggagccataagtgtttccagccttattctgtctcccatggccgaaggcttattccacaaagccgtcatggagagtggggtggccatcatcctttacctgaaggcccatgattatgagaagagtgaggacctgcaggtagttgcacaattctgtgggtacaatgcatcagactctgaggccccactgaggtgcctgaggacaaaatcctccaaggagctgctgaccctcagccagaaaacaaaggctttcactcaagtggttgatggtgctttctttcctaatgagcctctagatctattgtctcagaaagcatttaaagcaattccttccatcatcggagtcaataaccccgagtgtggcttcccgctgcctatgaaggaggctcctgagattctcagtggctccaacaagtcccttgccctctgtctgacacaaaacatcctgcacatcctgcccaattatttgcaccttgtggctggtgaatacttccatgacaagcactccctgactgaaatccgagacagtcttctggacttgcttggagatgtgttctttgtgatccctaagctgatcacagctcagtatcatagaggtg

SEQ ID NO: 17 - DNA, 549 bp, Homo sapiens
gccgcgatgcctaaaggagggagaaagggaggccacaaaggctgggcgaggcagtataggagccctgaggagatcgacgtacagctgcaggctgagaagcagaaggccagggaagaagaggagcaaaaagaaggtggagatggggctgcaggtgaccccaaaaaggagaagaaatctctagactcagatgagagtgaggatgaagaagatgactaccagcaaaaccgcaaaggcgttgaagggctcttcaacatcgagaaccccaaccaggtggcacagacaaccaaaaaggtcacacaactgtatctggatgggccaaaggagctttcgaggagagaacgagaagagattgagaagcagaaggcaaaagagcgttacatgaaaatgcacttggccgggaagacagagcaagccaaggctgacctggcccgcgccatcatccggaaacagcgggaggaggctgcccggaagaaggaagaggaaaggaaagcaaaagatgatgccacattgtcaggaaaacgaatgcagtcgctctccctgtataagtaa

SEQ ID NO: 18 - DNA, 2473 bp, Homo sapiens
atgaactaccagcagcagctggccaacttggctgccatcccggccgagatccagcacttcgagttggtccaccccaacgtctactcccatctaccagctgctggagcgcgtggaggagccggtgcttcagaaccagatccgggagcacgtcattgccatcgaagatgtctttgtgaacagctaggaatggacgctgagtcgatctgtcccggagctcaaagtggtggcaggttcaagaaagagattgttgttgatggacagagctatctgctgctgattaaatatgaaggggaccccctggaggcgcagtttgccatgtcggtggatgcagttatatttgttttcagcttggaggatgaaataagtttccagactgtttactacagtggaatggccaactatcggaacatgagcgagattcctctggttctggtgggaacccaggatgccataagttccactaacctgagggtcattgatgatgccagggtaaggaagtgctccaacgacctgaagcggtgcacgtactatgagacgtgtgctatgtatgggctgaacgtggaaagggacttccaggaggttgcccagaagattgttgccacaaggaagaagcagcagctgcccataggaccctgcaagttgctgcccagttctccaagccattccaccgtctgttctgcacaggtgtccgccatgcacatcaactagacaagtaatggaggtaggagtttaagcaactattcctcctctgttccatcgactcccagcaccagccagaaggaacttcagatggatgttcctcccactgccaacacgcccacacccattcacaagcagttcacaagcagttcaagtggggtcaaacctgttcacctctcagaaagggagcaacccaaagagtagaaaggcctagagagtcatgcatacggcattgggagcagccaagccatgccaattatacagggcatgctgttgaagcagagtggcaaatcgttgaataaagagtggtaaaagaaatatgtcaccctgtgtgacaatggtgtgcggacctatgatccaagtttacatgattacatgcagaatgtccatggtaaggagactgaccttctgagaaccactatgaaagtcccagggaagaggctaccccaagccacgtcggcctgcgtgcccatctccaggactaaaaccaatggcctatccaagcacatgaggagtttacacatctcacccaattcaggaaatgtcactagtgcatctgggtctcagttggcaggtggcatcagcctggtctccttcaacagccaaccagatggcatgcaccagcactcctactcagtctccagagccaaccagtggagtcaggctacagtcattgcaaactcggccattagcagtgacaaggggtgggtgactctgtatgctccagtcgcagtaactccagcaccaccagcccacctcccctcacaccaacagaaagaagcactataggcagaaagccaccagcaacttcaaagctgatggcctgtctgacactgctgaagaacaagaaaactttatcattgtgtccctcactggccaaacgtggcactttgaagccacgacgaatgaggagcaggacacctgggtccaagccatcgagagccagatcctggccatcctggccagcctgcagtcgtgcaagagcagcaagaacaagtcccagctgatgaaccagagtgaggccatggccctgcagtcaatctgacacatgcacggaactcccactgtgtgaactgcgagacccagaatcctaactgggccagtttgaacttgggagtgctcacgtgcatcaagtgcttaggaatccattggaatcttggcacccacctttcccgagtctgatctctggacctggatgactggccagttgagcttatcaagatgatgtcatccattgggaacgagatattcaacagcgtctgggaagagagcagccaggggcggatgaaacccttagtagactccacaaggaaagagaaggaacaatggatccatgccaactacgagcagaagctcttcctggccccgctaccctgcacggagctgtccctgggccagcacccgcttcgggccaccgcagacgaagaccggcagatggccatcctgctgctggtgcacagctcctgggacaaggtgaaagaaacctttggggagggagacagagatggccgcactgcactgcatctggcctgccacaagggaagtgtggtcctggtgcagctcctgatctggtaccgggtggacgtcatggcccaagatgcccatgggaacacagtgctggcctacacctggcaggcctcgaggcaggaatgcatcaatgtgccctggagtatggctgcccaacgagcactgagtgctcatggccacccctaacctgtccaggagaaacaataaccagaaaagcagcagcaggagggtgcccaccatcatctga

SEQ ID NO: 19 - DNA, 966 bp, Homo sapiens
ggcatgtctcggaggttggcatcacggaggcactgtgcagtccttgcaatgtcaaacttactcagtgccagcatagacggtggtctcatgcactccataggtttagggtactacaaggatctccagacagctacatttatggaagttctgatgaaaatccctcaacaaggcacagaatttgacacacttcagaaacggtattggctgatgggtttgagcaattggtggaactggtcacagtgatgggtgatcaaggagagctccctatagcgatggctctggccagtgtggttccttgttctcagtgggatgaactggcttgagttctggttactctatttggttcttggcatttactctaccaactgctctggaacatgttttgtaaagaagtagaattggcagactgactccattcagactctcctctgaggcaacagtttggccagtaaaataatgacactctgtttcaaggtgcccaagaattcccatccctccatggttcaaataatagagtattgccaatcatgttctcttcacaaaagaagagaatatgcggcctttcaatgattttgtgaaaagcagctttgatgcagcttgaagtgatgcagtaaatcatagcctttccttcagcagtgacggcaatgtgctcgctttacatcatctactccggaacaatcaggagaaaattggccagtgtctttctaacaacagggatcataaagctgttggaagatgaccttttgataaatggcaacacttcttgtatacctcggtcctccagagcacaaacctgtggcagatacataccggtccagccttaaccttaccagttaaaagtttgaggaatttatgatgaggcatcaggtatgtgaaaaagaagagatcaaggctttgaaaacattaagtattttttaccaagctgggaattccaaagctgggaagccaattttttatgttgcaagg

SEQ ID NO: 20 - DNA, 1218 bp, Homo sapiens
atgtcgctttctaacaagctgatgctggacaagctggacgtgaaagggaagccggtcgttatgagagtcgacttcaattttcctatgaagaacaaccaggtaacaaacaactggaggattaaggctgctgtcccaagcatcaaattctgcttggacaatggatccaagtcagtagtccttataagccacctaggttggcctgatggtgtccctatgcctgacaagtactcctcagagccagttgctgtagaactcaaatctctgctgggcaaggatgttctgttcttgaaggactgtgtaggcccagaagtggagaaagcctgtaccaacccagctgctgggtctgtcatcctgcgggagaacctccgctttcatgtggaggaagagggaagggaaaagatgcttctgggaacaaggctaatgctgatccagccaaattagaagctttccaagcttcacttcccaagctaggggaagtctatgtcaatgatgcttttggcactgctcacagagcccacagctccatggcagaagtcaatctgccacagaaggctggtggttttttgatgaagaaggagctgaactactttgccgaggccttggagagcccagagcgacccttcctggccatcctgggcagaactaaagttgcagacaagatccagctgatcaagaacatgctgggcaaagtcaatgagatgattattggtagtggaatggcttgtaccttccttaaggtgctcagcaacatggagattggcacttctctgtttgatgaagagggagccaagactgtcaaagacctaatgtccaaagctgagaagaatggtgtgaagattatcttgcctgttgactttgtcactgctggcaagtttgatgagaataccaagactggccaagccactgtggcttctggcatacctgctggctggctgggcttggactgtggccctgaaagcagcaagaagtatgctggggctgtcactcaggctaagcagattgtgtggaatggtcctgtggtgtaggggtttttgaatgggaagcttttgcccggggaaccaaagccctcatggatgaggtggtggagacactgccacttgctgtgtagaatggaacatggaggataaagtcagccaggtgagcactgggggtggtgccggtttagagctcctagaaggtaaagtccatcctggggttgatactcttggcaatatttag

SEQ ID NO: 21 - DNA, 229 bp, Homo sapiens
atgcccaagtgccccgcgtgcgacaaggtgtacttcgccgagagggtgacctctctaggcaaggactggcatcggccctgcctgaagtgtgagaaatgtgggaagacgctgacttccgggggccacgctgagcatgaaggcaaaccctacggcaaccacccctgctacgccgccatgtttgggcctaaaggctttgggcggggtggacccgagagccacactttcaagt

SEQ ID NO: 22 - DNA, 1813 bp, Homo sapiens
caccaatacatttattcatttatttgcaggagatggggtcaaatcttaccactaacttttttttttttttttgagacggagtcccgctgtgttgcccaggctggagtgcagtggcgcaatctcagctcactgcaacctctaactcctgggttcaagagattctcctgcctcagccttccgagtagctgggattatgggtgcccaccaccacgcccagctaatcttttttgtatttttagtagacatggggtttcactatatgttgaccagtctggtctcaaactcctgatctcaagtgatccacccacttcggcctcccaaagtgctgggattacaggcgtgagccactgcgcctggccttatgaacttttaagctaggtaggactagacacactgatctgcctaacctctgggtatttatcctgcacacaaggcatcaccagtaaatacatgagcattagatgagggggatgcattctgactaatcacatgattagctattcagaggaagatactgcccttgtatcaaaagtagtgttgagaaaaataaaggcagagcctagcatttgcctcctagcacaggctgaaacatgctagtggccttgtgcagaagaatgatgctttcagctgagctgcaagataggagctggaccaactgggggcttggacaggggtgggatctgacctgattaggctttccccatccaatctgggcctccaaaagccgttctctggccctctggacaaggcagactcagcaaatctgggaggtatggggattctgccaattccccacctagccccacctcccccaggtttggcaggcgatctaatccccagggtgcagccccacccccacctgaccccacatctggacaaacacatggcaaatatggaaactgaagcccagctgggctggagcacatctggttgttgctgcattggagtcatcttgcagatatcctgagataatcaggcctcacccacagtggccatgttgagacagcagttgtttttttttaaagatgattcacgttgctccaggcgaaagcatgatcctgatgacacgtggggaaaagcaggctggagcctcagaagagctggccctgcacactcaggtacttacagcagtaaatccaggatttgaacaccagggagtaaggagagggcccagcctgcggcctgcagttgttcacagccatgaggctgccaactggcaggaaagggtaggtcagcatgctcactgcgatctccatcacaaacttggtgtagctttggatggtcagggcctggcctaactgggaaccccgattctggtcatttctcagcccctctaggtgtcactcactctgtcatccgccaggtgggcattgataaagtgggccagcaggttatagcctcacaagaaaactacttcacccgggacatgagggatcaagccaacaaagaatcccagtggccctttctctttgaaaatcttcccagtggagctcagcacaccactgtacttaacctcccatcccacaaattagaccatgcagcacatcaagatggcatgcagggggtgggccaacatacaggacgcacactgcatcatcatcccctaggaggtctccttcactgcttgcttcagagaagtcttcatatccatccttattggaaacctgcttgatttcatctggagggaaaaccttcacgctgcccctggttacaaaggagaggggtcacatcagttgggggctcagacctcggaacagccctgtcttgccatccatcacacccatgcccagtgccacaaaaagcttcagtggtc

SEQ ID NO: 23 - DNA, 1242 bp, Homo sapiens
atggacgtcgacaggctccaggaggcgctggaagattttgagaagaggcaaaaaagaaagtctgtcctgtcctggatcagttccttttgtcatgtagccaagactggagaaacagattcagtggtcccaatttaaaggctattttattttcaaactggagaaagtgatggatgatttcagaacttcagctcctgcgccaagaggtcctcccaaccctaaagtcgaatatattccctttgatgaaacaaagggaagaatactgaaaactgtcactggatttaaccgtatcccttttactattcagcgattatgtgaattgttaacagatccaaggagaaactatacaggaacagacaaatttctcagaggagtagaaaagaacgtgatggttgttagctgtgtttatccttcttcagagaaaaacaattccaatagtttaaatcgaatgaatggtgtgatgtttcctggaaatgcaccaagctatactgagaggtctaatataaatgggcctgggacacccaggccacgtaatcgaccaaaggtttctctgtcagccccatgacaacaaatgggtggcctgagagcacagacagcaaagaggcaaatttgcagcaaaatgaagagaaaactcacagtgactcttcgacatctgaatcagaagtttcctcagtgagccctttgagaaataaacatccagatgaagatgctgtggaagctgaggggtatgaggtaaaaagactcaggtttgacaaaaaggcgaagtcggagaaacagccagtcaagagacttgcagcgaaatttcttcagttatggtagaagaaacagaagcatcaccttcatctcatgataaagacaaagaaagctgtggtacccggcagcgtgttcagaagaggatgaagaagatgaagaggaagaagaagggactgagagacatcaagagaaatgatcccagaaagaaataaacaagaaaaagaatctgatgatgcctcaactgtgaatgaagagacttctgaggaaaataatgaaatggaggaatctgatgtgtctcaagctgagaaagatttactacattctgaaggtagtgaaaacgaaggccctgaaagtagtggttcttctgactgccgtgaaacagaagaattagtaggatccaattccagtaaaactggagagattctttcagaatcatccatggaaaatgatgacgaagccacagaagtcaccgatgaaccactggaacaagac

SEQ ID NO: 24 - DNA, 1457 bp, Homo sapiens
ggtgtgataggtatacagctgttgttaccatggtgatggccagtgtcatgcagaagattatacctcgctattctcttgctcaatggctactctgtaatggcagtttgaggtggtatcaacattctacagtagaagaatcaagaattcttgcagggaatcagcaaaaagagaaaagcaaaaaagataggaaatataatggtcacactgaaagtaagccattaaccattccaaaggatattgaccttcgtctagaaacaaagtcagttacagaagtggatacattagcattgcattacttcccagaataccagtgactggtggatttcacagtggctgctacagtcgtgtatctagtaactgaagtcgactacaagtttatgaagcctacacaggaaataaatatcagcttagtctggtgcctgcttgtttcgtcttttgcaatcgaagttctattttcattaattacacactattttaaagtagaagatgctggtgaaagatctgtttgtgtcacctttggattttttttctttgtcaaagcaatggcagtgttgattgtaacagaaaattatctggaatttggacttgaaacagggtttacaaatttttcaggcagtgcgatgcagtttcttgaaaagcaaggtttagaatctcagagtcctgttttaaaacttacttccaaattttttcctggctattttctgttcactcactggggcttttttcacatttcctggattacggctggctcaaatgcatctggataccctgaatttggcaacagaaaaaattacacaaacattatttcatatcagcttcttggcacatttatttatggttctgctctgggtaaaaccagtcaccaaagactacattatgaacccaaccttgggtgaagaaagcatcactttaatgacagaagccacatttggtactctgcaaatctggttaataatcctgctgtgtgctttgcggttggccatgatgcatagtcacctgcaagcttatttaaatttagcccaaaaatgtgtggatcagatgaagaaagaagcagggtgaataagtacagttgagctacagaaaatggtggcttgagtcttttattatctttttgtcattgcactgcagtatgtggtgcctctggtaacactgtttcacacaactctgcttttgaaaacactaggtaatcattcctggggtatttatccagaatctgtctctaccttaccagtggataatagtctactctacaattctgtttactctgaattaccatcagctgaagagaagatgaaggttactgttacacaaataacagtggcactgagcagcttaaaaacatttttacacttcttgttttttgaggacttctgtcttttctgacctggtagattgctgcttgcctcttttctacaagcctttttgggcttttttatcaccagtatctgactgtggca

SEQ ID NO: 25 - DNA, 1024 bp, Homo sapiens
catgcccaccatcattgagcgggagttcaaggagttggatgctcagcatcgctggcagctgccgtacttggaaattcgaaatgagtcccatgactatcttcatagagtggccaagtttccagaaaacagaaatcgaaatagatacagagatgtaagcccatatgatcacagtcgtgttaaactgcagaatactgagaatgattatattaatgccagcttagttgacatagaagaggcacgaaggagttacatcttaacaccgggtccacttcctaacatgtgctgccatttctggcttacggtttggcagcagaagaccaaagcagttgttgtgctgaaccgcattgtggagaaagaatcggttaaatgtgcgcagtactggccaacagatgaccaaaagatgctgtttaaagaaacgggattcaatgtgaagctctttcagaagatgtgaagtcatattacacagtacatctactacgattagaaaatatcaatagtggtgaaaccagaacaatatctcacttgcattatacgacctggccagattttggagtccctgaatcaccagcttcatttctcaatttcttgttgaaagtgagagaatctggctccttgaaccctgaccatgggcctgcagtgatccattgtagcgaaggcactgggtgcacgggcaccttctctctggtagacgcttgtcttgttttgatgggaaaaggagattatattaacattaaacaagtgttattgaacatgagaaaatactgaatgggtcttattcagaccccagatcaactgagattctcatacatggctataatagaaggagcaaaatgtataaaggtagattctagtatacagaaacggtggaaagaattttctaaggaaaacttagctcctgcttttgatcattcaccgaacaaaataatgactgaaaaatacaatgggaacagaattggtctagaagaagaaaaactgacaggtgaccgatgtacagtaaatgcaagatacaatggaagagaacagt

SEQ ID NO: 26 - DNA, 2765 bp, Homo sapiens
atggagtccatgtttaataaattgaagagtactgttacagaagtaacagctgatgtcactagtgctgtaatgagaaatcctgtcactagagaatttgatgttggtcagcacattgccagtggtggcagtgggctagcttggaagatttttaatggcacaaaaaagtcaacaaagcagtggctgtttttgtctttgataaaaaatgattgacaaatatcaaaaatttgaaaaggatcaaataattgattctctaaaatgaggagtccaacacttaactcgacttcaacaccctcgacttcttactgtccagcattctttagaagaatccagggattgcttggtgttgtgtacagaaccagttttttgccagtttagccagtgttcttggtaactgggaaaatctccctttccctatatctccagacattaaggattataaactttatgatgtagaaaccaaatgtggtttgcttcaggtttctaaaggattgtcattcttgcatagcagtgtgaaaatggtacatggaaatattactcctgaaaatacaattttgaataaaagtggagcctggaaaataatgggttttgattgttgtgtatcatcaaccaatccttctgaacaagagcctaaatttccttgtaaagaatgggacccaaatttaccttcattgtgtcttccaaatcctgaatatttgcctcctgaatacatactttatgtgagctgtgaaacagtcagtgatatatattctttaggaactgttatgtatgctgtatttaataaaaggaaacatatatttgaaggcaataagcaagacatttataagagtttcagtaggcagttggatcagttgagttatttaggatctagttcacttataaatatacctgaggaagttcgtgaacatgtaaggctactgttaaatgtaactccaactgtaagaccagatgcagatcaaatgacaaagattcccttctttgatgatgttggtgcagtaacactgcaatattttgataccttattccaaagagataatcttcagaaatcacagtttttcaacggactgccaaaggttctactgcccaagcatgtcattgtacagagaattttgatttgacttcagaatttgtaaaccctgacatggtaccttttgttttgcccagtgttctactgatcgttgaggaatgcaccaaagaagaatatgtcacattaattccacctgaacttggccctgtgtttaagcagcaggagccaatccagattttgttaattttcctacaaaaaatggatttgctactaaccaaaacccctcccaatgagataaagaacagtgttctacccatggtttacagagcactagaagctccttccattcaataccaggagctctgtctaaacatcattccaacctttgcaaatcttatagactacccatcatgaaacatactttgataccaagaattaaaaatgcctgtctacaaatgtcttcccttgctgttcctgtaaattcattagtgtgcttaggaaagattttggaatacttggataagtggttggtacttgatggtatcctacccttcttacaacaaattccatccaaagagtctgcagtcctcatgggtattttaggcatttacaaatgtacttttactcataagttgggaatcaccaaagagcagctgactggaaaagtattgcctcatcttattcccctgagtattgaaaacaatcttaatcagttcaattctttcatttccatcataaaagagatgctgaataggttggagtctgaacataagactaagctggagcaacttcatataatgcaagaacaacagaaatctttggacataggaaatcaaatgaatgtttctgaggagacaaaagttacaaatattgggaatcagcaaattgacaaggtttttagccacattggagcagaccttctgactggcagtgattccgaaaataaagaggatgggtttcagaataaacataaaagagcatcacttacacttgaagaaaaacaaaaattagcaagagaacaagagcaggcatagaagttgaaaggccagcagcctcttaaaccccaagtgtacacacctattgctgctgttaaacagactaaggacttgacagacacattgatggataatatgtcatctttgactagcctttctgttagtacccctaaaccttctgcttcaagtacgttcacttctgttccttccatgggcattgacatgatgttttctacaccagttgataatacaaagagaaatttgacaaatggcctaaatgtcaatatgggctttcagacttcaggattcaacatgcccgttactacaaaccagaacttctacagtagtccaagcacagttggagtgaccaaaatgactctggaacacctcccactttgccaaacttcagtgctttgaatgttcctcctgctggtgcgaagccaacccaacaaagacccacagatatgtctgcccttaataatctctttggccctcagaaacccaaagttagcatgaaccaattatcacaacagaaaccaaatcagtggcttaatcagtttgtacctcctcaagtttctccagctacgggcagttcagtaatgggaacacagatgaacatgataggacaatctgcctttggtatgcagggtaatcctttcttcaacccacagaactttgcatagccaccaactgctatgaccaatagcagttcagctagcaatgattttaaagatctttttgggtga

SEQ ID NO: 27 - DNA, 1180 bp, Homo sapiens
gatcgctcgtctctggtagctgcagccaaatcataaacggcgaggactgcagcccgcactcgcagccctggcaggcggcactggtcatggaaaacgaattgttctgctcgggcgtcctggtgcatccgcagtgggtgctgtcagccgcacactgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacgatgctgtgattgccatccagtcccagactgtgggaggctgggagtgtgagaagctttcccaaccctggcagggttgtaccatttcggcaacttccagtgcaaggacgtcctgctgcatcctcactgggtgctcactactgctcactgcatcacccggaacactgtgatcaactagccagcaccatagttctccgaagtcagactatcatgattactgtgttgactgtgctgtctattgtactaaccatgccgatgtttaggtgaaattagcgtcacttggcctcaaccatcttggtatccagttatcctcactgaattgagatttcctgcttcagtgtcagccattcccacataatttctgacctacagaggtgagggatcatatagctcttcaaggatgctggtactcccctcacaaattcatttctcctgttgtagtgaaaggtgcgccctctggagcctcccagggtgggtgtgcaggtcacaatgatgaatgtatgatcgtgttcccattacccaaagcctttaaatccctcatgctcagtacaccagggcaggtctagcatttcttcatttagtgtatgctgtccattcatgcaaccacctcaggactcctggattctctgcctagttgagctcctgcatgctgcctccttggggaggtgagggagagggcccatggttcaatgggatctgtgcagttgtaacacattaggtgcttaataaacagaagctgtgatgttaaaaaaaaaaaaaaaaaaaaaaa

SEQ ID NO: 28 - DNA, 105 bp, Homo sapiens
ttgacaagtggtcatgctaccgactatttatttgttggaaatattgtttacacatatgttgttgttactgtttgtctgaaagctggtttggagaccacagcttgg

SEQ ID NO: 29 - DNA, 105 bp, Homo sapiens
ttgaccagtggtcatgccactgcctgttgatttgttgaaaatattgtttacacgtatgttcttgttactgtttgtcagaaagctggttttgagaccatggcttgg

SEQ ID NO: 30 - DNA, 42 bp, Homo sapiens
tgaaaatattgtttacacgtatgttcttgttactgtttgtca

SEQ ID NO: 31 - DNA, 41 bp, Homo sapiens
gtcatgccactgcctgttgatttgttgaaaatattgtttac

SEQ ID NO: 32 - DNA, 42 bp, Homo sapiens
gttactgtttgtcagaaagctggttttgagaccatggcttgg

SEQ ID NO: 33 - DNA, 40 bp, Homo sapiens
accagtggtcatgccactgcctgttgatttgttgaaaata

SEQ ID NO: 34 - DNA, 40 bp, Homo sapiens
gtatgttcttgttactgtttgtcagaaagctggttttgag

SEQ ID NO: 35 - DNA, 40 bp, Homo sapiens
ttgaccagtggtcatgccactgcctgttgatttgttgaaa

SEQ ID NO: 36 - DNA, 40 bp, Homo sapiens
cttgttactgtttgtcagaaagctggttttgagaccatgg

SEQ ID NO: 37 - DNA, 41 bp, Homo sapiens
actgcctgttgatttgttgaaaatattgtttacacgtatgt

SEQ ID NO: 38 - DNA, 40 bp, Homo sapiens
ttactgtttgtcacaaagctggttttgagaccatggcttg

SEQ ID NO: 39 - DNA, 40 bp, Homo sapiens
gaccagtggtcatgccactgcctgttgatttgttgaaaat

SEQ ID NO: 40 - DNA, 41 bp, Homo sapiens
acacgtatgttcttgttactgtttgtcagaaagctggtttt

SEQ ID NO: 41 - DNA, 106 bp, Homo sapiens
ttagaaaaatattataactcatttgttgtttgacacttatagattgaaatttcctaatttattctaaattttaagtggttctttggttccagtgctttatgttgtt

SEQ ID NO: 42 - DNA, 102 bp, Homo sapiens
ttagaaaaatattataactcacttattgtttgacaattatagattgaaatttcctaattctaaattttaagcggctctttggtttcagtgctctatgttgtt

SEQ ID NO: 43 - DNA, 38 bp, Homo sapiens
aattatagattgaaatttcctaattctaaattttaagc

SEQ ID NO: 44 - DNA, 43 bp, Homo sapiens
agaaaaatattataactcacttattgtttgacaattatagatt

SEQ ID NO: 45 - DNA, 41 bp, Homo sapiens
aaattttaagcggctctttggtttcagtgctctatgttgtt

SEQ ID NO: 46 - DNA, 43 bp, Homo sapiens
ttagaaaaatattataactcacttattgtttgacaattataga

SEQ ID NO: 47 - DNA, 38 bp, Homo sapiens
cctaattctaaattttaagcggctctttggtttcagtg

SEQ ID NO: 48 - DNA, 79 bp, Homo sapiens
cttattgtttgacaattatagattgaaatttcctaatttaaattttaagcggctctttggtttcagtgctctatgttgt

SEQ ID NO: 49 - DNA, 43 bp, Homo sapiens; identical to SEQ ID NO: 46

SEQ ID NO: 50 - DNA, 38 bp, Homo sapiens
attctaaattttaagcggctctttggtttcagtgctct

SEQ ID NO: 51 - DNA, 648 bp, Homo sapiens
cccagaatggcgaaggcttcaacacaaaggttttctggggtcactgcatcaacgccttggtccactccctcatcctcttctggtttcccatgaaagctctggagcatgatactgtgttgacaagtggtcatgctaccgactatttatttgttggaaatattgtttacacatatgttgttgttactgtttgtctgaaagctggtttggagaccacagcttggactaaattcagtcatctggctgtctggggaagcatgctgacctggctggtgttttttggcatctactcgaccatctggcccaccattcccattgctccagatatgagaggacaggcaactatggtcctgagctccgcacacttctggttgggattatttctggttcctactgcctgtttgattgaagatgtggcatggagagcagccaagcacacctgcaaaaagacattgctggaggaggtgcaggagctggaaaccaagtctcgagtcctgggaaaagcggtgctgcgggatagcaatggaaagaggctgaacgagcgcgaccgcctgatcaagaggctgggccggaagacgcccccgacgctgttccggggcagctccctgcagcagggcgtcccgcatgggtatgctttttctcaagaagaac

SEQ ID NO: 52 - DNA, 446 bp, Homo sapiens
cccagaatgccaaagacttcaacacaaggatactgtgttgaccagtggtcatgccactgcctgttgatttgttgaaaatattgtttacacgtatgttcttgttactgattgtcagaaagctggttttgagactgcagcttggactaaattcagtcatctggctgtctggggaagcatgctgaccagtctggtgttctttggcatctactcagccatctggtccaccattctcattgccccaaatatgagaggacagcaactgtgatcctgagctgtgcacacttctggttgggattatttctggtttctacttcctgtttgaagatgtggcatggagagtgccaagcacatctgcaaaaaagacattgctggaggaggtgcaaagagctggaaaccaagtctccagtcctgggaaaagcagtggtatggaaaagcaatggaaagag

SEQ ID NO: 53 - DNA, 444 bp, Homo sapiens
cccagaatgccaaagacttcaacacaaagatactgtgttgacaagtggtcatgccaccgactattgatttgttgaaaatattgtttacacatatgttcttgttactgattgtcagaaagctggtttggagaccacagcttggactaaattcagtcatctggctgtctggggaagcatgctgaccaggctggtgttctttggcatctactcaaccatctggcccaccattcccattgccccaaatatgagaggacagcaactatgatcctgagctccgcacacttctggttgggattatttctggttcctactgcctgtttgaagatgtggcatggagagagccaagcacacctgcaaaaagacattgctggaggaggtgcaagagctggaaaccaagtctccagtcctgggaaaagcagtgctacggaaaagcaatggaaagag

SEQ ID NO: 54 - DNA, 2492 bp, Homo sapiens
gggtgcagaggtgccgccgccgccgcgagccagtcgggagcgcgcgaggcgcggggagcctgggaccaggagcgagagccgcctacctgcagccgccgcccacggcacggcagccaccatggcgctcctgctgtgcttcgtgctcctgtgcggagtagtggatttcgccagaagtttgagtatcactactcctgaagagatgattgaaaaagccaaaggggaaactgcctatctgccatgcaaatttacgcttagtcccgaagaccagggaccgctggacatcgagtggctgatatcaccagctgataatcagaaggtggatcaagtgattattttatattctggagacaaaatttatgatgactactatccagatctgaaaggccgagtacattttacgagtaatgatctcaaatctggtgatgcatcaataaatgtaacgaatttacaactgtcagatattggcacatatcagtgcaaagtgaaaaaagctcctggtgttgcaaataagaagattcatctggtagttcttgttaagccttcaggtgcgagatgttacgttgatggatctgaagaaattggaagtgactttaagataaaatgtgaaccaaaagaaggttcacttccattacagtatgagtggcaaaaattgtctgactcacagaaaatgcccacttcatggttagcagaaatgacttcatctgttatatctgtaaaaaatgcctcttctgagtactctgggacatacagctgtacagtcagaaacagagtgggctctgatcagtgcctgttgcgtctaaacgttgtccctccttcaaataaagctggactaattgcaggagccattataggaactttgcttgctctagcgctcattggtcttatcatcttttgctgtcgtaaaaagcgcagagaagaaaaatatgaaaaggaagttcatcacgatatcagggaagatgtgccacctccaaagagccgtacgtccactgccagaagctacatcggcagtaatcattcatccctggggtccatgtctccttccaacatggaaggatattccaagactcagtataaccaagtaccaagtgaagactttgaacgcactcctcagagtccgactctcccacctgctaaggtagctgcccctaatctaagtcgaatgggtgcgattcctgtgatgattccagcacagagcaaggatgggtctatagtatagagcctccatatgtctcatctgtgctctccgtgttcctttcctttttttgatatatgaaaacctattctggtctaaattgtgttactagcctcaaaatacatcaaaaaataagttaatcaggaactgtacggaatatatttttaaaaatttttgtttggttatatcgaaatagttacaggcactaaagttagtaaagaaaagtttaccatctgaaaaagctggattttctttaagaggttgattataaagttttctaaatttatcagtacctaagtaagatgtagcgctttgaatatgaaatcataggtgaagacatgggtgaacttacttgcataccaagttgatacttgaataaccatctgaaagtggtacttgatcatttttaccattatttttaggatgtgtatttcatttatttatggcccaccagtctcccccaaattagtacagaaatatccatgacaaaattacttacgtatgtttgtacttggttttacagctcctttgaaaactctgtgtttggaatatctctaaaaacatagaaaacactacagtggtttagaaattactaattttacttctaagtcattcataaaccttgtctatgaaatgacttcttaaatatttagttgatagactgctacaggtaatagggacttagcaagctcttttatatgctaaaggagcatctatcagattaagttagaacatttgctgtcagccacatattgagatgacactaggtgcaatagcagggatagattttgttggtgagtagtctcatgccttgagatctgtggtggtcttcaaaatggtggccagccagatcaaggatgtagtatctcatagttcccaggtgatatttttcttattagaaaaatattataactcatttgttgtttgacacttatagattgaaatttcctaatttattctaaattttaagtggttctttggttccagtgctttatgttgttgttgtttttggatggtgttacatattatatgttctagaaacatgtaatcctaaatttaccctcttgaatataatccctggatgatattttttatcataaatgcagaataatcaaatacattttaagcaagttaagtgtcctccatcaattctgtattccagacttgggaggatgtacagttgctgttgtgtgatcaaacatgtctctgtgtagttccagcaaatcaagctgagctttgaaaaagtttgtcttagttttgtgaaggtgatttattcttaaaaaaaaaaaaaaaaaaaaaa

SEQ ID NO: 55 - DNA, 2445 bp, Homo sapiens
gagcgcgccaggcgcggggagcctaggaccaggagcgagagccgcctacctgcagccgccgcccacggcacggcagccaccatggcgctcctgctgcgcttcgtgctcctgtgcagagtagcggatttcaccagaagttggagtatcactactcctgaacagatgattgaaaaagccaaaggggaaactgcctatctgccatgcaaatttacgcttagtcccgaagaccagggaccactggacatcgagtggctgatatcaccagctgataatcagaaggtggatcaagtgattattttatattctggagacaaaaattatgatgactactatccagatctgaaaggccgagtacattttaagagtaatgatctcaaatctggtgatgcatcaataaatgtaacgaatttacaactgtcagatattggcacagatcagtgcaaagtgaaaaaagctcctggtgttgcaaataagaagattcagctggtagttcttggtaagccttcaggtacaagatgttacgttgatggatcagaagaaattggaagtgactttaaaataaaatgtgaaccaaaagaaggttcacttccattacagtatgagtggcaaaaattgtctgactcacagaaaatgcccacttcatggttagcagaaatgacttcatctgttatatctgtaaaaatgcctcttctgagtactctgggacatacagctgtacatcagaaacagagtgggctctgatcagtgcctgttgcgtctaaacgttgtccctccttcaaataaagctggactaattgcaggagccattataggaactttgcttgctctagcgctcattggtcttatcatcttttgctgtcgtaaaaagcgcagagaagaaaaatatgaaaaggaagttcatcacgatatcaaggaagatgtgccacctccaaagagccacacgtccactgccagaagctacataggcagtaatcattcatccctgggatccatatctccttccaacatggaaggatattccaagactcagtataaacaagtaccaagtgaagactttgaacgcactcctcagagtccgactctcccacctgctaaggtagctgcccctaatctaagtcgaatgggcgcgattcctgtgatgattccagcacagagcaaggatgggtctatagtatagagcctccatacatctcatctgtgctctccgtgttcctttcctttttttgatatatgaaaacctattctggtctaaattgtgttactagcctcaaaatacatcaaaaaataagttaatcaggaactgtaaggaatatatttttaaaaatttttgtttggttatatcgaaatagttacaggcactaaagttagtaaagaaaagtttaccatctgaaaaagctggattttctttaagaggctgattataaaggtttctaaatttatcagtacctaagtaagatgtagcactttgaatatgaaatcataggtgaagaaatcggtgaacttacttgcataccaagttgatacttgaataaccatctgaaagtggtacttgataatttttaccattatttttaggatgtgtatctcatttatttatggcccacaagtctcccccaaattagtacagaaacatccatgacaaaattacttacgtacgtttgtacttggtttcacagctcctcggaaaactctgtgttaggaatatctctaaaaacatagaaaacactacagtgatttagaaattactaattttacttctaagtcattcataaaccttgcctatgaaatgacttcttaaatatttagttgatagactgctacaggtaatagggacttagcaagctcttttatatgctaaaggagcatctatcagattaagttagaacatttgctgtcagccacatattgagatgacactaggtgcaatagcagggatagattttgttggtgagaagtctcatgccttgagatctgtggtggtcttcaaaatggtggccagccagaccaaggatgtagtatctcatagtccccaggtgatatttttctaattagaaaaatattataactcacttattgtttgacaattatagattgaaatttcctaattctaaattttaagtggctcttcggttccagtgctctatgttgttgttggttttggatggcattacatattatatgttctagaaacatgtaatcctaaatttaccctcttgaatataatccctggatgatatttttatcataaatgcagaataatcaaatacattttaagcaagtaagtgtcctccatcaattccgtattccagacttgggaggatgtacagttgctgttgtgtgatcaaacatgtctctgtgtagttccagcaaatcaagctgagcttcaaaaaagtttgtctcagttttgtgaaagtgatttattcttaaaaaaaaaaaaaaaaaaaa

SEQ ID NO: 56 - DNA, 286 bp, Homo sapiens
ttagaacatttgctgtctgccacatattgagatggcactaggtgcaatagcagggatagattttgttggtgagaggtctcatgccttgagatctgtggtggtctttaaaatggtggccagccagaccaaggatgtagtatctcatagtccccaactaaatgctggctttccactttaggtgatatttttctaattagaaaaatattataactcacttattgtttgacaattatagattgaaatttcctaattctaaattttaagtggctcttcggtttcagtgctc

SEQ ID NO: 57 - DNA, 285 bp, Homo sapiens
  misc_feature (202)..(202): n is a, c, g, or t
ttagaacatttgctgtctgccacatattgagatggcactaggtgcaatagcagggatagattttgttggtgagaggtctcatgccttgagatctgtggtggtctttaaaatggtggccagccagaccaaggatgtagtatctcatagtccccaactaaatgctggctttccactttaggtgatatttttctaattagaaaantattataactcacttattgtttgacaattatagattgaaatttcctaattctaaattttaagtggctcttcggtttcaggctc

SEQ ID NO: 58 - DNA, 265 bp, Homo sapiens
ttagaacatttgctgtcagccacatattgagatgacactaggtgcaatagcagggatagattttgttggtgagtagtctcatgccttgagatctgtggtggtcttcaaaatggtggccagccagatcaaggatgtagtatctcatagttcccaggtgatatttttcttattagaaaaatattataactcatttgttgtttgacacttatagattgaaatttcctaatttattctaaattttaagtggttctttggttccagtgct

SEQ ID NO: 59 - DNA, 284 bp, Homo sapiens
  misc_feature (202)..(202): n is a, c, g, or t
ttagaacatttgctgtctgccacatattgagatggcactaggtgcaatagcagggatagattttgttggtgagaggtctcatgccttgagatctgtggtggtctttaaaatggtggccagccagaccaaggatgtagtatctcatagtccccaactaaatgctggctttccactttaggtgatatttttctaattagaaaantattataactcacttattgtttgacaattatagattgaaatttcctaattctaaattttaagtggctcttcggtttcaggct

SEQ ID NO: 60 - protein, 164 aa, Homo sapiens
MENELFCSGVLVHPQWVLSAAHCFQNSYTIGLGLHSLEADQEPGSQMVEASLSVRHPEYNRPLLANDLMLIKLDESVSESDTIRSISIASQCPTAGNSCLVSGWGLLANDAVIAIQSQTVGGWECEKLSQPWQGCTISATSSARTSCCILTGCSLLLTASPGTL